(19) United States
(12) Patent Application Publication
(10) Pub. No.: US 2011/0190269 A1
(43) Pub. Date: Aug. 4, 2011

(54) GAMMA SECRETASE MODULATORS

(76) Inventors: Karlheinz Baumann, Efringen-Kirchen (DE); Erwin Goetschi, Reinach (CH); Luke Green, Basel (CH); Synese Jolidon, Blauen (CH); Henner Knust, Rheinfelden (DE); Anja Limberg, Basel (CH); Thomas Luebbers, Loerrach (DE); Andrew Thomas, Binningen (CH)

(21) Appl. No.: 13/011,951

(22) Filed: Jan. 24, 2011

(30) Foreign Application Priority Data
Feb. 1, 2010 (EP) 10152211.8

(51) Int. Cl. A61K 31/55; C07D 471/04; C07D 487/04; A61K 31/437; A61K 31/519; A61K 31/4985; A61K 31/5377; A61K 31/5025; A61K 31/538

(52) U.S. Cl. 514/217.07; 546/119; 544/263; 544/350; 544/118; 544/127; 540/597; 544/236; 514/303; 514/259.31; 514/249; 514/233.2; 514/248; 514/230.5

(57) ABSTRACT

The invention relates to compounds of formula

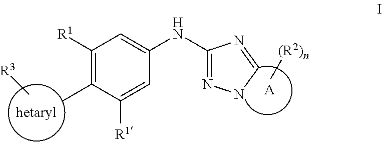

wherein R¹, R^{1′}, R², R³, n, A, and hetaryl are defined herein or to pharmaceutically active acid addition salts thereof. The present compounds of formula I are modulators for amyloid beta and thus, they may be useful for the treatment or prevention of a disease associated with the deposition of β-amyloid in the brain, in particular Alzheimer's disease, and other diseases such as cerebral amyloid angiopathy, hereditary cerebral hemorrhage with amyloidosis, Dutch-type (HCHWA-D), multi-infarct dementia, dementia pugilistica and Down syndrome.

### PRIORITY TO RELATED APPLICATION(S)

This application claims the benefit of European Patent Application No. 10152211.8, filed Feb. 1, 2010, which is hereby incorporated by reference in its entirety.

### BACKGROUND OF THE INVENTION

Alzheimer's disease (AD) is the most common cause of dementia in later life. Pathologically, AD is characterized by the deposition of amyloid in extracellular plaques and intracellular neurofibrillary tangles in the brain. The amyloid plaques are mainly composed of amyloid peptides (Aβ peptides) which originate from the β-Amyloid Precursor Protein (APP) by a series of proteolytic cleavage steps. Several forms of APP have been identified of which the most abundant are proteins of 695, 751 and 770 amino acids length. They all arise from a single gene through differential splicing. The Aβ peptides are derived from the same domain of the APP.

Aβ peptides are produced from APP through the sequential action of two proteolytic enzymes termed β- and γ-secretase. β-Secretase cleaves first in the extracellular domain of APP just outside of the trans-membrane domain (TM) to produce a C-terminal fragment of APP containing the TM- and cytoplasmatic domain (CTFβ). CTFβ is the substrate for γ-secretase which cleaves at several adjacent positions within the TM to produce the Aβ peptides and the cytoplasmic fragment. Various proteolytic cleavages mediated by γ-secretase result in Aβ peptides of different chain length, e.g. Aβ38, Aβ40 and Aβ42. The latter one is regarded to be the more pathogenic amyloid peptide because of its strong tendency to form neurotoxic aggregates.

The β-secretase is a typical aspartyl protease. The γ-secretase is a proteolytic activity consisting of several proteins, its exact composition is incompletely understood. However, the presenilins are essential components of this activity and may represent a new group of atypical aspartyl proteases which cleave within the TM of their substrates and which are themselves polytopic membrane proteins. Other essential components of γ-secretase may be nicastrin and the products of the aph1 and pen-2 genes. Proven substrates for γ-secretase are the APP and the proteins of the Notch receptor family, however, γ-secretase has loose substrate specificity and may cleave further membrane proteins unrelated to APP and Notch.

The γ-secretase activity is absolutely required for the production of Aβ peptides. This has been shown both by genetic means, i.e., ablation of the presenilin genes and by low-molecular-weight inhibitory compounds. Since according to the amyloid hypothesis for AD the production and deposition of Aβ is the ultimate cause for the disease, it is thought that selective and potent inhibitors of γ-secretase will be useful for the prevention and treatment of AD.

An alternative mode of treatment is the modulation of the γ-secretase activity which results in a selective reduction of the Aβ42 production. This will result in an increase of shorter Aβ isoforms, such as Aβ38, Aβ37 or others, which have reduced capability for aggregation and plaque formation, and hence less neurotoxic. Compounds which show this effect on modulating γ-secretase activity include certain non-steroidal anti-inflammatory drugs (NSAIDs) and related analogues (Weggen et al. Nature, 414 (2001) 212-16).

Numerous documents describe the current knowledge on γ-secretase modulation, for example the following publications:
Morihara et al, J. Neurochem., 83 (2002) 1009-12
Jantzen et al, J. Neuroscience, 22 (2002) 226-54
Takahashi et al, J. Biol. Chem., 278 (2003) 18644-70
Beher et al, J. Biol. Chem. 279 (2004) 43419-26
Lleo et al, Nature Med. 10 (2004) 1065-6
Kukar et al, Nature Med. 11 (2005) 545-50
Perretto et al, J. Med. Chem. 48 (2005) 5705-20
Clarke et al, J. Biol. Chem. 281 (2006) 31279-89
Stock et al, Bioorg. Med. Chem. Lett. 16 (2006) 2219-2223
Narlawar et al, J. Med. Chem. 49 (2006) 7588-91

### SUMMARY OF THE INVENTION

The invention provides compounds of formula I

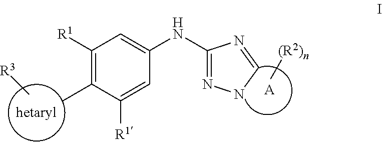

wherein
R¹ and R^{1′} are each independently hydrogen, halogen, lower alkoxy or cyano;
R² is lower alkyl, halogen, lower alkoxy, lower alkyl substituted by halogen, lower alkoxy substituted by halogen, lower alkyl substituted by OR, ═O, —C(O)O-lower alkyl, —C(O)NH-lower alkyl, cyano, CH₂—O-lower alkyl, cycloalkyl, NRR′ or is —O—(CH₂)ₒ-phenyl optionally substituted by halogen,
or is —(CH₂)ₒ-phenyl optionally substituted by one, two or three substituents, selected from halogen, —(CH₂)_{O}-cyano, lower alkyl, lower alkyl substituted by halogen, lower alkyl substituted by hydroxy, C(O)H, —CH₂—NH₂—, —CH₂—NH—C(O)O-lower alkyl,
—CH₂—NH—C(O)-lower alkyl, —CH₂—NH-lower alkyl, —CH₂—NH—S(O)₂-lower alkyl,
lower alkoxy and lower alkoxy substituted by halogen,
or is —(CH₂)ₒ-cycloalkyl,
or is —(CH₂)ₒ-heterocycloalkyl which is optionally substituted by halogen, CF₃, lower alkyl, —CH₂CN, —C(O)-lower alkyl, —C(O)O-lower alkyl or S(O)₂-lower alkyl,
or is heteroaryl selected from the group consisting of furanyl, pyrazinyl, pyridinyl, benzooxazolyl and benzoimidazolyl each of which is optionally substituted by lower alkyl,
or is 4-methyl-3,4-dihydro-2H-benzo[1,4]oxazine;
R and R′ are each independently from each other hydrogen or lower alkyl, and
o is 0 or 1;
R³ occurs once or twice and is lower alkyl;

### A is

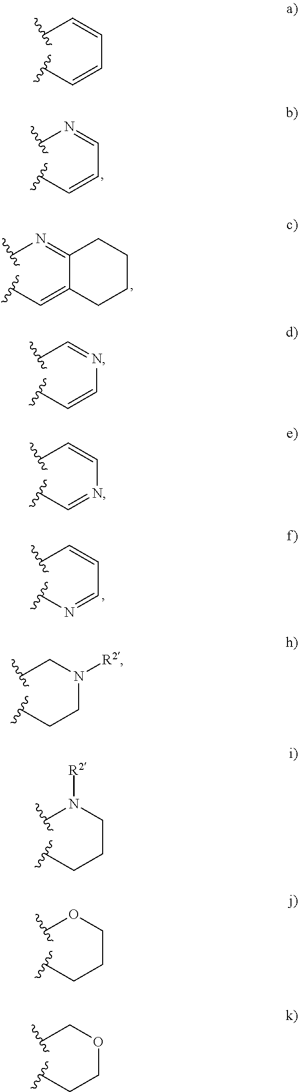

and
R^{2′} is hydrogen, lower alkyl, lower alkyl substituted by halogen, C(O)-lower alkyl, S(O)₂-lower alkyl or phenyl optionally substituted by halogen;
hetaryl is a 5 or 6 membered N, S or O-containing heteroaryl group; and
n is 0, 1, 2 or 3; when n is 2 or 3, R² is same or different;
or to pharmaceutically active acid addition salts thereof.

Further the invention provides are all forms of optically pure enantiomers, racemates or diastereomeric mixtures for compounds of formula I.

Compounds of formula I are modulators for amyloid beta and thus, and may be useful for the treatment or prevention of a disease associated with the deposition of β-amyloid in the brain, in particular Alzheimer's disease, and other diseases such as cerebral amyloid angiopathy, hereditary cerebral hemorrhage with amyloidosis, Dutch-type (HCHWA-D), multi-infarct dementia, dementia pugilistica and Down syndrome.

The present invention provides compounds of formula I per se, pharmaceutically acceptable salts of formula I, and pharmaceutical compositions containing such compounds. The invention further provides methods for the manufacture of the compounds and compositions of the invention. The invention further provides methods for the treatment of Alzheimer's disease, cerebral amyloid angiopathy, hereditary cerebral hemorrhage with amyloidosis, Dutch-type (HCHWA-D), multi-infarct dementia, dementia pugilistica or Down syndrome, their manufacture and medicaments based on a compound of formula I in accordance with the invention.

Thus, the compounds of this invention will be useful for the treatment or prevention of a disease associated with the deposition of β-amyloid in the brain, in particular Alzheimer's disease, and other diseases such as cerebral amyloid angiopathy, hereditary cerebral hemorrhage with amyloidosis, Dutch-type (HCHWA-D), multi-infarct dementia, dementia pugilistica and Down syndrome.

### DETAILED DESCRIPTION OF THE INVENTION

The following definitions of general terms used herein apply irrespective of whether the terms in question appear alone or in combination. It must be noted that, as used in the specification and the appended claims, the singular forms “a”, “an,” and “the” include plural forms unless the context clearly dictates otherwise.

As used herein, the term “lower alkyl” denotes a saturated straight- or branched-chain group containing from 1 to 7 carbon atoms, for example, methyl, ethyl, propyl, isopropyl, n-butyl, i-butyl, 2-butyl, t-butyl and the like. Preferred alkyl groups are groups with 1-4 carbon atoms.

As used herein, the term “lower alkoxy” denotes a group containing a lower alkyl residue is as defined above that is attached to the molecule via an oxygen atom.

As used herein, the term “lower alkyl substituted by halogen” denotes a lower alkyl group as defined above, wherein at least one hydrogen atom is replaced by halogen, for example CF₃, CHF₂, CH₂F, CH₂CF₃, CH₂CH₂CF₃, CF₂CHF₂, CH₂CF₂CF₃ and the like.

As used herein, the term “lower alkoxy substituted by halogen” denotes a lower alkoxy group as defined above, wherein at least one hydrogen atom is replaced by halogen, for example OCF₃, OCHF₂, OCH₂F, OCH₂CF₃, OCH₂CH₂CF₃, OCF₂CHF₂, OCH₂CF₂CF₃ and the like.

The term “halogen” denotes chlorine, iodine, fluorine and bromine.

The term “cycloalkyl” denotes a saturated carbocyclic ring with 3-7 carbon atoms.

The term “a 5 or 6 membered N, S or O-containing heteroaryl group,” is a group selected from the group consisting of

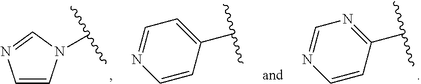

The term “heterocycloalkyl” denotes saturated ring, containing one or more heteroatoms, selected from N, O or S, for example pyrrolidinyl, morpholinyl, azepanyl, 1,2,3,6-tetrahydro-pyridine, 3,6-dihydro-2H-pyran or piperidinyl.

“Pharmaceutically acceptable,” such as pharmaceutically acceptable carrier, excipient, etc., means pharmacologically acceptable and substantially non-toxic to the subject to which the particular compound is administered.

The term “pharmaceutically acceptable acid addition salts” embraces salts with inorganic and organic acids, such as hydrochloric acid, nitric acid, sulfuric acid, phosphoric acid, citric acid, formic acid, fumaric acid, maleic acid, acetic acid, succinic acid, tartaric acid, methane-sulfonic acid, p-toluenesulfonic acid and the like.

“Therapeutically effective amount” means an amount that is effective to prevent, alleviate or ameliorate symptoms of disease or prolong the survival of the subject being treated.

One embodiment of the invention provides compounds of formula I, wherein hetaryl is imidazolyl, pyrimidinyl or pyridinyl.

An embodiment of the invention provides compounds of formula I, wherein A is the ring a), for example the compounds
5-(8-methoxy-5-phenyl-[1,2,4]triazolo[1,5-a]pyridin-2-ylamino)-2-(4-methyl-imidazol-1-yl)-benzonitrile;
[3-methoxy-4-(4-methyl-imidazol-1-yl)-phenyl]-(5-phenyl-[1,2,4]triazolo[1,5-a]pyridin-2-yl)-amine;
[5-(4-fluoro-phenyl)-[1,2,4]triazolo[1,5-a]pyridin-2-yl]-[3-methoxy-4-(4-methyl-imidazol-1-yl)-phenyl]-amine;
[8-(4-fluoro-phenyl)-[1,2,4]triazolo[1,5-a]pyridin-2-yl]-[3-methoxy-4-(4-methyl-imidazol-1-yl)-phenyl]-amine;
[3-methoxy-4-(4-methyl-imidazol-1-yl)-phenyl]-(5-pyrrolidin-1-yl-[1,2,4]triazolo[1,5-a]pyridin-2-yl)-amine;
[3-methoxy-4-(4-methyl-imidazol-1-yl)-phenyl]-(5-propyl-[1,2,4]triazolo[1,5-a]pyridin-2-yl)-amine;
[3-methoxy-4-(4-methyl-imidazol-1-yl)-phenyl]-(5-trifluoromethyl-[1,2,4]triazolo[1,5-a]pyridin-2-yl)-amine;
[5-(4-fluoro-phenyl)-8-methoxy-[1,2,4]triazolo[1,5-a]pyridin-2-yl]-[3-methoxy-4-(4-methyl-imidazol-1-yl)-phenyl]-amine;
[3-methoxy-4-(4-methyl-imidazol-1-yl)-phenyl]-(5-morpholin-4-yl-[1,2,4]triazolo[1,5-a]pyridin-2-yl)-amine;
[8-(4-fluoro-phenyl)-6-methyl-[1,2,4]triazolo[1,5-a]pyridin-2-yl]-[3-methoxy-4-(4-methyl-imidazol-1-yl)-phenyl]-amine;
(5,6-dimethyl-[1,2,4]triazolo[1,5-a]pyridin-2-yl)-[3-methoxy-4-(4-methyl-imidazol-1-yl)-phenyl]-amine;
8-(4-fluorophenyl)-N-(3-methoxy-4-(2-methylpyridin-4-yl)phenyl)-[1,2,4]triazolo[1,5-a]pyridin-2-amine;
5-(4-fluorophenyl)-N-(3-methoxy-4-(2-methylpyridin-4-yl)phenyl)-[1,2,4]triazolo[1,5-a]pyridin-2-amine;
[8-(3-chloro-4-fluoro-phenyl)-6-methyl-[1,2,4]triazolo[1,5-a]pyridin-2-yl]-[3-methoxy-4-(4-methyl-imidazol-1-yl)-phenyl]-amine;
[8-(3,4-difluoro-phenyl)-6-methyl-[1,2,4]triazolo[1,5-a]pyridin-2-yl]-[3-methoxy-4-(4-methyl-imidazol-1-yl)-phenyl]-amine;
[8-(4-fluoro-2-methoxy-phenyl)-6-methyl-[1,2,4]triazolo[1,5-a]pyridin-2-yl]-[3-methoxy-4-(4-methyl-imidazol-1-yl)-phenyl]-amine;
[8-(2-chloro-4-fluoro-phenyl)-6-methyl-[1,2,4]triazolo[1,5-a]pyridin-2-yl]-[3-methoxy-4-(4-methyl-imidazol-1-yl)-phenyl]-amine;
[8-(4-fluoro-2-methyl-phenyl)-6-methyl-[1,2,4]triazolo[1,5-a]pyridin-2-yl]-[3-methoxy-4-(4-methyl-imidazol-1-yl)-phenyl]-amine;
8-(2,4-difluorophenyl)-N-(3-methoxy-4-(4-methyl-1H-imidazol-1-yl)phenyl)-6-methyl-[1,2,4]triazolo[1,5-a]pyridin-2-amine;
8-(4-fluoro-3-(trifluoromethyl)phenyl)-N-(3-methoxy-4-(4-methyl-1H-imidazol-1-yl)phenyl)-6-methyl-[1,2,4]triazolo[1,5-a]pyridin-2-amine;
8-(4-fluoro-3-methylphenyl)-N-(3-methoxy-4-(4-methyl-1H-imidazol-1-yl)phenyl)-6-methyl-[1,2,4]triazolo[1,5-a]pyridin-2-amine;
2-fluoro-5-(2-(3-methoxy-4-(4-methyl-1H-imidazol-1-yl)phenylamino)-6-methyl-[1,2,4]triazolo[1,5-a]pyridin-8-yl)benzonitrile;
8-(3,4-difluorophenyl)-N-(3-methoxy-4-(4-methyl-1H-imidazol-1-yl)phenyl)-6-(trifluoromethyl)-[1,2,4]triazolo[1,5-a]pyridin-2-amine;
6-chloro-8-(3,4-difluorophenyl)-N-(3-methoxy-4-(4-methyl-1H-imidazol-1-yl)phenyl)-[1,2,4]triazolo[1,5-a]pyridin-2-amine;
N-(3-methoxy-4-(4-methyl-1H-imidazol-1-yl)phenyl)-6-methyl-8-(4-morpholinophenyl)-[1,2,4]triazolo[1,5-a]pyridin-2-amine;
2-(4-(2-(3-methoxy-4-(4-methyl-1H-imidazol-1-yl)phenylamino)-6-methyl-[1,2,4]triazolo[1,5-a]pyridin-8-yl)phenyl)acetonitrile;
8-(2,4-dimethoxyphenyl)-N-(3-methoxy-4-(4-methyl-1H-imidazol-1-yl)phenyl)-6-methyl-[1,2,4]triazolo[1,5-a]pyridin-2-amine;
8-(3,4-difluorophenyl)-6-fluoro-N-(3-methoxy-4-(4-methyl-1H-imidazol-1-yl)phenyl)-[1,2,4]triazolo[1,5-a]pyridin-2-amine;
8-(2-chloro-4-fluorophenyl)-6-fluoro-N-(3-methoxy-4-(4-methyl-1H-imidazol-1-yl)phenyl)-[1,2,4]triazolo[1,5-a]pyridin-2-amine;
N-(3-methoxy-4-(4-methyl-1H-imidazol-1-yl)phenyl)-6-methyl-8-(2-methylbenzo[d]oxazol-6-yl)-[1,2,4]triazolo[1,5-a]pyridin-2-amine;
N-(3-methoxy-4-(4-methyl-1H-imidazol-1-yl)phenyl)-6-methyl-8-(1-methyl-1H-benzo[d]imidazol-6-yl)-[1,2,4]triazolo[1,5-a]pyridin-2-amine;
N-(3-methoxy-4-(4-methyl-1H-imidazol-1-yl)phenyl)-6-methyl-8-(4-methyl-3,4-dihydro-2H-benzo[b][1,4]oxazin-7-yl)-[1,2,4]triazolo[1,5-a]pyridin-2-amine;
2-(2-fluoro-4-(2-(3-methoxy-4-(4-methyl-1H-imidazol-1-yl)phenylamino)-6-methyl-[1,2,4]triazolo[1,5-a]pyridin-8-yl)phenyl)propan-2-ol;
5-chloro-2-(2-(3-methoxy-4-(4-methyl-1H-imidazol-1-yl)phenylamino)-6-methyl-[1,2,4]triazolo[1,5-a]pyridin-8-yl)benzaldehyde;
(5-chloro-2-(2-(3-methoxy-4-(4-methyl-1H-imidazol-1-yl)phenylamino)-6-methyl-[1,2,4]triazolo[1,5-a]pyridin-8-yl)phenyl)methanol;
tert-butyl 3-(2-(3-methoxy-4-(4-methyl-1H-imidazol-1-yl)phenylamino)-6-methyl-[1,2,4]triazolo[1,5-a]pyridin-8-yl)benzylcarbamate;
tert-butyl 4-(2-(3-methoxy-4-(4-methyl-1H-imidazol-1-yl)phenylamino)-6-methyl-[1,2,4]triazolo[1,5-a]pyridin-8-yl)-5,6-dihydropyridine-1(2H)-carboxylate;
8-(3-(aminomethyl)phenyl)-N-(3-methoxy-4-(4-methyl-1H-imidazol-1-yl)phenyl)-6-methyl-[1,2,4]triazolo[1,5-a]pyridin-2-amine dihydrochloride;
N-(3-(2-(3-methoxy-4-(4-methyl-1H-imidazol-1-yl)phenylamino)-6-methyl-[1,2,4]triazolo[1,5-a]pyridin-8-yl)benzyl)methanesulfonamide;
N-(3-(2-(3-methoxy-4-(4-methyl-1H-imidazol-1-yl)phenylamino)-6-methyl-[1,2,4]triazolo[1,5-a]pyridin-8-yl)benzyl)acetamide;
N-(3-methoxy-4-(4-methyl-1H-imidazol-1-yl)phenyl)-6-methyl-8-(1-(methylsulfonyl)piperidin-4-yl)-[1,2,4]triazolo[1,5-a]pyridin-2-amine;
N-(3-methoxy-4-(4-methyl-1H-imidazol-1-yl)phenyl)-8-morpholino-[1,2,4]triazolo[1,5-a]pyridin-2-amine;
ethyl 4-(2-(3-methoxy-4-(4-methyl-1H-imidazol-1-yl)phenylamino)-6-methyl-[1,2,4]triazolo[1,5-a]pyridin-8-yl)-5,6-dihydropyridine-1(2H)-carboxylate;
2-(4-(2-(3-methoxy-4-(4-methyl-1H-imidazol-1-yl)phenylamino)-6-methyl-[1,2,4]triazolo[1,5-a]pyridin-8-yl)-5,6-dihydropyridin-1(2H)-yl)acetonitrile;
8-(2-chloro-4-fluorophenyl)-N-(3-fluoro-4-(4-methyl-1H-imidazol-1-yl)phenyl)-[1,2,4]triazolo[1,5-a]pyridin-2-amine;
8-(2-chloro-4-fluorophenyl)-N-(3,5-difluoro-4-(4-methyl-1H-imidazol-1-yl)phenyl)-[1,2,4]triazolo[1,5-a]pyridin-2-amine;
8-(2-chloro-4-fluorophenyl)-N-(3-fluoro-5-methoxy-4-(4-methyl-1H-imidazol-1-yl)phenyl)-[1,2,4]triazolo[1,5-a]pyridin-2-amine;
5-(8-(2-chloro-4-fluorophenyl)-[1,2,4]triazolo[1,5-a]pyridin-2-ylamino)-2-(4-methyl-1H-imidazol-1-yl)benzonitrile;
8-(2-chloro-4-fluorophenyl)-N-(3-methoxy-4-(6-methylpyrimidin-4-yl)phenyl)-[1,2,4]triazolo[1,5-a]pyridin-2-amine;
8-(2-chloro-4-fluorophenyl)-N-(4-(2,6-dimethylpyrimidin-4-yl)phenyl)-[1,2,4]triazolo[1,5-a]pyridin-2-amine;
2-{8-(4-chloro-phenyl)-2-[3-methoxy-4-(4-methyl-imidazol-1-yl)-phenylamino]-[1,2,4]triazolo[1,5-a]pyridin-5-yl}-propan-2-ol;
[8-(4-chloro-phenyl)-6-cyclopropyl-[1,2,4]triazolo[1,5-a]pyridin-2-yl]-[3-methoxy-4-(4-methyl-imidazol-1-yl)-phenyl]-amine;
[6-cyclopropyl-8-(4-fluoro-phenyl)-[1,2,4]triazolo[1,5-a]pyridin-2-yl]-[3-methoxy-4-(4-methyl-imidazol-1-yl)-phenyl]-amine;
2-{8-(4-fluoro-phenyl)-2-[3-methoxy-4-(4-methyl-imidazol-1-yl)-phenylamino]-[1,2,4]triazolo[1,5-a]pyridin-5-yl}-propan-2-ol;
[6-cyclopropyl-8-(2,3,4-trifluoro-phenyl)-[1,2,4]triazolo[1,5-a]pyridin-2-yl]-[3-methoxy-4-(4-methyl-imidazol-1-yl)-phenyl]-amine;
2-{8-(2-chloro-4-fluoro-phenyl)-2-[3-methoxy-4-(4-methyl-imidazol-1-yl)-phenylamino]-[1,2,4]triazolo[1,5-a]pyridin-5-yl}-propan-2-ol;
2-[2-[3-methoxy-4-(4-methyl-imidazol-1-yl)-phenylamino]-8-(2,3,4-trifluoro-phenyl)-[1,2,4]triazolo[1,5-a]pyridin-5-yl]-propan-2-ol;
2-{8-(3-chloro-4-fluoro-phenyl)-2-[3-methoxy-4-(4-methyl-imidazol-1-yl)-phenylamino]-[1,2,4]triazolo[1,5-a]pyridin-5-yl}-propan-2-ol;
[8-(4-chloro-phenyl)-[1,2,4]triazolo[1,5-a]pyridin-2-yl]-[3-methoxy-4-(4-methyl-imidazol-1-yl)-phenyl]-amine;
[8-(3,4-difluoro-phenyl)-[1,2,4]triazolo[1,5-a]pyridin-2-yl]-[3-methoxy-4-(4-methyl-imidazol-1-yl)-phenyl]-amine;
[8-(4-chloro-phenyl)-[1,2,4]triazolo[1,5-a]pyridin-2-yl]-[3-methoxy-4-(2-methyl-pyridin-4-yl)-phenyl]-amine;
8-(3,4-difluorophenyl)-N-(3-methoxy-4-(2-methylpyridin-4-yl)phenyl)-[1,2,4]triazolo[1,5-a]pyridin-2-amine;
8-(3-chloro-4-fluorophenyl)-N-(3-methoxy-4-(4-methyl-1H-imidazol-1-yl)phenyl)-[1,2,4]triazolo[1,5-a]pyridin-2-amine;
8-(2-chloro-4-fluorophenyl)-N-(3-methoxy-4-(4-methyl-1H-imidazol-1-yl)phenyl)-[1,2,4]triazolo[1,5-a]pyridin-2-amine;
8-(2,4-difluorophenyl)-N-(3-methoxy-4-(4-methyl-1H-imidazol-1-yl)phenyl)-[1,2,4]triazolo[1,5-a]pyridin-2-amine;
[8-(2-chloro-4-fluoro-phenyl)-[1,2,4]triazolo[1,5-a]pyridin-2-yl]-[3-methoxy-4-(2-methyl-pyridin-4-yl)-phenyl]-amine;
8-(3,4-difluorophenyl)-N-(3-methoxy-4-(4-methyl-1H-imidazol-1-yl)phenyl)-5-(trifluoromethyl)-[1,2,4]triazolo[1,5-a]pyridin-2-amine;
N-(3-methoxy-4-(4-methyl-1H-imidazol-1-yl)phenyl)-8-phenoxy-[1,2,4]triazolo[1,5-a]pyridin-2-amine;
8-(3-chlorophenoxy)-N-(3-methoxy-4-(4-methyl-1H-imidazol-1-yl)phenyl)-[1,2,4]triazolo[1,5-a]pyridin-2-amine;
8-(4-chlorophenoxy)-N-(3-methoxy-4-(4-methyl-1H-imidazol-1-yl)phenyl)-[1,2,4]triazolo[1,5-a]pyridin-2-amine;
8-(4-fluoropiperidin-1-yl)-N-(3-methoxy-4-(4-methyl-1H-imidazol-1-yl)phenyl)-[1,2,4]triazolo[1,5-a]pyridin-2-amine;
N-(3-methoxy-4-(4-methyl-1H-imidazol-1-yl)phenyl)-8-(4-(trifluoromethyl)piperidin-1-yl)-[1,2,4]triazolo[1,5-a]pyridin-2-amine;
8-(4,4-difluoropiperidin-1-yl)-N-(3-methoxy-4-(4-methyl-1H-imidazol-1-yl)phenyl)-[1,2,4]triazolo[1,5-a]pyridin-2-amine;
2-{8-(4-chloro-phenyl)-2-[3-methoxy-4-(4-methyl-imidazol-1-yl)-phenylamino]-6-methyl-[1,2,4]triazolo[1,5-a]pyridin-5-yl}-propan-2-ol;
[8-(3,6-dihydro-2H-pyran-4-yl)-[1,2,4]triazolo[1,5-a]pyridin-2-yl]-[3-methoxy-4-(4-methyl-imidazol-1-yl)-phenyl]-amine;
2-{8-(3,4-difluoro-phenyl)-2-[3-methoxy-4-(4-methyl-imidazol-1-yl)-phenylamino]-[1,2,4]triazolo[1,5-a]pyridin-5-yl}-propan-2-ol;
[6-cyclopropyl-8-(3,4-difluoro-phenyl)-[1,2,4]triazolo[1,5-a]pyridin-2-yl]-[3-methoxy-4-(4-methyl-imidazol-1-yl)-phenyl]-amine;
[8-(3-chloro-4-fluoro-phenyl)-6-cyclopropyl-[1,2,4]triazolo[1,5-a]pyridin-2-yl]-[3-methoxy-4-(4-methyl-imidazol-1-yl)-phenyl]-amine; and
[8-(2-chloro-4-fluoro-phenyl)-6-cyclopropyl-[1,2,4]triazolo[1,5-a]pyridin-2-yl]-[3-methoxy-4-(4-methyl-imidazol-1-yl)-phenyl]-amine.

A further embodiment of the invention provides compounds of formula I, wherein A is the ring b), for example the compounds
[3-methoxy-4-(4-methyl-imidazol-1-yl)-phenyl]-(5-methyl-7-phenyl-[1,2,4]triazolo[1,5-a]pyrimidin-2-yl)-amine;
[7-(4-chloro-phenyl)-5-trifluoromethyl-[1,2,4]triazolo[1,5-a]pyrimidin-2-yl]-[3-methoxy-4-(4-methyl-imidazol-1-yl)-phenyl]-amine;
[3-methoxy-4-(4-methyl-imidazol-1-yl)-phenyl]-[7-(4-trifluoromethoxy-phenyl)-[1,2,4]triazolo[1,5-a]pyrimidin-2-yl]-amine;
(5,7-bis-trifluoromethyl-[1,2,4]triazolo[1,5-a]pyrimidin-2-yl)-[3-methoxy-4-(4-methyl-imidazol-1-yl)-phenyl]-amine;
2-[3-methoxy-4-(4-methyl-imidazol-1-yl)-phenylamino]-7-phenyl-[1,2,4]triazolo[1,5-a]pyrimidine-6-carbonitrile;
7-(4-chloro-phenyl)-2-[3-methoxy-4-(4-methyl-imidazol-1-yl)-phenylamino]-[1,2,4]triazolo[1,5-a]pyrimidine-6-carbonitrile;
2-[3-methoxy-4-(4-methyl-imidazol-1-yl)-phenylamino]-7-m-tolyl-[1,2,4]triazolo[1,5-a]pyrimidine-6-carbonitrile;
2-(3-methoxy-4-(4-methyl-1H-imidazol-1-yl)phenylamino)-7-o-tolyl-[1,2,4]triazolo[1,5-a]pyrimidine-6-carbonitrile;
7-(2-fluorophenyl)-2-(3-methoxy-4-(4-methyl-1H-imidazol-1-yl)phenylamino)-[1,2,4]triazolo[1,5-a]pyrimidine-6-carbonitrile;
[7-(4-fluoro-phenyl)-[1,2,4]triazolo[1,5-a]pyrimidin-2-yl]-[3-methoxy-4-(4-methyl-imidazol-1-yl)-phenyl]-amine;
7-(3-chloro-4-fluorophenyl)-N-(3-methoxy-4-(4-methyl-1H-imidazol-1-yl)phenyl)-[1,2,4]triazolo[1,5-a]pyrimidin-2-amine hydrochloride;
7-(3-chloro-4-fluorophenyl)-N-(3-methoxy-4-(4-methyl-1H-imidazol-1-yl)phenyl)-5-methyl-[1,2,4]triazolo[1,5-a]pyrimidin-2-amine; and
ethyl 7-(3-chloro-4-fluorophenyl)-2-(3-methoxy-4-(4-methyl-1H-imidazol-1-yl)phenylamino)-[1,2,4]triazolo[1,5-a]pyrimidine-5-carboxylate.

A further embodiment of the invention provides compounds of formula I, wherein A is the ring c), for example the compound
[3-methoxy-4-(4-methyl-imidazol-1-yl)-phenyl]-(9-phenyl-5,6,7,8-tetrahydro-[1,2,4]triazolo[5,1-b]quinazolin-2-yl)-amine.

A further embodiment of the invention provides compounds of formula I, wherein A is the ring d), for example the compounds
[5-(4-fluoro-phenyl)-[1,2,4]triazolo[1,5-a]pyrazin-2-yl]-[3-methoxy-4-(4-methyl-imidazol-1-yl)-phenyl]-amine and
[3-methoxy-4-(4-methyl-imidazol-1-yl)-phenyl]-(5-phenyl-[1,2,4]triazolo[1,5-a]pyrazin-2-yl)-amine.

A further embodiment of the invention provides compounds of formula I, wherein A is the ring e), for example the compound
[3-methoxy-4-(4-methyl-imidazol-1-yl)-phenyl]-[8-methyl-5-(2,2,2-trifluoro-ethoxy)-[1,2,4]triazolo[1,5-c]pyrimidin-2-yl]-amine.

A further embodiment of the invention provides compounds of formula I, wherein A is the ring f), for example the compound
7-(3-chloro-4-fluorophenyl)-N-(3-methoxy-4-(4-methyl-1H-imidazol-1-yl)phenyl)-5-(trifluoromethyl)-[1,2,4]triazolo[1,5-a]pyrimidin-2-amine acetate.

A further embodiment of the invention provides compounds of formula I, wherein A is the ring h), for example the compounds
5-(4-fluorophenyl)-N-(3-methoxy-4-(4-methyl-1H-imidazol-1-yl)phenyl)-7-(methylsulfonyl)-5,6,7,8-tetrahydro-[1,2,4]triazolo[1,5-a]pyrazin-2-amine;
5-(4-fluorophenyl)-N-(3-methoxy-4-(4-methyl-1H-imidazol-1-yl)phenyl)-7-methyl-5,6,7,8-tetrahydro-[1,2,4]triazolo[1,5-a]pyrazin-2-amine;
8-(4-fluorophenyl)-N-(3-methoxy-4-(4-methyl-1H-imidazol-1-yl)phenyl)-7-methyl-5,6,7,8-tetrahydro-[1,2,4]triazolo[1,5-a]pyrazin-2-amine; and
1-(5-(4-fluorophenyl)-2-(3-methoxy-4-(4-methyl-1H-imidazol-1-yl)phenylamino)-5,6-dihydro-[1,2,4]triazolo[1,5-a]pyrazin-7(8H)-yl)-2-methylpropan-1-one.

A further embodiment of the invention provides compounds of formula I, wherein A is the ring i), for example the compounds
7-(4-chlorophenyl)-2-(3-methoxy-4-(4-methyl-1H-imidazol-1-yl)phenylamino)-4-propyl-6,7-dihydro-[1,2,4]triazolo[1,5-a]pyrimidin-5(4H)-one and

4-(3,4-difluorophenyl)-N-(3-methoxy-4-(4-methyl-1H-imidazol-1-yl)phenyl)-4,5,6,7-tetrahydro-[1,2,4]triazolo[1,5-a]pyrimidin-2-amine.

A further embodiment of the invention provides compounds of formula I, wherein A is the ring j), for example the compound
N-(3-methoxy-4-(4-methyl-1H-imidazol-1-yl)phenyl)-7-phenyl-6,7-dihydro-5H-[1,2,4]triazolo[5,1-b][1,3]oxazin-2-amine.

A further embodiment of the invention provides compounds of formula I, wherein A is the ring k), for example the compound
N-(3-methoxy-4-(4-methyl-1H-imidazol-1-yl)phenyl)-8-phenyl-6,8-dihydro-5H-[1,2,4]triazolo[5,1-c][1,4]oxazin-2-amine.

An embodiment of the invention provides further compounds of formula IA

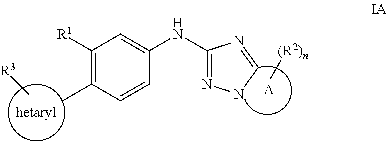

wherein
R¹ is lower alkoxy or cyano;
R² is lower alkyl, halogen, lower alkoxy, lower alkyl substituted by halogen, lower alkyl substituted by OR, ═O, —C(O)O-lower alkyl, —C(O)NH-lower alkyl, cyano, O—(CH₂)ₒ-phenyl, CH₂—O-lower alkyl or NRR′,
or is —(CH₂)ₒ-phenyl optionally substituted by one or two substituents, selected from halogen, cyano, lower alkyl, lower alkyl substituted by halogen, lower alkoxy or by lower alkoxy substituted by halogen,
or is —(CH₂)ₒ-cycloalkyl, —(CH₂)ₒ-heterocycloalkyl or —(CH₂)ₒ-hetaryl;
R and R′ are each independently hydrogen or lower alkyl, and
o is 0 or 1;
R³ is lower alkyl;

### A is

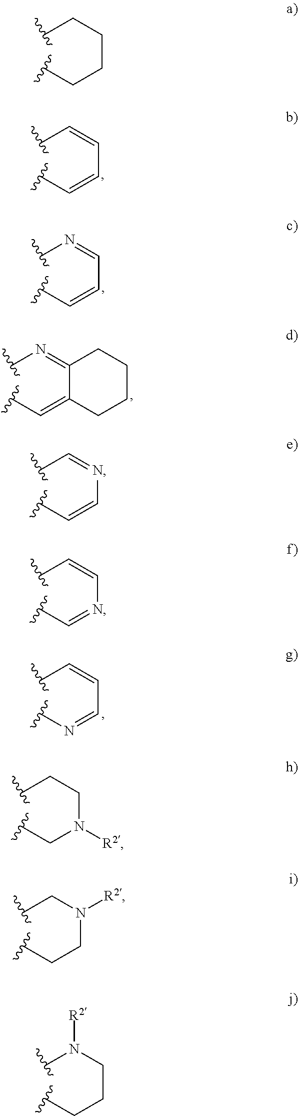

and
R^{2′} is hydrogen, lower alkyl, lower alkyl substituted by halogen, C(O)-lower alkyl or S(O)₂-lower alkyl;
Hetaryl is a 5 or 6 membered N, S or O-containing heteroaryl group;
N is 0, 1, 2 or 3; when n is 2 or 3, each R² is the same or different;
or pharmaceutically active acid addition salts thereof.

The present compounds of formula I and their pharmaceutically acceptable salts can be prepared by methods known in the art, for example, by processes described below, which processes comprise

a) reacting a compound of formula

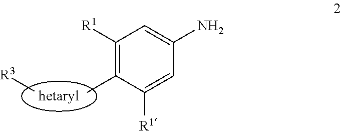

with a compound of formula

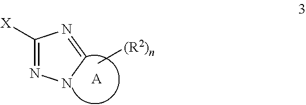

to obtain a compound of formula

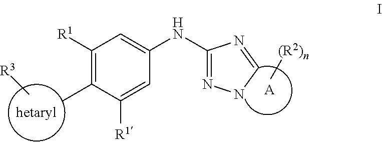

wherein X is halogen and the further groups have the meaning as described above and, if desired, converting the compounds obtained into pharmaceutically acceptable acid addition salts;
or
b) reacting a compound of formula

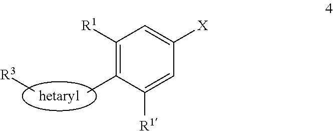

with a compound of formula

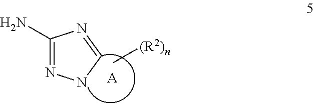

to obtain a compound of formula

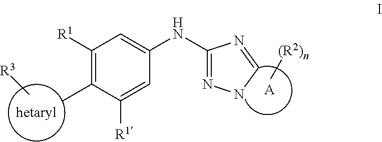

wherein X is halogen and the further groups have the meaning as described above, and, if desired converting the compounds obtained into pharmaceutically acceptable acid addition salts;
the preparation of compounds of formula I of the present invention may be carried out in sequential or convergent synthetic routes. Syntheses of the compounds of the invention are shown in the following schemes. The skills required for carrying out the reaction and purification of the resulting products are known to those skilled in the art. The substituents and indices used in the following description of the processes have the significance given herein before unless indicated to the contrary.

In more detail, the compounds of formula I can be manufactured by the methods given below, by the methods given in the examples or by analogous methods. Appropriate reaction conditions for the individual reaction steps are known to a person skilled in the art. The reaction sequence is not limited to the one displayed in the schemes, however, depending on the starting materials and their respective reactivity the sequence of reaction steps can be freely altered. Starting materials are either commercially available or can be prepared by methods analogous to the methods given below, by methods described in the examples, or by methods known in the art.

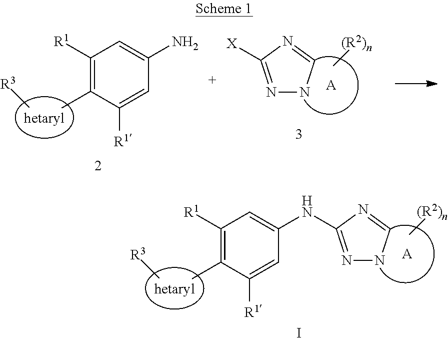

The present compounds of formula I and their pharmaceutically acceptable salts can be prepared by coupling of anilines of general formula 2 and halides of general formula 3 (see Scheme 1). This reaction can e.g. be accomplished using generally known procedures, e.g. displacement reactions under catalytic conditions (like e.g. palladium(0) or copper(II) catalysis) or under thermal conditions or under basic conditions.

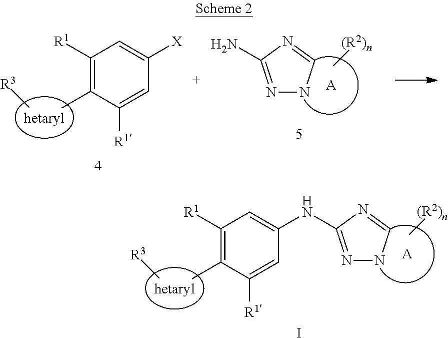

Alternatively halides of general formula 4 (X preferably equals I, triflate, Br or Cl, more preferably Br), and amines of general formula 5 can be coupled to provide compounds of general formula I (see Scheme 2). This reaction can e.g. be accomplished in the presence of a metal (for example Cu or Pd). A method for the coupling of heteroaryl amines with aryl halides is e.g. described by J. P. Schulte et al. (Synlett 2007, 2331-6) which employed sodium phenolate, Pd₂(dba)₃, Xantphos as reagents and dioxane as solvent.

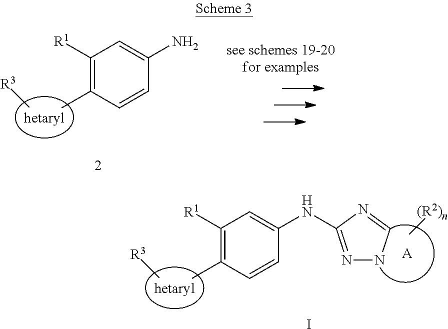

Compounds of general formula I can also be prepared starting from anilines 2 comprising the construction of the heteroaryl moiety (see Scheme 3).

Anilines of general formula 2, which can be used as starting materials for the preparation of compounds of formula I may be prepared as described in the following schemes.

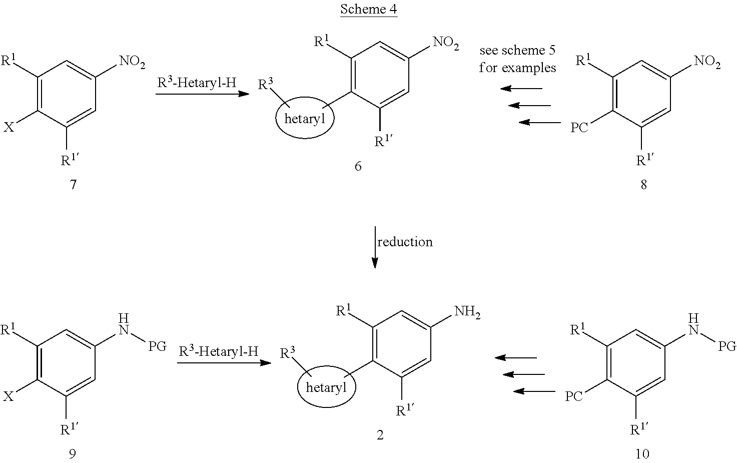

PG is a N-protecting group, such as tert-butoxycarbonyl (Boc) group, X is a halide, PC is —CHO, —(CO)R³ or —(CO)OR³, —(CS)NH₂, R³ is lower alkyl.

Nucleophilic substitution at room temperature or elevated temperature (e.g. reflux or under pressure using a microwave oven) under neutral conditions or in the presence of a base (like e.g. potassium carbonate), neat or in a polar solvent (like e.g. THF or DMSO etc.) of substituted 4-nitro-phenyl halides 7 (X═F, Cl, Br, I) with compounds Hetaryl-H, (like 4-methyl-imidazole) yield nitro derivatives 6 (see Scheme 4). Alternatively, nitro derivatives 6 can be prepared from suitable precursors 8 (PC=—CHO, —(CO)R′, —(CO)OR′ or —(CS)NH₂ with R′=lower alkyl), by applying standard reaction sequences for the formation of the heteroaryl substituent. Nitro compounds 6 can be reduced to anilines 2 using generally known procedures, e.g. hydrogenation in the presence of a catalyst (like e.g. 10% palladium on carbon) in a solvent (like e.g. ethanol or ethyl acetate) or, by using a metal (like e.g. iron) or a metal salt (like e.g. stannous chloride) in a polar solvent (like e.g. acetic acid or tetrahydrofurane). Alternatively, anilines 2 can be prepared by introducing a heteroaryl substituent into N-protected aniline derivatives 9 (PG=protecting group) using generally known procedures, e.g. displacement reactions under catalytic conditions (like e.g. palladium(0) or copper(II) catalysis) or, by forming a heteroaryl group in N-protected aniline derivatives 10, respectively, and subsequently cleaving off the protecting group.

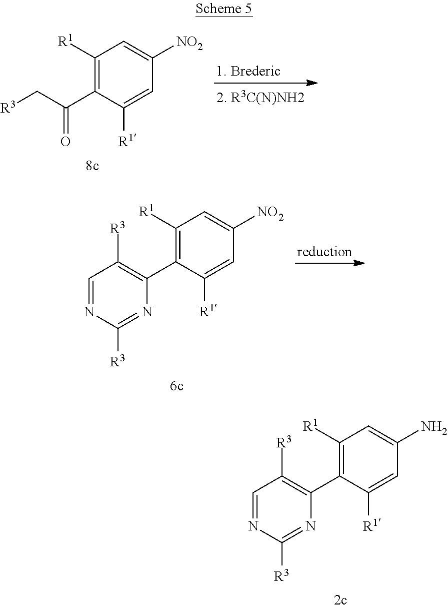

R³ is lower alkyl.
Pyrimidines 6c can be prepared by building up the pyrimidine ring for example by reacting the 4-nitro-acetophenone derivative 8c with an ortho ester derivative (like e.g. the Brederick reagent) and subsequent condensation with an amidine derivative (R³C(N)NH₂) to yield the nitro derivative 6c (see Scheme 5).

Reduction of nitro derivatives 6c provides the respective anilines 2c.

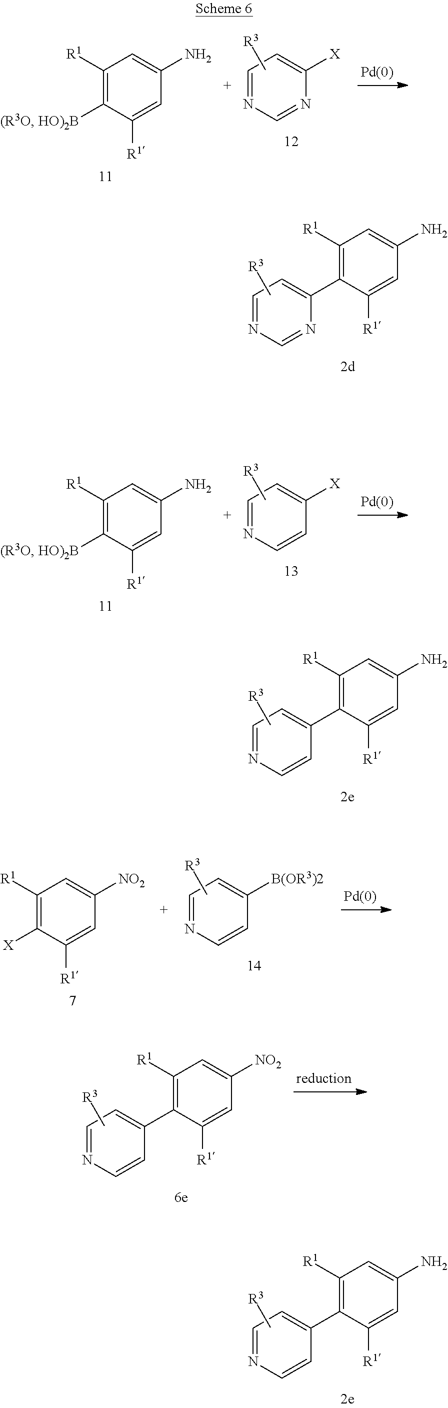

X is halide (like e.g. bromine or iodine), R³ is lower alkyl or hydrogen.

Heterocyclic anilines like the pyrimidine derivative 2d or pyridine 2e (see Scheme 6) maybe prepared by Suzuki coupling of the corresponding pyrimidine respectively pyridine halide (12, 13) with the corresponding aniline boronic acid respectively ester 11 or by Suzuki coupling of the pyridine boronic acid or ester (like e.g. the pinacol ester) 14 with the 4-halo-nitro-benzene derivative 7 and subsequent reduction of the nitro derivative 6e to the aniline or directly with the 4-halo-aniline. Aryl boronic acids and esters used as starting materials are either commercially available or readily prepared by methods known to one skilled in the art of organic synthesis such as treatment of the corresponding aryl bromides with bis(pinacolato)diboron in the presence of a palladium catalyst.

Halides of general formula 4 (X preferably equals Br or Cl, more preferably Br), which can be used as starting materials for the preparation of compounds of formula I may be prepared as described in the following schemes.

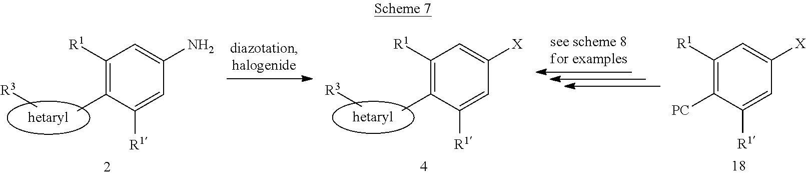

X is halide, PC is —NH₂, —CHO or —(CO)R³ with R³=lower alkyl.

Amines 2 with suitable substituents R¹ and heteroaryl can be subjected to a diazotation reaction in the presence of an appropriate halide source which provides the desired halides 4 (see Scheme 7). Suitable reagents for preparation of bromides (X═Br) are e.g. t-butyl nitrite or isoamyl nitrite and copper(II) bromide in acetonitrile. Alternatively sodium nitrite in aqueous HBr solution in the presence of sodium bromide, copper bromide or copper sulphate can be used. Analogously the chlorides 4 (X═Cl) can be obtained by employing the corresponding chloride sources (copper chloride, HCl etc).

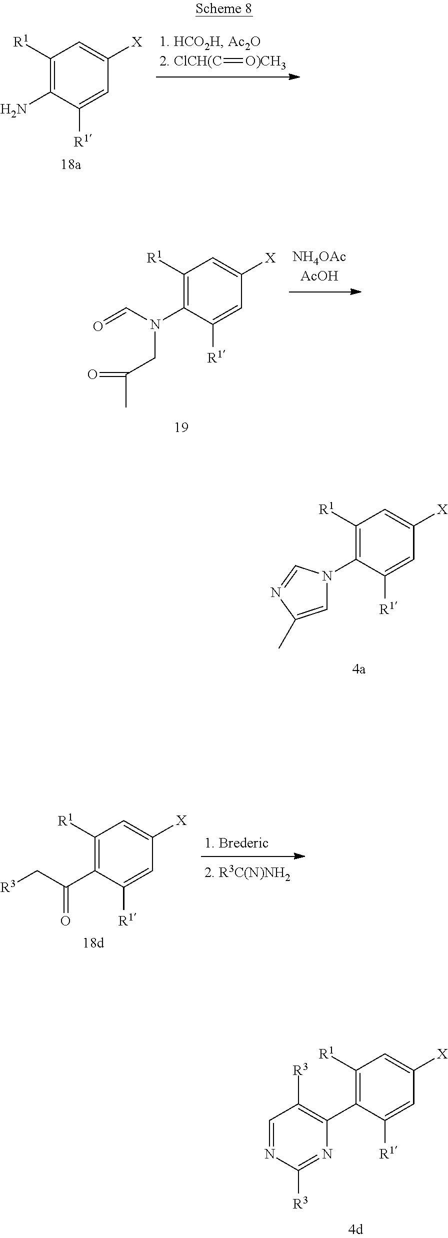

Alternatively, halides 4 can be prepared from a suitable precursor 18 (PC═—NH₂, —CHO or —(CO)R³ with R³=lower alkyl), by applying standard reaction sequences for the formation of the heteroaryl substituent (see Scheme 8).

Anilines 18a can be converted into imidazoles 4a (as described for example in EP1950211 A1, Exp 1.3-1.5) e.g. by sequential formylation (with acetic anhydride and formic acid) and alkylation (with chloroacetone in the presence of a base e.g. cesium carbonate and potassium iodide in DMF). Ring closure of intermediate 19 can then be achieved by heating with ammonium acetate and acetic acid neat or in xylene.

Pyrimidines 4d can be prepared by building up the pyrimidine ring for example by reacting the acetophenone derivative 18d with a ortho ester derivative (like e.g. the Brederick reagent) and subsequent condensation with an amidine derivative (R³C(N)NH₂) to yield pyrimidine 4d.

The starting materials 18 are either commercially available or readily prepared by methods known to one skilled in the art of organic synthesis.

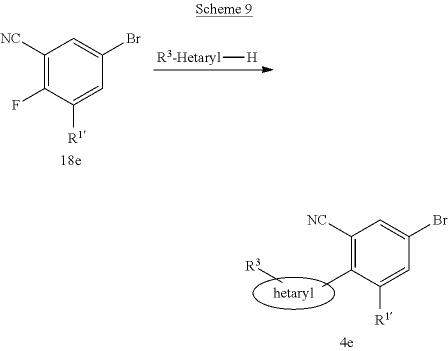

For R¹═CN an alternative method of producing bromides 4e (X═Br, see Scheme 9) useful to this invention is by a nucleophilic substitution at room temperature or elevated temperature (e.g. reflux or under pressure using a microwave oven) under neutral conditions or in the presence of a base (like e.g. potassium carbonate), neat or in a polar solvent (like e.g. THF or DMSO etc.) of 5-bromo-2-fluoro-benzonitrile with compounds R³-Hetaryl-H, (like 4-methyl-imidazole, see US20060004013, Exp. 9).

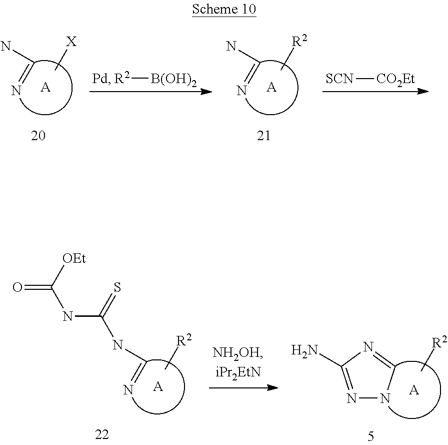

For compounds 5 in which A is heteroaryl (see Scheme 10) the annelated triazole moiety can be constructed from the corresponding amino derivative 21, which are either commercially available or can be obtained from the corresponding halides 20 (X═Cl or Br; A=heteroaryl) by palladium catalyzed Suzuki coupling with boronic acids or boronic esters (e.g. pinacol ester). Amines 21 can be reacted with ethoxycarbonyl isothiocyanate to yield thiourea derivatives 22 which undergo a cyclization reaction upon treatment with hydroxylamine in the presence of a base under liberation of carbon dioxide to yield annelated triazoles 5 (as e.g. described by M. Nettekoven et al., Synthesis 2003, 11, 1649-1652).

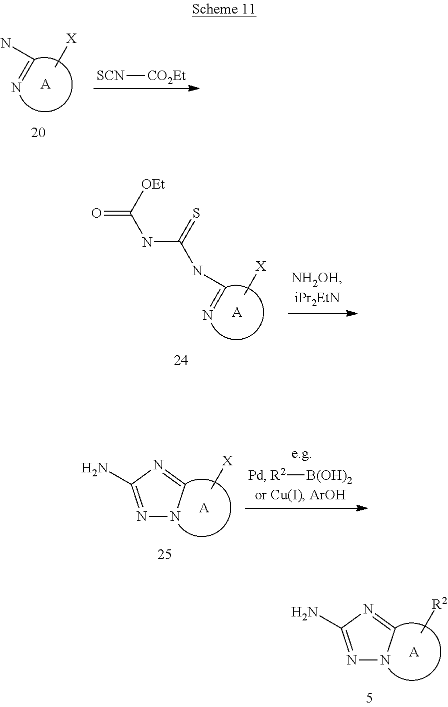

Alternatively the order of steps in Scheme 10 can be changed (see Scheme 11). Halides 20 (which are either commercially available or can be synthesized by methods known in the art e.g. in analogy to Example 135a by bromination of a suitable aminopyridine) can be reacted with ethoxycarbonyl isothiocyanate followed by treatment with hydroxylamine to provide annelated triazoles 25. These halides can then be subjected e.g. to palladium catalyzed Suzuki coupling with boronic acids or copper (I) catalyzed coupling with phenols (e.g. according to D. Maiti et al. JOC 2010, 75, 1791-1794) to provide substituted aminotriazoles 5.

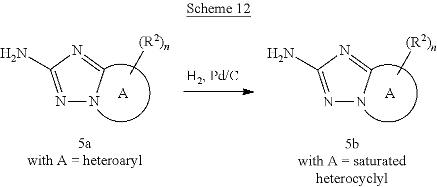

Compounds 5a with A=heteroaryl can be hydrogenated with palladium on charcoal as catalyst to yield the corresponding partly saturated compounds 5b (see Scheme 12). Depending on the nature of ring A this reaction may require elevated temperature or hydrogen pressure or the presence of acid (e.g. HCl). Alternatively compounds 5a can be reduced with metals e.g. magnesium in alcoholic solution (like ethanol) with or without activation of the metal (e.g. activation with catalytic amounts of iodine).

If ring A of compound 5b contains a NH group this can be modified e.g. by reductive amination with aldehydes or ketones in the presence of a reducing agent like sodium triacetoxy borohydride to give the alkylated amines, by acylation with anhydrides or acid chlorides in the presence of a base to give the amides, by reaction with sulfonylchlorides to give the sulfonamides, by reaction with carbonyldiimidazole or triphosgene and alcohols or an amines to give the carbamates or ureas.

To accomplish these modifications it might be necessary to protect the amino group on the triazole 5a prior to the hydrogenation step e.g. by protection with Boc group which can be introduced e.g. with Boc anhydride and can be cleaved after hydrogenation and the modifications with e.g. trifluoroacetic acid.

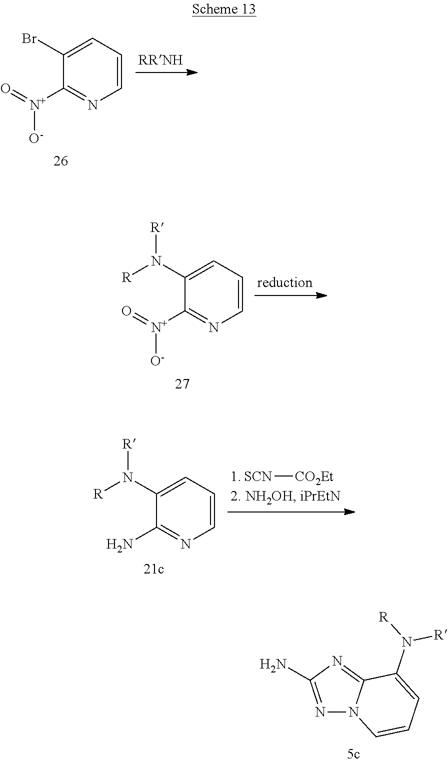

Introduction of an amine substituent (R²═RR′N) in 8-position of triazolopyridine 5c (see Scheme 13) can be accomplished by treating the 3-bromo-2-nitropyridine with an amine RR′NH in the presence of a base (e.g. potassium carbonate), a catalyst (e.g. TBAI) at ambient to higher temperature in a polar solvent (e.g. DMSO). Reduction of the nitro group either by metal, metal salts or hydrogen in the presence of a catalyst (e.g. Pd on carbon) yields the aminopyridine 21c which can be converted according to Scheme 10 to the corresponding aminotriazole derivative 5c.

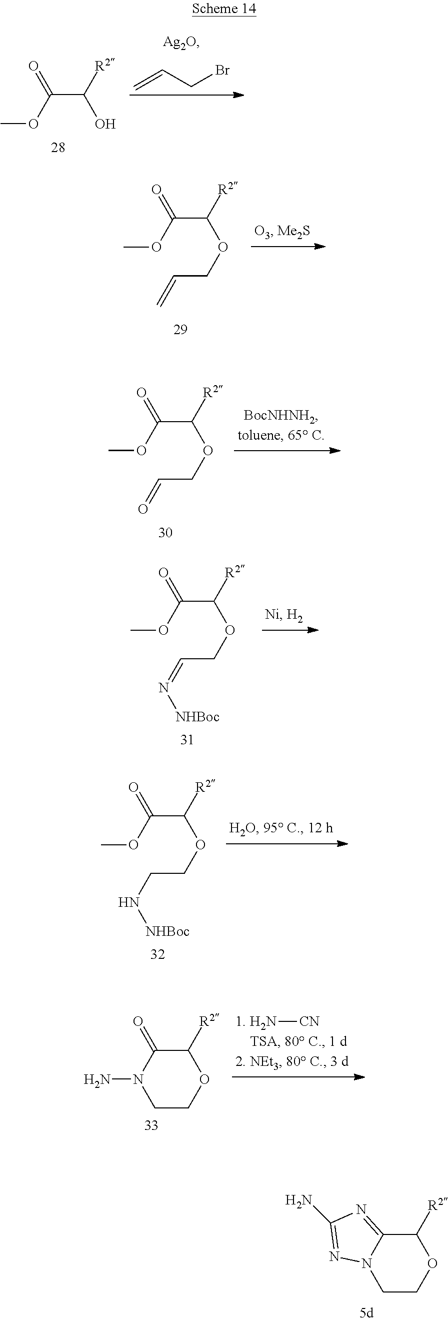

R^{2″} is phenyl optionally substituted by halogen or lower alkyl.

Aminotriazoles of general formula 5d can be prepared starting from mandelate derivative 28 (see Scheme 14). Allylation followed by ozonolysis of the double bond provides aldehyde 30 which forms hydrazone 31 upon treatment with Boc-protected hydrazine. Catalytic hydrogenation in the presence of Nickel gives compound 32. Heating in water causes lactamization and deprotection (in analogy to J. W. Nilsson et al. J. Med. Chem. 2003, 46, 3985-4001). Hydrazide 33 undergoes a cyclization reaction with cyanamide by heating under acidic conditions first followed by heating under basic conditions (in analogy to WO2010/098487, Preparation Example 2-7) to provide aniline 5d.

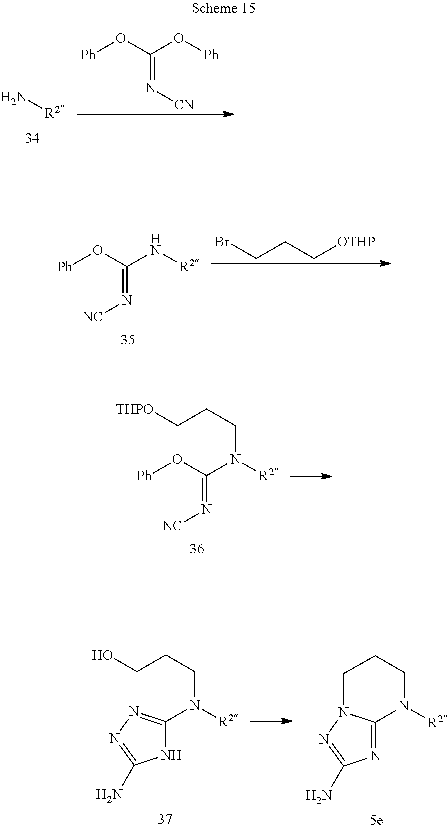

R^{2″} is phenyl optionally substituted by halogen or lower alkyl.

Amines 34 can be acylated with N-cyanodiphenoxyimidocarbonate (see Scheme 15) and alkylated with a suitable protected 3-halo-propanol (e.g. bromo-alcohol protected with a THP ether) in the presence of a base (e.g. potassium carbonate) at ambient or higher temperature in a polar solvent (e.g. DMF). After deprotection of the alcohol the compound 37 is cyclized for example under Mitsunobo conditions or with tetrabromomethane and triphenylphosphine to yield the amine 5e.

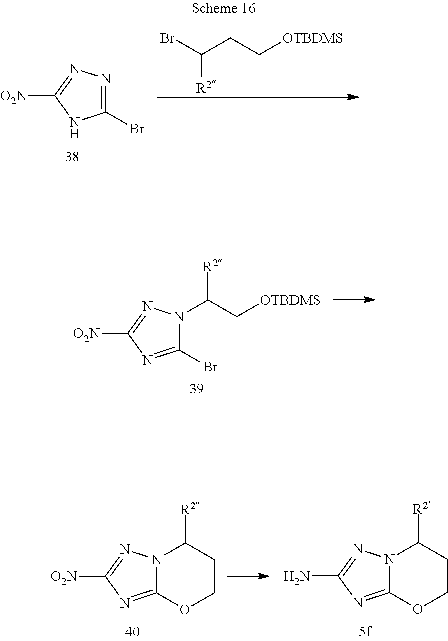

R² is phenyl optionally substituted by halogen or lower alkyl.

3-Bromo-5-nitro-4H-[1,2,4]triazole 38 can be alkylated with a suitable protected bromo-alcohol (e.g. with the tert.-butyldimethylsilyl group) in the presence of base (e.g. potassium carbonate). Deprotection of the alcohol may lead to spontaneous cyclization of the liberated alcohol onto the bromide or may be catalyzed by a base to give compound 40. Reduction of the nitro-group by hydrogen catalyzed by a metal catalyst (e.g. Pd on carbon) or by metal salts or metals provides the amine 5f (see Scheme 16).

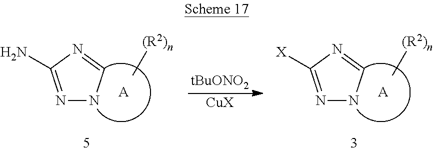

The halotriazole 3 can be prepared from the aniline 5 (see Scheme 17) via formation of the corresponding diazonium salt and subsequent decomposition in the presence of a halide source like copper (I) halide or hydrogenhalide (X=chlorine or bromine).

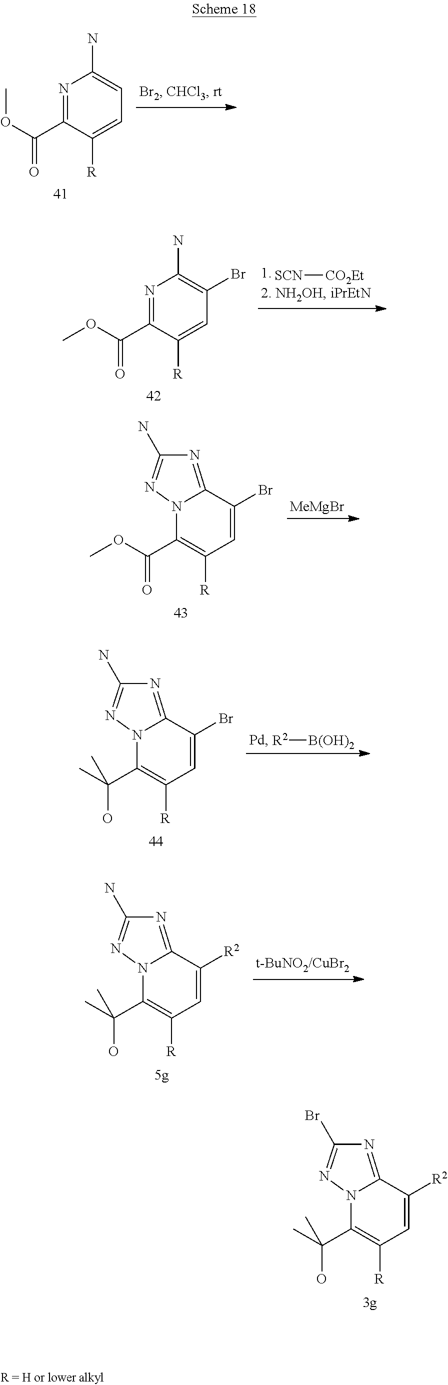

Anilines of general formula 5 g or the corresponding bromides 3 g with an 2-propan-2-ol group in 5-position of the triazolopyridine (see Scheme18) can be prepared starting from ester 41 by bromination in chloroform followed by cyclization as already described in Scheme 10 to give 2-amino-triazolopyridine 43. The ester 43 can then be treated with methyl magnesium bromide to provide the tertiary alcohol 44. Conversion of the bromide by e.g. Suzuki reaction gives aniline 5g or after Sandmeyer reaction bromide 3g. The starting material 41 is either commercially available or can be synthesized by methods known in the art, e.g. for R=Me, 41 can be prepared from the corresponding bromide by reaction with trimethyl boroxine in the presence of a palladium catalyst.

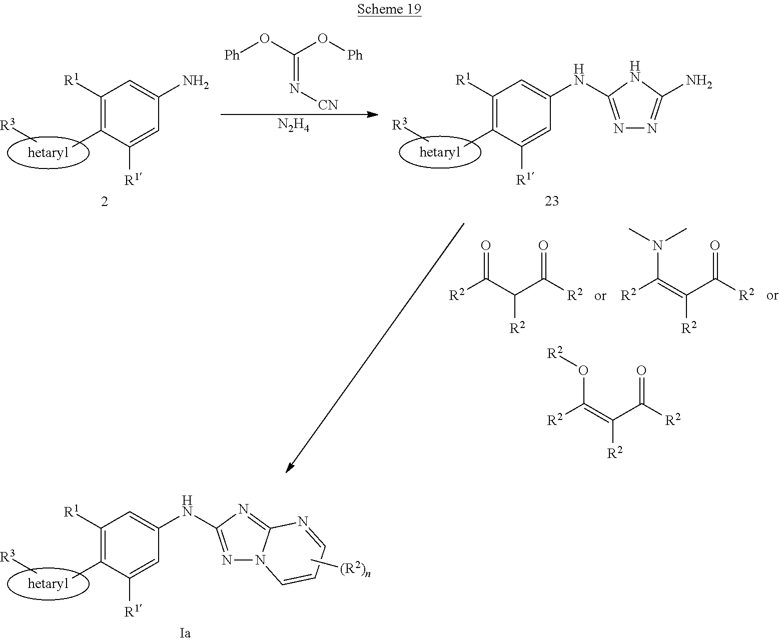

Anilines 2 can be acylated with N-cyanodiphenoxyimidocarbonate and then cyclized with hydrazine to yield the diaminotriazole derivative 23. On heating with 1,3-diketones or its analogues like 2-methyleneamino or 2-alkoxymethylene ketones under acidic conditions (like acetic acid or solvent in the presence of an acid like TFA, HCl, TosOH etc.) the diaminotriazolo derivative 23 is converted to the triazolopyrimidine derivative Ia (Scheme19).

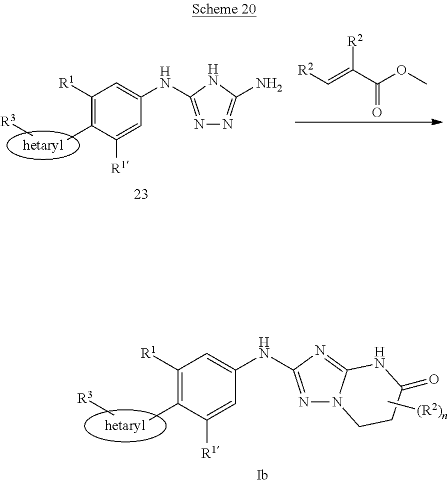

Heating of the diaminotriazole derivative 23. with an alpha, beta-unsaturated ester in polar solvents (like e.g. DMF) yields the saturated amide derivative Ib (Scheme 20).

The compounds were investigated in accordance with the test given hereinafter.

### Description of γ-Secretase Assay

### Cellular γ-Secretase Assay

Human neuroglioma H4 cells overexpressing human APP were plated at 30,000 cells/well/200 μA in 96-well plates in IMDM media containing 10% FCS, 0.2 mg/l Hygromycin B and incubated for 2 h at 37° C., 5% CO₂ prior to adding test compounds.

Compounds for testing were dissolved in 100% Me₂SO yielding in a 10 mM stock solution. Typically 12 μl of these solutions were further diluted in 1000 μl of IMDM media (w/o FCS). Subsequent 1:1 dilutions gave a ten point dose response curve. 100 μl of each dilution was added to the cells in 96-well plates. Appropriate controls using vehicle only and reference compound were applied to this assay. The final concentration of Me₂S0 was 0.4%.

After incubation for 22 hrs at 37° C., 5% CO₂, 50 μl supernatant was transferred into round-bottom 96-well polypropylene plates for detection of Aβ42. 50 μl assay buffer (50 mM Tris/Cl, pH 7.4, 60 mM NaCl, 0.5% BSA, 1% TWEEN 20) was added to the wells followed by the addition of 100 μl of detection antibody (ruthenylated BAP15 0.0625 μg/ml in assay buffer). 50 μl of a premix of capture antibody (biotinylated 6E10 antibody, 1 μg/ml) and Steptavidin-coated magnetic beads (Dynal M-280, 0.125 mg/ml) were preincubated for 1 hr at room temperature before adding the assay plates. Assay plates were incubated on a shaker for 3 hrs at room temperature and finally read in the Bioveris M8 Analyser according to the manufacturer's instructions (Bioveris).

Toxicity of compounds was monitored by a cell viability test of the compound-treated cells using a colorimetric assay (CellTiter 96™ AQ assay, Promega) according to the manufacturer's instructions. Briefly, after removal of 50 μl cell culture supernatant for detection of Aβ42, 20 μl of 1× MTS/PES solution was added to the cells and incubated for 30 min at 37° C., 5% CO₂. Optical density was then recorded at 490 nm.

IC₅₀ values for inhibition of Aβ42 secretion were calculated by nonlinear regression fit analysis using XLfit 4.0 software (IDBS).

The preferred compounds show a IC₅₀<0.5 (μM). In the list below are described the data for all compounds to the inhibition of Aβ42 secretion:

| | EC₅₀ Aβ42 |
|---|---|
| Example No. | (μM) |
| 1 | 0.33 |
| 2 | 0.32 |
| 3 | 3.35 |
| 4 | 1.28 |
| 5 | 0.09 |
| 6 | 0.12 |
| 7 | 0.93 |
| 8 | 0.05 |
| 9 | 0.20 |
| 10 | 0.06 |
| 11 | 0.58 |
| 12 | 0.4 |
| 13 | 0.1 |
| 14 | 0.679 |
| 15 | 0.572 |
| 16 | 0.197 |
| 17 | 0.285 |
| 18 | 0.129 |
| 19 | 0.953 |
| 20 | 0.107 |
| 21 | 0.201 |
| 22 | 0.074 |
| 23 | 0.191 |
| 24 | 0.592 |
| 25 | 0.601 |
| 26 | 0.312 |
| 27 | 0.742 |
| 28 | 1.493 |
| 29 | 1.268 |
| 30 | 0.247 |
| 31 | 0.587 |
| 32 | 0.864 |
| 33 | 0.556 |
| 34 | 0.64 |
| 35 | 5.931 |
| 36 | 0.573 |
| 37 | 0.26 |
| 38 | 0.578 |
| 39 | 0.145 |
| 40 | 0.417 |
| 41 | 4.309 |
| 42 | 1.32 |
| 43 | 0.68 |
| 44 | 1.56 |
| 45 | 1.40 |
| 46 | 0.18 |
| 47 | 1.34 |
| 48 | 4.51 |
| 49 | 1.35 |
| 50 | 0.40 |
| 51 | 0.44 |
| 52 | 0.28 |
| 53 | 0.12 |
| 54 | 0.11 |
| 55 | — |
| 56 | — |
| 57 | 0.431 |
| 58 | 0.538 |
| 59 | 0.59 |
| 60 | 0.672 |
| 61 | 0.831 |
| 62 | 0.01 |
| 63 | 0.01 |
| 64 | 0.073 |
| 65 | 0.024 |
| 66 | 0.238 |
| 67 | 0.45 |
| 68 | 1.167 |
| 69 | 0.485 |
| 70 | 0.673 |
| 71 | 0.27 |
| 72 | 0.355 |
| 73 | 0.359 |
| 74 | 0.035 |
| 75 | 0.019 |
| 76 | 0.019 |
| 78 | 0.027 |
| 79 | 0.073 |
| 80 | 0.121 |
| 81 | 0.022 |
| 82 | 0.242 |
| 83 | 0.408 |
| 84 | 0.321 |
| 85 | 0.032 |
| 86 | 0.143 |
| 87 | 0.044 |
| 88 | 0.188 |
| 89 | 0.290 |
| 90 | 0.475 |
| 91 | 0.310 |
| 92 | 0.375 |
| 93 | 0.068 |
| 94 | 0.039 |
| 95 | 0.084 |
| 96 | 0.093 |
| 97 | 0.470 |
| 98 | 0.390 |
| 101 | 0.227 |
| 107 | 0.356 |
| 108 | 0.488 |
| 109 | 0.270 |
| 110 | 0.029 |
| 111 | 0.029 |
| 112 | 0.046 |
| 113 | 0.081 |
| 114 | 0.238 |
| 115 | 0.242 |
| 116 | 0.117 |
| 117 | 0.065 |
| 118 | 0.072 |
| 119 | 0.031 |
| 120 | 0.096 |
| 121 | 0.076 |
| 122 | 0.079 |
| 123 | 0.029 |
| 124 | 0.357 |
| 125 | 0.050 |
| 126 | 0.032 |
| 127 | 0.039 |
| 128 | 0.030 |
| 129 | 0.300 |
| 130 | 0.016 |
| 131 | 0.038 |
| 132 | 0.137 |
| 133 | 0.045 |
| 135 | 0.141 |
| 136 | 0.152 |
| 137 | 0.266 |
| 139 | 0.311 |
| 140 | 0.180 |
| 141 | 0.375 |
| 142 | 0.232 |
| 143 | 0.185 |
| 144 | 0.069 |
| 145 | 0.027 |
| 146 | 0.069 |
| 147 | 0.033 |
| 148 | 0.09 |
| 149 | 0.37 |

The present invention also provides pharmaceutical compositions containing compounds of the invention, for example, compounds of formula I or pharmaceutically acceptable salts thereof and a pharmaceutically acceptable carrier. Such pharmaceutical compositions can be in the form of tablets, coated tablets, dragées, hard and soft gelatin capsules, solutions, emulsions or suspensions. The pharmaceutical compositions also can be in the form of suppositories or injectable solutions.

The pharmaceutical compositions of the invention, in addition to one or more compounds of the invention, contain a pharmaceutically acceptable carrier. Suitable pharmaceutically acceptable carriers include pharmaceutically inert, inorganic or organic carriers. Lactose, corn starch or derivatives thereof, talc, stearic acids or its salts and the like can be used, for example, as such carriers for tablets, coated tablets, dragées and hard gelatin capsules. Suitable carriers for soft gelatin capsules are, for example, vegetable oils, waxes, fats, semi-solid and liquid polyols and the like. Depending on the nature of the active substance no carriers are, however, usually required in the case of soft gelatin capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Such pharmaceutical compositions containing a compound of formula I or a pharmaceutically acceptable salt thereof and a therapeutically inert carrier are also an object of the present invention, as is a process for their production, which comprises bringing one or more compounds of formula I and/or pharmaceutically acceptable acid addition salts and, if desired, one or more other therapeutically valuable substances into a galenical administration form together with one or more therapeutically inert carriers.

In accordance with the invention compounds of formula I as well as their pharmaceutically acceptable salts are useful in the control or prevention of illnesses based on the inhibition of Aβ42 secretion, such as of Alzheimer's disease.

The dosage at which compounds of the invention can be administered can vary within wide limits and will, of course, have to be adjusted to the individual requirements in each particular case. In the case of oral administration the dosage for adults can vary from about 0.01 mg to about 1000 mg per day of a compound of general formula I or of the corresponding amount of a pharmaceutically acceptable salt thereof. The daily dosage can be administered as single dose or in divided doses and, in addition, the upper limit can also be exceeded when this is found to be indicated.

### Tablet Formulation

### Wet Granulation

| | | mg/tablet | | | |
|---|---|---|---|---|---|
| Item | Ingredients | 5 | 25 | 100 | 500 |
| 1. | Compound of formula I | 5 | 25 | 100 | 500 |
| 2. | Lactose Anhydrous DTG | 125 | 105 | 30 | 150 |
| 3. | Sta-Rx 1500 | 6 | 6 | 6 | 30 |
| 4. | Microcrystalline Cellulose | 30 | 30 | 30 | 150 |
| 5. | Magnesium Stearate | 1 | 1 | 1 | 1 |
| | Total | 167 | 167 | 167 | 831 |

### Manufacturing Procedure

1. Mix items 1, 2, 3 and 4 and granulate with purified water.
2. Dry the granules at 50° C.
3. Pass the granules through suitable milling equipment.
4. Add item 5 and mix for three minutes; compress on a suitable press.

### Capsule Formulation

| | | mg/capsule | | | |
|---|---|---|---|---|---|
| Item | Ingredients | 5 | 25 | 100 | 500 |
| 1. | Compound of formula I | 5 | 25 | 100 | 500 |
| 2. | Hydrous Lactose | 159 | 123 | 148 | — |
| 3. | Corn Starch | 25 | 35 | 40 | 70 |
| 4. | Talc | 10 | 15 | 10 | 25 |
| 5. | Magnesium Stearate | 1 | 2 | 2 | 5 |
| Total | | 200 | 200 | 300 | 600 |

### Manufacturing Procedure

1. Mix items 1, 2 and 3 in a suitable mixer for 30 minutes.
2. Add items 4 and 5 and mix for 3 minutes.
3. Fill into a suitable capsule.

### Example 1

### [8-(4-Fluoro-phenyl)-5,6,7,8-tetrahydro-[1,2,4]triazolo[1,5-a]pyridin-2-yl]-[3-methoxy-4-(4-methyl-imidazol-1-yl)-phenyl]-amine

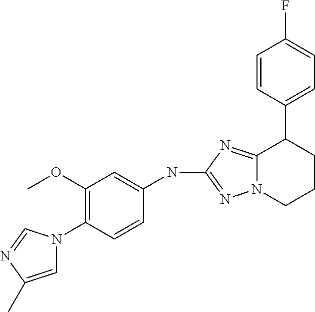

### a) 3-(4-Fluoro-phenyl)-pyridin-2-ylamine

A mixture of 2-amino-3-bromopyridine (2.0 g, 11.2 mmol), 4-fluorophenyl boronic acid (3.23 g, 22.4 mmol), dichloro[1,1′-bis(diphenylphosphino)-ferrocene]palladium(II) dichloromethane adduct (733 mg, 0.001 mmol) and an aqueous solution of Na₂CO₃ (2N, 11.2 mL, 22.4 mmol) in dioxane (30 mL) was stirred at 110° C. for 2 hours. The reaction mixture was diluted with water and extracted with diethyl ether, the combined organic phases were dried over sodium sulfate, the solvent was evaporated and the residue purified by silica gel chromatography using n-heptane/diethyl ether as eluent. The title compound was obtained as a light yellow solid (1.95 g, 92%).

MS ISP (m/e): 189.3 (100) [(M+H)⁺].

¹H NMR (CDCl₃, 300 MHz): δ (ppm)=8.08-8.06 (m, 1H), 7.44-7.39 (m, 2H), 7.34-7.31 (m, 1H), 7.17-7.12 (m, 2H), 6.76-6.72 (m, 1H), 4.57 (br s, 2H).

### b) N-(3-(4-Fluoro-phenyl)-pyridin-2-yl)-N′-ethoxycarbonyl-thiourea

To a solution of 3-(4-fluoro-phenyl)-pyridin-2-ylamine (200 mg, 1.06 mmol) in dioxane (10 mL) was added ethoxycarbonyl isothiocyanate (141 μL, 1.17 mmol) and stirred at room temperature for 12 hours. The solvent was evaporated and the residue was used for the next step without purification. The title compound was obtained as a light yellow solid (340 mg, 100%).

MS ISP (m/e): 320.1 (100) [(M+H)⁺].

### c) 8-(4-Fluoro-phenyl)-[1,2,4]triazolo[1,5-a]pyridin-2-ylamine

To a solution of hydroxylamine hydrochloride (370 mg, 5.32 mmol) and N,N-diisopropylethylamine (543 μL, 3.19 mmol) in MeOH (2 mL) and EtOH (2 mL) was added a solution of N-(3-(4-fluoro-phenyl)-pyridin-2-yl)-N′-ethoxycarbonyl-thiourea (340 mg, 1.06 mmol) in MeOH (2 mL) and EtOH (2 mL). The reaction mixture was stirred at room temperature for 1 hour and then at 60° C. for 3 hours. The solvents were evaporated and saturated NaHCO₃ solution was added to the residue. The aqueous phase was extracted with CH₂Cl₂, the combined organic phases were dried over sodium sulfate, the solvent was evaporated and the residue purified by silica gel chromatography using CH₂Cl₂/MeOH (with 10% ammonia) as eluent. The title compound was obtained as a white solid (205 mg, 84%).

MS ISP (m/e): 229.2 (100) [(M+H)⁺].

¹H NMR (CDCl₃, 300 MHz): δ (ppm)=8.31-8.28 (m, 1H), 7.96-7.91 (m, 2H), 7.50-7.47 (m, 1H), 7.22-7.16 (m, 2H), 6.94-6.89 (m, 1H), 4.51 (br s, 2H).

### d) 8-(4-Fluoro-phenyl)-5,6,7,8-tetrahydro-[1,2,4]triazolo[1,5-a]pyridin-2-ylamine

8-(4-Fluoro-phenyl)-[1,2,4]triazolo[1,5-a]pyridin-2-ylamine (232 mg, 1.02 mmol) in EtOH (10 mL) and HCl (25%, 162 μl, 1.12 mmol) was hydrogenated in the presence of Pd on charcoal (10%, 232 mg, 0.218 mmol) at 50 bar and 50° C. for 18 hours. The catalyst was filtered off, washed thoroughly with EtOH and the solvent was removed from the combined filtrates. Saturated aqueous NaHCO₃ solution was added to the residue. The aqueous phase was extracted with CH₂Cl₂, the combined organic phases were dried over sodium sulfate, the solvent was evaporated and the residue purified by silica gel chromatography using CH₂Cl₂/MeOH (with 10% ammonia) as eluent. The title compound was obtained as a white solid (174 mg, 74%).

MS ISP (m/e): 233.1 (100) [(M+H)⁺].

¹H NMR (CDCl₃, 300 MHz): δ (ppm)=7.14-7.09 (m, 2H), 7.04-6.98 (m, 2H), 4.14-4.03 (m, 5H), 2.30-2.24 (m, 1H), 2.15-1.90 (m, 3H).

### e) [8-(4-Fluoro-phenyl)-5,6,7,8-tetrahydro-[1,2,4]-triazolo[1,5-a]pyridin-2-yl]-[3-methoxy-4-(4-methyl-imidazol-1-yl)-phenyl]-amine

A mixture of 1-(4-bromo-2-methoxy-phenyl)-4-methyl-1H-imidazole (WO2009076352, Example 1; 69 mg, 0.258 mmol), 8-(4-fluoro-phenyl)-5,6,7,8-tetrahydro-[1,2,4]triazolo[1,5-a]pyridin-2-ylamine (50 mg, 0.215 mmol), sodium phenoxide (37.5 mg, 0.323 mmol), tris(dibenzylideneacetone)dipalladium chloroform complex (8.9 mg, 0.009 mmol) and 4,5-bis(diphenylphosphino)-9,9-dimethylxanthene (10.0 mg, 0.017 mmol) in dioxane (3 mL) was heated under an argon atmosphere in the microwave to 130° C. for 45 min. The mixture was purified by silica gel chromatography using CH₂Cl₂/MeOH (with 10% ammonia) as eluent. The title compound was obtained as a white solid (66.5 mg, 59%).

MS ISP (m/e): 419.3 (100) [(M+H)⁺].

¹H NMR (CDCl₃, 300 MHz): δ (ppm)=7.58 (m, 1H), 7.34-7.33 (m, 1H), 7.18-7.00 (m, 5H), 6.91-6.87 (m, 1H), 6.83 (m, 1H), 6.61 (m, 1H), 4.22-4.02 (m, 3H), 3.81 (s, 3H), 2.38-1.91 (m, 4H), 2.29 (s, 3H).

### Example 2

### [5-(4-Fluoro-phenyl)-5,6,7,8-tetrahydro-[1,2,4]triazolo[1,5-a]pyridin-2-yl]-[3-methoxy-4-(4-methyl-imidazol-1-yl)-phenyl]-amine

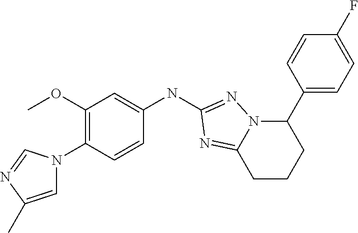

Prepared in analogy to example 1 steps a-e) starting from 2-amino-6-bromo-pyridine. The title compound was obtained as a white solid.

MS ISP (m/e): 419.3 (100) [(M+H)₊].

¹H NMR (CDCl₃, 300 MHz): δ (ppm)=7.61 (m, 1H), 7.35-7.34 (m, 1H), 7.09-7.04 (m, 5H), 6.81-6.76 (m, 2H), 6.59 (m, 1H), 5.32-5.28 (m, 1H), 3.63 (s, 3H), 2.99-2.95 (m, 2H), 2.47-2.37 (m, 1H), 2.29 (s, 3H), 2.14-1.88 (m, 3H).

### Example 3

### 5-(8-Methoxy-[1,2,4]triazolo[1,5-a]pyridin-2-ylamino)-2-(4-methyl-imidazol-1-yl)-benzonitrile

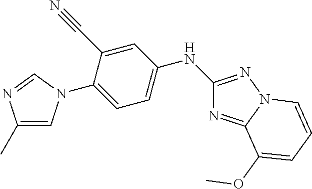

A suspension of 5-bromo-2-(4-methyl-imidazol-1-yl)-benzonitrile (WO2009103652; 100 mg, 0.38 mmol), 8-methoxy-[1,2,4]triazolo[1,5-a]pyridin-2-ylamine (Synthesis 2003, 1649; 63 mg, 0.38 mmol), Xanthpos (13 mg, 0.02 mmol), tris(dibenzylideneacetone)dipalladium chloroform complex (12 mg, 0.01 mmol) and sodium phenolate (132 mg, 1.14 mmol) in dioxane (5 mL) were heated under argon overnight at 85° C. The reaction mixture was cooled, diluted with water and extracted with ethyl acetate. The organic phase was dried, concentrated and the solid residue triturated 2 times with diethyl ether to yield the title compound as a yellowish solid (29 mg, 22%).

MS ISN (m/e): 344.3 (100) [(M−H)⁻].

¹H NMR (DMSO-D₆, 300 MHz): δ (ppm)=10.28 (s, 1H), 8.46 (d, 1H); 8.25 (d, 1H), 7.99 (dd, 1H), 7.88 (s, 1H), 7.58 (d, 1H); 7.25 (s, 1H), 7.09 (d, 1H); 7.00 (t, 1H), 3.99 (s, 3H), 2.19 (s, 3H).

### Example 4

### 2-(4-Methyl-imidazol-1-yl)-5-(7-methyl-5-propyl-[1,2,4]triazolo[1,5-c]pyrimidin-2-ylamino)-benzonitrile

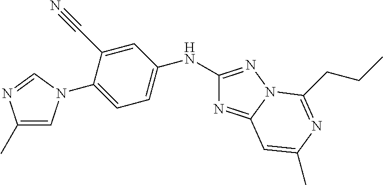

Prepared in analogy to example 3, starting from 5-bromo-2-(4-methyl-imidazol-1-yl)-benzonitrile (WO2009103652) and 7-methyl-5-propyl-[1,2,4]triazolo[1,5-c]pyrimidin-2-ylamine (J. Chem. Soc. 1965, 3357). The title compound was obtained as a colorless solid (yield: 47%).

MS ISP (m/e): 373.1 (100) [(M+H)⁺].

¹H NMR (DMSO-D₆, 300 MHz): δ (ppm)=10.49 (s, 1H), 8.28 (d, 1H), 8.01 (dd, 1H), 7.89 (d, 1H), 7.61 (d, 1H), 7.42 (s, 1H), 7.26 (s, 1H), 4.10-4.00 (m, 2H), 2.19 (s, 3H), 1.93 (s, 3H), 1.91 (m, 2H), 1.03 (t, 3H).

### Example 5

### 5-(8-Methoxy-5-phenyl-[1,2,4]triazolo[1,5-a]pyridin-2-ylamino)-2-(4-methyl-imidazol-1-yl)-benzonitrile

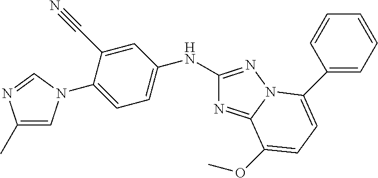

Prepared in analogy to example 3, starting from 5-bromo-2-(4-methyl-imidazol-1-yl)-benzonitrile (WO2009103652) and 8-methoxy-5-phenyl-[1,2,4]triazolo[1,5-a]pyridin-2-ylamine (Synthesis 2003, 1649)). The title compound was obtained as a slightly brown solid (yield: 10%).

MS ISP (m/e): 422.1 (100) [(M+H)⁺].

¹H NMR ((DMSO-D₆, 300 MHz): δ (ppm)=10.35 (s, 1H), 8.34 (d, 1H), 7.99 (dd, 2H), 7.93 (dd, 2H), 7.88 (d, 2H), 7.60-7.45 (m, 4H), 7.30-7.15 (m, 3H), 4.04 (s, 3H), 2.18 (s, 3H).

### Example 6

### [3-Methoxy-4-(4-methyl-imidazol-1-yl)-phenyl]-(5-phenyl-[1,2,4]triazolo[1,5-a]pyridin-2-yl)-amine

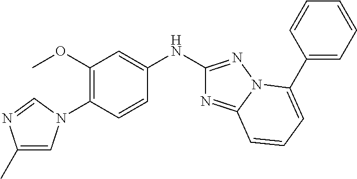

### a) 5-Phenyl-[1,2,4]triazolo[1,5-a]pyridin-2-ylamine

Prepared in analogy to example 1 steps b-c) starting from 2-amino-6-phenylpyridine. The title compound was obtained as a white solid.

MS ISP (m/e): 211.1 (100) [(M+H)⁺].

¹H NMR (DMSO-D₆, 300 MHz): δ (ppm)=7.94 (m, 2H), 7.57-7.48 (m, 4H), 7.37 (d, 1H), 7.01 (d, 1H), 6.02 (br s, 2H).

### b) 2-Bromo-5-phenyl-[1,2,4]triazolo[1,5-a]pyridine

To a solution of copper (II) bromide (231.7 mg, 1.4 mmol) and tert.-butylnitrite (137 μl, 1.04 mmol) in acetonitrile (3.2 mL) was added portion wise under nitrogen and stirring at 60° C. 5-phenyl-[1,2,4]triazolo[1,5-a]pyridin-2-ylamine (145.4 mg, 0.69 mmol). The reaction was heated 75° C. for 2 hours. After cooling to room temperature 1N aqueous HCl solution was added and the reaction was extracted three times with CH₂Cl₂. The combined organic layers were dried over sodium sulfate, filtered and the solvent was evaporated under reduced pressure. The residue was purified by silica gel chromatography using n-heptane/ethyl acetate (v/v 4:1 to 1:1) as eluent. The title compound was obtained as a light yellow solid (121 mg, 64%).

MS ISP (m/e): 274.2/276.2 (100/72) [(M+H)⁺].

¹H NMR (CDCl₃, 300 MHz): δ (ppm)=7.94-7.90 (m, 2H), 7.70-7.60 (m, 2H), 7.56-7.11 (m, 3H), 7.13 (d, 1H).

### c) [3-Methoxy-4-(4-methyl-imidazol-1-yl)-phenyl]-(5-phenyl-[1,2,4]triazolo[1,5-a]pyridin-2-yl)-amine

Palladium (II) acetat (3.6 mg, 0.016 mmol) and 2-(dicyclohexylphosphino)biphenyl (11.6 mg, 0.032 mmol) were dissolved under nitrogen in dioxane (3 mL) and stirred for 10 minutes. The solution decolorized partly. Sodium-tert.-butylate (29.4 mg, 0.3 mmol), 3-methoxy-4-(4-methylimidazole-1-yl)phenylamine (40.6 mg, 0.2 mmol) and 2-bromo-5-phenyl-[1,2,4]triazolo[1,5-a]pyridine (54.8 mg, 0.2 mmol) were added and the reaction was reacted at 160° C. for 60 minutes in a microwave oven. The reaction was poured onto water and extracted twice with EtOAc. The organic layer was washed with saturated aqueous NaCl solution, dried over sodium sulfate, filtered and the solvent was removed under reduced pressure. The residue was purified by silica gel chromatography using CH₂Cl₂/MeOH (v/v 19:1) as eluent. The crude product was stirred with acetonitrile, filtered and dried to yield the title compound as a light yellow solid (7 mg, 9%).

MS ISP (m/e): 397.3 (100) [(M+H)⁺].

¹H NMR (CDCl₃, 300 MHz): δ (ppm)=7.98 (m, 2H), 7.82 (s, 1H), 7.61 (s, 1H), 7.57-7.51 (m, 5H), 7.24 (d, 1H), 7.13 (d, 1H), 7.01 (d, 1H), 6.85 (s, 1H), 6.83 (d, 1H), 3.76 (s, 3H), 2.29 (s, 3H).

### Example 7

### [3-Methoxy-4-(4-methyl-imidazol-1-yl)-phenyl]-(8-methoxy-[1,2,4]triazolo[1,5-a]pyridin-2-yl)-amine

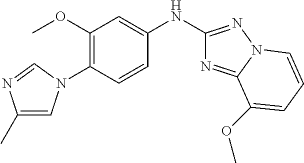

### a) 2-Bromo-8-methoxy-[1,2,4]triazolo[1,5-a]pyridine

Prepared in analogy to example 6b, starting from 8-methoxy-[1,2,4]triazolo[1,5-a]pyridin-2-ylamine. The title compound was obtained as a light yellow solid (yield: 84%).

MS ISP (m/e): 228.1/230.1 (100/92) [(M+H)⁺].
¹H NMR (DMSO-D₆, 300 MHz): δ (ppm)=8.53 (m, 1H), 7.17 (d, 1H), 3.99 (d, 3H).

### b) [3-Methoxy-4-(4-methyl-imidazol-1-yl)-phenyl]-(8-methoxy-[1,2,4]triazolo[1,5-a]pyridin-2-yl)-amine

Prepared in analogy to example 6c, starting from 2-bromo-8-methoxy-[1,2,4]triazolo[1,5-a]pyridine and 3-methoxy-4-(4-methylimidazole-1-yl)phenylamine. The title compound was obtained as a light grey solid (yield: 7%).

MS ISP (m/e): 351.4 (100) [(M+H)⁺], 336.4 (75).

¹H NMR (CDCl₃, 300 MHz): δ (ppm)=8.10 (d, 1H), 7.62 (s, 1H), 7.47 (s, 1H), 7.21 (d, 1H), 7.14 (d, 1H), 7.04 (s, 1H), 6.87-6.80 (m, 3H), 4.04 (s, 3H), 3.90 (s, 3H), 2.30 (s, 3H).

### Example 8

### [5-(4-Fluoro-phenyl)-[1,2,4]triazolo[1,5-a]pyridin-2-yl]-[3-methoxy-4-(4-methyl-imidazol-1-yl)-phenyl]-amine

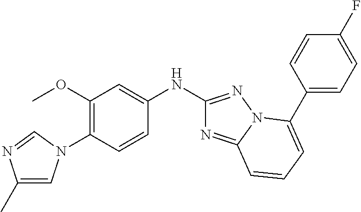

### a) 6-(4-Fluoro-phenyl)-pyridin-2-ylamine

2-Amino-6-chloropyridine (128.6 mg, 1 mmol), 4-fluorobenzeneboronic acid (216.4 mg, 1.5 mmol) and potassium phosphate tribasic (433.2 mg, 2 mmol) were suspended in dioxane (3.9 mL) The suspension was evacuated and flushed with nitrogene for 3 times. Palladium (II) acetat (11.2 mg, 0.05 mmol) and (D-t-BPF) (1,1′-bis(di-tert.-butylphosphino) ferrocene, (24.2 mg, 0.05 mmol) were added and again the suspension was evacuated and flushed with nitrogen twice. The reaction was heated to reflux for 4 hours. After cooling to room temperature the reaction was partitionated between water and EtOAc and extracted once with EtOAc. The combined organic layers were washed with 1N aqueous NaOH solution and once with saturated aqueous NaCl solution, dried over sodium sulfate, and the solvent was evaporated under reduced pressure. The residue was purified by silica gel chromatography using CH₂Cl₂/MeOH (v/v 19:1) as eluent to yield the title compound as a light brown oil (142 mg, 68%).

MS ISP (m/e): 189.4 (59) [(M+H)⁺], 173.2 (100).

¹H NMR (DMSO-D₆, 300 MHz): δ (ppm)=8.01 (m, 2H), 7.45 (t, 1H), 7.24 (t, 2H), 7.03 (d, 1H), 6.41 (d, 1H), 5.98 (br s, 2H).

b) N-(6-(4-Fluoro-phenyl)-pyridin-2-yl)-N′-ethoxycarbonyl-thiourea

Prepared in analogy to example 1b, starting from 6-(4-fluoro-phenyl)-pyridin-2-ylamine. The title compound was purified by stirring of the crude product in n-heptane/diethyl ether and was obtained after filtration and drying as a light brown solid (yield: 87%).

MS ISP (m/e): 320.2 (100) [(M+H)⁺], 274.2 (39), 172.3 (38).

¹H NMR (DMSO-D₆, 300 MHz): δ (ppm)=8.10 (m, 2H), 7.97 (t, 1H), 7.81 (d, 1H), 7.35 (t, 2H), 4.24 (q, 2H), 1.29 (t, 3H).

### c) 5-(4-Fluoro-phenyl)-[1,2,4]triazolo[1,5-a]pyridin-2-ylamine

Prepared in analogy to example 1c, starting from N-(6-(4-fluoro-phenyl)-pyridin-2-yl)-N′-ethoxycarbonyl-thiourea. The title compound was purified by stirring the crude product with water, filtration and washing with MeOH/Et₂O 4:1 and then with Et₂O. The title compound was obtained after drying as a light brown solid (yield: 84%).

MS ISP (m/e): 229.4 (100) [(M+H)⁺].

¹H NMR (DMSO-D₆, 300 MHz): δ (ppm)=8.02 (m, 2H), 7.51 (dd, 1H), 7.38 (t, 3H), 7.02 (d, 1H), 6.04 (br s, 2H).

### d) 2-Bromo-5-(4-fluoro-phenyl)-[1,2,4]triazolo[1,5-a]pyridine

Prepared in analogy to example 6b, starting from 5-(4-fluoro-phenyl)-[1,2,4]triazolo[1,5-a]pyridin-2-ylamine. The title compound was obtained as a light yellow solid after stirring with diethyl ether, filtration and drying (yield: 67%).

MS ISP (m/e): 294.1/292.2 (68/100) [(M+H)⁺].

¹H NMR (DMSO-D₆, 300 MHz): δ (ppm)=8.02 (dd, 2H), 7.85-7.83 (m, 2H), 7.47-7.40 (m, 3H).

### e) [5-(4-Fluoro-phenyl)-[1,2,4]triazolo[1,5-a]pyridin-2-yl]-[3-methoxy-4-(4-methyl-imidazol-1-yl)-phenyl]-amine

To a solution of 3-methoxy-4-(4-methyl-imidazol-1-yl)-phenylamine (122 mg, 0.6 mmol), 2-bromo-5-(4-fluoro-phenyl)-[1,2,4]triazolo[1,5-a]pyridine (196 mg, 0.67 mmol) and sodium tert.-butoxide (87 mg, 0.90 mmol) in dioxane (3 mL) was added under nitrogen a dark solution of palladium acetate (6.7 mg, 0.03 mmol) and 1,1-bis(ditertbutylphosphino)ferrocene (D-tBPF, 14.2 mg, 0.03 mmol) in dioxane (1 mL). The reaction was heated to 150° C. in a microwave oven for 1 hour. It was partitionated between ethyl acetate and water and extracted with ethyl acetate. The combined organic layers were washed with saturated aqueous sodium chloride solution, dried over sodium sulfate, filtered and the solvent was evaporated under reduced pressure. The residue was purified by column chromatography on silica gel using CH₂Cl₂/MeOH (v/v=1/19) as eluent. A 1:1 mixture of product and aniline was obtained. The mixture was taken up in methylene chloride and diethyl ether and stirred for 15 minutes. The title compound was filtered off, washed with diethyl ether, dried and was obtained as a white solid (50 mg, 20%).

MS ISP (m/e)=415.3 (100) [(M+H)⁺].

¹H NMR (DMSO-D₆, 300 MHz): δ (ppm)=9.90 (s, 1H), 8.17 (m, 2H), 7.78 (s, 1H), 7.59-7.71 (m, 3H), 7.42 (t, 2H), 7.15-7.26 (m, 3H), 7.01 (s, 1H), 3.75 (s, 1H), 2.14 (s, 1H).

### Example 9

### [5-(4-Fluoro-phenyl)-[1,2,4]triazolo[1,5-a]pyrazin-2-yl]-[3-methoxy-4-(4-methyl-imidazol-1-yl)-phenyl]-amine

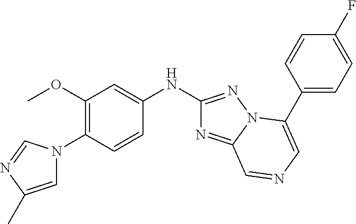

### a) 6-(4-Fluoro-phenyl)-pyrazin-2-ylamine

Prepared in analogy to example 8a, starting from 2-amino-6-chloropyrazine and 4-fluorobenzeneboronic acid. The title compound was obtained as a slightly brown solid (yield: 91%).

MS ISP (m/e): 190.3 (100) [(M+H)⁺].

¹H NMR (DMSO-D₆, 300 MHz): δ (ppm)=8.27 (s, 1H), 8.04 (dd, 2H), 7.84 (s, 1H), 7.30 (t, 2H), 6.52 (br s, 2H).

b) N-(6-(4-Fluoro-phenyl)-pyrazin-2-yl)-N′-ethoxycarbonyl-thiourea

Prepared in analogy to example 1b, starting from 6-(4-fluoro-phenyl)-pyrazin-2-ylamine.

The title compound precipitated from the reaction, was filtered and washed with n-heptane, dried and was obtained as white crystals (yield: 80%).

MS ISP (m/e): 321.2 (100) [(M+H)⁺], 232.2 (34), 275.2 (25).

¹H NMR (DMSO-D₆, 300 MHz): δ (ppm)=12.18 (br s, 1H), 11.92 (br s, 1H), 9.56 (br s, 1H), 9.10 (s, 1H), 8.19 (dd, 2H), 7.40 (t, 2H), 4.25 (q, 2H), 1.28 (t, 3H).

### c) 5-(4-Fluoro-phenyl)-[1,2,4]triazolo[1,5-a]pyrazin-2-ylamine

Prepared in analogy to example 1c, starting from N-(6-(4-fluoro-phenyl)-pyrazin-2-yl)-N′-ethoxycarbonyl-thiourea. The reaction was diluted with water and the title compound was filtered and washed with MeOH/Et₂O 4:1 and then with Et₂O. The product was purified by column chromatography on silica gel using CH₂Cl₂/MeOH (v/v=19:1) as eluent to yield the title compound as white crystals (yield: 73%).

MS ISP (m/e):230.3 (100) [(M+H)⁺].

¹H NMR (DMSO-D₆, 300 MHz): δ (ppm)=8.83 (s, 1H), 8.19 (s, 1H), 8.12 (dd, 2H), 7.43 (t, 2H), 6.54 (br s, 2H).

### d) [5-(4-Fluoro-phenyl)-[1,2,4]triazolo[1,5-a]pyrazin-2-yl]-[3-methoxy-4-(4-methyl-imidazol-1-yl)-phenyl]-amine

Prepared in analogy to example 8e, starting from 5-(4-fluoro-phenyl)-[1,2,4]triazolo[1,5-a]pyrazin-2-ylamine and 1-(4-bromo-2-methoxy-phenyl)-4-methyl-1H-imidazole. The reaction was diluted with water and EtOAc. The title compound precipitated, was filtered and washed with water and EtOAc, dried, and was obtained as a light brown solid (yield: 70%).

MS ISP (m/e): 416.4 (100) [(M+H)⁺].

¹H NMR (DMSO-D₆, 300 MHz): δ (ppm)=8.10 (s, 1H), 8.37 (s, 1H), 8.22 (m, 2H), 7.77 (s, 1H), 7.65 (s, 1H), 7.47 (m, 2H), 7.26 (m, 2H), 7.02 (s, 1H), 3.79 (s, 3H), 2.14 (s, 3H).

### Example 10

### [8-(4-Fluoro-phenyl)-[1,2,4]triazolo[1,5-a]pyridin-2-yl]-[3-methoxy-4-(4-methyl-imidazol-1-yl)-phenyl]-amine

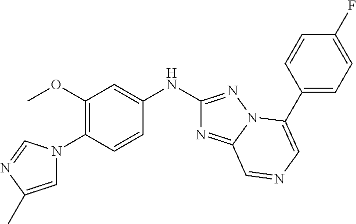

Prepared in analogy to example 8e, starting from 8-(4-fluoro-phenyl)-[1,2,4]triazolo[1,5-a]pyridin-2-ylamine and 1-(4-bromo-2-methoxy-phenyl)-4-methyl-1H-imidazole. After workup the crude product was stirred in CH₂Cl₂ and the title compound was filtered and washed with little Et₂O, dried, and was obtained as a yellow solid (yield: 35%). Chromatography of the mother liqueurs yield another batch (35% yield).

MS ISP (m/e): 415.3 (100) [(M+H)⁺].

¹H NMR (DMSO-D₆, 300 MHz): δ (ppm)=10.00 (s, 1H), 8.81 (d, 1H), 8.23 (m, 2H), 7.87 (d, 1H), 7.82 (s, 1H), 7.65 (s, 1H), 7.37 (t, 2H), 7.27 (m, 2H), 7.16 (t, 1H), 7.03 (s, 1H), 3.84 (s, 3H), 2.15 (s, 3H).

### Example 11

### [8-(4-Fluoro-phenyl)-[1,2,4]triazolo[1,5-a]pyrazin-2-yl]-[3-methoxy-4-(4-methyl-imidazol-1-yl)-phenyl]-amine

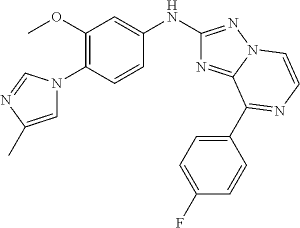

### a) 3-(4-Fluoro-phenyl)-pyrazin-2-ylamine

Prepared in analogy to example 8a, starting from 2-amino-3-chloropyrazine and 4-fluorobenzeneboronic acid. The title compound was obtained as brown solid (yield: 96%).

MS ISP (m/e): 190.3 (100) [(M+H)⁺], 173.2 (37).

¹H NMR (DMSO-D₆, 300 MHz): δ (ppm)=7.94 (s, 1H), 7.87 (s, 1H), 7.72 (m, 2H), 7.30 (t, 2H), 6.16 (br s, 2H).

b) N-(3-(4-Fluoro-phenyl)-pyrazin-2-yl)-N′-ethoxycarbonyl-thiourea

Prepared in analogy to example 1b, starting from 3-(4-fluoro-phenyl)-pyrazin-2-ylamine. The title compound was purified by column chromatography on silica gel using Et₂O/n-heptane 9:1 and was obtained as off-white crystals (yield: 97%).

MS ISP (m/e): 321.2 (100) [(M+H)⁺].

¹H NMR (DMSO-D₆, 300 MHz): δ (ppm)=11.62 (s, 1H), 11.28 (s, 1H), 8.70 (s, 1H), 8.57 (s, 1H), 7.82 (m, 2H), 7.29 (t, 2H), 4.21 (q, 2H), 1.25 (t, 3H).

### c) 8-(4-Fluoro-phenyl)-[1,2,4]triazolo[1,5-a]pyrazin-2-ylamine

Prepared in analogy to example 1c, starting from N-(3-(4-fluoro-phenyl)-pyrazin-2-yl)-N′-ethoxycarbonyl-thiourea. The reaction was diluted with water and the title compound was filtered and washed with MeOH:Et₂O 4:1 and then with Et₂O. The product was purified by column chromatography on silica gel using CH₂Cl₂/MeOH (v/v=19:1) as eluent to yield the title compound as white crystals (yield: 75%).

MS ISP (m/e): 230.3 (100) [(M+H)⁺].

¹H NMR (DMSO-D₆, 300 MHz): δ (ppm)=8.76 (m, 2H), 8.68 (d, 1H), 8.09 (d, 1H), 7.40 (t, 2H), 6.61 (br s, 2H).

### d) [8-(4-Fluoro-phenyl)-[1,2,4]-triazolo[1,5-a]pyrazin-2-yl]-[3-methoxy-4-(4-methyl-imidazol-1-yl)-phenyl]-amine

Prepared in analogy to example 8e, starting from 8-(4-fluoro-phenyl)-[1,2,4]triazolo[1,5-a]pyrazin-2-ylamine and 1-(4-bromo-2-methoxy-phenyl)-4-methyl-1H-imidazole. The reaction was diluted with water and EtOAc. The title compound precipitated, was filtered and washed with water and EtOAc, dried, and was obtained as a brown solid (yield: 88%).

MS ISP (m/e): 416.2 (100) [(M+H)⁺].

¹H NMR (DMSO-D₆, 300 MHz): δ (ppm)=10.33 (br s, 1H), 8.94 (br s, 1H), 8.82 (m, 2H), 8.27 (br s, 1H), 7.84 (s, 1H), 7.67 (s, 1H), 7.44 (t, 2H), 7.30 (s, 2H), 7.05 (s, 1H), 3.88 (s, 3H), 2.16 (s, 3H).

### Example 12

### [3-Methoxy-4-(4-methyl-imidazol-1-yl)-phenyl]-(5-pyrrolidin-1-yl-[1,2,4]triazolo[1,5-a]pyridin-2-yl)-amine

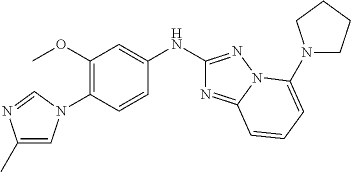

### a) 6-Pyrrolidin-1-yl-pyridin-2-ylamine

To a solution of 2-amino-6-bromopyridine (1.76 g, 10 mmol) in NMP (10 mL) was added at room temperature piperidine (1.65 mL, 20 mmol) and Cs₂CO₃ (4.89 g, 15 mmol). The reaction was heated to 200° C. for 30 minutes in a microwave oven. It was poured onto water, extracted three times with diethyl ether. The combined organic layers were washed with water, dried over sodium sulfate, filtered and the solvent was removed under reduced pressure. The residue was purified by column chromatography on silica gel with Et₂O. The title compound was obtained as a brown solid (1.48 g, 90%).

¹H NMR (DMSO-D₆, 300 MHz) δ (ppm)=7.09 (t, 1H), 5.65 (d, 1H), 5.54 (d, 1H), 5.37 (br s, 2H), 3.27 (m, 4H), 1.88 (m, 4H).

### b) N-(6-(Pyrrolidin-1-yl)-pyridin-2-yl)-N′-ethoxycarbonyl-thiourea

Prepared in analogy to example 1b, starting from 6-pyrrolidin-1-yl-pyridin-2-ylamine.

The title compound precipitated from the reaction, was filtered, washed with n-heptane and was obtained as yellow crystals (yield: 88%).

MS ISP (m/e): 295.3 (83) [(M+H)⁺], 249.2 (72), 206.2 (100).

¹H NMR (DMSO-D₆, 300 MHz): δ (ppm)=7.51 (t, 1H), 6.48-6.12 (br m, 2H), 4.16 (br m, 2H), 3.38 (br m, 4H), 1.95 (br s, 4H), 1.23 (t, 3H).

### c) 5-Pyrrolidin-1-yl-[1,2,4]triazolo[1,5-a]pyridin-2-ylamine

Prepared in analogy to example 1c, starting from N-(6-(pyrrolidin-1-yl)-pyridin-2-yl)-N′-ethoxycarbonyl-thiourea. The solvent was removed under reduced pressure and the residue was stirred with water, filtered and washed with MeOH/Et₂O 4:1 and then with Et₂O. The product was purified by column chromatography on silica gel using CH₂Cl₂/MeOH (v/v 19:1) as eluent to yield the title compound as off-white crystals (yield: 78%).

MS ISP (m/e): 204.3 (100) [(M+H)⁺].

¹H NMR (DMSO-D₆, 300 MHz): δ (ppm)=7.27 (t, 1H), 6.64 (d, 1H), 5.97 (d, 1H), 5.71 (br s, 2H), 3.64 (m, 4H), 1.92 (m, 4H).

### d) [3-Methoxy-4-(4-methyl-imidazol-1-yl)-phenyl]-(5-pyrrolidin-1-yl-[1,2,4]triazolo[1,5-a]pyridin-2-yl)-amine

Prepared in analogy to example 8e, starting from 5-pyrrolidin-1-yl-[1,2,4]triazolo[1,5-a]pyridin-2-ylamine and 1-(4-bromo-2-methoxy-phenyl)-4-methyl-1H-imidazole. The title compound was obtained as a brown solid (yield: 53%) after column chromatography on silica gel using a gradient from CH₂Cl₂ to CH₂Cl₂/MeOH 19:1 (v/v) as eluent.

MS ISP (m/e): 390.3 (100) [(M+H)⁺].

¹H NMR (DMSO-D₆, 300 MHz): δ (ppm)=9.65 (s, 1H), 7.74 (s, 1H), 7.64 (s, 1H), 7.39 (t, 1H), 7.22 (d, 1H), 7.19 (d, 1H), 7.02 (s, 1H), 6.81 (d, 1H), 6.09 (d, 1H), 3.82 (m, 7H), 2.14 (s, 3H), 1.97 (m, 4H).

### Example 13

### [3-Methoxy-4-(4-methyl-imidazol-1-yl)-phenyl]-(5-phenyl-[1,2,4]triazolo[1,5-a]pyrazin-2-yl)-amine

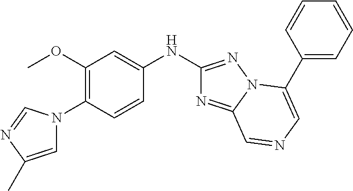

Prepared in analogy to example 9b)-d), starting from 6-phenyl-pyrazin-2-ylamine. The title compound was obtained as a light yellow solid (yield: 76%).

MS ISP (m/e): 398.3 (100) [(M+H)⁺].

¹H NMR (DMSO-D₆, 300 MHz): δ (ppm)=10.25 (s, 1H), 9.10 (s, 1H), 8.16 (s, 1H), 8.16 (m, 2H), 7.82 (s, 1H), 7.65-7.60 (m, 4H), 7.26 (d, 1H), 7.21 (d, 1H), 7.02 (s, 1H), 3.78 (s, 3H), 2.14 (s, 3H).

### Example 14

### (5,7-Dimethyl-[1,2,4]triazolo[1,5-a]pyridin-2-yl)-[3-methoxy-4-(4-methyl-imidazol-1-yl)-phenyl]-amine

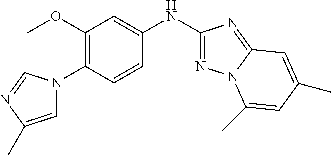

### a) 5,7-Dimethyl-[1,2,4]triazolo[1,5-a]pyridin-2-ylamine

Prepared in analogy to example 1b-c, starting from 2-amino-4,6-dimethylpyridine. The title compound was purified by column chromatography on silica gel using a gradient from CH₂Cl₂ to CH₂Cl₂/MeOH (19:1 v/v) as eluent as an offwhite solid (yield: 50% over 2 steps).

¹H NMR (DMSO-D₆, 300 MHz): δ (ppm)=7.01 (s, 1H), 6.59 (s, 1H), 5.87 (br s, 2H), 2.50 (s, 3H), 2.32 (s, 3H).

### b) (5,7-Dimethyl-[1,2,4]triazolo[1,5-a]pyridin-2-yl)-[3-methoxy-4-(4-methyl-imidazol-1-yl)-phenyl]-amine

Prepared in analogy to example 8e, starting from 5,7-dimethyl-[1,2,4]triazolo[1,5-a]pyridin-2-ylamine and 1-(4-bromo-2-methoxy-phenyl)-4-methyl-1H-imidazole. The title compound was obtained as a light yellow solid (yield: 56%) after column chromatography on silica gel using a gradient from CH₂Cl₂ to CH₂Cl₂/MeOH 19:1 (v/v) as eluent.

MS ISP (m/e): 349.3 (100) [(M+H)⁺].

¹H NMR (DMSO-D₆, 300 MHz): δ (ppm)=9.79 (s, 1H), 7.79 (s, 1H), 7.64 (s, 1H), 7.28-7.21 (m 3H9, 7.02 (s, 1H), 6.80 (s, 1H), 3.83 (s, 3H), 2.67 (s, 3H), 2.39 (s, 3H), 2.15 (s, 3H).

### Example 15

### (7-tert-Butyl-5-methyl-[1,2,4]triazolo[1,5-a]pyrimidin-2-yl)-[3-methoxy-4-(4-methyl-imidazol-1-yl)-phenyl]-amine acetate

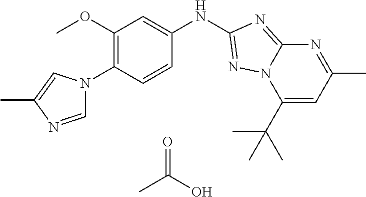

### a) N-(3-ethoxy-4-(4-methyl-imidazol-1-yl)-N′-cyano-O-phenylisourea

3-Methoxy-4-(4-methyl-imidazol-1-yl)-phenylamine (1016 mg, 5.0 mmol) and N-cyanodiphenoxyimidocarbonate (1191 mg, 5.0 mmol) were suspended in 2-propanol (10 mL). The suspension was stirred at room temperature for 2 hours. The precipitate was filtered and washed with 2-propanol and dried to yield the title compound as a light yellow solid (1.29 g, 74%). The compound was used directly in the next step without further purification.

### b) N3-[3-Methoxy-4-(4-methyl-imidazol-1-yl)-phenyl]-1H-[1,2,4]triazole-3,5-diamine

N-(3-Ethoxy-4-(4-methyl-imidazol-1-yl)-N′-cyano-O-phenylisourea (695 mg, 2 mmol) was suspended in methanol (5 mL) and hydrazine hydrate (100 mg, 2 mmol) was added. After a few minutes a white precipitate was formed. The suspension was stirred at room temperature for 1 hour. The solid was filtered and washed twice with isopropanol and dried in vacuo to yield the title compound as a white solid (550 mg, 96%).

MS ISP (m/e): 286.3 (100) [(M+H)⁺].

¹H NMR (DMSO-D₆, 300 MHz): δ (ppm)=11.22 (s, 1H), 8.85 (s, 1H), 7.58 (s, 1H), 7.48 (s, 1H), 7.11 (m 2H), 6.99 (s, 1H), 5.90 (br s, 2H), 3.74 (s, 3H), 2.13 (s, 3H).

### c) (7-tert-Butyl-5-methyl-[1,2,4]triazolo[1,5-a]pyrimidin-2-yl)-[3-methoxy-4-(4-methyl-imidazol-1-yl)-phenyl]-amine acetate

A solution of N3-[3-methoxy-4-(4-methyl-imidazol-1-yl)-phenyl]-1H-[1,2,4]triazole-3,5-diamine (57 mg, 0.2 mmol) and 2,2-dimethyl-3,5-hexanedione (31 mg, 0.22 mmol) in acetic acid (1 mL) was heated to 100° C. over night. The solvent was evaporated and the residue treated with diethyl ether. The title compound precipitated, was filtered, washed with diethyl ether, dried to yield a white solid (40.1 mg, 44%).

MS ISP (m/e): 392.3 (100) [(M+H)⁺].

¹H NMR (DMSO-D₆, 300 MHz): δ (ppm)=11.95 (br s, 1H), 10.06 (s, 1H), 7.92 (s, 1H), 7.67 (s, 1H), 7.26 (d, 1H), 7.15 (d, 1H), 7.04 (s, 1H), 6.98 (s, 1H), 3.85 (s, 3H), 2.57 (s, 3H), 2.15 (s, 3H), 1.91 (s, 3H), 1.60 (s, 9H).

### Example 16

### [3-Methoxy-4-(4-methyl-imidazol-1-yl)-phenyl]-(5-propyl-[1,2,4]triazolo[1,5-a]pyridin-2-yl)-amine

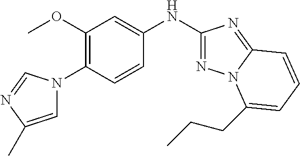

### a) 5-Propyl-[1,2,4]triazolo[1,5-a]pyridin-2-ylamine

Prepared in analogy to example 1b-c, starting from 2-amino-6-propylpyridine. The title compound was purified by column chromatography on silica gel using a gradient from CH₂Cl₂ to CH₂Cl₂/MeOH 19:1 (v/v) as eluent as an off-white solid (yield: 80% over 2 steps).

¹H NMR (DMSO-D₆, 300 MHz): δ (ppm)=7.36 (t, 1H), 7.21 (d, 1H), 6.73 (d, 1H), 5.98 (br s, 2H), 2.93 (t, 2H), 1.76 (sext, 2H), 0.95 (t, 3H).

### b) [3-Methoxy-4-(4-methyl-imidazol-1-yl)-phenyl]-(5-propyl-[1,2,4]triazolo[1,5-a]pyridin-2-yl)-amine

Prepared in analogy to example 8e, starting from 5-propyl-[1,2,4]triazolo[1,5-a]pyridin-2-ylamine and 1-(4-bromo-2-methoxy-phenyl)-4-methyl-1H-imidazole. The title compound was obtained as a light yellow solid (yield: 69%) after column chromatography on silica gel using a gradient from CH₂Cl₂ to CH₂Cl₂/MeOH 19:1 (v/v) as eluent.

MS ISP (m/e): 363.3 (100) [(M+H)⁺].

¹H NMR (DMSO-D₆, 300 MHz): δ (ppm)=9.86 (s, 1H), 7.85 (s, 1H), 7.65 (s, 1H), 7.54 (t, 1H), 7.46 (d, 1H), 7.25 (s, 2H), 7.03 (s, 1H), 6.92 (d, 1H), 3.84 (s, 3H), 3.08 (t, 2H), 2.15 (s, 3H), 1.87 (sext, 2H), 0.98 (t, 3H).

### Example 17

### [3-Methoxy-4-(4-methyl-imidazol-1-yl)-phenyl]-(5-trifluoromethyl-[1,2,4]triazolo[1,5-a]pyridin-2-yl)-amine

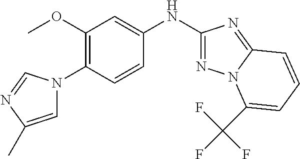

### a) 5-Trifluoromethyl-[1,2,4]triazolo[1,5-a]pyridin-2-ylamine

Prepared in analogy to example 1b-c, starting from 2-amino-6-trifluoromethylpyridine. The title compound was purified by column chromatography on silica gel using a gradient from CH₂Cl₂ to CH₂Cl₂/MeOH 19:1 (v/v) as eluent as an off-white solid (yield: 55% over 2 steps).

¹H NMR (DMSO-D₆, 300 MHz): δ (ppm)=7.69 (d, 1H), 7.58 (t, 1H), 7.45 (d, 1H), 6.42 (br s, 2H).

### b) [3-Methoxy-4-(4-methyl-imidazol-1-yl)-phenyl]-(5-trifluoromethyl-[1,2,4]triazolo[1,5-a]pyridin-2-yl)-amine

Prepared in analogy to example 8e, starting from 5-trifluoromethyl-[1,2,4]triazolo[1,5-a]pyridin-2-ylamine and 1-(4-bromo-2-methoxy-phenyl)-4-methyl-1H-imidazole. The title compound was obtained as a light yellow solid (yield: 72%) after column chromatography on silica gel using a gradient from CH₂Cl₂ to CH₂Cl₂/MeOH 19:1 (v/v) as eluent.

MS ISP (m/e): 389.2 (100) [(M+H)⁺].

¹H NMR (DMSO-D₆, 300 MHz): δ (ppm)=10.17 (s, 1H), 7.96 (d, 1H), 7.81 (s, 1H), 7.75 (t, 1H), 7.66 (s, 1H), 7.65 (d, 1H), 7.27 (m, 2H), 7.04 (s, +H), 3.83 (s, 3H), 2.15 (s, 3H).

### Example 18

### [3-Methoxy-4-(4-methyl-imidazol-1-yl)-phenyl]-(5-methyl-7-phenyl-[1,2,4]triazolo[1,5-a]pyrimidin-2-yl)-amine acetate

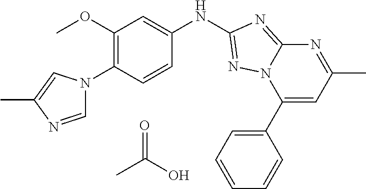

Prepared in analogy to example 15c, starting from N3-[3-methoxy-4-(4-methyl-imidazol-1-yl)-phenyl]-1H-[1,2,4]triazole-3,5-diamine and 1-benzoylacetone. The title compound was precipitated from diethyl ether and obtained after drying as a yellow solid (yield: 22%).

MS ISP (m/e): 412.3 (100) [(M+H)⁺].

¹H NMR (DMSO-D₆, 300 MHz): δ (ppm)=11.96 (br s, 1H), 10.10 (s, 1H), 8.24 (br d, 2H), 7.89 (s, 1H), 7.70 (d, 1H), 7.69-7.56 (m, 3H), 7.38 (s, 1H), 7.22 (d, 1H), 7.12 (d, 1H), 7.02 (s, 1H), 3.78 (s, 3H), 2.14 (s, 1H), 1.91 (s, 3H).

### Example 19

### 7-(3-Difluoromethoxy-phenyl)-2-[3-methoxy-4-(4-methyl-imidazol-1-yl)-phenylamino]-[1,2,4]triazolo[1,5-a]pyrimidine-5-carboxylic acid methyl ester acetate

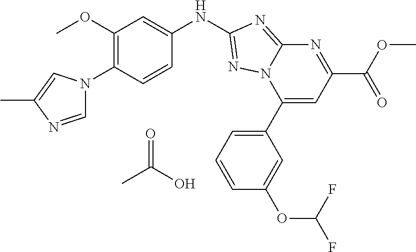

Prepared in analogy to example 15c, starting from N3-[3-methoxy-4-(4-methyl-imidazol-1-yl)-phenyl]-1H-[1,2,4]triazole-3,5-diamine and 4-(3-difluoromethoxy-phenyl)-2,4-dioxo-butyric acid methyl ester. The title compound was precipitated from diethyl ether and obtained after drying as an orange solid (yield: 83%).

MS ISP (m/e): 522.2 (100) [(M+H)⁺].

### Example 20

### [7-(4-Chloro-phenyl)-5-trifluoromethyl-[1,2,4]triazolo[1,5-a]pyrimidin-2-yl]-[3-methoxy-4-(4-methyl-imidazol-1-yl)-phenyl]-amine acetate

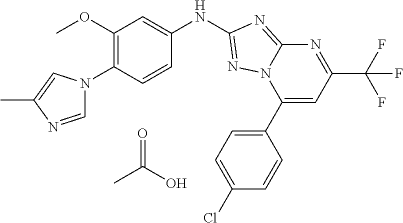

Prepared in analogy to example 15c, starting from N3-[3-methoxy-4-(4-methyl-imidazol-1-yl)-phenyl]-1H-[1,2,4]triazole-3,5-diamine and 1-(4-chlorophenyl)-4,4,4-trifluoro-1,3-butadione. The title compound was precipitated from diethyl ether and obtained after drying as a yellow solid (yield: 56%).

MS ISP (m/e): 500.2/502.2 (100/47) [(M+H)⁺].
¹H NMR (DMSO-D₆, 300 MHz): δ (ppm)=11.95 (br s, 1H), 8.41 (d, 2H), 8.31 (s, 1H), 7.83 (s, 1H), 7.73 (s, 1H), 7.68 (d, 2H), 7.32 (s, 1H), 7.08 (s, 1H), 3.84 (s, 3H), 2.16 (s, 3H), 1.91 (s, 3H).

### Example 21

### [3-Methoxy-4-(4-methyl-imidazol-1-yl)-phenyl]-(9-phenyl-5,6,7,8-tetrahydro-[1,2,4]triazolo[5,1-b]quinazolin-2-yl)-amine acetate

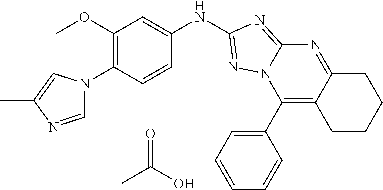

Prepared in analogy to example 15c, starting from N3-[3-methoxy-4-(4-methyl-imidazol-1-yl)-phenyl]-1H-[1,2,4]triazole-3,5-diamine and 2-benzoylcyclohexanone. The title compound was precipitated from diethyl ether and obtained after drying as a light yellow solid (yield: 60%).

MS ISP (m/e): 452.2 (100) [(M+H)⁺].

¹H NMR (DMSO-D₆, 300 MHz): δ (ppm)=11.94 (br s, 1H), 9.98 (s, 1H), 7.77 (s, 1H), 7.69-7.50 (m, 5H), 7.16 (d, 1H), 6.98 (m, 2H), 3.57 (s, 3H), 2.99 (t, 2H), 2.60 (m, 2H), 2.13 (s, 3H), 1.91 (s, 3H), 1.90 (m, 2H), 1.72 (m, 2H).

### Example 22

### [5-(4-Fluoro-phenyl)-8-methoxy-[1,2,4]triazolo[1,5-a]pyridin-2-yl]-[3-methoxy-4-(4-methyl-imidazol-1-yl)-phenyl]-amine

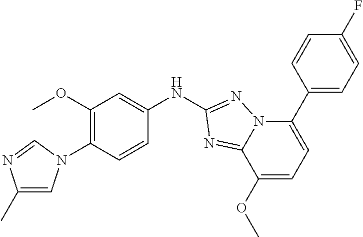

Prepared in analogy to example 8e, starting from 3-methoxy-4-(4-methyl-imidazol-1-yl)-phenylbromide and 5-(4-fluoro-phenyl)-8-methoxy-[1,2,4]triazolo[1,5-a]pyridin-2-ylamine. The title compound was obtained as a light brown solid (yield: 78%) after column chromatography on silica gel using CH₂Cl₂/MeOH 19:1 (v/v) as eluent and stirring with diethyl ether.

MS ISP (m/e): 445.3 (100) [(M+H)⁺].

¹H NMR (DMSO-D₆, 300 MHz): δ (ppm)=9.92 (s, 1H), 8.05 (dd, 2H), 7.74 (s, 1), 7.63 (s, 1H), 7.38 (t, 2H), 7.24 (d, 1H), 7.24-7.11 (m, 4H), 7.00 (s, 1H), 4.02 (s, 3H), 3.74 (s, 3H), 2.14 (s, 3H).

### Example 23

### [3-Methoxy-4-(4-methyl-imidazol-1-yl)-phenyl]-[7-(4-trifluoromethoxy-phenyl)-[1,2,4]triazolo[1,5-a]pyrimidin-2-yl]-amine acetate

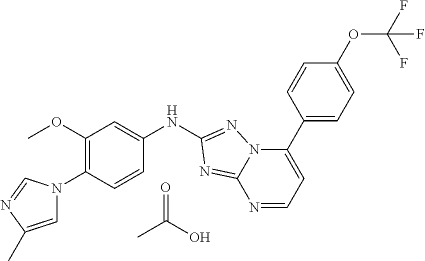

Prepared in analogy to example 15c, starting from N3-[3-methoxy-4-(4-methyl-imidazol-1-yl)-phenyl]-1H-[1,2,4]triazole-3,5-diamine and (E)-3-dimethylamino-1-(4-trifluoromethoxy-phenyl)-propenone. The title compound was precipitated from diethyl ether and obtained after drying as a yellow solid (yield: 61%).

MS ISP (m/e): 482.2 (100) [(M+H)⁺].

¹H NMR (DMSO-D₆, 300 MHz): δ (ppm)=10.21 (s, 1H), 8.76 (d, 1H), 8.36 (d, 2H), 7.74 (s, 1H), 7.69-7.60 (m, 3H), 7.47 (d, 1H), 7.28 (d, 1H), 7.15 (d, 1H), 7.02 (s, 1H), 3.76 (s, 1H), 2.14 (s, 3H), 1.90 (3H).

### Example 24

### (7-Furan-2-yl-5-trifluoromethyl-[1,2,4]triazolo[1,5-a]pyrimidin-2-yl)-[3-methoxy-4-(4-methyl-imidazol-1-yl)-phenyl]-amine

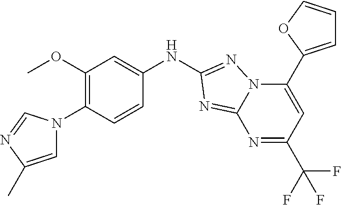

Prepared in analogy to example 15c, starting from N3-[3-methoxy-4-(4-methyl-imidazol-1-yl)-phenyl]-1H-[1,2,4]triazole-3,5-diamine and 4,4,4-trifluoro-1-(2-furyl)-butane-1,3-dione. The title compound was precipitated from diethyl ether and obtained after drying as a yellow solid (yield: 47%).

MS ISP (m/e): 456.2 (100) [(M+H)⁺].
¹H NMR (DMSO-D₆, 300 MHz): δ (ppm)=10.4.2 (s, 1H), 8.10 (s, 1H), 7.96 (s, 1H), 7.84 (s, 1H), 7.74 (d, 1H), 7.69 (s, 1H), 7.29 (m, 2H), 7.06 (s, 1H), 6.84 (s, 1H), 3.83 (s, 3H), 2.15 (s, 3H).

### Example 25

### (5,7-Diisopropyl-[1,2,4]triazolo[1,5-a]pyrimidin-2-yl)-[3-methoxy-4-(4-methyl-imidazol-1-yl)-phenyl]-amine acetate

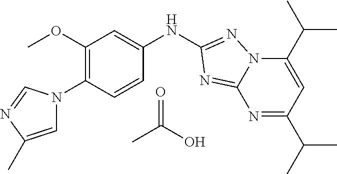

Prepared in analogy to example 15c, starting from N3-[3-methoxy-4-(4-methyl-imidazol-1-yl)-phenyl]-1H-[1,2,4]triazole-3,5-diamine and 2,6-dimethyl-3,5-heptanedione. The title compound was precipitated from diethyl ether and obtained after drying as a yellow solid (yield: 53%).

MS ISP (m/e): 406.4 (100) [(M+H)⁺].

¹H NMR (DMSO-D₆, 300 MHz): δ (ppm)=11.95 (br s, 1H), 10.02 (s, 1H), 7.82 (s, 1H), 7.66 (s, 1H), 7.27 (s, 2H), 7.03 (s, 2H), 3.83 (s, 3H), 3.61 (sept, 1H), 3.12 (sept, 1H), 2.15 (s, 3H), 1.91 (s, 3H), 1.42 (d, 6H), 1.29 (d, 2H).

### Example 26

### (5,7-Bis-trifluoromethyl-[1,2,4]triazolo[1,5-a]pyrimidin-2-yl)-[3-methoxy-4-(4-methyl-imidazol-1-yl)-phenyl]-amine

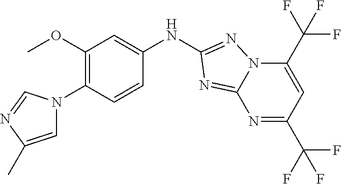

Prepared in analogy to example 15c, starting from N3-[3-methoxy-4-(4-methyl-imidazol-1-yl)-phenyl]-1H-[1,2,4]triazole-3,5-diamine and hexafluoroacetylacetone. The title compound was precipitated from diethyl ether and obtained after drying as a yellow solid (yield: 14%).

MS ISP (m/e): 458.3 (100) [(M+H)⁺].

¹H NMR (DMSO-D₆, 300 MHz): δ (ppm)=10.76 8s, 1H), 8.21 (s, 1H), 7.83 (s, 1H), 7.69 (s, 1H), 7.34 (m, 2H), 7.07 (s, 1H), 3.84 (s, 3H), 2.15 (s, 3H).

### Example 27

### [3-Methoxy-4-(4-methyl-imidazol-1-yl)-phenyl]-(8-methoxymethyl-5-phenyl-[1,2,4]triazolo[1,5-c]pyrimidin-2-yl)-amine

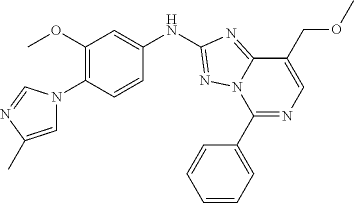

### a) 8-Methoxymethyl-5-phenyl-[1,2,4]triazolo[1,5-c]pyrimidin-2-ylamine

Prepared in analogy to example 1b-c, starting from 5-methoxymethyl-2-phenyl-pyrimidin-4-ylamine. The crude product was purified by column chromatography on silica gel using ethyl acetate as the eluent. The title compound was obtained as a white solid (yield: 65% over 2 steps).

MS ISP (m/e): 256.2 (88) [(M+H)⁺], 224.1 (100) [(M−OMe+H⁺].

¹H NMR (DMSO-D₆, 300 MHz): δ (ppm)=8.52 (m, 2H), 8.20 (s, 1H), 7.60 (m, 3H), 6.61 (br s, 2H), 4.65 (s, 2H), 3.39 (s, 3H).

### b) [3-Methoxy-4-(4-methyl-imidazol-1-yl)-phenyl]-(8-methoxymethyl-5-phenyl-[1,2,4]triazolo[1,5-c]pyrimidin-2-yl)-amine

Prepared in analogy to example 8e, starting from 8-methoxymethyl-5-phenyl-[1,2,4]triazolo[1,5-c]pyrimidin-2-ylamine and 1-(4-bromo-2-methoxy-phenyl)-4-methyl-1H-imidazole. The title compound was obtained as a brown solid (yield: 62%) after column chromatography on silica gel using CH₂Cl₂/MeOH 19:1 (v/v) as eluent.

MS ISP (m/e): 442.3 (100) [(M+H)⁺].

¹H NMR (DMSO-D₆, 300 MHz): δ (ppm)=10.39 (s, 1H), 8.58 (d, 2H), 8.36 (s, 1H), 7.94 (s, 1H), 7.66-7.61 (m, 4H), 7.28 (d, 1H), 7.10 (d, 1H), 7.04 (s, 1H), 4.74 (s, 2H), 3.85 (s, 3H), 3.42 (s, 3H), 2.15 (s, 3H).

### Example 28

### (5-Cyclohexyl-8-methoxymethyl-[1,2,4]triazolo[1,5-c]pyrimidin-2-yl)-[3-methoxy-4-(4-methyl-imidazol-1-yl)-phenyl]-amine

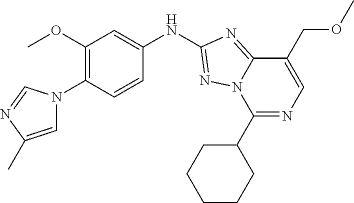

### a) 5-Cyclohexyl-8-methoxymethyl-[1,2,4]triazolo[1,5-c]pyrimidin-2-ylamine

Prepared in analogy to example 1b-c, starting from 2-cyclohexyl-5-methoxymethyl-pyrimidin-4-ylamine. The crude product was purified by column chromatography on silica gel using ethyl acetate as the eluent. The title compound was obtained as a light grey solid (yield: 25% over 2 steps).

MS ISP (m/e): 262.2 (100) [(M+H)⁺],230.4 (74) [(M−OMe+H)⁺].

¹H NMR (DMSO-D₆, 300 MHz): δ (ppm)=8.08 (s, 1H), 4.69 (s, 2H), 4.67 (s, 2H), 3.49 (s, 3H), 2.05 (br d, 2H), 1.88 (br d, 2H), 1.70-1.26 (m, 11H).

### b) (5-Cyclohexyl-8-methoxymethyl-[1,2,4]triazolo[1,5-c]pyrimidin-2-yl)-[3-methoxy-4-(4-methyl-imidazol-1-yl)-phenyl]-amine

Prepared in analogy to example 8e, starting from 5-cyclohexyl-8-methoxymethyl-[1,2,4]triazolo[1,5-c]pyrimidin-2-ylamine and 1-(4-bromo-2-methoxy-phenyl)-4-methyl-1H-imidazole. The title compound was obtained as a light brown solid (yield: 22%) after column chromatography on silica gel using CH₂Cl₂/MeOH 19:1 (v/v) as the eluent.

MS ISP (m/e): 448.3 (100) [(M+H)⁺].

¹H NMR (CDCl₃, 300 MHz): δ (ppm)=8.15 (s, 1H), 7.86 (s, 1H), 7.64 (s, 1H), 7.21 (d, 1H), 7.15 (s, 1H), 6.88 (m, 2H), 4.71 (s, 2H), 3.94 (s, 3H), 3.51 (s, 3H), 2.31 (s, 3H), 2.17 (br d, 2H), 1.75 (br s, 2H), 1.85-1.30 (m, 7H).

### Example 29

### [3-Methoxy-4-(4-methyl-imidazol-1-yl)-phenyl]-(8-morpholin-4-ylmethyl-5-phenyl-[1,2,4]triazolo[1,5-c]pyrimidin-2-yl)-amine

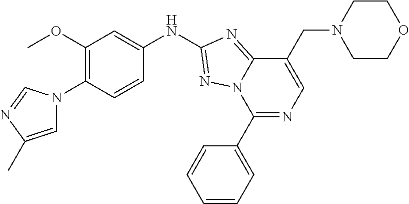

### a) 8-Morpholin-4-ylmethyl-5-phenyl-[1,2,4]triazolo[1,5-c]pyrimidin-2-ylamine

Prepared in analogy to example 1b-c, starting from 5-morpholin-4-ylmethyl-2-phenyl-pyrimidin-4-ylamine. The title compound was purified by column chromatography on silica gel using CH₂Cl₂/MeOH 19:1 (v/v) as eluent and was obtained as a white solid (yield: 65% over 2 steps).

MS ISP (m/e): 311.3 (71) [(M+H)⁺], 224.3 (100) [(M−morpholine+H)⁺].

¹H NMR (DMSO-D₆, 300 MHz): δ (ppm)=8.53 (m, 2H), 8.18 (s, 1H), 7.59 (m, 4H), 6.59 (s, 2H), 3.72 (s, 2H), 3.59 (m, 4H).

### b) [3-Methoxy-4-(4-methyl-imidazol-1-yl)-phenyl]-(8-morpholin-4-ylmethyl-5-phenyl-[1,2,4]triazolo[1,5-c]pyrimidin-2-yl)-amine

Prepared in analogy to example 8e, starting from 8-morpholin-4-ylmethyl-5-phenyl-[1,2,4]triazolo[1,5-c]pyrimidin-2-ylamine and 1-(4-bromo-2-methoxy-phenyl)-4-methyl-1H-imidazole. The title compound was obtained as a light yellow foam (yield: 78%) after column chromatography on silica gel using CH₂Cl₂/MeOH 19:1 (v/v) as eluent.

MS ISP (m/e): 497.4 (100) [(M+H)⁺].

¹H NMR (DMSO-D₆, 300 MHz): δ (ppm)=10.39 (s, 1H), 8.57 (d, 2H), 8.33 (s, 1H), 7.96 (s, 1H), 7.65 (d, 2H), 7.61 (m, 2H), 7.27 (d, 1H), 7.10 (d, 1H), 7.04 (s, 1H), 3.85 (s, 3H), 3.81 (s, 2H), 3.60 (m, 4H), 2.15 (s, 3H).

### Example 30

### [3-Methoxy-4-(4-methyl-imidazol-1-yl)-phenyl]-(5-morpholin-4-yl-[1,2,4]triazolo[1,5-a]pyridin-2-yl)-amine

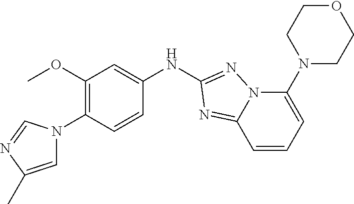

### a) 6-Morpholin-4-yl-pyridin-2-ylamine

Prepared in analogy to example 12a, starting from 2-amino-6-bromopyridine and morpholine. The title compound was purified by column chromatography on silica gel using a gradient from n-heptane to n-heptane/ethyl acetate 4:1 (v/v) as eluent and was obtained as a white solid (yield: 74%).

MS ISP (m/e): 180.2 (100) [(M+H)⁺]

¹H NMR (CDCl₃, 300 MHz): δ (ppm)=7.30 (t, 1H), 5.98 (d, 1H), 5.91 (d, 1H), 4.19 (br s, 2H), 3.79 (t, 4H), 3.43 (t, 4H).

### b) 5-Morpholin-4-yl-[1,2,4]triazolo[1,5-a]pyridin-2-ylamine

Prepared in analogy to example 1b-c, starting from 6-morpholin-4-yl-pyridin-2-ylamine. The title was purified by column chromatography on silica gel using a gradient from CH₂Cl₂ to CH₂Cl₂/MeOH 19:1 (v/v) as eluent and was obtained as an off-white foam (yield: 67% over two steps).

MS ISP (m/e): 220.2 (100) [(M+H)⁺].

¹H NMR (CDCl₃, 300 MHz): δ (ppm)=7.36 (dd, 1H), 7.09 (d, 1H), 6.23 (d, 1H), 4.45 (br s, 2H), 3.97 (m, 4H), 3.43 (m, 4H).

### c) [3-Methoxy-4-(4-methyl-imidazol-1-yl)-phenyl]-(5-morpholin-4-yl-[1,2,4]triazolo[1,5-a]pyridin-2-yl)-amine

Prepared in analogy to example 8e, starting from 5-morpholin-4-yl-[1,2,4]triazolo[1,5-a]pyridin-2-ylamine and 1-(4-bromo-2-methoxy-phenyl)-4-methyl-1H-imidazole. The title compound was obtained as an off-white solid (yield: 51%) after column chromatography on silica gel using a gradient from CH₂Cl₂ to CH₂Cl₂/MeOH 19:1 (v/v) as eluent.

MS ISP (m/e): 406.3 (100) [(M+H)⁺].

¹H NMR (CDCl₃, 300 MHz): δ (ppm)=7.62 (s, 1H), 7.55 (s, 1H), 7.45 (t, 1H), 7.19 (m, 2H), 7.07 (m, 2H), 6.87 (s, 1H), 6.31 (d, 1H), 3.99 (t, 4H), 3.89 (s, 3H), 3.52 (t, 4H), 2.30 (s, 3H).

### Example 31

### (5-Azepan-1-yl-[1,2,4]triazolo[1,5-a]pyridin-2-yl)-[3-methoxy-4-(4-methyl-imidazol-1-yl)-phenyl]-amine

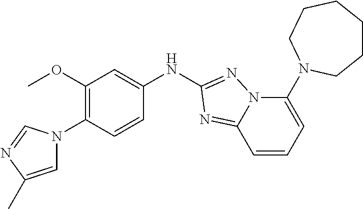

### a) 6-Azepan-1-yl-pyridin-2-ylamine

Prepared in analogy to example 12a, starting from 2-amino-6-bromopyridine and azepane.

The title compound was purified by column chromatography on silica gel using a gradient from n-heptane to n-heptane/ethyl acetate 4:1 (v/v) as eluent and was obtained as an yellow oil (yield: 79%).

MS ISP (m/e): 192.4 (100) [(M+H)⁺].

¹H NMR (CDCl₃, 300 MHz): δ (ppm)=7.21 (t, 1H), 5.86 (d, 1H), 5.74 (d, 1H), 4.09 (br s, 2H), 3.57 (t, 4H), 1.75 (m, 4H), 1.53 (m, 4H).

### b) 5-Azepan-1-yl-[1,2,4]triazolo[1,5-a]pyridin-2-ylamine

Prepared in analogy to example 1b-c, starting from 6-azepan-1-yl-pyridin-2-ylamine. The title was purified by column chromatography on silica gel using a gradient from CH₂Cl₂ to CH₂Cl₂/MeOH 19:1 (v/v) as eluent and was obtained as a yellow solid (yield: 82 and 84% for the two steps).

MS ISP (m/e): 232.1 (100) [(M+H)⁺].

¹H NMR (CDCl₃, 300 MHz): δ (ppm)=7.28 (t, 1H), 6.89 (d, 1H), 6.16 (d, 1H), 4.37 (br s, 2H), 3.66 (m, 4H), 1.89 (m, 4H), 1.68 (m, 4H).

### c) (5-Azepan-1-yl-[1,2,4]triazolo[1,5-a]pyridin-2-yl)-[3-methoxy-4-(4-methyl-imidazol-1-yl)-phenyl]-amine

Prepared in analogy to example 8e, starting 5-azepan-1-yl-[1,2,4]triazolo[1,5-a]pyridin-2-ylamine and 1-(4-bromo-2-methoxy-phenyl)-4-methyl-1H-imidazole. The title compound was obtained as a light brown solid (yield: 73%) after column chromatography on silica gel using a gradient from CH₂Cl₂ to CH₂Cl₂/MeOH 19:1 (v/v) as eluent.

MS ISP (m/e): 418.3 (100) [(M+H)⁺].

¹H NMR (CDCl₃, 300 MHz): δ (ppm)=7.62 (s, 1H), 7.40 (s, 1H), 7.35 (t, 1H), 7.18 (s, 2H), 7.12 (s, 1H), 6.97 (d, 1H), 6.87 (s, 1H), 3.87 (s, 3H), 3.78 (t, 4H), 2.30 (s, 3H), 1.95 (m, 4H), 1.71 (m, 4H).

### Example 32

### [3-Methoxy-4-(4-methyl-imidazol-1-yl)-phenyl]-(8-morpholin-4-ylmethyl-5-propyl-[1,2,4]triazolo[1,5-c]pyrimidin-2-yl)-amine

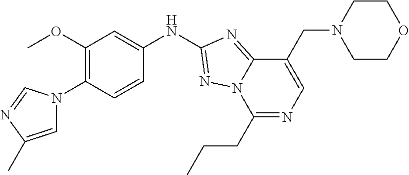

### a) 8-Morpholin-4-ylmethyl-5-propyl-[1,2,4]triazolo[1,5-c]pyrimidin-2-ylamine

Prepared in analogy to example 1b-c, starting from 5-morpholin-4-ylmethyl-2-propyl-pyrimidin-4-ylamine. The title compound was purified by column chromatography on silica gel using CH₂Cl₂/MeOH 19:1 (v/v) as eluent and was obtained as a light grey solid (yield: 86 and 32% for 2 steps).

MS ISP (m/e): 277.3 (39) [(M+H)⁺], 190.4 (100)[M−morpholine+H)⁺].

¹H NMR (CDCl₃, 300 MHz): δ (ppm)=8.08 (s, 1H), 4.9 (s, 2H), 3.74 (m, 6H), 3.13 (t, 2H), 2.57 (m, 4H), 1.94 (sext, 2H), 1.07 (t, 3H).

### b) [3-Methoxy-4-(4-methyl-imidazol-1-yl)-phenyl]-(8-morpholin-4-ylmethyl-5-propyl-[1,2,4]triazolo[1,5-c]pyrimidin-2-yl)-amine

Prepared in analogy to example 8e, starting from 8-morpholin-4-ylmethyl-5-propyl-[1,2,4]triazolo[1,5-c]pyrimidin-2-ylamine and 1-(4-bromo-2-methoxy-phenyl)-4-methyl-1H-imidazole. The title compound was obtained as a light yellow foam (yield: 78%) after column chromatography on silica gel using CH₂Cl₂/MeOH 9:1 (v/v) as eluent.

MS ISP (m/e): 463.3 (100) [(M+H)⁺].

¹H NMR (DMSO-D₆, 300 MHz): δ (ppm)=10.27 (s, 1H), 8.11 (s, 1H), 7.89 (s, 1H), 7.66 (s, 1H), 7.29 (d, 1H), 7.21 (d, 1H), 7.04 (s, 1H), 3.85 (s, 3H), 3.72 (s, 2H), 3.57 (m, 4H), 3.19 (t, 2H), 2.50 (m, 4H), 2.15 (s, 3H), 1.93 (sext, 2H), 1.01 (t, 3H).

### Example 33

### [3-Methoxy-4-(4-methyl-imidazol-1-yl)-phenyl]-(5-piperidin-1-yl-[1,2,4]triazolo[1,5-a]pyridin-2-yl)-amine

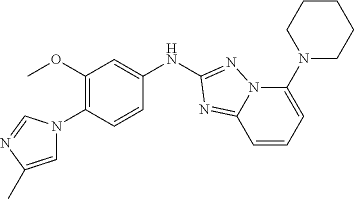

### a) 6-Piperidin-1-yl-pyridin-2-ylamine

Prepared in analogy to example 12a, starting from 2-amino-6-bromopyridine and piperidine. The title compound was purified by column chromatography on silica gel using a gradient from n-heptane to n-heptane/ethyl acetate 4:1 (v/v) as eluent and was obtained as a yellow oil (yield: 75%).

MS ISP (m/e): 178.2 (100) [(M+H)⁺].

¹H NMR (CDCl₃, 300 MHz): δ (ppm)=7.24 (t, 1H), 6.00 (d, 1H), 5.82 (d, 1H), 4.14 (br s, 2H), 3.45 (br s, 4H), 1.61 (br s, 6H).

### b) 5-Piperidin-1-yl-[1,2,4]triazolo[1,5-a]pyridin-2-ylamine

Prepared in analogy to example 1b-c, starting from 6-piperidin-1-yl-pyridin-2-ylamine. The title was purified by column chromatography on silica gel using a gradient from CH₂Cl₂ to CH₂Cl₂/MeOH 19:1 (v/v) as eluent and was obtained as a yellow oil (yield: 100% over two steps).

¹H NMR (CDCl₃, 300 MHz): δ (ppm)=7.33 (dd, 1H), 7.03 (d, 1H), 6.21 (d, 1H), 4.47 (br s, 2H), 3.33 (t, 4H), 1.82 (m, 4H), 1.68 (m, 2H).

### c) (5-Piperidin-1-yl-[1,2,4]triazolo[1,5-a]pyridin-2-yl)-[3-methoxy-4-(4-methyl-imidazol-1-yl)-phenyl]-amine

Prepared in analogy to example 8e, starting 5-piperidin-1-yl-[1,2,4]triazolo[1,5-a]pyridin-2-ylamine and 1-(4-bromo-2-methoxy-phenyl)-4-methyl-1H-imidazole. The title compound was obtained as a light brown solid (yield: 48%) after column chromatography on silica gel using a gradient from CH₂Cl₂ to CH₂Cl₂/MeOH 19:1 (v/v) as eluent.

MS ISP (m/e): 404.4 (100) [(M+H)⁺].

¹H NMR (CDCl₃, 300 MHz): δ (ppm)=7.62 (s, 1H), 7.55 (s, 1H), 7.42 (t, 1H), 7.19-7.12 (m, 3H), 6.87 (s, 1H), 6.30 (d, 1H), 3.89 (s, 3H), 3.44 (t, 4H), 2.30 (s, 3H), 1.85 (m, 4H), 1.72 (m, 2H).

### Example 34

N5,N5-Diethyl-N2-[3-methoxy-4-(4-methyl-imidazol-1-yl)-phenyl]-[1,2,4]triazolo[1,5-a]pyridine-2,5-diamine

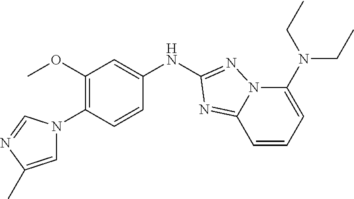

### a) N,N-Diethyl-pyridine-2,6-diamine

Prepared in analogy to example 12a, starting from 2-amino-6-bromopyridine and diethyl amine. The title compound was purified by column chromatography on silica gel using a gradient from n-heptane to n-heptane/ethyl acetate 4:1 (v/v) as eluent and was obtained as a yellow oil (yield: 21%).

MS ISP (m/e): 166.2 (100) [(M+H)⁺].

¹H NMR (CDCl₃, 300 MHz): δ (ppm)=7.20 (t, 1H), 5.85 (d, 1H), 5.73 (d, 1H), 4.09 (br s, 2H), 3.45 (q, 4H), 1.14 (t, 6H).

b) N,N5-Diethyl-[1,2,4]triazolo[1,5-a]pyridine-2,5-diamine

Prepared in analogy to example 1b-c, starting from N,N-diethyl-pyridine-2,6-diamine. The title was purified by column chromatography on silica gel using a gradient from CH₂Cl₂ to CH₂Cl₂/MeOH 19:1 (v/v) as eluent and was obtained as a white solid (yield: 69 and 77% for two steps).

MS ISP (m/e): 206.2 (100) [(M+H)⁺].

¹H NMR (CDCl₃, 300 MHz): δ (ppm)=7.32 (dd, 1H), 7.02 (d, 1H), 6.23 (d, 1H), 4.43 (br s, 2H), 3.52 (q, 4H), 1.13 (t, 6H).

### c) N5,N5-Diethyl-N2-[3-methoxy-4-(4-methyl-imidazol-1-yl)-phenyl]-[1,2,4]-triazolo[1,5-a]pyridine-2,5-diamine

Prepared in analogy to example 8e, starting N5,N5-diethyl-[1,2,4]triazolo[1,5-a]pyridine-2,5-diamine and 1-(4-bromo-2-methoxy-phenyl)-4-methyl-1H-imidazole. The title compound was obtained as a light brown solid (yield: 61%) after column chromatography on silica gel using a gradient from CH₂Cl₂ to CH₂Cl₂/MeOH 19:1 (v/v) as eluent.

MS ISP (m/e): 392.3 (100) [(M+H)⁺].

¹H NMR (CDCl₃, 300 MHz): δ (ppm)=7.63 (s, 1H), 7.53 (s, 1H), 7.40 (t, 1H), 7.19-7.12 (m, 3H), 7.08 (d, 1H), 6.87 (s, 1H), 6.29 (d, 1H), 3.88 (s, 3H), 3.63 (q, 4H), 2.30 (s, 3H), 1.20 (t, 6H).

### Example 35

### 4-{2-[3-Methoxy-4-(4-methyl-imidazol-1-yl)-phenylamino]-[1,2,4]triazolo[1,5-a]pyrimidin-7-yl}-benzonitrile

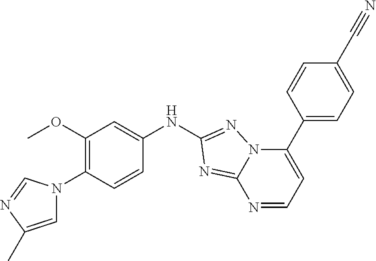

A solution of 4-cyano-acetophenone (43 mg, 0.3 mmol) and Bredereck reagent (52 mg, 0.3 mmol) in dioxane (2 mL) was heated to reflux for 4 hours. The solvent was evaporated under reduced pressure. To the residue a solution of N3-[3-methoxy-4-(4-methyl-imidazol-1-yl)-phenyl]-1H-[1,2,4]triazole-3,5-diamine (69 mg, 0.25 mmol) in acetic acid (1 mL) was added and the reaction was heated to 100° C. over night. The solvent was evaporated and the residue treated with CH₂Cl₂/diethyl ether. The precipitate was filtered, washed with diethyl ether, dried to yield the title compound as a yellow solid (61 mg, 72%).

MS ISP (m/e): 423.2 (100) [(M+H)⁺].

¹H NMR (DMSO-D₆, 300 MHz): δ (ppm)=10.22 (s, 1H), 8.78 (d, 1H), 8.43 (d, 2H), 8.12 (d, 2H), 7.76 (s, 1H), 7.65 (s, 1H), 7.52 (d, 1H), 7.30-7.18 (m, 2H), 7.02 (s, 1H), 3.78 (s, 3H), 2.15 (s, 3H).

### Example 36

### [3-Methoxy-4-(4-methyl-imidazol-1-yl)-phenyl]-[7-(4-trifluoromethyl-phenyl)-[1,2,4]triazolo[1,5-a]pyrimidin-2-yl]-amine

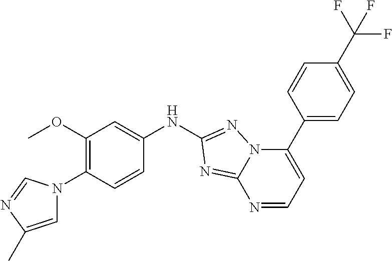

A solution of 4-trifluoromethyl-acetophenone (56 mg, 0.3 mmol) and Bredereck reagent (52 mg, 0.3 mmol) in dioxane (2 mL) was heated to reflux for 4 hours. The solvent was evaporated under reduced pressure. To the residue a solution of N3-[3-methoxy-4-(4-methyl-imidazol-1-yl)-phenyl]-1H-[1,2,4]triazole-3,5-diamine (57 mg, 0.2 mmol) in acetic acid (1 mL) was added and the reaction was heated to 100° C. over night. The solvent was evaporated and the residue treated with CH₂Cl₂/diethyl ether. The precipitate was filtered, washed with diethyl ether, dried to yield the title compound as a yellow solid (44 mg, 47%).

MS ISP (m/e): 466.3 (100) [(M+H)⁺].

¹H NMR (DMSO-D₆, 300 MHz): δ (ppm)=10.20 (s, 1H), 8.79 (d, 1H), 8.41 (d, 2H), 8.03 (d, 2H), 7.80 (s, 1H), 7.65 (s, 1H), 7.50 (d, 1H), 7.26 (d, 1H), 7.18 (d, 1H), 7.02 (s, 1H), 3.74 (s, 3H), 2.14 (s, 3H).

### Example 37

### 2-[3-Methoxy-4-(4-methyl-imidazol-1-yl)-phenylamino]-7-phenyl-[1,2,4]triazolo[1,5-a]pyrimidine-6-carbonitrile

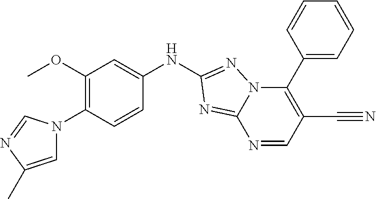

A solution of N3-[3-methoxy-4-(4-methyl-imidazol-1-yl)-phenyl]-1H-[1,2,4]triazole-3,5-diamine (71 mg, 0.25 mmol) and (E)-2-benzoyl-3-dimethylamino-acrylonitrile (50 mg, 0.25 mmol) in acetic acid (1 mL) was heated to 100° C. over night. The solvent was evaporated and the residue treated with CH₂Cl₂/diethyl ether. The precipitate was filtered, washed with diethyl ether, dried and purified by column chromatography on silica gel using CH₂Cl₂/MeOH (v/v 19:1) as eluent to yield the title compound as a yellow solid (18 mg, 17%).

MS ISP (m/e): 423.2 (100) [(M+H)⁺].

### Example 38

### 7-(2-Chloro-phenyl)-2-[3-methoxy-4-(4-methyl-imidazol-1-yl)-phenylamino]-[1,2,4]triazolo[1,5-a]pyrimidine-6-carbonitrile

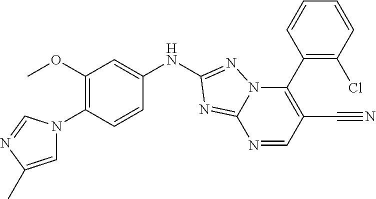

A solution of N3-[3-methoxy-4-(4-methyl-imidazol-1-yl)-phenyl]-1H-[1,2,4]triazole-3,5-diamine (61 mg, 0.21 mmol) and (E)-2-(2-chloro-benzoyl)-3-dimethylamino-acrylonitrile (50 mg, 0.21 mmol) in acetic acid (1 mL) was heated to 100° C. over night. The solvent was evaporated and the residue treated with CH₂Cl₂/diethyl ether. The precipitate was filtered, washed with diethyl ether, dried and purified by column chromatography on silica gel using CH₂Cl₂/MeOH (v/v 19:1) as eluent to yield the title compound as a yellow solid (4.5 mg, 4.6%).

MS ISP (m/e): 457.2/459.3 (100/33) [(M+H)⁺].

### Example 39

### 7-(4-Chloro-phenyl)-2-[3-methoxy-4-(4-methyl-imidazol-1-yl)-phenylamino]-[1,2,4]triazolo[1,5-a]pyrimidine-6-carbonitrile

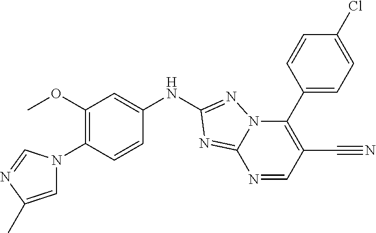

A solution of N3-[3-methoxy-4-(4-methyl-imidazol-1-yl)-phenyl]-1H-[1,2,4]triazole-3,5-diamine (61 mg, 0.21 mmol) and (E)-2-(4-chloro-benzoyl)-3-dimethylamino-acrylonitrile (50.7 mg, 0.21 mmol) in acetic acid (1 mL) was heated to 100° C. over night. The solvent was evaporated and the residue treated with CH₂Cl₂/diethyl ether. The precipitate was filtered, washed with diethyl ether, dried and purified by column chromatography on silica gel using CH₂Cl₂/MeOH (v/v 19:1) as eluent to yield the title compound as a yellow solid (23.1 mg, 23%).

MS ISP (m/e): 457.2/459.2 (100/35) [(M+H)⁺].

### Example 40

### 2-[3-Methoxy-4-(4-methyl-imidazol-1-yl)-phenylamino]-7-m-tolyl-[1,2,4]triazolo[1,5-a]pyrimidine-6-carbonitrile

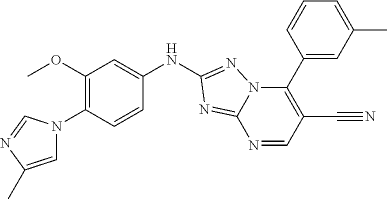

A solution of N3-[3-methoxy-4-(4-methyl-imidazol-1-yl)-phenyl]-1H-[1,2,4]triazole-3,5-diamine (69 mg, 0.25 mmol) and (E)-2-(3-methyl-benzoyl)-3-dimethylamino-acrylonitrile (50.4 mg, 0.25 mmol) in acetic acid (1 mL) was heated to 100° C. over night. The solvent was evaporated and the residue treated with CH₂Cl₂/diethyl ether. The precipitate was filtered, washed with diethyl ether, dried and purified by column chromatography on silica gel using CH₂Cl₂/MeOH (v/v 19:1) as eluent to yield the title compound as a yellow solid (24 mg, 23%).

MS ISP (m/e): 437.2 (100) [(M+H)⁺].

### Example 41

### [3-Methoxy-4-(4-methyl-imidazol-1-yl)-phenyl]-(7-pyrazin-2-yl-5-trifluoromethyl-[1,2,4]triazolo[1,5-a]pyrimidin-2-yl)-amine

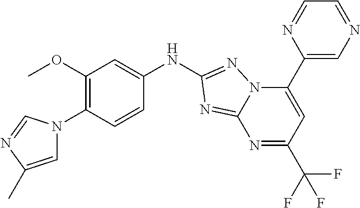

A solution of N3-[3-methoxy-4-(4-methyl-imidazol-1-yl)-phenyl]-1H-[1,2,4]triazole-3,5-diamine (61 mg, 0.21 mmol) and 4,4,4-trifluoro-1-(pyrazine-2-yl)butane-1,3-dione (46 mg, 0.21 mmol) in acetic acid (1 mL) was heated to 100° C. over night. The solvent was evaporated and the residue treated with CH₂Cl₂/diethyl ether. The precipitate was filtered, washed with diethyl ether, dried and purified by column chromatography on silica gel using CH₂Cl₂/MeOH (v/v 19:1) as eluent to yield the title compound as a yellow solid (41 mg, 42%).

MS ISP (m/e): 468.2 (100) [(M+H)⁺].

### Example 42

### [3-Methoxy-4-(4-methyl-imidazol-1-yl)-phenyl]-(6-methoxy-[1,2,4]triazolo[1,5-b]pyridazin-2-yl)-amine

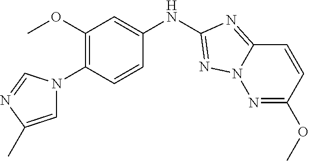

### a) 6-Methoxy-[1,2,4]triazolo[1,5-b]pyridazin-2-ylamine

Prepared in analogy to example 1b-c, starting from 3-amino-6-methoxypyridazine. The title was purified by column chromatography on silica gel using a gradient from CH₂Cl₂ to CH₂Cl₂/MeOH 19:1 (v/v) as eluent. The crude product was stirred with diethyl ether, filtered and dried to yield the title compound as a light yellow solid (yield: 87% over two steps).

MS ISP (m/e): 166.3 (100) [(M+H)⁺].

¹H NMR (DMSO-D₆, 300 MHz): δ (ppm)=7.87 (d, 1H), 7.07 (d, 1H), 5.98 (br s, 2H), 3.92 (s, 3H).

### b) [3-Methoxy-4-(4-methyl-imidazol-1-yl)-phenyl]-(6-methoxy-[1,2,4]triazolo[1,5-b]pyridazin-2-yl)-amine

Prepared in analogy to example 8e, starting 6-methoxy-[1,2,4]triazolo[1,5-b]pyridazin-2-ylamine and 1-(4-bromo-2-methoxy-phenyl)-4-methyl-1H-imidazole. The title compound was obtained as an off-white solid (yield: 64%) after column chromatography on silica gel using a gradient from CH₂Cl₂ to CH₂Cl₂/MeOH 19:1 (v/v) as eluent.

MS ISP (m/e): 352.3 (100) [(M+H)⁺].

¹H NMR (DMSO-D₆, 300 MHz): δ (ppm)=9.80 (s, 1H), 8.09 (d, 1H), 7.65 (s, 1H), 7.47 (s, 1H), 7.43 (d, 1H), 7.28-7.23 (m, 2H), 7.02 (s, 1H), 3.99 (s, 3H), 3.80 (s, 3H), 2.15 (s, 3H).

### Example 43

### (8-Benzyloxy-[1,2,4]triazolo[1,5-a]pyridin-2-yl)-[3-methoxy-4-(4-methyl-imidazol-1-yl)-phenyl]-amine

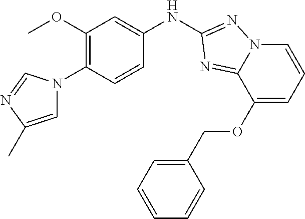

### a) 8-Benzyloxy-[1,2,4]triazolo[1,5-a]pyridin-2-ylamine

Prepared in analogy to example 1b-c, starting from 2-amino-3-benzyloxypyridine. The crude product was purified by column chromatography on silica gel using a gradient from CH₂Cl₂ to CH₂Cl₂/MeOH 19:1 (v/v) as eluent. The title compound was obtained as an off-white solid (yield: 72% over two steps).

MS ISP (m/e): 241.2 (100) [(M+H)⁺].

¹H NMR (DMSO-D₆, 300 MHz): δ (ppm)=8.15 (d, 1H), 7.53-7.30 (m, 5H), 6.99 (d, 1H), 6.76 (t, 1H), 5.91 (br s, 2H), 5.28 (s, 2H).

### b) (8-Benzyloxy-[1,2,4]triazolo[1,5-a]pyridin-2-yl)-[3-methoxy-4-(4-methyl-imidazol-1-yl)-phenyl]-amine

Prepared in analogy to example 8e, starting 8-benzyloxy-[1,2,4]triazolo[1,5-a]pyridin-2-ylamine and 1-(4-bromo-2-methoxy-phenyl)-4-methyl-1H-imidazole. The title compound was obtained as a light brown (yield: 50%) after column chromatography on silica gel using a gradient from CH₂Cl₂ to CH₂Cl₂/MeOH 19:1 (v/v) as eluent.

MS ISP (m/e): 427.2 (100) [(M+H)⁺].

¹H NMR (DMSO-D₆, 300 MHz): δ (ppm)=9.87 (s, 1H), 8.42 (d, 1H), 7.64 (s, 1H), 7.60 (s, 1H), 7.51 (d, 2H), 7.43-7.33 (m, 5H), 7.25 (d, 1H), 7.15 (d, 1H), 7.02 (s, 1H), 6.96 (t, 1H), 5.36 (s, 2H), 3.81 (s, 3H), 2.14 (s, 3H).

### Example 44

### (6,8-Dichloro-[1,2,4]triazolo[1,5-a]pyridin-2-yl)-[3-methoxy-4-(4-methyl-imidazol-1-yl)-phenyl]-amine

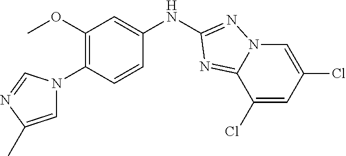

### a) 6,8-Dichloro-[1,2,4]triazolo[1,5-a]pyridin-2-ylamine

Prepared in analogy to example 1b-c, starting from 2-amino-3,5-dichloropyridine. The crude product was purified by column chromatography on silica gel using a gradient from CH₂Cl₂ to CH₂Cl₂/MeOH 19:1 (v/v) as eluent. The title compound was obtained as an off-white solid (yield: 8% over two steps).

MS ISP (m/e): 203.1/205.0 (100/81) [(M+H)⁺].

¹H NMR (DMSO-D₆, 300 MHz): δ (ppm)=8.93 (s, 1H), 7.82 (s, 1H), 6.39 (br s, 2H).

### b) (6,8-Dichloro-[1,2,4]triazolo[1,5-a]pyridin-2-yl)-[3-methoxy-4-(4-methyl-imidazol-1-yl)-phenyl]-amine

Prepared in analogy to example 8e, starting 6,8-dichloro-[1,2,4]triazolo[1,5-a]pyridin-2-ylamine and 1-(4-bromo-2-methoxy-phenyl)-4-methyl-1H-imidazole. The title compound was obtained as a light brown (yield: 47%) after column chromatography on silica gel using a gradient from CH₂Cl₂ to CH₂Cl₂/MeOH 19:1 (v/v) as eluent.

MS ISP (m/e): 389.2 (100) [(M+H)⁺].

¹H NMR (DMSO-D₆, 300 MHz): δ (ppm)=10.18 (s, 1H), 9.22 (s, 1H), 8.02 (s, 1H), 7.67 (s, 1H), 7.59 (s, 1H), 7.33 (d, 1H), 7.28 (d, 1H), 7.03 (s, 1H9, 3.82 (s, 3H), 2.15 (s, 3H).

### Example 45

### [5-(4-Fluoro-phenyl)-[1,2,4]triazolo[1,5-c]pyrimidin-2-yl]-[3-methoxy-4-(4-methyl-imidazol-1-yl)-phenyl]-amine

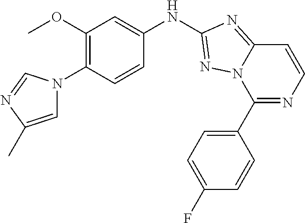

### a) 5-(4-Fluoro-phenyl)-[1,2,4]triazolo[1,5-c]pyrimidin-2-ylamine

Prepared in analogy to example 1b-c, starting from 2-(4-fluorophenyl)-4-pyrimidine. The crude product was purified by crystallization from hot EtOAc. The title compound was obtained as a white solid (yield: 43% over two steps).

MS ISP (m/e): 230.3 (100) [(M+H)⁺].

¹H NMR (DMSO-D₆, 300 MHz): δ (ppm)=8.65 (m, 2H), 8.22 (d, 1H), 7.47-7.39 (m, 3H), 6.58 (br s, 2H).

### b) [5-(4-Fluoro-phenyl)-[1,2,4]triazolo[1,5-c]pyrimidin-2-yl]-[3-methoxy-4-(4-methyl-imidazol-1-yl)-phenyl]-amine

Prepared in analogy to example 8e, starting 5-(4-fluoro-phenyl)-[1,2,4]triazolo[1,5-c]pyrimidin-2-ylamine and 1-(4-bromo-2-methoxy-phenyl)-4-methyl-1H-imidazole. The title compound was obtained as a light yellow solid (yield: 43%) after column chromatography on silica gel using CH₂Cl₂/MeOH 19:1 (v/v) as eluent.

MS ISP (m/e): 416.2 (100) [(M+H)⁺].

¹H NMR (DMSO-D₆, 300 MHz): δ (ppm)=10.25 (s, 1H), 8.69 (m, 2H), 8.37 (d, 1H), 7.82 (s, 1H), 7.67 (s, 1H), 7.63 (d, 1H), 7.47 (t, 2H), 7.29 (d, 1H), 7.22 (d, 1H), 7.04 (s, 1H), 3.84 (s, 3H), 2.15 (s, 3H).

### Example 46

### [8-(4-Fluoro-phenyl)-6-methyl-[1,2,4]triazolo[1,5-a]pyridin-2-yl]-[3-methoxy-4-(4-methyl-imidazol-1-yl)-phenyl]-amine

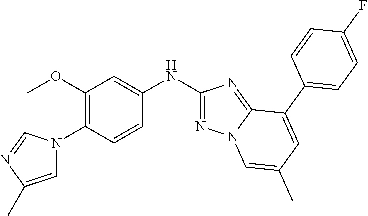

### a) 8-Bromo-6-methyl-[1,2,4]triazolo[1,5-a]pyridin-2-ylamine

Prepared in analogy to example 1b-c, starting from 2-amino-3-bromo-5-methylpyridine. The crude product was purified by crystallization from hot EtOAc. Most of the product was not soluble and precipitated during work-up. This material was filtered off, washed with water and CH₂Cl₂, dried and combined with the other material. The title compound was obtained as a white solid (yield: 73% over two steps).

MS ISP (m/e): 227.1/229.2 (100/84) [(M+H)⁺].

¹H NMR (DMSO-D₆, 300 MHz): δ (ppm)=8.43 (s, 1H), 7.63 (s, 1H), 6.13 (br s, 2H), 2.27 (s, 3H).

### b) 8-(4-Fluoro-phenyl)-6-methyl-[1,2,4]triazolo[1,5-a]pyridin-2-ylamine

A mixture of 8-bromo-6-methyl-[1,2,4]triazolo[1,5-a]pyridin-2-ylamine (68.1 mg, 0.3 mmol), 4-fluorophenyl boronic acid (47.6 mg, 0.33 mmol), dichloro[1,1′-bis(diphenylphosphino)-ferrocene]palladium(II) dichloromethane adduct (11.2 mg, 0.015 mmol) and an aqueous solution of Na₂CO₃ (2N, 0.75 mL, 1.5 mmol) in 1,2-dimethoxyethane (3 mL) was stirred at 80° C. over night. The reaction mixture was diluted with water and extracted with EtOAc, the combined organic phases were washed with 1N aqueous NaOH solution, brine, dried over sodium sulfate, the solvent was evaporated and the residue purified by silica gel chromatography using EtOAc as eluent. The title compound was obtained after stirring with diethyl ether, filtration and drying as a white solid (56 mg, 78%).

MS ISP (m/e): 243.3 (100) [(M+H)⁺].

¹H NMR (DMSO-D₆, 300 MHz) δ (ppm)=8.40 (s, 1H), 8.18 (m, 2H), 7.60 (s, 1H), 7.32 (t, 2H), 6.01 (br s, 2H), 2.34 (s, 3H).

### c) [8-(4-Fluoro-phenyl)-6-methyl-[1,2,4]triazolo[1,5-a]pyridin-2-yl]-[3-methoxy-4-(4-methyl-imidazol-1-yl)-phenyl]-amine

Prepared in analogy to example 8e, starting from 8-(4-fluoro-phenyl)-6-methyl-[1,2,4]triazolo[1,5-a]pyridin-2-ylamine and 1-(4-bromo-2-methoxy-phenyl)-4-methyl-1H-imidazole. The crude product was purified by column chromatography on silica gel using a gradient from CH₂Cl₂ to CH₂Cl₂/MeOH 19:1 (v/v) as the eluent. The title compound was obtained as a light yellow solid (yield: 75%).

MS ISP (m/e): 429.3 (100) [(M+H)⁺].

¹H NMR (DMSO-D₆, 300 MHz): δ (ppm)=9.92 (s, 1H), 8.67 (s, 1H), 8.25 (m, 2H), 7.85 (s, 1H), 7.77 (s, +H), 7.64 (s, 1H), 7.36 (t, 2H), 7.24 (s, 2H), 7.02 (s, 1H), 3.84 (s, 3H), 2.41 (s, 3H), 2.15 (s, 3H).

### Example 47

### [3-Methoxy-4-(4-methyl-imidazol-1-yl)-phenyl]-(7-trifluoromethyl-[1,2,4]triazolo[1,5-a]pyridin-2-yl)-amine

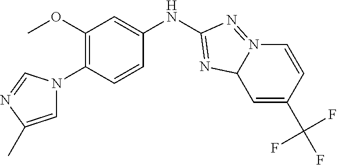

### a) 7-Trifluoromethyl-[1,2,4]triazolo[1,5-a]pyridin-2-ylamine

Prepared in analogy to example 1b-c, starting from 2-amino-4-(trifluoromethyl)pyridine. The crude product was purified by column chromatography on silica gel using a gradient from CH₂Cl₂ to CH₂Cl₂/MeOH 19:1 (v/v) as eluent. The title compound was obtained as an off-white solid (yield: 72% over two steps).

MS ISP (m/e): 203.2 (100) [(M+H)⁺].

¹H NMR (DMSO-D₆, 300 MHz): δ (ppm)=8.77 (d, 1H), 7.82 (s, 1H), 7.16 (d, 1H), 6.34 (br s, 2H).

### b) [3-Methoxy-4-(4-methyl-imidazol-1-yl)-phenyl]-(7-trifluoromethyl-[1,2,4]triazolo[1,5-a]pyridin-2-yl)-amine

Prepared in analogy to example 8e, starting 7-trifluoromethyl-[1,2,4]triazolo[1,5-a]pyridin-2-ylamine and 1-(4-bromo-2-methoxy-phenyl)-4-methyl-1H-imidazole. The title compound was obtained as a light brown (yield: 67%) after column chromatography on silica gel using a gradient from CH₂Cl₂ to CH₂Cl₂/MeOH 19:1 (v/v) as eluent.

MS ISP (m/e): 389.2 (100) [(M+H)⁺].

¹H NMR (DMSO-D₆, 300 MHz): δ (ppm)=10.07 (s, 1H), 9.04 (d, 1H), 8.12 (s, 1H), 7.67 (s, 1H), 7.59 (s, 1H), 7.43 (d, 1H), 7.34 (d, 1H), 7.29 (d, 1H), 7.04 (s, 1H), 3.83 (s, 3H), 2.14 (s, 3H).

### Example 48

### (8-Chloro-6-trifluoromethyl-[1,2,4]triazolo[1,5-a]pyridin-2-yl)-[3-methoxy-4-(4-methyl-imidazol-1-yl)-phenyl]-amine

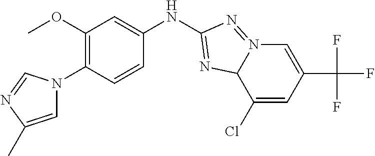

### a) 8-Chloro-6-trifluoromethyl-[1,2,4]triazolo[1,5-a]pyridin-2-ylamine

Prepared in analogy to example 1b-c, starting from 2-amino-3-chloro-5-(trifluoromethyl)pyridine. The crude product was purified by column chromatography on silica gel using a gradient from CH₂Cl₂ to CH₂Cl₂/MeOH 19:1 (v/v) as eluent. The title compound was obtained as an off-white solid (yield: 63% over two steps).

MS ISP (m/e): 237.0/239.0 (100/42) [(M+H)⁺].

¹H NMR (DMSO-D₆, 300 MHz): δ (ppm)=9.21 (s, 1H), 7.99 (s, 1H), 6.61 (br s, 2H).

### b) (8-Chloro-6-trifluoromethyl-[1,2,4]triazolo[1,5-a]pyridin-2-yl)-[3-methoxy-4-(4-methyl-imidazol-1-yl)-phenyl]-amine

Prepared in analogy to example 8e, starting 8-chloro-6-trifluoromethyl-[1,2,4]triazolo[1,5-a]pyridin-2-ylamine and 1-(4-bromo-2-methoxy-phenyl)-4-methyl-1H-imidazole. The title compound was obtained as a light brown (yield: 24%) after column chromatography on silica gel using a gradient from CH₂Cl₂ to CH₂Cl₂/MeOH 19:1 (v/v) as eluent.

MS ISP (m/e): 423.2 (100) [(M+H)⁺].

¹H NMR (DMSO-D₆, 300 MHz): δ (ppm)=10.36 (s, 1H), 9.59 (s, 1H), 8.19 (s, 1H), 7.67 (s, 1H), 7.64 (s, 1H), 7.31 (d, 1H), 7.30 (d, 1H), 7.05 (s, 1H), 3.84 (s, 3H), 2.15 (s, 3H).

### Example 49

### (6-Chloro-8-methyl-[1,2,4]triazolo[1,5-a]pyridin-2-yl)-[3-methoxy-4-(4-methyl-imidazol-1-yl)-phenyl]-amine

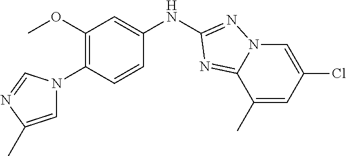

### a) 6-Chloro-8-methyl-[1,2,4]triazolo[1,5-a]pyridin-2-ylamine

Prepared in analogy to example 1b-c, starting from 2-amino-5-chloro-3-methylpyridine. The crude product was purified by column chromatography on silica gel using a gradient from CH₂Cl₂ to CH₂Cl₂/MeOH 19:1 (v/v) as eluent. The title compound was obtained as an off-white solid (yield: 28% over two steps).

MS ISP (m/e): 183.1/185.1 (100/40) [(M+H)⁺].

¹H NMR (DMSO-D₆, 300 MHz): δ (ppm)=8.70 (s, 1H), 7.35 (s, 1H), 6.08 (br s, 2H), 2.39 (s, 3H).

### b) (6-Chloro-8-methyl-[1,2,4]triazolo[1,5-a]pyridin-2-yl)-[3-methoxy-4-(4-methyl-imidazol-1-yl)-phenyl]-amine

Prepared in analogy to example 8e, starting 6-chloro-8-methyl-[1,2,4]triazolo[1,5-a]pyridin-2-ylamine and 1-(4-bromo-2-methoxy-phenyl)-4-methyl-1H-imidazole. The title compound was obtained as a light brown (yield: 26%) after column chromatography on silica gel using a gradient from CH₂Cl₂ to CH₂Cl₂/MeOH 19:1 (v/v) as eluent.

MS ISP (m/e): 369.1 (100) [(M+H)⁺].

¹H NMR (DMSO-D₆, 300 MHz): δ (ppm)=9.97 (s, 1H), 9.00 (s, 1H), 7.65 (s, 2H), 7.54 (s, 1H), 7.32 (d, 1H), 7.26 (d, 1H), 7.02 (s, 1H), 3.82 (s, 3H), 2.50 (s, 3H), 2.14 (s, 3H).

### Example 50

### (5,6-Dimethyl-[1,2,4]triazolo[1,5-a]pyridin-2-yl)-[3-methoxy-4-(4-methyl-imidazol-1-yl)-phenyl]-amine

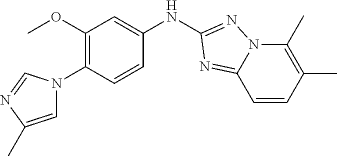

### a) 5,6-Dimethyl-[1,2,4]triazolo[1,5-a]pyridin-2-ylamine

Prepared in analogy to example 1b-c, starting from 2-amino-5,6-dimethylpyridine. The crude product was purified by column chromatography on silica gel using a gradient from CH₂Cl₂ to CH₂Cl₂/MeOH 19:1 (v/v) as eluent. The title compound was obtained as an off-white solid (yield: 42% over two steps).

MS ISP (m/e): 163.2 (100) [(M+H)⁺].

¹H NMR (DMSO-D₆, 300 MHz): δ (ppm)=7.27 (d, 1H), 7.13 (d, 1H), 5.88 (br s, 2H), 2.54 (s, 3H), 2.28 (s, 3H).

### b) (5,6-Dimethyl-[1,2,4]triazolo[1,5-a]pyridin-2-yl)-[3-methoxy-4-(4-methyl-imidazol-1-yl)-phenyl]-amine

Prepared in analogy to example 8e, starting 5,6-dimethyl-[1,2,4]triazolo[1,5-a]pyridin-2-ylamine and 1-(4-bromo-2-methoxy-phenyl)-4-methyl-1H-imidazole. The title compound was obtained as a light brown (yield: 69%) after column chromatography on silica gel using a gradient from CH₂Cl₂ to CH₂Cl₂/MeOH 19:1 (v/v) as eluent.

MS ISP (m/e): 349.3 (100) [(M+H)⁺].

¹H NMR (DMSO-D₆, 300 MHz): δ (ppm)=9.78 (s, 1H), 7.78 (s, 1H), 7.64 (s, 1H), 7.44 (d, 1H), 7.38 (d, 1H), 7.32 (d, 1H), 7.24 (d, 1H), 7.02 (s, 1H), 3.83 (s, 3H), 2.69 (s, 3H), 2.35 (s, 3H), 2.14 (s, 3H).

### Example 51 and 52

### (R)- and (S)-[8-(4-Fluoro-phenyl)-5,6,7,8-tetrahydro-[1,2,4]triazolo[1,5-a]pyridin-2-yl]-[3-methoxy-4-(4-methyl-imidazol-1-yl)-phenyl]-amine

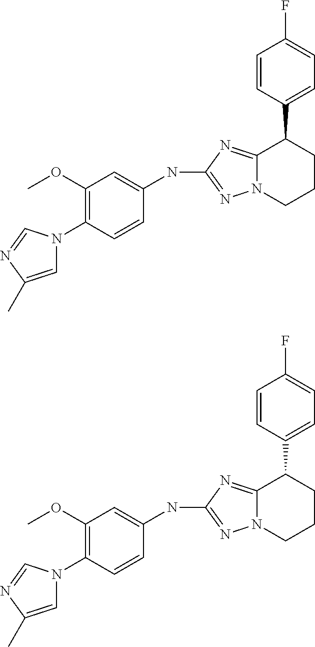

Separation of racemic [8-(4-fluoro-phenyl)-5,6,7,8-tetrahydro-[1,2,4]triazolo[1,5-a]pyridin-2-yl]-[3-methoxy-4-(4-methyl-imidazol-1-yl)-phenyl]-amine (example 1, 175 mg) by chiral HPLC (Reprosil Chiral NR) using ethanol/n-heptane 2:3 as eluent provided both enantiomers (without assignment of absolute configuration to the enantiomers):

### Example 51

### Enantiomer 1(+), retention time: 17.00 minutes (69 mg)

MS ISP (m/e): 419.3 (100) [(M+H)⁺].

¹H NMR (CDCl₃, 300 MHz): δ (ppm)=7.60-7.59 (m, 1H), 7.34-7.33 (m, 1H), 7.18-7.00 (m, 5H), 6.92-6.88 (m, 1H), 6.83 (m, 1H), 6.71 (m, 1H), 4.22-4.16 (m, 3H), 3.81 (s, 3H), 2.38-1.90 (m, 4H), 2.29 (s, 3H).

### Example 52

### Enantiomer 2(−), retention time: 22.00 minutes (58 mg)

MS ISP (m/e): 419.3 (100) [(M+H)⁺].

¹H NMR (CDCl₃, 300 MHz): δ (ppm)=7.59-7.58 (m, 1H), 7.34-7.33 (m, 1H), 7.18-7.00 (m, 5H), 6.91-6.88 (m, 1H), 6.84 (m, 1H), 6.64 (m, 1H), 4.22-4.16 (m, 3H), 3.81 (s, 3H), 2.38-1.93 (m, 4H), 2.29 (s, 3H).

### Example 53

### 8-(4-Fluorophenyl)-N-(3-methoxy-4-(2-methylpyridin-4-yl)phenyl)-[1,2,4]triazolo[1,5-a]pyridin-2-amine

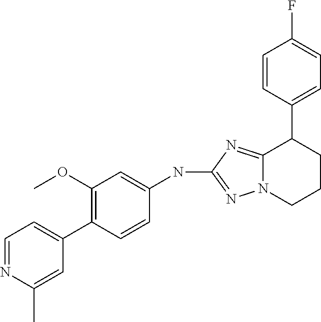

### a) 2-Methyl-4-(4,4,5,5-tetramethyl-[1,3,2]dioxaborolan-2-yl)-pyridine

To a solution of 4-bromo-2-methyl-pyridine (2.0 g, 12 mmol) in dioxane (20 mL) was added Pd(dppf)Cl₂ (0.3 g, 1.0 mmol), potassium acetate (3.4 g, 35 mmol) and bis(pinacolato)diboron (3.8 g, 15 mmol) under an argon atmosphere and the reaction was heated at 80° C. for 12 hours. The resulting black suspension is diluted with dichloromethane, filtered and concentrated under vacuum to afford a black oil (1.53 g, 60%) which is used crude for the next step.
¹H NMR (CDCl₃, 300 MHz): δ (ppm)=8.53-8.51 (m, 1H), 7.51 (m, 1H), 7.43-7.41 (m, 1H), 2.56 (s, 3H), 1.35 (s, 12H).

### b) 4-(2-Methoxy-4-nitro-phenyl)-2-methyl-pyridine

To a solution of 1-bromo-2-methoxy-4-nitro-benzene (6.35 g, 27 mmol), 2-methyl-4-(4,4,5,5-tetramethyl-[1,3,2]dioxaborolan-2-yl)-pyridine (6.60 g, 30 mmol) and tetrakis(triphenylphosphine)palladium(0) (1.58 g, 1.4 mmol) in dimethoxyethane (150 mL) under an argon atmosphere was added a solution of cesium carbonate (26.7 g, 82 mmol) dissolved in water (90 mL). The mixture was heated to 80° C. for 18 hours.

The volatiles were removed under vacuum, water was added and the aqueous phase was extracted three times with dichloromethane. The combined organic layers were dried over Na₂SO₄, filtered and the solvents were evaporated. The residue was purified by silica gel chromatography using n-heptane/TBME as eluent to give the title compound as a dark red solid (2.9 g, 43%).

MS ISP (m/e): 245.2 (100) [(M+H)⁺].

¹H NMR (CDCl₃, 300 MHz): δ (ppm)=8.58-8.56 (m, 1H), 7.95-7.92 (m, 1H), 7.85-7.84 (m, 1H), 7.47-7.45 (m, 1H), 7.29 (m, 1H), 7.25-7.24 (m, 1H), 3.94 (s, 3H), 2.63 (s, 3H).

### c) 3-Methoxy-4-(2-methyl-pyridin-4-yl)-phenylamine

4-(2-Methoxy-4-nitro-phenyl)-2-methyl-pyridine (2.9 g, 12 mmol) was hydrogenated (H₂ at 1 bar) in a solution of ammonia in methanol (7.0 M, 80 mL) in the presence of Raney-Nickel (515 mg, 4.1 mmol) at 30° C. for 18 hours. The catalyst was filtered, the solvent was evaporated and the residue was purified by silica gel chromatography using n-heptane/TBME as eluent to give the title compound as a brown solid (1.02 g, 40%).

MS ISP (m/e): 215.3 (100) [(M+H)⁺].

¹H NMR (CDCl₃, 300 MHz): δ (ppm)=8.45-8.44 (m, 1H), 7.30-7.26 (m, 2H), 7.17-7.14 (m, 1H), 6.38-6.32 (m, 2H), 3.80 (s, 3H), 2.57 (s, 3H).

### d) 2-Bromo-8-(4-fluorophenyl)-[1,2,4]triazolo[1,5-a]pyridine

A solution of copper(II) bromide (108 mg, 482 μmol) and t-butyl nitrite (55.2 mg, 63.9 μL, 482 μmol) in acetonitrile (3 mL) was heated to 60° C. and 8-(4-fluorophenyl)-[1,2,4]triazolo[1,5-a]pyridin-2-amine (see example 1c, 100 mg, 438 μmol) was added in small portions. After complete addition the reaction mixture was heated to 75° C. After 3 hours t-butyl nitrite (55.2 mg, 63.9 μL, 482 μmol) and copper(II) bromide (108 mg, 482 μmol) was added and heating continued 75° C. for another 2 hours. The reaction mixture was cooled to room temperature, water was added to the reaction mixture and the aqueous phase extracted with dichloromethane. The organic layers were combined, dried over Na₂SO₄, filtered and the solvents were evaporated. The residue was purified by silica gel chromatography using diethyl ether/n-pentane as eluent. The title compound was obtained as off-white solid (67.5 mg, 53%)

MS ISP (m/e): 292.0/294.0 (100/89) [(M+H)⁺].

¹H NMR (CDCl₃, 300 MHz): δ (ppm)=8.53-8.50 (m, 1H), 8.02-7.97 (m, 2H), 7.68-7.65 (m, 1H), 7.24-7.12 (m, 3H).

### e) 8-(4-Fluorophenyl)-N-(3-methoxy-4-(2-methylpyridin-4-yl)phenyl)-[1,2,4]triazolo[1,5-a]pyridin-2-amine

A mixture of 2-bromo-8-(4-fluorophenyl)-[1,2,4]triazolo[1,5-a]pyridine (88.3 mg, 0.30 mmol), 3-methoxy-4-(2-methyl-pyridin-4-yl)-phenylamine (54 mg, 0.25 mmol), sodium phenoxide (43.9 mg, 0.38 mmol), tris(dibenzylideneacetone)dipalladium chloroform complex (10.4 mg, 0.010 mmol) and 4,5-bis(diphenylphosphino)-9,9-dimethylxanthene (11.7 mg, 0.020 mmol) in dioxane (3 mL) was heated under an argon atmosphere in the microwave to 130° C. for 45 min. Further tris(dibenzylideneacetone)dipalladium chloroform complex (10.4 mg, 0.010 mmol) and 4,5-bis(diphenylphosphino)-9,9-dimethylxanthene (11.7 mg, 0.020 mmol) was added to the reaction mixture and irradiated for another 30 minutes. The mixture was purified by silica gel chromatography using dichloromethane/methanol (with 10% ammonia) as eluent. The title compound was obtained as a light yellow solid (90 mg, 84%).

MS ISP (m/e): 426.1 (100) [(M+H)⁺].

¹H NMR (CDCl₃, 300 MHz): δ (ppm)=8.50-8.48 (m, 1H), 8.46-8.43 (m, 1H), 8.05-8.00 (m, 2H), 7.65-7.64 (m, 1H), 7.60-7.57 (m, 1H), 7.34-7.17 (m, 5H), 7.08-6.98 (m, 3H), 3.92 (s, 3H), 2.60 (s, 3H).

### Example 54

### 5-(4-Fluorophenyl)-N-(3-methoxy-4-(2-methylpyridin-4-yl)phenyl)-[1,2,4]triazolo[1,5-a]pyridin-2-amine

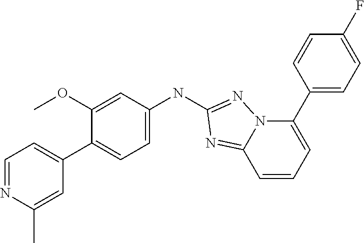

Prepared in analogy to example 53e) employing 2-bromo-5-(4-fluoro-phenyl)-[1,2,4]triazolo[1,5-a]pyridine (see example 8d) and 3-methoxy-4-(2-methyl-pyridin-4-yl)-phenylamine. The title compound was obtained as a light brown solid.

MS ISP (m/e): 426.2 (100) [(M+H)⁺].

¹H NMR (CDCl₃, 300 MHz): δ (ppm)=8.49-8.47 (m, 1H), 8.04-8.00 (m, 2H), 7.68 (m, 1H), 7.58-7.50 (m, 2H), 7.33-7.19 (m, 5H), 7.08 (m, 1H), 6.99-6.92 (m, 2H), 3.80 (s, 3H), 2.59 (s, 3H).

### Example 55

### [8-(4-Fluoro-phenyl)-5,6,7,8-tetrahydro-[1,2,4]triazolo[1,5-a]pyridin-2-yl]-[3-methoxy-4-(2-methyl-pyridin-4-yl)-phenyl]-amine

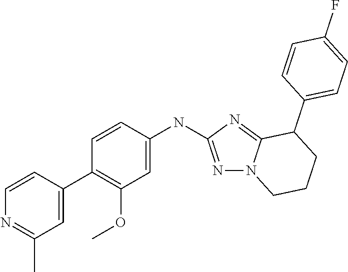

### a) 2-Bromo-8-(4-fluoro-phenyl)-5,6,7,8-tetrahydro-[1,2,4]triazolo[1,5-a]pyridine

Prepared in analogy to example 53d) employing 8-(4-fluoro-phenyl)-5,6,7,8-tetrahydro-[1,2,4]triazolo[1,5-a]pyridin-2-ylamine (see example 1d). The title compound was obtained as a light brown solid.

MS ISP (m/e): 296.1/298.1 (94/100) [(M+H)⁺].

¹H NMR (CDCl₃, 300 MHz): δ (ppm)=7.11-6.99 (m, 4H), 4.26-4.22 (m, 3H), 2.37-1.94 (m, 4H).

### b) [8-(4-Fluoro-phenyl)-5,6,7,8-tetrahydro-[1,2,4]triazolo[1,5-a]pyridin-2-yl]-[3-methoxy-4-(2-methyl-pyridin-4-yl)-phenyl]-amine

Prepared in analogy to example 53e) employing 2-bromo-8-(4-fluoro-phenyl)-5,6,7,8-tetrahydro-[1,2,4]triazolo[1,5-a]pyridine and 3-methoxy-4-(2-methyl-pyridin-4-yl)-phenylamine. The title compound was obtained as a white solid.

MS ISP (m/e): 430.4 (100) [(M+H)⁺].

¹H NMR (CDCl₃, 300 MHz): δ (ppm)=8.47-8.45 (m, 1H), 7.31-7.23 (m, 4H), 7.18-7.13 (m, 2H), 7.06-7.00 (m, 2H), 6.96-6.93 (m, 1H), 6.71 (m, 1H), 4.23-4.17 (m, 3H), 3.83 (s, 3H), 2.58 (s, 3H), 2.38-1.93 (m, 4H).

### Example 56

### [5-(4-Fluoro-phenyl)-5,6,7,8-tetrahydro-[1,2,4]triazolo[1,5-a]pyridin-2-yl]-[3-methoxy-4-(2-methyl-pyridin-4-yl)-phenyl]-amine

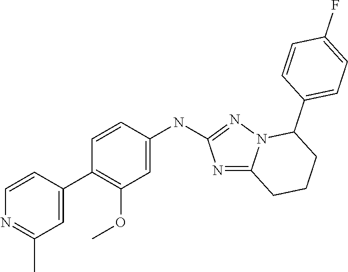

Prepared in analogy to example 55 steps a-b) starting from 5-(4-fluoro-phenyl)-5,6,7,8-tetrahydro-[1,2,4]triazolo[1,5-a]pyridin-2-ylamine (see example 2). The title compound was obtained as a white solid.

MS ISP (m/e): 430.4 (100) [(M+H)⁺].

¹H NMR (CDCl₃, 300 MHz): δ (ppm)=8.45-8.43 (m, 1H), 7.32-7.19 (m, 4H), 7.09-7.05 (m, 4H), 6.85-6.82 (m, 1H), 6.67 (m, 1H), 5.33-5.29 (m, 1H), 3.64 (s, 3H), 2.99-2.95 (m, 2H), 2.56 (s, 3H), 2.47-2.37 (m, 1H), 2.15-1.87 (m, 3H).

### Example 57

### 5-(4-Fluorophenyl)-N-(3-methoxy-4-(4-methyl-1H-imidazol-1-yl)phenyl)-7-(methylsulfonyl)-5,6,7,8-tetrahydro-[1,2,4]triazolo[1,5-a]pyrazin-2-amine

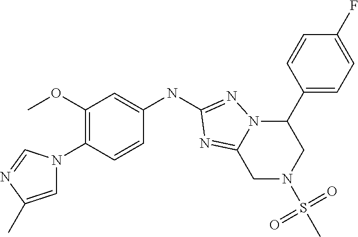

### a) [5-(4-Fluoro-phenyl)-[1,2,4]triazolo[1,5-a]pyrazin-2-yl]-[di-tert-butoxycarbonyl)]-amine

To a solution of 5-(4-fluorophenyl)-[1,2,4]triazolo[1,5-a]pyrazin-2-amine (see example 9c, 875 mg, 3.82 mmol) in THF (40 mL) were added Boc₂O (2.5 g, 2.66 mL, 11.5 mmol) and DMAP (23 mg, 191 μmol) and the reaction mixture was stirred at rt. After 12 hours further Boc₂O (2.5 g, 2.66 mL, 11.5 mmol) and DMAP (23 mg, 191 μmol) were added and stirred at 50° C. for 2 hours. The solvent was evaporated and the residue was purified by silica gel chromatography using diethyl ether/n-pentane as eluent. The title compound was obtained as light yellow solid (1.5 g, 91%).

MS ISP (m/e): 430.4 (14) [(M+H)⁺], 452.1 (100) [(M+Na)⁺].

¹H NMR (CDCl₃, 300 MHz): δ (ppm)=9.22 (s, 1H), 8.32 (s, 1H), 8.03-7.99 (m, 2H), 7.31-7.25 (m, 2H), 1.48 (s, 18H).

### b) [5-(4-Fluoro-phenyl)-5,6,7,8-tetrahydro-[1,2,4]triazolo[1,5-a]pyrazin-2-yl]-[di-(tert-butoxycarbonyl)]-amine

[5-(4-Fluoro-phenyl)-[1,2,4]triazolo[1,5-a]pyrazin-2-yl]-[di-(tert-butoxycarbonyl)]-amine (1.5 g, 3.49 mmol) in EtOH (120 mL) was hydrogenated in the presence of Pd on charcoal (10%, 1.5 g, 1.41 mmol) at 25 bar and 60° C. for 18 hours. The catalyst was filtered off, washed thoroughly with EtOH and the solvent was removed from the combined filtrates. The residue was purified by silica gel chromatography using dichloromethane/methanol (with 10% ammonia) as eluent. The title compound was obtained as a white solid (1.03 g, 68%).

MS ISP (m/e): 434.4 (50) [(M+H)⁺], 334.2 (100) [(M−Boc+H)⁺].

¹H NMR (CDCl₃, 300 MHz): δ (ppm)=7.05-7.02 (m, 4H), 5.40-5.37 (m, 1H), 4.24-4.23 (m, 2H), 3.64-3.23 (m, 2H), 1.45 (s, 18H).

### c) [5-(4-Fluoro-phenyl)-7-methanesulfonyl-5,6,7,8-tetrahydro-[1,2,4]triazolo[1,5-a]pyrazin-2-yl]-[di-(tert-butoxycarbonyl)]-amine

To a solution of [5-(4-fluoro-phenyl)-5,6,7,8-tetrahydro-[1,2,4]triazolo[1,5-a]pyrazin-2-yl]-[di-(tert-butoxycarbonyl)]-amine (100 mg, 231 μmol) and DIPEA (59.6 mg, 80.6 μL, 461 μmol) in THF (1 mL) at 0° C. was added methanesulfonyl chloride (29.1 mg, 19.8 μL, 254 μmol) and the reaction mixture was stirred at room temperature for 4 hours. The solvent was evaporated and the residue was purified by silica gel chromatography using dichloromethane/methanol (with 10% ammonia) as eluent. The title compound was obtained as a white solid (110 mg, 93%).

MS ISP (m/e): 512.3 (100) [(M+H)⁺], 412.2 (97) [(M−Boc+H)⁺].

¹H NMR (CDCl₃, 300 MHz): δ (ppm)=7.09-7.06 (m, 4H), 5.58-5.55 (m, 1H), 4.74-4.72 (m, 2H), 4.07-3.76 (m, 2H), 2.79 (s, 3H), 1.45 (s, 18H).

### d) 5-(4-Fluorophenyl)-7-(methylsulfonyl)-5,6,7,8-tetrahydro-[1,2,4]triazolo[1,5-a]pyrazin-2-amine

To a solution of [5-(4-fluoro-phenyl)-7-methanesulfonyl-5,6,7,8-tetrahydro-[1,2,4]triazolo[1,5-a]pyrazin-2-yl]-[di-(tert-butoxycarbonyl)]-amine (108 mg, 211 μmol) in dry CH₂Cl₂ (2 mL) at 0° C. was added TFA (169 mg, 114 μL, 1.48 mmol) added and the reaction mixture was stirred at room temperature. After 3 hours further TFA (169 mg, 114 μL, 1.48 mmol) was added and stirred at 50° C. for two hours. The reaction mixture was evaporated, sat. NaHCO₃ solution was added to the residue and the aqueous phase was extracted with ethyl acetate. The combined organic layers were dried over Na₂SO₄, filtered, and the solvents evaporated. The residue was purified by silica gel chromatography using dichloromethane/methanol (with 10% ammonia) as eluent. The title compound was obtained as a white solid (28 mg, 43%).

MS ISP (m/e): 312.1 (100) [(M+H)⁺].

¹H NMR (CDCl₃, 300 MHz): δ (ppm)=7.14-7.05 (m, 4H), 5.34-5.31 (m, 1H), 4.61-4.59 (m, 2H), 4.13 (bs, 2H), 4.03-3.97 (m, 1H), 3.68-3.62 (m, 1H), 2.78 (s, 3H).

### e) 5-(4-Fluorophenyl)-N-(3-methoxy-4-(4-methyl-1H-imidazol-1-yl)phenyl)-7-(methylsulfonyl)-5,6,7,8-tetrahydro-[1,2,4]triazolo[1,5-a]pyrazin-2-amine

A mixture of 1-(4-bromo-2-methoxy-phenyl)-4-methyl-1H-imidazole (WO2009076352, Example 1; 27.8 mg, 104 μmol), 5-(4-fluorophenyl)-7-(methylsulfonyl)-5,6,7,8-tetrahydro-[1,2,4]triazolo[1,5-a]pyrazin-2-amine (27 mg, 86.7 μmol), sodium phenoxide (15.1 mg, 130 μmol), tris(dibenzylideneacetone)dipalladium chloroform complex (3.59 mg, 3.47 μmol) and 2-(dicyclohexylphosphino)biphenyl (2.43 mg, 6.94 μmol) in dioxane (3 mL) was heated under an argon atmosphere in the microwave to 140° C. for 60 min. Further sodium phenoxide (15.1 mg, 130 μmol), tris(dibenzylideneacetone)dipalladium chloroform complex (3.59 mg, 3.47 μmol) and 2-(dicyclohexylphosphino)biphenyl (2.43 mg, 6.94 μmol) were added and the reaction mixture irradiated in the microwave at 140° C. for 45 min. The mixture was purified by silica gel chromatography using dichloromethane/methanol (with 10% ammonia) as eluent. After further purification by preparative HPLC the title compound was obtained as a white solid (14 mg, 32%).

MS ISP (m/e): 498.2 (100) [(M+H)₊].

¹H NMR (CDCl₃, 300 MHz): δ (ppm)=7.57 (m, 1H), 7.31-7.30 (m, 1H), 7.21-7.07 (m, 5H), 6.84-6.76 (m, 3H), 5.46-5.42 (m, 1H), 4.77-4.62 (m, 2H), 4.12-4.06 (m, 1H), 3.75-3.68 (m, 1H), 3.66 (s, 3H), 2.83 (s, 3H), 2.28 (s, 3H).

### Example 58

### 5-(4-fluorophenyl)-N-(3-methoxy-4-(4-methyl-1H-imidazol-1-yl)phenyl)-7-(2,2,2-trifluoroethyl)-5,6,7,8-tetrahydro-[1,2,4]triazolo[1,5-a]pyrazin-2-amine

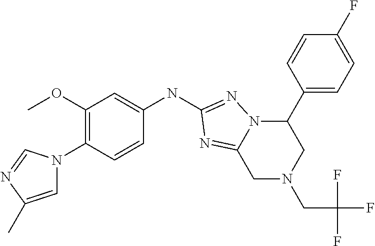

### a) [5-(4-Fluoro-phenyl)-7-(2,2,2-trifluoro-ethyl)-5,6,7,8-tetrahydro-[1,2,4]triazolo[1,5-a]pyrazin-2-yl]-[di-(tert-butoxycarbonyl)]-amine

To a solution of [5-(4-fluoro-phenyl)-5,6,7,8-tetrahydro-[1,2,4]triazolo[1,5-a]pyrazin-2-yl]-[di-(tert-butoxycarbonyl)]-amine (see example 57b, 100 mg, 231 μmol) and diisopropylethylamine (149 mg, 201 μA, 1.15 mmol) in dry THF (2 ml) at 0° C. was added 2,2,2-trifluoroethyl trifluoromethanesulfonate (69.6 mg, 43.2 μA, 300 μmol). The reaction mixture was allowed to warm to rt. After 4 h further diisopropylethylamine (149 mg, 201 μl, 1.15 mmol) and 2,2,2-trifluoroethyl trifluoromethanesulfonate (69.6 mg, 43.2 μA, 300 μmol) were added and the reaction mixture was stirred at 50° C. for 12 h. Further diisopropylethylamine (149 mg, 201 μl, 1.15 mmol) and 2,2,2-trifluoroethyl trifluoromethanesulfonate (69.6 mg, 43.2 μl, 300 μmol) were added and the reaction mixture was stirred at 70° C. for 48 h. The reaction mixture was evaporated to dryness. The crude material was purified by silica gel chromatography using dichloromethane/methanol (with 10% ammonia) as eluent. The title compound was obtained as a light brown foam (110 mg, 93%).

MS ISP (m/e): 516.2 (60) [(M+H)⁺], 416.3 (100) [(M−Boc+H)⁺].

¹H NMR (CDCl₃, 300 MHz): δ (ppm)=7.17-7.12 (m, 2H), 7.06-7.00 (m, 2H), 5.43-5.39 (m, 1H), 4.26-4.14 (m, 2H), 3.55-3.49 (m, 1H), 3.29-3.17 (m, 3H), 1.43 (s, 18H).

### b) 5-(4-fluorophenyl)-N-(3-methoxy-4-(4-methyl-1H-imidazol-1-yl)phenyl)-7-(2,2,2-trifluoroethyl)-5,6,7,8-tetrahydro-[1,2,4]triazolo[1,5-a]pyrazin-2-amine

Prepared in analogy to example 57d-e) employing [5-(4-fluoro-phenyl)-7-(2,2,2-trifluoro-ethyl)-5,6,7,8-tetrahydro-[1,2,4]triazolo[1,5-a]pyrazin-2-yl]-[di-(tert-butoxycarbonyl)]-amine. The title compound was obtained as a white solid.

MS ISP (m/e): 502.2 (100) [(M+H)⁺].

¹H NMR (CDCl₃, 300 MHz): δ (ppm)=7.56-7.55 (m, 1H), 7.32-7.31 (m, 1H), 7.25-7.22 (m, 2H), 7.09-7.03 (m, 3H), 6.81-6.77 (m, 2H), 6.58 (bs, 1H), 5.32-5.28 (m, 1H), 4.15-4.13 (m, 2H), 3.66 (s, 3H), 3.53-3.47 (m, 1H), 3.31-3.16 (m, 3H), 2.28 (s, 3H).

### Example 59

### 1-(8-(4-Fluorophenyl)-2-(3-methoxy-4-(4-methyl-1H-imidazol-1-yl)phenylamino)-5,6-dihydro-[1,2,4]triazolo[1,5-a]pyrazin-7(8H)-yl)-2-methylpropan-1-one

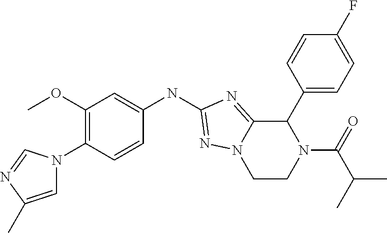

### a) [8-(4-Fluoro-phenyl)-5,6,7,8-tetrahydro-[1,2,4]triazolo[1,5-a]pyrazin-2-yl]-[di-(tert-butoxycarbonyl)]-amine

Prepared in analogy to example 57a-b) employing 8-(4-fluoro-phenyl)-[1,2,4]triazolo[1,5-a]pyrazin-2-ylamine (see example 11c). The title compound was obtained as a white solid.

MS ISP (m/e): 434.3 (22) [(M+H)⁺], 278.3 (100) [(M−Boc−tBu+H)⁺].

¹H NMR (CDCl₃, 300 MHz): δ (ppm)=7.36-7.32 (m, 2H), 7.06-7.00 (m, 2H), 5.24 (s, 1H), 4.34-4.19 (m, 2H), 3.48-3.31 (m, 2H), 2.09 (bs, 1H), 1.44 (s, 18H).

### b) [8-(4-Fluoro-phenyl)-7-isobutyryl-5,6,7,8-tetrahydro-[1,2,4]triazolo[1,5-a]pyrazin-2-yl]-[di-(tert-butoxycarbonyl)]-amine

To a solution of [8-(4-fluoro-phenyl)-5,6,7,8-tetrahydro-[1,2,4]triazolo[1,5-a]pyrazin-2-yl]-[di-(tert-butoxycarbonyl)]-amine (100 mg, 231 μmol) and diisopropylethylamine (149 mg, 201 μl, 1.15 mmol) in dry THF (2 ml) at 0° C. was added isobutyryl chloride (34.4 mg, 33.8 μl, 323 μmol). The reaction was allowed to warm to rt. After 4 h further diisopropylethylamine (149 mg, 201 μA, 1.15 mmol) and isobutyryl chloride (34.4 mg, 33.8 μA, 323 μmol) were added and the reaction mixture was stirred at 50° C. for 12 h. The reaction mixture was extracted with ethyl acetate and 2M Na2CO3, the organic layers combined, dried over Na2SO4 and evaporated to dryness. The crude material was purified by silica gel chromatography using dichloromethane/methanol (with 10% ammonia) as eluent. The title compound was obtained as a white solid (116 mg, 100%).

MS ISP (m/e): 504.3 (100) [(M+H)⁺], 404.4 (80) [(M−Boc+H)⁺].

### c) 1-(8-(4-Fluorophenyl)-2-(3-methoxy-4-(4-methyl-1H-imidazol-1-yl)phenylamino)-5,6-dihydro-[1,2,4]triazolo[1,5-a]pyrazin-7(8H)-yl)-2-methylpropan-1-one

Prepared in analogy to example 57d-e) employing [8-(4-fluoro-phenyl)-7-isobutyryl-5,6,7,8-tetrahydro-[1,2,4]triazolo[1,5-a]pyrazin-2-yl]-[di-(tert-butoxycarbonyl)]-amine. The title compound was obtained as a white solid.

MS ISP (m/e): 490.3 (100) [(M+H)⁺].

¹H NMR (CDCl₃, 300 MHz): δ (ppm)=7.61 (m, 1H), 7.35-7.30 (m, 2H), 7.17-6.97 (m, 5H), 6.85 (m, 1H), 6.77-6.73 (m, 1H), 4.27-4.13 (m, 3H), 3.85 (s, 3H), 3.71-3.58 (m, 1H), 2.92-2.83 (m, 1H), 2.30 (s, 3H), 1.23-1.18 (m, 6H).

### Example 60

### [3-Methoxy-4-(4-methyl-imidazol-1-yl)-phenyl]-(8-methyl-5-pyrrolidin-1-yl-[1,2,4]triazolo[1,5-c]pyrimidin-2-yl)-amine

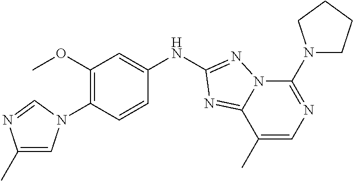

### a) 5-Methyl-2-pyrrolidin-1-yl-pyrimidin-4-ylamine

Prepared in analogy to example 12a, starting from 4-amino-2-chloro-5-methylpyrimidine and pyrrolidine. The title compound was obtained as white crystals (yield: 87%).

MS ISP (m/e): 179.2 (100) [(M+H)⁺].

### b) N-(5-methyl-2-(pyrrolidin-1-yl)-pyrimidin-4-yl)-N′-ethoxycarbonyl-thiourea

Prepared in analogy to example 1b, starting from 5-methyl-2-pyrrolidin-1-yl-pyrimidin-4-ylamine. The crude title compound was obtained as a yellow solid (yield: 110%) and was used directly in the next step without further purification.

MS ISP (m/e): 310.4 (100) [(M+H)⁺], 264.2 (48),221.3 (64).

### c) 8-Methyl-5-pyrrolidin-1-yl-[1,2,4]triazolo[1,5-c]pyrimidin-2-ylamine

Prepared in analogy to example 1c, starting from N4-[5-methyl-2-pyrrolidin-4-pyrimidinyl]-N′-carboethoxy-thiourea. The product was purified by column chromatography on silica gel using CH₂Cl₂/MeOH (v/v 19:1) as eluent to yield the title compound as white crystals (yield: 62%).

MS ISP (m/e): 219.3 (100) [(M+H)⁺].

### d) [3-Methoxy-4-(4-methyl-imidazol-1-yl)-phenyl]-(8-methyl-5-pyrrolidin-1-yl-[1,2,4]triazolo[1,5-c]pyrimidin-2-yl)-amine

Prepared in analogy to example 8e, starting from 8-methyl-5-pyrrolidin-1-yl-[1,2,4]triazolo[1,5-c]pyrimidin-2-ylamine and 1-(4-bromo-2-methoxy-phenyl)-4-methyl-1H-imidazole. The title compound was obtained as a beige solid (yield: 82%) after column chromatography on silica gel using a gradient from CH₂Cl₂ to CH₂Cl₂/MeOH 19:1 (v/v) as eluent.

MS ISP (m/e): 405.4 (100) [(M+H)⁺].

¹H NMR (DMSO-D₆, 300 MHz): δ (ppm)=9.98 (s, 1H), 7.78 (s, 1H), 7.66 (s, 1H), 7.23 (d, 1H), 7.08 (d, 1H), 7.02 (s, 1H), 3.99 (m, 4H), 3.82 (s, 3H), 2.23 (s, 3H), 2.14 (s, 3H), 1.94 (m, 4H).

### Example 61

### 7-(4-Chloro-phenyl)-2-[3-methoxy-4-(4-methyl-imidazol-1-yl)-phenylamino]-6,7-dihydro-4H-[1,2,4]triazolo[1,5-a]pyrimidin-5-one

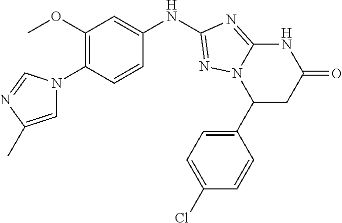

To a solution of N-[3-methoxy-4-(4-methyl-imidazol-1-yl)-phenyl]-4H-[1,2,4]triazole-3,5-diamine (511 mg, 2.0 mmol) in DMF (2 mL) was added methyl 4-chlorocinnamate (430 mg, 2.0 mmol) and was heated for 3 days to 160° C. Water was added and the reaction was extracted twice with ethyl acetate. The combined organic layers were dried over sodium sulfate, filtered and the solvent was evaporated under reduced pressure. The product was purified by column chromatography on silica gel using CH₂Cl₂/MeOH (v/v 19:1) as eluent to yield the title compound as a light brown solid (43 mg, 62%).

MS ISP (m/e): 450.2/452.1 (100/27) [(M+H)⁺].

¹H NMR (DMSO-D₆, 300 MHz): δ (ppm)=8.96 (s, 1H), 7.55 (s, 1H), 7.46 (s, 1H), 7.38 (d, 2H), 7.15 (d, 2H), 7.06 (s, 2H), 6.94 (s, 1H), 5.25 (t, 1H), 3.64 (s, 3H), 2.92 (dd, 1H), 2.40 (m, 1H), 2.12 (s, 3H).

### Example 62

### [8-(3-Chloro-4-fluoro-phenyl)-6-methyl-[1,2,4]triazolo[1,5-a]pyridin-2-yl]-[3-methoxy-4-(4-methyl-imidazol-1-yl)-phenyl]-amine

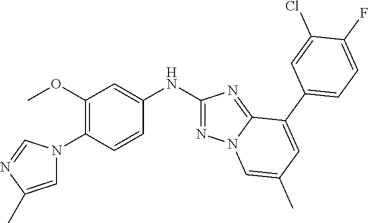

### a) 8-Methyl-5-pyrrolidin-1-yl-[1,2,4]triazolo[1,5-c]pyrimidin-2-ylamine

Prepared in analogy to example 1a, starting from 8-bromo-6-methyl-[1,2,4]triazolo[1,5-a]pyridin-2-ylamine and 3-chloro-4-fluoro phenylboronic acid in dimethoxyethane. The product was purified by column chromatography on silica gel using EtOAc as eluent to yield the title compound as a white solid (yield: 64%).

MS ISP (m/e): 277.2 (100) [(M+H)⁺].

### b) [3-Methoxy-4-(4-methyl-imidazol-1-yl)-phenyl]-(8-methyl-5-pyrrolidin-1-yl-[1,2,4]triazolo[1,5-c]pyrimidin-2-yl)-amine

Prepared in analogy to example 8e, starting from 8-(3-chloro-4-fluoro-phenyl)-6-methyl-[1,2,4]triazolo[1,5-a]pyridin-2-ylamine and 1-(4-bromo-2-methoxy-phenyl)-4-methyl-1H-imidazole. The title compound was obtained as a yellow solid (yield: 59%) after column chromatography on silica gel using a gradient from CH₂Cl₂ to CH₂Cl₂/MeOH 19:1 (v/v) as eluent and stirring with diethyl ether.

MS ISP (m/e): 463.2/465.2 (100/42) [(M+H)⁺].

¹H NMR (DMSO-D₆, 300 MHz): δ (ppm)=9.94 (s, 1H), 8.71 (s, 1H), 8.48 (d, 1H), 8.23 (m, 1H), 7.87 (s, 1H), 7.79 (s, 1H), 7.65 (s, 1H), 7.59 (t, 1H), 7.25 (s, 2H), 7.03 (s, 1H), 3.83 (s, 3H), 2.41 (s, 3H), 2.15 (s, 3H).

### Example 63

### [8-(3,4-Difluoro-phenyl)-6-methyl-[1,2,4]triazolo[1,5-a]pyridin-2-yl]-[3-methoxy-4-(4-methyl-imidazol-1-yl)-phenyl]-amine

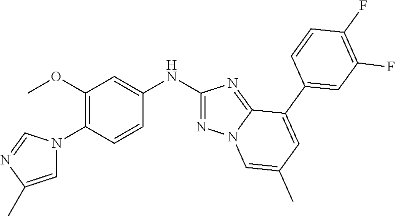

### a) 8-(3,4-Difluoro-phenyl)-6-methyl-[1,2,4]triazolo[1,5-a]pyridin-2-ylamine

Prepared in analogy to example 1a, starting from 8-bromo-6-methyl-[1,2,4]triazolo[1, 5-a]pyridin-2-ylamine and 3,4-difluorophenylboronic acid in dimethoxyethane. The product was purified by column chromatography on silica gel using EtOAc as eluent to yield the crude product, which was used directly in the next step.

### b) [8-(3,4-Difluoro-phenyl)-6-methyl-[1,2,4]triazolo[1,5-a]pyridin-2-yl]-[3-methoxy-4-(4-methyl-imidazol-1-yl)-phenyl]-amine

Prepared in analogy to example 8e, starting from 8-(3,4-difluoro-phenyl)-6-methyl-[1,2,4]triazolo[1,5-a]pyridin-2-ylamine and 1-(4-bromo-2-methoxy-phenyl)-4-methyl-1H-imidazole. The title compound was obtained as a yellow solid (yield: 20%) after column chromatography on silica gel using a gradient from CH₂Cl₂ to CH₂Cl₂/MeOH 19:1 (v/v) as eluent.

MS ISP (m/e): 447.2 (100) [(M+H)⁺].

¹H NMR (CDCl₃, 300 MHz): δ (ppm)=8.27 (s, 1H), 8.04 (m, 1H), 7.74 (m, 2H), 7.63 (s, 1H), 7.46 (s, 1H), 7.30 (t, 1H), 7.16 (d, 1H), 7.03 (s, 1H), 6.95 (d, 1H), 6.87 (s, 1H), 3.91 (s, 3H), 2.46 (s, 3H), 2.31 (s, 3H).

### Example 64

### [8-(4-Fluoro-2-methoxy-phenyl)-6-methyl-[1,2,4]triazolo[1,5-a]pyridin-2-yl]-[3-methoxy-4-(4-methyl-imidazol-1-yl)-phenyl]-amine

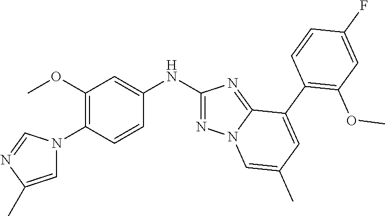

### a) 8-(4-Fluoro-2-methoxy-phenyl)-6-methyl-[1,2,4]triazolo[1,5-a]pyridin-2-ylamine

Prepared in analogy to example 1a, starting from 8-bromo-6-methyl-[1,2,4]triazolo[1,5-a]pyridin-2-ylamine and 4-fluoro-2-methoxy phenylboronic acid in dimethoxyethane. The product was purified by column chromatography on silica gel using a mixture of methylene chloride/dioxane 4:1 (v/v) as eluent to yield the crude product, which was used directly in the next step.

### b) [8-(4-Fluoro-2-methoxy-phenyl)-6-methyl-[1,2,4]triazolo[1,5-a]pyridin-2-yl]-[3-methoxy-4-(4-methyl-imidazol-1-yl)-phenyl]-amine

Prepared in analogy to example 8e, starting from 8-(4-fluoro-2-methoxy-phenyl)-6-methyl-[1,2,4]triazolo[1,5-a]pyridin-2-ylamine and 1-(4-bromo-2-methoxy-phenyl)-4-methyl-1H-imidazole. The title compound was obtained as a light brown solid (yield: 59%) after column chromatography on silica gel using a gradient from CH₂Cl₂ to CH₂Cl₂/MeOH 19:1 (v/v) as eluent and stirring with diethyl ether and little methylene chloride.

MS ISP (m/e): 459.3 (100) [(M+H)⁺].

¹H NMR (CDCl₃, 300 MHz): δ (ppm)=8.24 (s, 1H), 7.59 (m, 2H), 7.40 (s, 1H), 7.14 (d, 1H), 7.05 (s, 1H), 6.93 (d, 1H), 6.86 (s, 1H), 6.77 (m, 2H), 3.85 (s, 3H), 3.79 (s, 3H), 2.42 (s, 3H), 2.30 (s, 3H).

### Example 65

### [8-(2-Chloro-4-fluoro-phenyl)-6-methyl-[1,2,4]triazolo[1,5-a]pyridin-2-yl]-[3-methoxy-4-(4-methyl-imidazol-1-yl)-phenyl]-amine

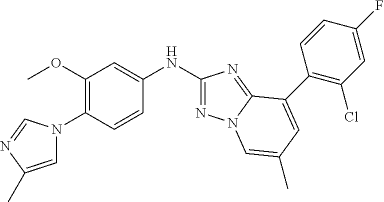

### a) 8-(2-Chloro-4-fluoro-phenyl)-6-methyl-[1,2,4]triazolo[1,5-a]pyridin-2-ylamine

Prepared in analogy to example 1a, starting from 8-bromo-6-methyl-[1,2,4]triazolo[1,5-a]pyridin-2-ylamine and 2-chloro-4-fluorophenylboronic acid in dimethoxyethane. The product was purified by column chromatography on silica gel using a mixture of methylene chloride/dioxane 4:1 (v/v) as eluent to yield the title compound as a white solid (yield: 36%).

MS ISP (m/e): 277.2 (100) [(M+H)⁺].

### b) [8-(2-Chloro-4-fluoro-phenyl)-6-methyl-[1,2,4]triazolo[1,5-a]pyridin-2-yl]-[3-methoxy-4-(4-methyl-imidazol-1-yl)-phenyl]-amine

Prepared in analogy to example 8e, starting from 8-(2-chloro-4-fluoro-phenyl)-6-methyl-[1,2,4]triazolo[1,5-a]pyridin-2-ylamine and 1-(4-bromo-2-methoxy-phenyl)-4-methyl-1H-imidazole. The title compound was obtained as a light yellow solid (yield: 56%) after column chromatography on silica gel using a gradient from CH₂Cl₂ to CH₂Cl₂/MeOH 19:1 (v/v) as eluent.

MS ISP (m/e): 463.2/465.2 (100/44) [(M+H)⁺].

¹H NMR (CDCl₃, 300 MHz): δ (ppm)=8.31 (s, 1H), 7.61 (m, 2H), 7.53 (dd, 1H), 7.34 (s, 1H), 7.29 (m, 1H), 7.14 (d, 1H), 7.13 (d, 1H), 6.99 (s, 1H), 6.94 (d, 1H), 6.86 (s, 1H), 3.86 (s, 3H), 2.45 (s, 3H), 2.30 (s, 3H).

### Example 66

### [8-(4-Fluoro-2-methyl-phenyl)-6-methyl-[1,2,4]triazolo[1,5-a]pyridin-2-yl]-[3-methoxy-4-(4-methyl-imidazol-1-yl)-phenyl]-amine

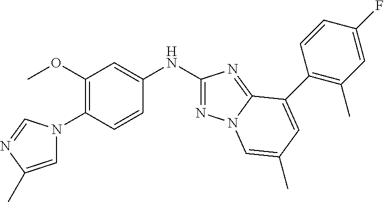

### a) 8-(4-Fluoro-2-methyl-phenyl)-6-methyl-[1,2,4]triazolo[1,5-a]pyridin-2-ylamine

Prepared in analogy to example 1a, starting from 8-bromo-6-methyl-[1,2,4]triazolo[1,5-a]pyridin-2-ylamine and 4-fluoro-2-methyl phenylboronic acid in dimethoxyethane. The product was purified by column chromatography on silica gel using a mixture of methylene chloride/dioxane 4:1 (v/v) as eluent to yield the title compound as a light yellow solid (yield: 36%).

MS ISP (m/e): 257.3 (100) [(M+H)⁺].

### b) [8-(4-Fluoro-2-methyl-phenyl)-6-methyl-[1,2,4]triazolo[1,5-a]pyridin-2-yl]-[3-methoxy-4-(4-methyl-imidazol-1-yl)-phenyl]-amine

Prepared in analogy to example 8e, starting from 8-(4-fluoro-2-methyl-phenyl)-6-methyl-[1,2,4]triazolo[1,5-a]pyridin-2-ylamine and 1-(4-bromo-2-methoxy-phenyl)-4-methyl-1H-imidazole. The title compound was obtained as a light brown solid (yield: 58%) after column chromatography on silica gel using a gradient from CH₂Cl₂ to CH₂Cl₂/MeOH 19:1 (v/v) as eluent.

MS ISP (m/e): 443.3 (100) (100/44) [(M+H)⁺].

¹H NMR (CDCl₃, 300 MHz): δ (ppm)=8.29 (s, 1H), 7.60 (m, 2H), 7.29 (m, 1H), 7.20 (s, 1H), 7.14 (d, 1H), 7.02 (d, 1H), 6.96 (m, 3H), 6.86 (s, 1H), 3.85 (s, 3H), 2.44 (s, 3H), 2.30 (s, 3H), 2.26 (s, 3H).

### Example 67

### 2-(3-Methoxy-4-(4-methyl-1H-imidazol-1-yl)phenylamino)-7-o-tolyl-[1,2,4]triazolo[1,5-a]pyrimidine-6-carbonitrile

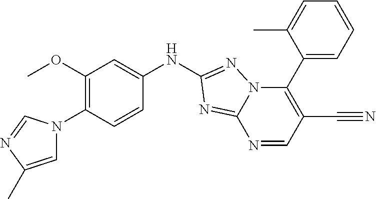

A solution of N3-(3-methoxy-4-(4-methyl-1H-imidazol-1-yl)phenyl)-4H-1,2,4-triazole-3,5-diamine (67.3 mg, 236 μmol) and (E)-3-(dimethylamino)-2-(2-methylbenzoyl)acrylonitrile (50.6 mg, 236 μmol) in acetic acid (1 mL) was heated to 100° C. over night. The solvent was evaporated in vacuo and the residue was purified by column chromatography on silica gel using a mixture of MeOH/methylene chloride 1:9 (v/v) as eluent to yield the title compound as a yellow solid (40 mg, 39%).

MS ISP (m/e): 437.2 (100) [(M+H)⁺].

¹H NMR (DMSO-D₆, 300 MHz): δ (ppm)=11.96 (s, 1H), 10.49 (s, 1H), 9.15 (s, 1H), 7.69 (s, 1H), 7.64-7.52 (m, 3H), 7.47-7.45 (m, 2H), 7.25 (d, 1H), 7.06 (d, 1H) 7.01 (s, 1H), 3.55 (s, 3H), 2.24 (s, 3H), 2.13 (s, 3H).

### Example 68

### N-(3-Methoxy-4-(4-methyl-1H-imidazol-1-yl)phenyl)-7-(pyridin-4-yl)-[1,2,4]triazolo[1,5-a]pyrimidin-2-amine

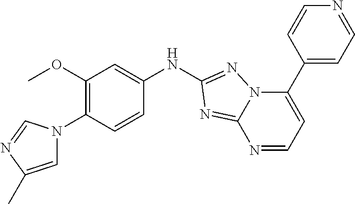

A solution of N3-(3-methoxy-4-(4-methyl-1H-imidazol-1-yl)phenyl)-4H-1,2,4-triazole-3,5-diamine (67.3 mg, 236 μmol) and (E)-3-(dimethylamino)-1-(pyridin-4-yl)prop-2-en-1-one (50.0 mg, 284 μmol) in acetic acid (1 mL) was heated to 100° C. over night. The solvent was evaporated in vacuo and the residue was purified by column chromatography on silica gel using a mixture of MeOH/methylene chloride 1:9 (v/v) as eluent to yield the title compound as a yellow solid (30 mg, 32%).

MS ISP (m/e): 399.1 (100) [(M+H)⁺].

¹H NMR (DMSO-D₆, 300 MHz): δ (ppm)=10.23 (br s, 1H), 8.89 (d, 2H), 8.81 (d, 1H), 8.24 (d, 2H), 7.84 (s, 1H), 7.65 (s, 1H), 7.58 (d, 1H), 7.28 (d, 1H), 7.18 (d, 1H), 7.03 (s, 1H), 3.8 (s, 3H), 2.14 (s, 3H).

### Example 69

### 7-(2-Fluorophenyl)-2-(3-methoxy-4-(4-methyl-1H-imidazol-1-yl)phenylamino)-[1,2,4]triazolo[1,5-a]pyrimidine-6-carbonitrile

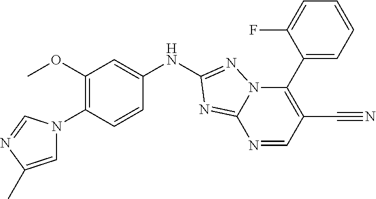

A solution of N3-(3-methoxy-4-(4-methyl-1H-imidazol-1-yl)phenyl)-4H-1,2,4-triazole-3,5-diamine (67.3 mg, 236 μmol) and (E)-3-(dimethylamino)-2-(2-fluorobenzoyl)acrylonitrile (50.0 mg, 229 μmol) in acetic acid (1 mL) was heated to 100° C. over night. The solvent was evaporated in vacuo and the residue was purified by column chromatography on silica gel using a mixture of MeOH/methylene chloride 1:9 (v/v) as eluent to yield the title compound as a yellow solid (40 mg, 38%).

MS ISP (m/e): 441.2 (100) [(M+H)⁺].

¹H NMR (DMSO-D₆, 300 MHz): δ (ppm)=11.96 (s, 1H), 10.52 (s, 1H), 9.17 (s, 1H), 7.95 (m, 1H), 7.84 (m, 1H), 7.72 (s, 1H), 7.65 (s, 1H), 7.59 (d, 1H), 7.55 (d, 1H), 7.24 (d, 1H), 7.07 (d, 1H), 7.02 (s, 1H), 3.63 (s, 3H), 2.13 (s, 3H).

### Example 70

### 2-(3-Methoxy-4-(4-methyl-1H-imidazol-1-yl)phenylamino)-7-(2-methoxyphenyl)-[1,2,4]triazolo[1,5-a]pyrimidine-6-carbonitrile

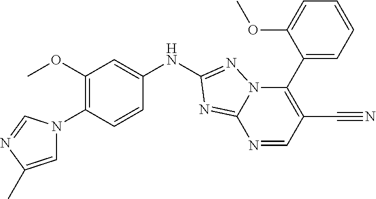

A solution of N3-(3-methoxy-4-(4-methyl-1H-imidazol-1-yl)phenyl)-4H-1,2,4-triazole-3,5-diamine (67.3 mg, 236 μmol) and (E)-3-(dimethylamino)-2-(2-methoxybenzoyl)acrylonitrile (50 mg, 217 μmol) in acetic acid (1 mL) was heated to 100° C. over night. The solvent was evaporated in vacuo and the residue was purified by column chromatography on silica gel using a mixture of MeOH/methylene chloride 1:9 (v/v) as eluent to yield the title compound as a yellow solid (30 mg, 28%).

MS ISN (m/e): 451.2 (100) [(M−H)⁺].
¹H NMR (DMSO-D₆, 300 MHz): δ (ppm)=11.95 (s, 1H), 10.46 (s, 1H), 9.1 (s, 1H), 7.76 (d, 1H), 7.7 (m, 2H), 7.64 (s, 1H), 7.37 (d, 1H), 7.26-7.23 (m, 2H), 7.11 (d, 1H), 7.01 (s, 1H), 3.84 (s, 3H), 3.6 (s, 3H), 2.13 (s, 3H).

### Example 71

### [7-(4-Fluoro-phenyl)-[1,2,4]triazolo[1,5-a]pyrimidin-2-yl]-[3-methoxy-4-(4-methyl-imidazol-1-yl)-phenyl]-amine

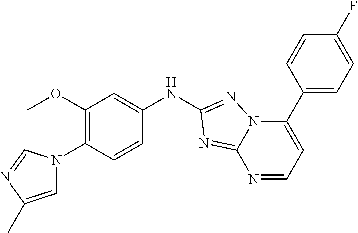

Prepared in analogy to example 35, starting from 4-fluoro-acetophenone and N-[3-Methoxy-4-(4-methyl-imidazol-1-yl)-phenyl]-4H-[1,2,4]triazole-3,5-diamine. The title compound was obtained as a brown solid (yield: 22%).

MS ISP (m/e): 416.3 (100) [(M+H)₊].

¹H NMR (DMSO-D₆, 300 MHz): δ (ppm)=10.27 (s, 1H), 8.75 (d, 1H), 8.51 (s, 1H), 8.35 (dd, 2H), 7.87 (s, 1H), 7.53 (m, 3H), 7.39 (m, 2H), 7.25 (d, 1H), 3.82 (s, 3H), 2.25 (s, 3H).

### Example 72

### [3-Methoxy-4-(4-methyl-imidazol-1-yl)-phenyl]-[8-methyl-5-(2,2,2-trifluoro-ethoxy)-[1,2,4]triazolo[1,5-c]pyrimidin-2-yl]-amine

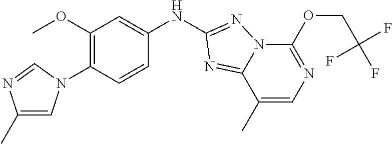

### a) 5-Methyl-2-(2,2,2-trifluoro-ethoxy)-pyrimidin-4-ylamine

Sodium (69 mg, 2 mmol) was added under an atmosphere of nitrogen at room temperature to 2,2,2-trifluoroethanol (3 mL, 40 mmol). The reaction was stirred for 1 hour. To this colorless solution 4-amino-2-chloro-5-methylpyrimidine (287.2 mg, 2.0 mmol) was added and the reaction was heated to 90° C. over night. Water was added and the reaction was extracted twice with ethyl acetate. The combined organic layer was washed with water and with saturated aqueous sodium chloride solution, dried over sodium sulfate, filtered and the solvent was evaporated under reduced pressure to yield the title compound as a white solid (414 mg, 100%).

MS ISP (m/e): 208.2 (100) [(M+H)⁺].

¹H NMR (DMSO-D₆, 300 MHz): δ (ppm)=7.74 (s, 1H), 7.92 (br s, 2H), 4.84 (q, 2H), 1.92 (s, 3H).

### b) 8-Methyl-5-(2,2,2-trifluoro-ethoxy)-[1,2,4]triazolo[1,5-c]pyrimidin-2-ylamine

Prepared in analogy to example 1 steps b-c) starting from 5-methyl-2-(2,2,2-trifluoro-ethoxy)-pyrimidin-4-ylamine. The title compound was obtained as a white solid (yield: 4%) after column chromatography on silica gel using a mixture of CH₂Cl₂/MeOH 19:1 (v/v) as eluent.

MS ISP (m/e): 248.2 (100) [(M+H)⁺].

¹H NMR (CDCl₃, 300 MHz): δ (ppm)=7.60 (s, 1H), 5.00 (q, 2H), 4.77 (br s, 2H), 2.39 (s, 3H).

### c) [3-Methoxy-4-(4-methyl-imidazol-1-yl)-phenyl]-[8-methyl-5-(2,2,2-trifluoro-ethoxy)-[1,2,4]triazolo[1,5-c]pyrimidin-2-yl]-amine

Prepared in analogy to example 8e, starting from 8-methyl-5-(2,2,2-trifluoro-ethoxy)-[1,2,4]triazolo[1,5-c]pyrimidin-2-ylamine and 1-(4-bromo-2-methoxy-phenyl)-4-methyl-1H-imidazole. The title compound was obtained as a light brown solid (yield: 53%) after column chromatography on silica gel using a gradient from CH₂Cl₂ to CH₂Cl₂/MeOH 19:1 (v/v) as eluent.

MS ISP (m/e): 434.3 (100) [(M+H)⁺].

¹H NMR (DMSO-D₆, 300 MHz): δ (ppm)=10.25 (s, 1H), 7.84 (s, 1H), 7.80 (s, 1H), 7.66 (s, 1H), 7.32 (d, 1H), 7.27 (d, 1H), 7.04 (s, 1H), 5.31 (q, 2H), 3.82 (s, 3H), 2.37 (s, 3H), 2.15 (s, 3H).

### Example 73

### 7-(3-Chloro-4-fluorophenyl)-N-(3-methoxy-4-(4-methyl-1H-imidazol-1-yl)phenyl)-[1,2,4]triazolo[1,5-a]pyrimidin-2-amine hydrochloride

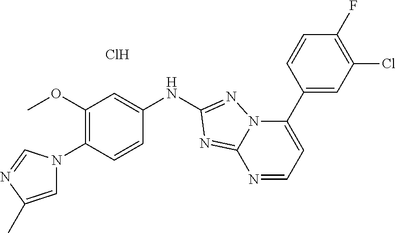

A solution of 1-(3-chloro-4-fluorophenyl)ethanone (51.8 mg, 300 μmmol) and tert.-butoxy-bis(dimethylamino)methane (Bredereck reagent) (52.3 mg, 300 μmol) in 1,4-dioxane (2 mL) was heated to reflux for 4 hours. The solvent was evaporated under reduced pressure and to the residue a solution of N3-(3-methoxy-4-(4-methyl-1H-imidazol-1-yl)phenyl)-4H-1,2,4-triazole-3,5-diamine (57.1 mg, 0.2 mmol) in acetic acid (1.0 mL) was added and the reaction was heated to 100° C. over night. On cooling to room temperature a light yellow solid precipitated. The precipitate was filtered and washed thoroughly with acetic acid. The solid was suspended in isopropanol and 37% aqueous hydrogen chloride solution was added. The solvent was removed under reduced pressure and the product dried in vacuo. The title compound was obtained as a light yellow solid (60 mg, 62%).

MS ISP (m/e): 450.1/452.2 (100/27) [(M+H)⁺], 228.2 (51), 179.2 (36).

¹H NMR (DMSO-D₆, 300 MHz): δ (ppm)=10.40 (s, 1H), 9.29 (s, 1H), 8.79 (d, 1H), 8.55 (d, 1H), 8.28 (m, 1H), 7.84 (s, 1H), 7.75 (t, 1H), 7.67 (s, 1H), 7.54 (d, 1H), 7.48 (d, 1H), 7.33 (d, 1H), 3.83 (s, 3H), 2.35 (s, 3H), 1.91 (s, 3H).

### Example 74

### 8-(2,4-Difluorophenyl)-N-(3-methoxy-4-(4-methyl-1H-imidazol-1-yl)phenyl)-6-methyl-[1,2,4]triazolo[1,5-a]pyridin-2-amine

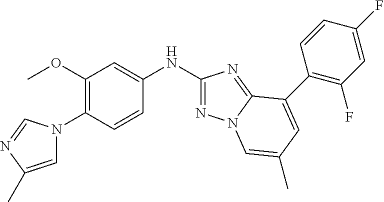

### a) 8-(2,4-Difluoro-phenyl)-6-methyl-[1,2,4]triazolo[1,5-a]pyridin-2-ylamine

Prepared in analogy to example 46b, starting from 8-bromo-6-methyl-[1,2,4]triazolo[1,5-a]pyridin-2-amine and 2,4-difluorophenylboronic acid. The crude product was purified by column chromatography on silica gel using ethyl acetate as eluent. The title compound was obtained as an off-white solid (yield: 36%).

MS ISP (m/e): 261.2 (100) [(M+H)⁺]

### b) 8-(2,4-Difluorophenyl)-N-(3-methoxy-4-(4-methyl-1H-imidazol-1-yl)phenyl)-6-methyl-[1,2,4]triazolo[1,5-a]pyridin-2-amine

Prepared in analogy to example 8e, starting from 8-(2,4-difluoro-phenyl)-6-methyl-[1,2,4]triazolo[1,5-a]pyridin-2-ylamine and 1-(4-bromo-2-methoxy-phenyl)-4-methyl-1H-imidazole. The title compound was obtained as a yellow solid (yield: 47%) after column chromatography on silica gel using a gradient from CH₂Cl₂ to CH₂Cl₂/MeOH 19:1 (v/v) as eluent.

MS ISP (m/e): 447.2 (100) [(M+H)⁺].

¹H NMR (CDCl₃, 300 MHz): δ (ppm)=8.28 (s, 1H), 7.86 (q, 1H), 7.63 (m, 2H), 7.44 (s, 1H), 7.16 (d, 1H), 7.03-6.93 (m, 4H), 6.86 (s, 1H), 3.87 (s, 3H), 2.45 (s, 3H), 2.30 (s, 3H).

### Example 75

### 8-(4-Fluoro-3-(trifluoromethyl)phenyl)-N-(3-methoxy-4-(4-methyl-1H-imidazol-1-yl)phenyl)-6-methyl-[1,2,4]triazolo[1,5-a]pyridin-2-amine

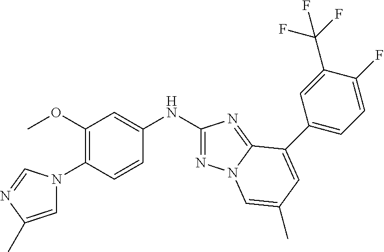

### a) 8-(4-Fluoro-3-trifluoromethyl-phenyl)-6-methyl-[1,2,4]triazolo[1,5-a]pyridin-2-ylamine

Prepared in analogy to example 46b, starting from 8-bromo-6-methyl-[1,2,4]triazolo[1,5-a]pyridin-2-amine and 4-fluoro-3-(trifluoromethyl)phenylboronic acid. The crude product was purified by column chromatography on silica gel using ethyl acetate as eluent. The title compound was obtained as a grey solid (yield: 82%).

MS ISP (m/e): 311.3 (100) [(M+H)⁺].

### b) 8-(4-Fluoro-3-(trifluoromethyl)phenyl)-N-(3-methoxy-4-(4-methyl-1H-imidazol-1-yl)phenyl)-6-methyl-[1,2,4]triazolo[1,5-a]pyridin-2-amine

Prepared in analogy to example 8e, starting from 8-(4-fluoro-3-trifluoromethyl-phenyl)-6-methyl-[1,2,4]triazolo[1,5-a]pyridin-2-ylamine and 1-(4-bromo-2-methoxy-phenyl)-4-methyl-1H-imidazole. The title compound was obtained as a yellow solid (yield: 54%) after column chromatography on silica gel using a gradient from CH₂Cl₂ to CH₂Cl₂/MeOH 19:1 (v/v) as eluent.

MS ISP (m/e): 497.4 (100) [(M+H)⁺].

¹H NMR (CDCl₃, 300 MHz): δ (ppm)=8.38-8.30 (m, 3H), 7.63 (s, 1H), 7.58 (s, 1H), 7.47 (s, 1H), 7.33 (t, 1H), 7.18 (d, 1H), 7.17-7.03 (m, 2H), 6.87 (s, 1H), 3.88 (s, 3H), 2.47 (s, 3H), 2.31 (s, 3H).

### Example 76

### 8-(4-Fluoro-3-methylphenyl)-N-(3-methoxy-4-(4-methyl-1H-imidazol-1-yl)phenyl)-6-methyl-[1,2,4]triazolo[1,5-a]pyridin-2-amine

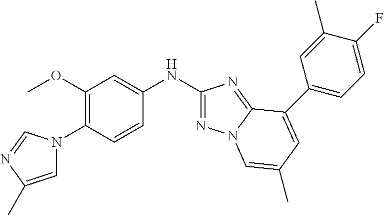

### a) 8-(4-Fluoro-3-methyl-phenyl)-6-methyl-[1,2,4]triazolo[1,5-a]pyridin-2-ylamine

Prepared in analogy to example 46b, starting from 8-bromo-6-methyl-[1,2,4]triazolo[1,5-a]pyridin-2-amine and 4-fluoro-3-methyl-phenylboronic acid. The crude product was purified by column chromatography on silica gel using ethyl acetate as eluent. The title compound was obtained as a white solid (yield: 89%).

MS ISP (m/e): 257.3 (100) [(M+H)⁺].

### b) 8-(4-Fluoro-3-methylphenyl)-N-(3-methoxy-4-(4-methyl-1H-imidazol-1-yl)phenyl)-6-methyl-[1,2,4]triazolo[1,5-a]pyridin-2-amine

Prepared in analogy to example 8e, starting from 8-(4-fluoro-3-methyl-phenyl)-6-methyl-[1,2,4]triazolo[1,5-a]pyridin-2-ylamine and 1-(4-bromo-2-methoxy-phenyl)-4-methyl-1H-imidazole. The title compound was obtained as a light brown solid (yield: 43%) after column chromatography on silica gel using a gradient from CH₂Cl₂ to CH₂Cl₂/MeOH 19:1 (v/v) as eluent.

MS ISP (m/e): 443.3 (100) [(M+H)⁺].

¹H NMR (CDCl₃, 300 MHz): δ (ppm)=8.24 (s, 1H), 7.80 (m, 2H), 7.65 (s, 1H), 7.62 (s, 1H), 7.41 (s, 1H), 7.17 (d, 1H), 7.11-7.09 (m, 3H), 6.98 (dd, 1H), 6.87 (s, 1H), 3.88 (s, 3H), 2.44 (s, 3H), 2.37 (s, 3H), 2.31 (s, 3H).

### Example 77

### 7-(4-Chlorophenyl)-2-(3-methoxy-4-(4-methyl-1H-imidazol-1-yl)phenylamino)-4-propyl-6,7-dihydro-[1,2,4]triazolo[1,5-a]pyrimidin-5(4H)-one

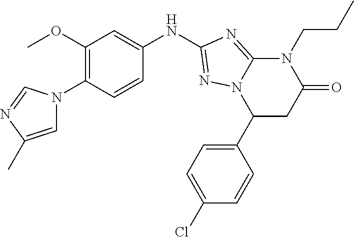

### a) N3-[3-Methoxy-4-(4-methyl-imidazol-1-yl)-phenyl]-N-5-propyl-1H-[1,2,4]triazole-3,5-diamine

To a suspension of N3-(3-methoxy-4-(4-methyl-1H-imidazol-1-yl)phenyl)-4H-1,2,4-triazole-3,5-diamine (285 mg, 1 mmol) and propionaldehyde (72.6 mg, 91.0 μA, 1.2 mmol) in a mixture of ethanol (2 mL), tetrahydrofurane (2 mL) and acetic acid (3 mL) was added at room temperature under an atmosphere of nitrogen sodium borohydride (56.7 mg, 1.5 mmol). The mixture was stirred at room temperature over night. Water and 1N aqueous NaOH solution was added and the reaction was extracted twice with ethyl acetate and twice with CH₂Cl₂/MeOH 19:1. The combined organic layers were dried over sodium sulfate, filtered and the solvent was removed under reduced pressure. The title compound was obtained as an off-white solid (123 mg, 37%) after column chromatography on silica gel using a gradient from CH₂Cl₂ to CH₂Cl₂/MeOH 19:1 (v/v) as eluent.

MS ISP (m/e): 328.4 (100) [(M+H)⁺].
¹H NMR (DMSO-D₆, 300 MHz): δ (ppm)=11.41 (br s, 1H), 8.98 (s, 1H), 7.58 (s, 1H), 7.56 (s, 1H), 7.10 (d, 1H), 7.07 (d, 1H), 6.97 (s, 1H), 6.44 (br t, 1H), 3.74 (s, 3H), 3.05 (q, 2H), 2.13 (s, 3H), 1.53 (sept, 2H), 0.89 (t, 3H).

### b) 7-(4-Chlorophenyl)-2-(3-methoxy-4-(4-methyl-1H-imidazol-1-yl)phenylamino)-4-propyl-6,7-dihydro-[1,2,4]triazolo[1,5-a]pyrimidin-5(4H)-one

A solution of N3-(3-methoxy-4-(4-methyl-1H-imidazol-1-yl)phenyl)-N5-propyl-4H-1,2,4-triazole-3,5-diamine (110 mg, 336 μmol) and methyl 4-chlorocinnamate (67.4 mg, 336 μmol) in DMF (2 mL) was heated to 150° C. for 6 hours under nitrogen. Water was added and the reaction was extracted twice with ethyl acetate. The combined organic layers were washed with saturated aqueous sodium chloride solution, dried over sodium sulfate, filtered and the solvent was removed under reduced pressure. The title compound was obtained as a light yellow solid (6 mg, 3%) after column chromatography on silica gel using a gradient from CH₂Cl₂ to CH₂Cl₂/MeOH 19:1 (v/v) as eluent.

MS ISP (m/e): 492.2 (100) [(M+H)⁺].

¹H NMR (CDCl₃, 300 MHz): δ (ppm)=7.58 (s, 1H), 7.39-7.35 (m, 3H), 7.14-7.09 (m, 3H), 6.83 (s, 1H), 6.81 (d, 1H), 6.63 (s, 1H), 5.42 (t, 1H), 3.92 (m, 2H), 3.72 (s, 3H), 3.36 (dd, 1H), 3.11 (dd, 1H), 2.29 (s, 3H), 1.74 (q, 2H), 0.95 (t, 3H).

### Example 78

### 2-Fluoro-5-(2-(3-methoxy-4-(4-methyl-1H-imidazol-1-yl)phenylamino)-6-methyl-[1,2,4]triazolo[1,5-a]pyridin-8-yl)benzonitrile

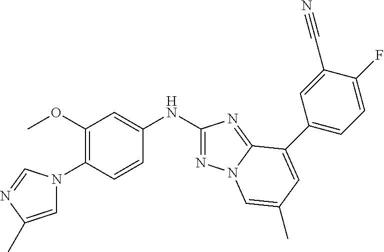

### a) 5-(2-Amino-6-methyl-[1,2,4]triazolo[1,5-a]pyridin-8-yl)-2-fluoro-benzonitrile

Prepared in analogy to example 46b, starting from 8-bromo-6-methyl-[1,2,4]triazolo[1,5-a]pyridin-2-amine and 3-cyano-4-fluorophenylboronic acid. The crude product was purified by column chromatography on silica gel using a gradient from CH₂Cl₂ to CH₂Cl₂/MeOH 19:1 (v/v) as eluent. The title compound was obtained as a light grey solid (yield: 92%).

MS ISP (m/e): 268.2 (100) [(M+H)⁺].

### b) 2-Fluoro-5-(2-(3-methoxy-4-(4-methyl-1H-imidazol-1-yl)phenylamino)-6-methyl-[1,2,4]triazolo[1,5-a]pyridin-8-yl)benzonitrile

Prepared in analogy to example 8e, starting from 5-(2-amino-6-methyl-[1,2,4]triazolo [1,5-a]pyridin-8-yl)-2-fluoro-benzonitrile and 1-(4-bromo-2-methoxy-phenyl)-4-methyl-1H-imidazole. The title compound was obtained as a yellow solid (yield: 54%) after column chromatography on silica gel using a gradient from CH₂Cl₂ to CH₂Cl₂/MeOH 19:1 (v/v) as eluent.

MS ISP (m/e): 454.3 (100) [(M+H)⁺].

¹H NMR (DMSO-D₆, 300 MHz): δ (ppm)=9.96 (s, 1H), 8.74 (m, 2H), 8.65 (m, 1H), 7.93 (s, 1H), 7.77 (s, 1H), 7.71 (t, 1H), 7.65 (s, 1H), 7.25 (s, 2H), 7.02 (s, 1H), 3.83 (s, 3H), 2.42 (s, 3H), 2.15 (s, 3H).

### Example 79

### 7-(3-Chloro-4-fluorophenyl)-N-(3-methoxy-4-(4-methyl-1H-imidazol-1-yl)phenyl)-5-(trifluoromethyl)-[1,2,4]triazolo[1,5-a]pyrimidin-2-amine acetate

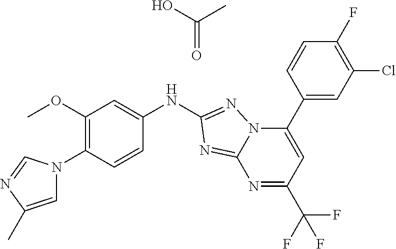

A solution of 1-(3-chloro-4-fluorophenyl)-4,4,4-trifluorobutane-1,3-dione (80.6 mg, 300 mmol) and of N3-(3-methoxy-4-(4-methyl-1H-imidazol-1-yl)phenyl)-4H-1,2,4-triazole-3,5-diamine (57.1 mg, 0.2 mmol) in acetic acid (1 mL) was heated to 100° C. over night. The solvent was removed under reduced pressure and the residue was treated with diethyl ether. The precipitate was filtered off, washed with diethyl ether and dried to yield the title compound as a yellow solid (60 mg, 52%).

MS ISP (m/e): 518.1/520.1 (100/37) [(M+H)⁺].

¹H NMR (DMSO-D₆, 300 MHz): δ (ppm)=11.96 (br s, 1H), 10.48 (d, 1H), 8.62 (m, 2H), 7.87 (s, 1H), 7.82 (s, 1H), 7.66 (t, 1H), 7.33 (br s, 2H), 7.13 (s, 1H), 3.84 (s, 3H), 2.17 (s, 3H), 1.91 (s, 3H).

### Example 80

### 8-(3,4-Difluorophenyl)-N-(3-methoxy-4-(4-methyl-1H-imidazol-1-yl)phenyl)-6-(trifluoromethyl)-[1,2,4]triazolo[1,5-a]pyridin-2-amine

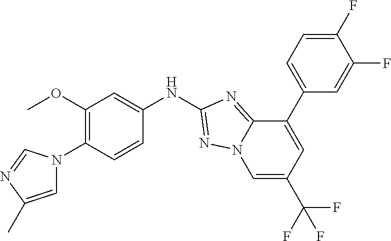

### a) 8-(3,4-Difluoro-phenyl)-6-trifluoromethyl-[1,2,4]triazolo[1,5-a]pyridin-2-ylamine

Prepared in analogy to example 1a-1c), starting from 3-bromo-5-(trifluoromethyl)pyridin-2-amine and 3,4-difluorophenylboronic acid. The crude product was purified by column chromatography on silica gel using a gradient from heptane/ethyl acetate 1:1 (v/v) to ethyl acetate as eluent. The title compound was obtained as a light grey solid (yield: 36%, 3 steps).

MS ISP (m/e): 315.1 (100) [(M+H)⁺].

¹H NMR (DMSO-D₆, 300 MHz): δ (ppm)=9.22 (s, 1H), 8.37 (m, 1H), 8.11 (m, 1H), 8.05 (s, 1H), 7.60 (q, 1H), 6.57 (br s, 2H).

### b) 8-(3,4-Difluorophenyl)-N-(3-methoxy-4-(4-methyl-1H-imidazol-1-yl)phenyl)-6-(trifluoromethyl)-[1,2,4]triazolo[1,5-a]pyridin-2-amine

Prepared in analogy to example 8e, starting from 8-(3,4-difluoro-phenyl)-6-trifluoromethyl-[1,2,4]triazolo[1,5-a]pyridin-2-ylamine and 1-(4-bromo-2-methoxy-phenyl)-4-methyl-1H-imidazole. The title compound was obtained as a brown solid (yield: 27%) after column chromatography on silica gel using a gradient from CH₂Cl₂ to CH₂Cl₂/MeOH 19:1 (v/v) as eluent.

MS ISP (m/e): 501.1 (100) [(M+H)⁺].

¹H NMR (DMSO-D₆, 300 MHz): δ (ppm)=10.24 (s, 1H), 9.54 (s, 1H), 8.43 (m, 1H), 8.21 (s, 1H), 8.19 (m, 1H), 7.83 (s, 1H), 7.66-7.58 (m, 2H), 7.28 (s, 2H), 7.04 (s, 1H), 3.86 (s, 3H), 2.15 (s, 3H).

### Example 81

### 6-Chloro-8-(3,4-difluorophenyl)-N-(3-methoxy-4-(4-methyl-1H-imidazol-1-yl)phenyl)-[1,2,4]triazolo[1,5-a]pyridin-2-amine

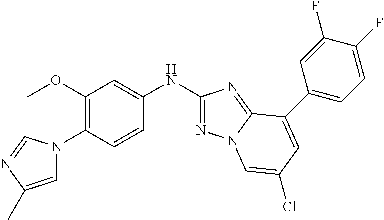

### a) 6-Chloro-8-(3,4-difluoro-phenyl)-[1,2,4]triazolo[1,5-a]pyridin-2-ylamine

Prepared in analogy to example 1a-1c), starting from 3-bromo-5-chloropyridin-2-amine and 3,4-difluorophenylboronic acid. The crude product was purified by column chromatography on silica gel using a gradient from heptane/ethyl acetate 1:1 (v/v) to ethyl acetate as eluent. The title compound was obtained as a light grey solid (yield: 68%, 3 steps).

MS ISP (m/e): 281.1/283.1 (100/39) [(M+H)⁺].

¹H NMR (DMSO-D₆, 300 MHz): δ (ppm)=8.93 (s, 1H), 8.36 (m, 1H), 8.07 (m, 1H), 7.92 (s, 1H), 7.60 (q, 1H), 6.35 (br s, 2H).

### b) 6-Chloro-8-(3,4-difluorophenyl)-N-(3-methoxy-4-(4-methyl-1H-imidazol-1-yl)phenyl)-[1,2,4]triazolo[1,5-a]pyridin-2-amine

Prepared in analogy to example 8e, starting from 6-chloro-8-(3,4-difluoro-phenyl)-[1,2,4]triazolo[1,5-a]pyridin-2-ylamine and 1-(4-bromo-2-methoxy-phenyl)-4-methyl-1H-imidazole. The title compound was obtained as a yellow solid (yield: 56%) after column chromatography on silica gel using a gradient from CH₂Cl₂ to CH₂Cl₂/MeOH 19:1 (v/v) as eluent.

¹H NMR (DMSO-D₆, 300 MHz): δ (ppm)=9.22 (s, 1H), 8.42 (m, 1H), 8.12 (m, 1H), 8.08 (s, 1H), 7.81 (s, 1H), 7.66 (s, 1H), 7.64 (t, 1H), 7.25 (s, 2H), 7.03 (s, 1H), 3.84 (s, 3H), 2.15 (s, 3H).

### Example 82

### N-(3-Methoxy-4-(4-methyl-1H-imidazol-1-yl)phenyl)-6-methyl-8-(4-morpholinophenyl)-[1,2,4]triazolo[1,5-a]pyridin-2-amine

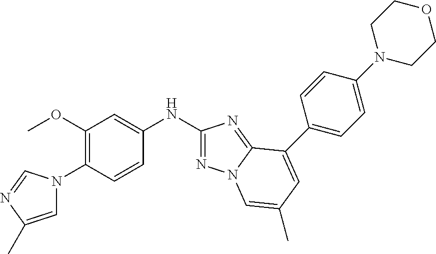

### a) 6-Methyl-8-(4-morpholin-4-yl-phenyl)-[1,2,4]triazolo[1,5-a]pyridin-2-ylamine

Prepared in analogy to example 46b, starting from 8-bromo-6-methyl-[1,2,4]triazolo[1,5-a]pyridin-2-amine and 4-morpholinophenylboronic acid. The crude product was purified by column chromatography on silica gel using a gradient from CH₂Cl₂ to CH₂Cl₂/MeOH 19:1 (v/v) as eluent. The title compound was obtained as a light brown solid (yield: 68%).

MS ISP (m/e): 310.4 (100) [(M+H)⁺].

### b) N-(3-Methoxy-4-(4-methyl-1H-imidazol-1-yl)phenyl)-6-methyl-8-(4-morpholinophenyl)-[1,2,4]triazolo[1,5-a]pyridin-2-amine

Prepared in analogy to example 8e, starting from 6-methyl-8-(4-morpholin-4-yl-phenyl)-[1,2,4]triazolo[1,5-a]pyridin-2-ylamine and 1-(4-bromo-2-methoxy-phenyl)-4-methyl-1H-imidazole. The title compound was obtained as an off-white solid (yield: 44%) after column chromatography on silica gel using a gradient from CH₂Cl₂ to CH₂Cl₂/MeOH 19:1 (v/v) as eluent.

MS ISP (m/e): 496.4 (100) [(M+H)⁺].

¹H NMR (DMSO-D₆, 300 MHz): δ (ppm)=9.89 (s, 1H), 8.57 (s, 1H), 8.14 (d, 2H), 7.88 (s, 1H), 7.69 (s, 1H), 7.64 (s, 1H), 7.24 (s, 2H), 7.06 (d, 2H), 7.02 (s, 1H), 3.85 (s, 3H), 3.78 (m, 4H), 3.21 (m, 4H), 2.39 (s, 3H), 2.15 (s, 3H).

### Example 83

### 2-(4-(2-(3-Methoxy-4-(4-methyl-1H-imidazol-1-yl)phenylamino)-6-methyl-[1,2,4]triazolo[1,5-a]pyridin-8-yl)phenyl)acetonitrile

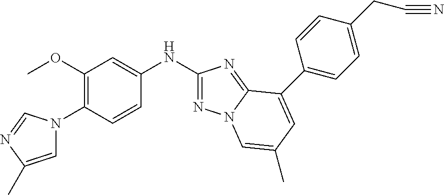

### a) [4-(2-Amino-6-methyl-[1,2,4]triazolo[1,5-a]pyridin-8-yl)-phenyl]-acetonitrile

Prepared in analogy to example 46b, starting from 8-bromo-6-methyl-[1,2,4]triazolo[1,5-a]pyridin-2-amine and 4-(cyanomethyl)phenylboronic acid. The crude product was purified by column chromatography on silica gel using a gradient from CH₂Cl₂ to CH₂Cl₂/MeOH 19:1 (v/v) as eluent. The title compound was obtained as a yellow solid (yield: 78%).

MS ISP (m/e): 264.2 (100) [(M+H)⁺].

### b) 2-(4-(2-(3-Methoxy-4-(4-methyl-1H-imidazol-1-yl)phenylamino)-6-methyl-[1,2,4]triazolo[1,5-a]pyridin-8-yl)phenyl)acetonitrile

Prepared in analogy to example 8e, starting from [4-(2-amino-6-methyl-[1,2,4]triazolo[1,5-a]pyridin-8-yl)-phenyl]-acetonitrile and 1-(4-bromo-2-methoxy-phenyl)-4-methyl-1H-imidazole. The title compound was obtained as a light yellow solid (yield: 29%) after column chromatography on silica gel using a gradient from CH₂Cl₂ to CH₂Cl₂/MeOH 19:1 (v/v) as eluent.

MS ISP (m/e): 450.2 (100) [(M+H)⁺].

¹H NMR (DMSO-D₆, 300 MHz): δ (ppm)=9.92 (s, 1H), 8.68 (s, 1H), 8.22 (d, 2H), 7.86 (s, 1H), 7.79 (s, 1H), 7.65 (s, 1H), 7.50 (d, 2H), 7.24 (s, 2H), 7.03 (s, 1H), 4.13 (s, 2H), 3.84 (s, 3H), 2.42 (s, 3H), 2.15 (s, 3H).

### Example 84

### 8-(2,4-Dimethoxyphenyl)-N-(3-methoxy-4-(4-methyl-1H-imidazol-1-yl)phenyl)-6-methyl-[1,2,4]triazolo[1,5-a]pyridin-2-amine

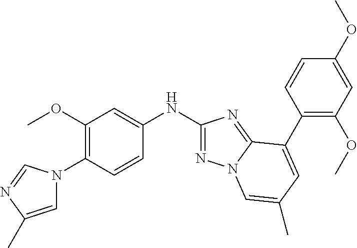

### a) 8-(2,4-Dimethoxy-phenyl)-6-methyl-[1,2,4]-triazolo[1,5-a]pyridin-2-ylamine

Prepared in analogy to example 46b, starting from 8-bromo-6-methyl-[1,2,4]triazolo[1,5-a]pyridin-2-amine and 2,4-dimethoxyphenylboronic acid. The crude product was purified by column chromatography on silica gel using a gradient from CH₂Cl₂ to CH₂Cl₂/MeOH 19:1 (v/v) as eluent. The title compound was obtained as a light brown solid (yield: 81%).

MS ISP (m/e): 285.2 (100) [(M+H)⁺].

### b) 8-(2,4-Dimethoxyphenyl)-N-(3-methoxy-4-(4-methyl-1H-imidazol-1-yl)phenyl)-6-methyl-[1,2,4]triazolo[1,5-a]pyridin-2-amine

Prepared in analogy to example 8e, starting from 8-(2,4-dimethoxy-phenyl)-6-methyl-[1,2,4]triazolo[1,5-a]pyridin-2-ylamine and 1-(4-bromo-2-methoxy-phenyl)-4-methyl-1H-imidazole. The title compound was obtained as a light orange solid (yield: 77%) after column chromatography on silica gel using a gradient from CH₂Cl₂ to CH₂Cl₂/MeOH 19:1 (v/v) as eluent.

MS ISP (m/e): 471.4 (100) [(M+H)⁺].

¹H NMR (DMSO-D₆, 300 MHz): δ (ppm)=9.82 (s, 1H), 8.58 (s, 1H), 7.73 (s, 1H), 7.63 (s, 1H), 7.56 (d, 1H), 7.44 (s, 1H), 7.22 (s, 2H), 7.01 (s, 1H), 6.71 (s, 1H), 6.65 (d, 1H), 3.83 (s, 3H), 3.78 (s, 3H), 3.76 (s, 3H), 2.36 (s, 3H), 2.14 (s, 3H).

### Example 85

### 8-(3,4-Difluorophenyl)-6-fluoro-N-(3-methoxy-4-(4-methyl-1H-imidazol-1-yl)phenyl)-[1,2,4]triazolo[1,5-a]pyridin-2-amine

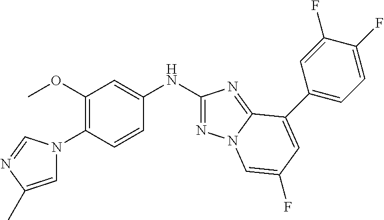

### a) 8-(3,4-Difluoro-phenyl)-6-fluoro-[1,2,4]triazolo[1,5-a]pyridin-2-ylamine

Prepared in analogy to example 1a-1c), starting 3-bromo-5-fluoropyridin-2-amine and 3,4-difluorophenylboronic acid. The crude product was purified by column chromatography on silica gel using a gradient from heptane/ethyl acetate 1:1 (v/v) to ethyl acetate as eluent. The title compound was obtained as a white solid (yield: 53%, 3 steps).

MS ISP (m/e): 265.2 (100) [(M+H)⁺].

### b) 8-(3,4-Difluorophenyl)-6-fluoro-N-(3-methoxy-4-(4-methyl-1H-imidazol-1-yl)phenyl)-[1,2,4]triazolo[1,5-a]pyridin-2-amine

Prepared in analogy to example 8e, starting from 8-(3,4-difluoro-phenyl)-6-fluoro-[1,2,4]triazolo[1,5-a]pyridin-2-ylamine and 1-(4-bromo-2-methoxy-phenyl)-4-methyl-1H-imidazole. The title compound was obtained as a yellow solid (yield: 59%) after column chromatography on silica gel using a gradient from CH₂Cl₂ to CH₂Cl₂/MeOH 19:1 (v/v) as eluent.

¹H NMR (DMSO-D₆, 300 MHz): δ (ppm)=9.22 (t, 1H), 8.46 (m, 1H), 8.15 (m, 2H), 7.80 (s, 1H), 7.65 (s, 1H), 7.62 (q, 1H), 7.22 (s, 2H), 7.03 (s, 1H), 3.84 (s, 3H), 2.15 (s, 3H).

### Example 86

### 8-(2-Chloro-4-fluorophenyl)-6-fluoro-N-(3-methoxy-4-(4-methyl-1H-imidazol-1-yl)phenyl)-[1,2,4]triazolo[1,5-a]pyridin-2-amine

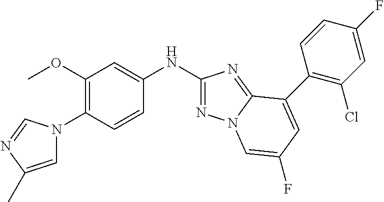

### a) 8-(2-Chloro-4-fluoro-phenyl)-6-fluoro-[1,2,4]triazolo[1,5-a]pyridin-2-ylamine

Prepared in analogy to example 1a-1c), starting 3-bromo-5-fluoropyridin-2-amine and 2-chloro-4-fluorophenylboronic acid. The crude product was purified by column chromatography on silica gel using a gradient from heptane/ethyl acetate 1:1 (v/v) to ethyl acetate as eluent. The title compound was obtained as a white solid (yield: 34%, 3 steps).

MS ISP (m/e): 281.2/283.2 (100/42) [(M+H)⁺].

### b) 8-(2-Chloro-4-fluorophenyl)-6-fluoro-N-(3-methoxy-4-(4-methyl-1H-imidazol-1-yl)phenyl)-[1,2,4]triazolo[1,5-a]pyridin-2-amine

Prepared in analogy to example 8e, starting from 8-(2-chloro-4-fluoro-phenyl)-6-fluoro-[1,2,4]triazolo[1,5-a]pyridin-2-ylamine and 1-(4-bromo-2-methoxy-phenyl)-4-methyl-1H-imidazole. The title compound was obtained as a yellow solid (yield: 61%) after column chromatography on silica gel using a gradient from CH₂Cl₂ to CH₂Cl₂/MeOH 19:1 (v/v) as eluent.

MS ISP (m/e): 467.2/469.2 (100/38) [(M+H)⁺].

¹H NMR (DMSO-D₆, 300 MHz): δ (ppm)=9.27 (t, 1H), 7.82 (dd, 1H), 7.74-7.64 (m, 4H), 7.40 (dt, 1H), 7.25-7.21 (m, 2H), 7.01 (s, 1H), 3.76 (s, 3H), 2.14 (s, 3H).

### Example 87

### N-(3-Methoxy-4-(4-methyl-1H-imidazol-1-yl)phenyl)-6-methyl-8-(2-methylbenzo[d]oxazol-6-yl)-[1,2,4]triazolo[1,5-a]pyridin-2-amine

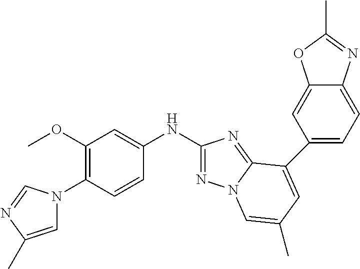

### a) 6-Methyl-8-(2-methyl-benzooxazol-6-yl)-[1,2,4]triazolo[1,5-a]pyridin-2-ylamine

Prepared in analogy to example 46b, starting from 8-bromo-6-methyl-[1,2,4]triazolo[1,5-a]pyridin-2-amine and 1-methyl-1H-benzo[d]imidazol-6-ylboronic acid. The crude product was purified by column chromatography on silica gel using a gradient from CH₂Cl₂ to CH₂Cl₂/MeOH 9:1 (v/v) as eluent. The title compound was obtained as a white solid (yield: 37%).

MS ISP (m/e): 280.2 (100) [(M+H)⁺].

### b) N-(3-Methoxy-4-(4-methyl-1H-imidazol-1-yl)phenyl)-6-methyl-8-(2-methylbenzo[d]oxazol-6-yl)-[1,2,4]triazolo[1,5-a]pyridin-2-amine

Prepared in analogy to example 8e, starting from 6-methyl-8-(2-methyl-benzooxazol-6-yl)-[1,2,4]triazolo[1,5-a]pyridin-2-ylamine and 1-(4-bromo-2-methoxy-phenyl)-4-methyl-1H-imidazole. The title compound was obtained as a light brown solid (yield: 48%) after column chromatography on silica gel using a gradient from CH₂Cl₂ to CH₂Cl₂/MeOH 19:1 (v/v) as eluent.

MS ISP (m/e): 466.3 (100) [(M+H)⁺].

¹H NMR (DMSO-D₆, 300 MHz): δ (ppm)=9.95 (s, 1H), 8.68 (s, 1H), 8.60 (s, 1H), 8.15 (d, 1H), 7.95 (s, 1H), 7.87 (s, 1H), 7.77 (d, 1H), 7.65 (s, 1H), 7.24 (d, 1H), 7.20 (d, 2H), 7.03 (s, 1H), 3.87 (s, 3H), 2.67 (s, 3H), 2.15 (s, 3H).

### Example 88

### N-(3-Methoxy-4-(4-methyl-1H-imidazol-1-yl)phenyl)-6-methyl-8-(1-methyl-1H-benzo[d]imidazol-6-yl)-[1,2,4]triazolo[1,5-a]pyridin-2-amine

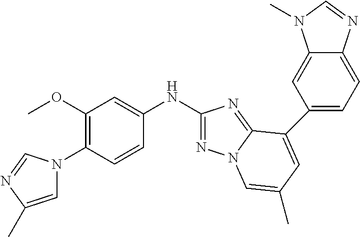

### a) 6-Methyl-8-(3-methyl-3H-benzoimidazol-5-yl)-[1,2,4]triazolo[1,5-a]pyridin-2-ylamine

Prepared in analogy to example 46b, starting from 8-bromo-6-methyl-[1,2,4]triazolo[1,5-a]pyridin-2-amine and 1-methyl-1H-benzo[d]imidazol-6-ylboronic acid. The crude product was purified by column chromatography on silica gel using a gradient from CH₂Cl₂ to CH₂Cl₂/MeOH 9:1 (v/v) as eluent. The title compound was obtained as a white solid (yield: 37%).

MS ISP (m/e): 279.2 (100) [(M+H)⁺].

### b) N-(3-Methoxy-4-(4-methyl-1H-imidazol-1-yl)phenyl)-6-methyl-8-(1-methyl-1H-benzo[d]imidazol-6-yl)-[1,2,4]triazolo[1,5-a]pyridin-2-amine

Prepared in analogy to example 8e, starting from 6-methyl-8-(3-methyl-3H-benzoimidazol-5-yl)-[1,2,4]triazolo[1,5-a]pyridin-2-ylamine and 1-(4-bromo-2-methoxy-phenyl)-4-methyl-1H-imidazole. The title compound was obtained as a light brown solid (yield: 49%) after column chromatography on silica gel using a gradient from CH₂Cl₂ to CH₂Cl₂/MeOH/aqueous saturated ammonia solution 19:1:0.1 (v/v) as eluent and subsequent precipitation from diethyl ether.

MS ISP (m/e): 465.3 (100) [(M+H)⁺].

¹H NMR (CDCl₃, 300 MHz): δ (ppm)=8.27 (s, 1H), 8.17 (s, 1H), 7.93-7.86 (m, 3H), 7.62 (s, 1H), 7.58 (s, 1H), 7.55 (s, 1H), 7.20 (s, 1H), 7.15 (d, 2H), 7.04 (d, 1H), 6.87 (s, 1H), 3.93 (s, 3H), 3.86 (s, 3H), 2.48 (s, 3H), 2.31 (s, 3H).

### Example 89

### N-(3-Methoxy-4-(4-methyl-1H-imidazol-1-yl)phenyl)-6-methyl-8-(4-methyl-3,4-dihydro-2H-benzo[b][1,4]oxazin-7-yl)-[1,2,4]triazolo[1,5-a]pyridin-2-amine

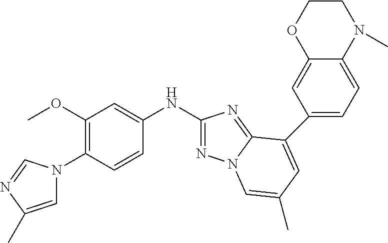

### a) 6-Methyl-8-(4-methyl-3,4-dihydro-2H-benzo[1,4]oxazin-7-yl)-[1,2,4]triazolo[1,5-a]pyridin-2-ylamine

Prepared in analogy to example 46b, starting from 8-bromo-6-methyl-[1,2,4]triazolo[1,5-a]pyridin-2-amine and 4-methyl-7-(4,4,5,5-tetramethyl-1,3,2-dioxaborolan-2-yl)-3,4-dihydro-2H-benzo[b][1,4]oxazine. The crude product was purified by column chromatography on silica gel using ethyl acetate as eluent. The title compound was obtained as a dark yellow solid (yield: 97%).

MS ISP (m/e): 296.3 (100) [(M+H)⁺].

### b) N-(3-Methoxy-4-(4-methyl-1H-imidazol-1-yl)phenyl)-6-methyl-8-(4-methyl-3,4-dihydro-2H-benzo[b][1,4]oxazin-7-yl)-[1,2,4]triazolo[1,5-a]pyridin-2-amine

Prepared in analogy to example 8e, starting from 6-methyl-8-(4-methyl-3,4-dihydro-2H-benzo[1,4]oxazin-7-yl)-[1,2,4]triazolo[1,5-a]pyridin-2-ylamine and 1-(4-bromo-2-methoxy-phenyl)-4-methyl-1H-imidazole. The title compound was obtained as a light yellow solid (yield: 39%) after column chromatography on silica gel using a gradient from CH₂Cl₂ to CH₂Cl₂/MeOH 19:1 (v/v) as eluent and subsequent precipitation from diethyl ether.

MS ISP (m/e): 482.1 (100) [(M+H)⁺].

¹H NMR (DMSO-D₆, 300 MHz): δ (ppm)=9.89 (s, 1H), 8.53 (s, 1H), 7.96 (s, 1H), 7.75 (s, 1H), 7.65 (m, 3H), 7.25 (d, 1H), 7.20 (d, 2H), 7.02 (s, 1H), 6.79 (d, 1H), 4.27 (m, 2H), 3.86 (s, 3H), 3.32 (m, 2H), 2.91 (s, 3H), 2.38 (s, 3H), 2.15 (s, 3H).

### Example 90

### 2-(2-Fluoro-4-(2-(3-methoxy-4-(4-methyl-1H-imidazol-1-yl)phenylamino)-6-methyl-[1,2,4]triazolo[1,5-a]pyridin-8-yl)phenyl)propan-2-ol

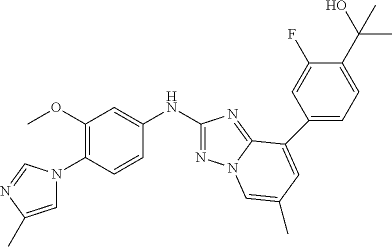

### a) 2-[4-(2-Amino-6-methyl-[1,2,4]triazolo[1,5-a]pyridin-8-yl)-2-fluoro-phenyl]-propan-2-ol

Prepared in analogy to example 46b, starting from 8-bromo-6-methyl-[1,2,4]triazolo[1,5-a]pyridin-2-amine and 1-methyl-1H-benzo[d]imidazol-6-ylboronic acid. The crude product was purified by column chromatography on silica gel using a gradient from CH₂Cl₂ to CH₂Cl₂/MeOH 9:1 (v/v) as eluent. The title compound was obtained as a white solid (yield: 37%).

MS ISP (m/e): 301.2 (100) [(M+H)⁺].

### b) 2-(2-Fluoro-4-(2-(3-methoxy-4-(4-methyl-1H-imidazol-1-yl)phenylamino)-6-methyl-[1,2,4]triazolo[1,5-a]pyridin-8-yl)phenyl)propan-2-ol

Prepared in analogy to example 8e, starting from 2-[4-(2-amino-6-methyl-[1,2,4]triazolo[1,5-a]pyridin-8-yl)-2-fluoro-phenyl]-propan-2-ol and 1-(4-bromo-2-methoxy-phenyl)-4-methyl-1H-imidazole. The title compound was obtained as a yellow solid (yield: 58%) after column chromatography on silica gel using a gradient from CH₂Cl₂ to CH₂Cl₂/MeOH 19:1 (v/v) as eluent.

MS ISP (m/e): 487.4 (100) [(M+H)⁺], 469.3 (67) [(M−H₂O+H)⁺].

¹H NMR (DMSO-D₆, 300 MHz): δ (ppm)=9.92 (s, 1H), 8.69 (s, 1H), 8.08 (d, 2H), 7.96 (d, 1H), 7.86 (m, 2H), 7.74 (t, 1H), 7.24 (s, 2H), 7.03 (s, 1H), 5.36 (s, 1H), 3.85 (s, 3H), 2.42 (s, 3H), 2.15 (s, 3H), 1.54 (s, 6H).

### Example 91

### 5-Chloro-2-(2-(3-methoxy-4-(4-methyl-1H-imidazol-1-yl)phenylamino)-6-methyl-[1,2,4]triazolo[1,5-a]pyridin-8-yl)benzaldehyde

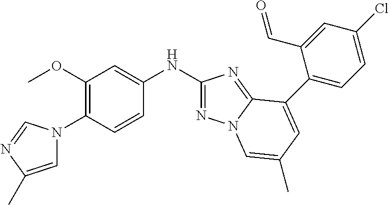

### a) 2-(2-Amino-6-methyl-[1,2,4]triazolo[1,5-a]pyridin-8-yl)-5-chloro-benzaldehyde

Prepared in analogy to example 46b, starting from 8-bromo-6-methyl-[1,2,4]triazolo[1,5-a]pyridin-2-amine and 4-chloro-2-formylphenylboronic acid. The crude product was purified by column chromatography on silica gel using a gradient from CH₂Cl₂ to CH₂Cl₂/MeOH 9:1 (v/v) as eluent. The title compound was obtained as a brown solid (yield: 96%) after precipitation from diethylether.

MS ISP (m/e): 287.1/289.2 (100/30) [(M+H)⁺].

### b) 5-Chloro-2-(2-(3-methoxy-4-(4-methyl-1H-imidazol-1-yl)phenylamino)-6-methyl-[1,2,4]triazolo[1,5-a]pyridin-8-yl)benzaldehyde

Prepared in analogy to example 8e, starting from 2-(2-amino-6-methyl-[1,2,4]triazolo[1,5-a]pyridin-8-yl)-5-chloro-benzaldehyde and 1-(4-bromo-2-methoxy-phenyl)-4-methyl-1H-imidazole. The title compound was obtained as a yellow solid (yield: 22%) after column chromatography on silica gel using a gradient from CH₂Cl₂ to CH₂Cl₂/MeOH 19:1 (v/v) as eluent.

MS ISP (m/e): 473.2/475.2 (100/43) [(M+H)⁺].

¹H NMR (CDCl₃, 300 MHz): δ (ppm)=9.85 (s, 1H), 8.36 (s, 1H), 8.04 (s, 1H), 7.65 (d, 1H), 7.61 (s, 1H), 7.54 (d, 1H), 7.51 (m, 1H), 7.33 (s, 1H), 7.15 (d, 1H), 7.08 (s, 1H), 6.91 (d, 1H), 6.86 (s, 1H), 3.85 (s, 3H), 2.48 (s, 3H), 2.30 (s, 3H).

### Example 92

### (5-Chloro-2-(2-(3-methoxy-4-(4-methyl-1H-imidazol-1-yl)phenylamino)-6-methyl-[1,2,4]triazolo[1,5-a]pyridin-8-yl)phenyl)methanol

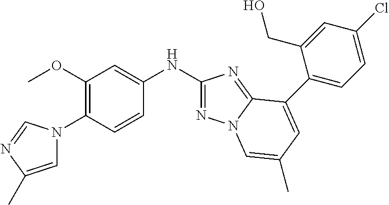

To a solution of 5-chloro-2-(2-(3-methoxy-4-(4-methyl-1H-imidazol-1-yl)phenylamino)-6-methyl-[1,2,4]triazolo[1,5-a]pyridin-8-yl)benzaldehyde (29 mg, 61.3 μmol) in methanol (1 mL) was added at room temperature under an athmosphere of nitrogen portion wise sodium borohydride (3.5 mg, 92 μmol). The reaction was stirred at room temperature over night. Water was added and the reaction was extracted twice with dichloromethane. The combined organic layers were washed with saturated aqueous sodium chloride solution, dried over sodium sulfate, filtered and the solvent was evaporated under reduced pressure. The crude product was purified by column chromatography on silica gel using a gradient from CH₂Cl₂ to CH₂Cl₂/MeOH 19:1 (v/v) as eluent. The title compound was obtained as a light yellow solid (yield: 48%).

MS ISP (m/e): 475.2/477.2 (100/41) [(M+H)⁺].

¹H NMR (CDCl₃, 300 MHz): δ (ppm)=8.31 (s, 1H), 7.62 (s, 2H), 7.42 (s, 1H), 7.39 (d, 1H), 7.26 (m, 2H), 7.17 (d, 1H), 7.02 (d, 1H), 6.89 (s, 1H), 6.86 (s, 1H), 4.37 (s, 2H), 3.85 (s, 3H), 2.46 (s, 3H), 2.30 (s, 3H).

### Example 93

### 7-(3-Chloro-4-fluorophenyl)-N-(3-methoxy-4-(4-methyl-1H-imidazol-1-yl)phenyl)-5-methyl-[1,2,4]triazolo[1,5-a]pyrimidin-2-amine

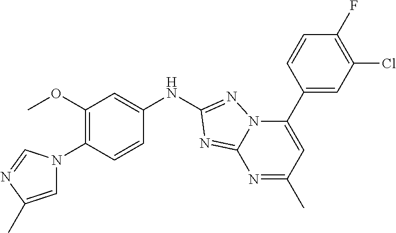

A solution of 1-(3-chloro-4-fluorophenyl)butane-1,3-dione (64.4 mg, 300 μmol) and of N3-(3-methoxy-4-(4-methyl-1H-imidazol-1-yl)phenyl)-4H-1,2,4-triazole-3,5-diamine (57.1 mg, 0.2 mmol) in acetic acid (1.0 mL) was heated to 100° C. over night. The solution was diluted with diethyl ether and the crude product was filtered off. The precipitate was purified by column chromatography on silica gel using a gradient from methylene chloride to methylene chloride/MeOH 9:1 (v/v) as eluent to yield the title compound as a yellow solid (17.2 mg, 18%).

MS ISP (m/e): 464.2/466.3 (100/51) [(M+H)⁺].

¹H NMR (DMSO-D₆, 300 MHz): δ (ppm)=10.11 (s, 1H), 8.51 (d, 1H), 8.29 (m, 1H), 7.78 (s, 1H), 7.71 (t, 1H), 7.64 (s, 1H), 7.45 (s, 1H), 7.23 (d, 1H), 7.19 (d, 1H), 7.02 (s, 1H), 3.78 (s, 3H), 2.63 (s, 3H), 2.14 (s, 3H).

### Example 94

### Ethyl 7-(3-chloro-4-fluorophenyl)-2-(3-methoxy-4-(4-methyl-1H-imidazol-1-yl)phenylamino)-[1,2,4]triazolo[1,5-a]pyrimidine-5-carboxylate

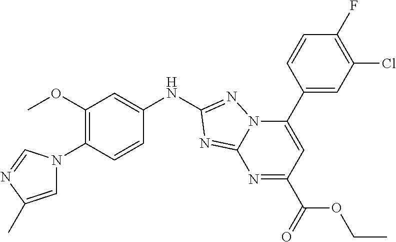

A solution of ethyl 4-(3-chloro-4-fluorophenyl)-2,4-dioxobutanoate (409 mg, 1.5 mmol) and of N3-(3-methoxy-4-(4-methyl-1H-imidazol-1-yl)phenyl)-4H-1,2,4-triazole-3,5-diamine (285 mg, 1 mmol) in acetic acid (5.0 mL) was heated to 100° C. over night. A solid precipitated. the reaction was diluted with diethyl ether and the crude product was filtered off, washed with diethyl ether and dried. The residue was purified by column chromatography on silica gel using a gradient from methylene chloride to methylene chloride/MeOH 9:1 (v/v) as eluent to yield the title compound as an orange solid (15.8 mg, 3.0%).

MS ISP (m/e): 522.2/524.3 (100/28) [(M+H)⁺], 450.4 (41).

### Example 95

### tert-Butyl 3-(2-(3-methoxy-4-(4-methyl-1H-imidazol-1-yl)phenylamino)-6-methyl-[1,2,4]triazolo[1,5-a]pyridin-8-yl)benzylcarbamate

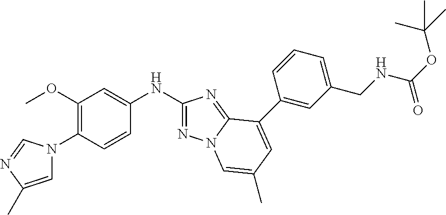

### a) [3-(2-Amino-6-methyl-[1,2,4]triazolo[1,5-a]pyridin-8-yl)-benzyl]-carbamic acid tert-butyl ester

Prepared in analogy to example 46b, starting from 8-bromo-6-methyl-[1,2,4]triazolo[1,5-a]pyridin-2-amine and 3-((tert-butoxycarbonylamino)methyl)phenylboronic acid. The crude product was purified by precipitation from a mixture of methylene chloride/diethylether. The title compound was obtained as an off-white solid (yield: 99%) after precipitation from diethyl ether.

MS ISP (m/e): 354.4 (80) [(M+H)⁺], 298.4 (100), 237.2 (99).

### b) tert-Butyl 3-(2-(3-methoxy-4-(4-methyl-1H-imidazol-1-yl)phenylamino)-6-methyl-[1,2,4]triazolo[1,5-a]pyridin-8-yl)benzylcarbamate

Prepared in analogy to example 8e, starting from [3-(2-amino-6-methyl-[1,2,4]triazolo[1,5-a]pyridin-8-yl)-benzyl]-carbamic acid tert-butyl ester and 1-(4-bromo-2-methoxy-phenyl)-4-methyl-1H-imidazole. The title compound was obtained as a light yellow solid (yield: 58%) after column chromatography on silica gel using a gradient from CH₂Cl₂ to CH₂Cl₂/MeOH 19:1 (v/v) as eluent.

MS ISP (m/e): 540.5 (79) [(M+H)⁺], 484.4 (100), 440.4 (61).

¹H NMR (CDCl₃, 300 MHz): δ (ppm)=8.25 (s, 1H), 7.94 (d, 1H), 7.84 (m, 1H), 7.62 (m, 2H), 7.45 (m, 2H), 7.35 (m, 1H), 7.16 (d, 1H), 7.12 (m, 1H), 7.02 (m, 1H), 6.87 (s, 1H), 4.95 (m, 1H), 4.39 (m, 2H), 3.89 (s, 3H), 2.45 (s, 3H), 2.30 (s, 3H), 1.47 (s, 9H).

### Example 96

### tert-Butyl 4-(2-(3-methoxy-4-(4-methyl-1H-imidazol-1-yl)phenylamino)-6-methyl-[1,2,4]triazolo[1,5-a]pyridin-8-yl)-5,6-dihydropyridine-1(2H)-carboxylate

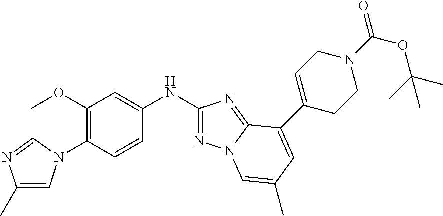

### 4-(2-Amino-6-methyl-[1,2,4]triazolo[1,5-a]pyridin-8-yl)-3,6-dihydro-2H-pyridine-1-carboxylic acid tert-butyl ester

Prepared in analogy to example 46b, starting from 8-bromo-6-methyl-[1,2,4]triazolo[1,5-a]pyridin-2-amine and tert-butyl 4-(4,4,5,5-tetramethyl-1,3,2-dioxaborolan-2-yl)-5,6-dihydropyridine-1(2H)-carboxylate. The crude product was purified by column chromatography on silica gel using a gradient from CH₂Cl₂ to CH₂Cl₂/MeOH 9:1 (v/v) as eluent. The title compound was obtained as a dark yellow solid (yield: 67%).

MS ISP (m/e): 330.3 (6) [(M+H)⁺], 274.3 (10), 230.3 (13), 201.2 (100).

### b) tert-Butyl 4-(2-(3-methoxy-4-(4-methyl-1H-imidazol-1-yl)phenylamino)-6-methyl-[1,2,4]triazolo[1,5-a]pyridin-8-yl)-5,6-dihydropyridine-1(2H)-carboxylate

Prepared in analogy to example 8e, starting 4-(2-amino-6-methyl-[1,2,4]triazolo[1,5-a]pyridin-8-yl)-3,6-dihydro-2H-pyridine-1-carboxylic acid tert-butyl ester and 1-(4-bromo-2-methoxy-phenyl)-4-methyl-1H-imidazole. The title compound was obtained as a yellow solid (yield: 40%) after column chromatography on silica gel using a gradient from CH₂Cl₂ to CH₂Cl₂/MeOH 19:1 (v/v) as eluent.

MS ISP (m/e): 516.5 (90) [(M+H)⁺], 460.4 (100), 416.4 (48).

¹H NMR (CDCl₃, 300 MHz): δ (ppm)=8.15 (s, 1H), 7.67 (s, 1H), 7.62 (s, 1H), 7.21-7.11 (m, 2H), 6.98 (d, 1H), 6.95 (s, 1H), 6.87 (s, 1H), 4.40 (br s, 1H), 4.19 (m, 2H), 3.90 (s, 3H), 3.70 (q, 2H), 2.67 (m, 2H), 2.39 (s, 3H), 2.30 (s, 3H), 1.50 (s, 9H).

### Example 97

### 8-(3-(Aminomethyl)phenyl)-N-(3-methoxy-4-(4-methyl-1H-imidazol-1-yl)phenyl)-6-methyl-[1,2,4]triazolo[1,5-a]pyridin-2-amine dihydrochloride

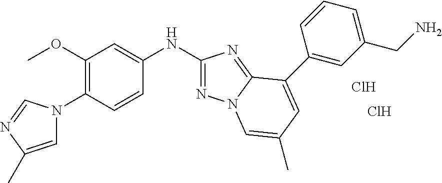

To a solution of tert-butyl 3-(2-(3-methoxy-4-(4-methyl-1H-imidazol-1-yl)phenylamino)-6-methyl-[1,2,4]triazolo[1,5-a]pyridin-8-yl)benzylcarbamate (311 mg, 576 μmol) in dichloromethane (5.8 mL) was added a 2M solution of hydrogen chloride in diethyl ether (2.9 mL). The reaction was stirred at room temperature for 3 hours. It was diluted with diethyl ether and the precipitate was filtered off, washed with diethyl ether and dried under reduced pressure to yield the title compound as a light brown solid (251 mg, 85%).

MS ISP (m/e): 440.3 (22) [(M+H)³⁰ ], 306.2 (100).

¹H NMR (DMSO-D₆, 300 MHz): δ (ppm)=9.37 (s, 1H), 8.73 (s, 1H), 8.59 (br s, 2H), 8.40 (d, 1H), 8.18 (s, 1H), 7.96 (s, 1H), 7.87 (s, 1H), 7.66 (s, 1H), 7.57 (m, 2H), 7.47 (d, 1H), 7.33 (d, 1H), 3.89 (s, 3H), 2.44 (s, 3H), 2.35 (s, 3H).

### Example 98

### N-(3-(2-(3-Methoxy-4-(4-methyl-1H-imidazol-1-yl)phenylamino)-6-methyl-[1,2,4]triazolo[1,5-a]pyridin-8-yl)benzyl)methanesulfonamide

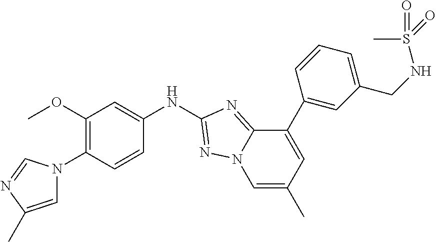

To a solution of 8-(3-(aminomethyl)phenyl)-N-(3-methoxy-4-(4-methyl-1H-imidazol-1-yl)phenyl)-6-methyl-[1,2,4]triazolo[1,5-a]pyridin-2-amine dihydrochloride (76.9 mg, 0.15 mmol) and diisopropyl amine (77.5 mg, 105 μA, 600 μmol) in dichloromethane (1.5 mL) was added methanesulfonyl chloride (18.9 mg, 12.8 μA, 165 μmol). The reaction was stirred at room temperature over night, diluted with methylene chloride, washed with water and saturated aqueous sodium chloride solution, dried over sodium sulfate, filtered and the solvent was evaporated under reduced pressure. The title compound was obtained as a yellow solid (52 mg, 67%) after column chromatography on silica gel using a gradient from CH₂Cl₂ to CH₂Cl₂/MeOH 19:1 (v/v) as eluent.

MS ISP (m/e): 518.3 (100) [(M+H)⁺].

¹H NMR (DMSO-D₆, 300 MHz): δ (ppm)=9.91 (s, 1H), 8.69 (s, 1H), 8.20 (d, 1H), 7.99 (s, 1H), 7.79 (s, 1H), 7.74 (s, 1H), 7.65 (s, 1H), 7.63 (t, 1H), 7.51 (t, 1H), 7.45 (d, 1H), 7.29 (d, 1H), 7.25 (d, 1H), 7.02 (s, 1H), 4.26 (d, 2H), 3.83 (s, 3H), 2.90 (s, 3H), 2.45 (s, 3H), 2.15 (s, 3H).

### Example 99

### N-(3-(2-(3-Methoxy-4-(4-methyl-1H-imidazol-1-yl)phenylamino)-6-methyl-[1,2,4]triazolo[1,5-a]pyridin-8-yl)benzyl)acetamide

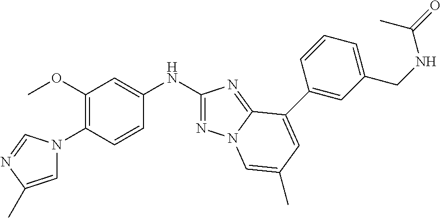

To a solution of 8-(3-(aminomethyl)phenyl)-N-(3-methoxy-4-(4-methyl-1H-imidazol-1-yl)phenyl)-6-methyl-[1,2,4]triazolo[1,5-a]pyridin-2-amine dihydrochloride (76.9 mg, 0.15 mmol) and diisopropyl amine (77.5 mg, 105 μA, 600 μmol) in dichloromethane (1.5 mL) was added acetyl chloride (13.2 mg, 12.0 μA, 165 μmol). The reaction was stirred at room temperature over night, diluted with methylene chloride, washed with water and saturated aqueous sodium chloride solution, dried over sodium sulfate, filtered and the solvent was evaporated under reduced pressure. The title compound was obtained as a light yellow solid (55 mg, 76%) after column chromatography on silica gel using a gradient from CH₂Cl₂ to CH₂Cl₂/MeOH 19:1 (v/v) as eluent.

MS ISP (m/e): 482.4 (100) [(M+H)⁺].

¹H NMR (DMSO-D₆, 300 MHz): δ (ppm)=9.91 (s, 1H), 8.68 (s, 1H), 8.40 (br t, 1H), 8.12 (d, 1H), 7.92 (s, 1H), 7.80 (s, 1H), 7.71 (s, 1H), 7.64 (s, 1H), 7.47 (t, 1H), 7.34-7.25 (m, 3H), 7.02 (s, 1H), 4.35 (d, 2H), 3.83 (s, 3H), 2.42 (s, 3H), 2.15 (s, 3H), 1.89 (s, 3H).

### Example 100

### N-(3-Methoxy-4-(4-methyl-1H-imidazol-1-yl)phenyl)-6-methyl-8-(1-(methylsulfonyl)piperidin-4-yl)-[1,2,4]triazolo[1,5-a]pyridin-2-amine

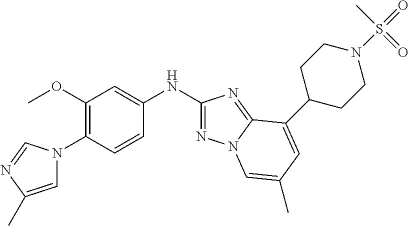

Prepared in analogy to example 98, starting from 8-(3-(aminomethyl)phenyl)-N-(3-methoxy-4-(4-methyl-1H-imidazol-1-yl)phenyl)-6-methyl-[1,2,4]triazolo[1,5-a]pyridin-2-amine dihydrochloride and methanesulfonyl chloride. The title compound was obtained as a white solid (yield: 32%) after column chromatography on silica gel using a gradient from CH₂Cl₂ to CH₂Cl₂/MeOH 19:1 (v/v) as eluent.

MS ISP (m/e): 496.4 (100) [(M+H)⁺].

¹H NMR (DMSO-D₆, 300 MHz): δ (ppm)=8.15 (s, 1H), 7.62 (s, 2H), 7.17 (d, 1H), 7.11 (s, 1H), 7.00 (d, 1H), 6.92 (s, 1H), 6.87 (s, 1H), 4.02 (br d, 2H), 3.90 (s, 3H), 3.16 (tt, 1H), 2.91 (d, 1H), 2.84 (s, 3H), 2.79 (d, 1H), 2.38 (s, 3H), 2.30 (s, 3H), 2.13 (br d, 2H), 2.05 (dt, 2H).

### Example 101

### N-(3-Methoxy-4-(4-methyl-1H-imidazol-1-yl)phenyl)-8-morpholino-[1,2,4]triazolo[1,5-a]pyridin-2-amine

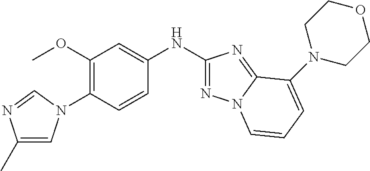

### 4-(2-Nitropyridin-3-yl)morpholine

To a solution of 3-bromo-2-nitropyridine (207 mg, 1 mmol) in DMSO (2 mL) was added at room temperature under stirring and an athmosphere of nitrogen morpholine (95.8 mg, 95.8 μl, 1.1 mmol), tetrabutyl ammonium iodide (18.5 mg, 50.0 μmol) and potassium carbonate (152 mg, 1.1 mmol). The reaction was stirred at 80° C. over night. Water was added and the reaction was extracted twice with diethyl ether. The combined organic layers were washed with water and with saturated aqueous sodium chloride solution, dried over sodium sulfate, filtered and the solvent was evaporated under reduced pressure. The title compound was obtained as a yellow oil (57 mg, 27%) after column chromatography on silica gel using a gradient from heptane/ethyl acetate 4:1 to 1:1 (v/v) as eluent.

¹H NMR (DMSO-D₆, 300 MHz): δ (ppm)=8.45 (d, 1H), 7.96 (d, 1H), 7.71 (dd, 1H), 3.67 (t, 4H), 3.00 (t, 4H).

### b) 3-Morpholinopyridin-2-amine

To a solution of 4-(2-nitropyridin-3-yl)morpholine (155 mg, 741 μmol) in ethyl acetate was added Pd/C 10% (15.5 mg, 146 μmol) and the reaction was hydrogenated under an athmosphere of hydrogen for 3 hours at room temperature. The catalyst was filtered off, washed with ethyl acetate. The title compound was obtained as a purple solid (128 mg, 96%) after evaporation of the solvent under reduced pressure.

MS ISP (m/e): 180.1 (100) [(M+H)⁺].

¹H NMR (DMSO-D₆, 300 MHz): δ (ppm)=7.66 (d, 1H), 7.14 (d, 1H), 6.54 (dd, 1H), 5.59 (br s, 2H), 3.75 (t, 4H), 2.79 (t, 4H).

### c) 8-Morpholin-4-yl-[1,2,4]triazolo[1,5-a]pyridin-2-ylamine

Prepared in analogy to example 1b-c), starting from 3-morpholinopyridin-2-amine. The crude product was purified by column chromatography on silica gel using ethyl acetate as eluent. The title compound was obtained as a light brown solid (yield: 85% over 2 steps).

MS ISP (m/e): 220.2 (100) [(M+H)⁺].

¹H NMR (DMSO-D₆, 300 MHz): δ (ppm)=8.10 (d, 1H), 6.76 (t, 1H), 6.68 (d, 1H), 5.92 (br s, 2H), 3.77 (t, 4H), 3.38 (t, 4H).

### d) N-(3-Methoxy-4-(4-methyl-1H-imidazol-1-yl)phenyl)-8-morpholino-[1,2,4]triazolo[1,5-a]pyridin-2-amine

Prepared in analogy to example 8e, starting 8-morpholin-4-yl-[1,2,4]triazolo[1,5-a]pyridin-2-ylamine and 1-(4-bromo-2-methoxy-phenyl)-4-methyl-1H-imidazole. The title compound was obtained as a white solid (yield: 32%) after column chromatography on silica gel using a gradient from CH₂Cl₂ to CH₂Cl₂/MeOH 19:1 (v/v) as eluent and precipitation from diethyl ether.

MS ISP (m/e): 406.3 (100) [(M+H)⁺].

¹H NMR (CDCl₃, 300 MHz): δ (ppm)=8.10 (d, 1H), 7.64 (s, 2H), 7.17 (d, 1H), 7.01 (m, 2H), 6.87 (s, 1H), 6.83 (t, 1H), 6.71 (d, 1H), 3.96 (t, 4H), 3.89 (s, 3H), 3.51 (t, 4H), 2.31 (s, 3H).

### Example 102

### 8-(3-((Isopropylamino)methyl)phenyl)-N-(3-methoxy-4-(4-methyl-1H-imidazol-1-yl)phenyl)-6-methyl-[1,2,4]triazolo[1,5-a]pyridin-2-amine

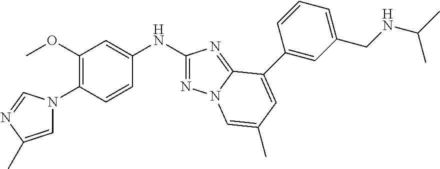

To a suspension of 8-(3-(aminomethyl)phenyl)-N-(3-methoxy-4-(4-methyl-1H-imidazol-1-yl)phenyl)-6-methyl-[1,2,4]triazolo[1,5-a]pyridin-2-amine dihydrochloride (71.7 mg, 140 mmol) and diisopropyl amine (54.3 mg, 73.3 μA, 420 μmol) in tetrahydrofurane (1.4 mL) was added propan-2-one (9.75 mg, 12.3 μA, 168 μmol), sodium triacetoxyborohydride (91.7 mg, 420 μmol) and acetic acid (16.8 mg, 16.0 μA, 280 μmol). Tetrahydrofurane (1.4 mL) was added and the reaction was stirred at room temperature over night. The reaction was diluted with 1N aqueous sodium hydroxide solution and extracted twice with diethyl ether. The combined organic layers were washed with saturated aqueous sodium chloride solution, dried over sodium sulfate, filtered and the solvent was evaporated under reduced pressure. The title compound was obtained as a light yellow solid (51 mg, 76%) after column chromatography on silica gel using a gradient from CH₂Cl₂/MeOH 19:1 to 9:1 (v/v) as eluent.

MS ISP (m/e): 482.4 (100) [(M+H)₊], 423.3 (52).

¹H NMR (CDCL₃, 300 MHz): δ (ppm)=8.24 (s, 1H), 7.91 (d, 1H), 7.89 (s, 1H), 7.64 (s, 1H), 7.62 (s, 1H), 7.48 (s, 1H), 7.42 (t, 1H), 7.40 (d, 1H), 7.17 (d, 1H), 7.15 (s, 1H), 7.03 (d, 1H), 6.86 (s, 1H), 3.89 (s, 3H), 2.95 (sept, 1H), 2.45 (s, 3H), 2.30 (s, 3H), 1.14 (d, 6H).

### Example 103

### N-(3-Methoxy-4-(4-methyl-1H-imidazol-1-yl)phenyl)-6-methyl-8-(1,2,3,6-tetrahydropyridin-4-yl)-[1,2,4]triazolo[1,5-a]pyridin-2-amine dihydrochloride

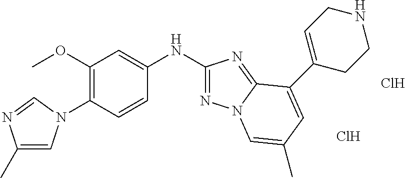

To suspension of tert-butyl 4-(2-(3-methoxy-4-(4-methyl-1H-imidazol-1-yl)phenylamino)-6-methyl-[1,2,4]triazolo[1,5-a]pyridin-8-yl)-5,6-dihydropyridine-1(2H)-carboxylate (514 mg, 997 μmmol) in dichloromethane (10 mL) was added 2M hydrogen chloride in diethyl ether (5.0 mL). The reaction was stirred at room temperature over night. The precipitate was filtered off, washed with diethyl ether and dried under reduced pressure. The title compound was obtained as a light brown solid (504 mg, 104%).

MS ISP (m/e): 416.4 (100) [(M+H)⁺], 387.3 (63).

¹H NMR (DMSO-D₆, 300 MHz): δ (ppm)=9.56 (s, 1H), 8.64 (s, 1H), 7.89 (s, 1H), 7.66 (s, 1H), 7.52 (s, 1H), 7.48 (s, 1H), 7.46 (d, 1H), 7.31 (d, 1H), 3.89 (br s, 5H), 3.35 (br m, 2H), 2.89 (br m, 2H), 2.38 (s, 3H), 2.35 (s, 3H).

### Example 104

### 8-(1-Isopropyl-1,2,3,6-tetrahydropyridin-4-yl)-N-(3-methoxy-4-(4-methyl-1H-imidazol-1-yl)phenyl)-6-methyl-[1,2,4]triazolo[1,5-a]pyridin-2-amine

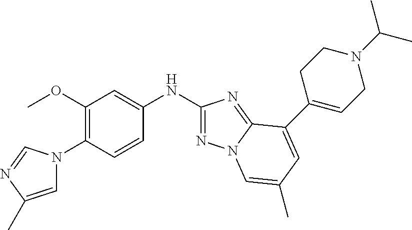

Prepared in analogy to example 102, starting from N-(3-methoxy-4-(4-methyl-1H-imidazol-1-yl)phenyl)-6-methyl-8-(1,2,3,6-tetrahydropyridin-4-yl)-[1,2,4]triazolo[1,5-a]pyridin-2-amine dihydrochloride and propan-2-one. The crude product was purified by column chromatography on silica gel using a mixture of CH₂Cl₂/MeOH 9:1 as eluent. The title compound was obtained as a yellow solid (yield: 31%).

MS ISP (m/e): 458.5 (93) [(M+H)⁺], 387.3 (100).
¹H NMR (CDCl₃, 300 MHz): δ (ppm)=8.12 (s, 1H), 7.69 (s, 1H), 7.61 (s, 1H), 7.33 (br s, 1H), 7.20 (s, 1H), 7.16 (d, 1H), 7.09 (s, 1H), 6.97 (d, 1H), 6.86 (s, 1H), 3.89 (s, 3H), 3.39 (br m, 2H), 2.90-2.80 (br m, 3H), 2.73 (br m, 2H), 2.37 (s, 3H), 2.30 (s, 3H), 1.14 (d, 6H).

### Example 105

### N-(3-Methoxy-4-(4-methyl-1H-imidazol-1-yl)phenyl)-6-methyl-8-(1-(methylsulfonyl)-1,2,3,6-tetrahydropyridin-4-yl)-[1,2,4]triazolo[1,5-a]pyridin-2-amine

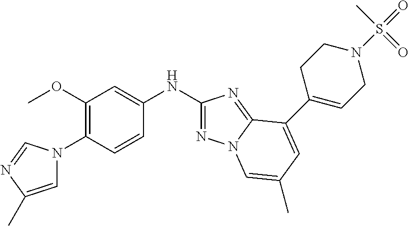

Prepared in analogy to example 98, starting from N-(3-methoxy-4-(4-methyl-1H-imidazol-1-yl)phenyl)-6-methyl-8-(1,2,3,6-tetrahydropyridin-4-yl)-[1,2,4]triazolo[1,5-a]pyridin-2-amine dihydrochloride and methanesulfonyl chloride. The title compound was obtained as a yellow solid (yield: 45%) after column chromatography on silica gel using a gradient from CH₂Cl₂ to CH₂Cl₂/MeOH 19:1 (v/v) as eluent.

MS ISP (m/e): 494.3 (100) [(M+H)⁺].

¹H NMR (CDCl₃, 300 MHz): δ (ppm)=8.18 (s, 1H), 7.67 (s, 1H), 7.60 (s, 1H), 7.23-7.17 (m, 2H), 7.04 (d, 1H), 7.00 (s, 1H), 6.88 (s, 1H), 4.09 (m, 2H), 3.89 (s, 3H), 3.57 (m, 2H), 2.89 (s, 3H), 2.83 (m, 2H), 2.40 (s, 3H), 2.31 (s, 3H).

### Example 106

### 1-(4-(2-(3-Methoxy-4-(4-methyl-1H-imidazol-1-yl)phenylamino)-6-methyl-[1,2,4]triazolo[1,5-a]pyridin-8-yl)-5,6-dihydropyridin-1(2H)-yl)ethanone

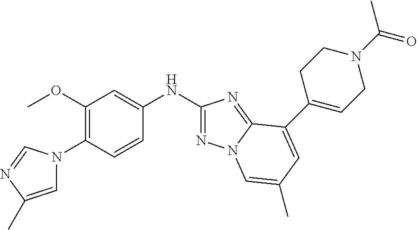

Prepared in analogy to example 99, starting from N-(3-methoxy-4-(4-methyl-1H-imidazol-1-yl)phenyl)-6-methyl-8-(1,2,3,6-tetrahydropyridin-4-yl)-[1,2,4]triazolo[1,5-a]pyridin-2-amine dihydrochloride and acetyl chloride. The title compound was obtained as a light yellow solid (yield: 85%) after column chromatography on silica gel using a gradient from CH₂Cl₂ to CH₂Cl₂/MeOH 19:1 (v/v) as eluent.

MS ISP (m/e): 458.3 (100) [(M+H)⁺].

¹H NMR (CDCl₃, 300 MHz): δ (ppm)=8.17 (s, 1H), 7.63 (s, 1H), 7.62 (d, 1H), 7.22-7.17 (m, 3H), 7.05-7.00 (m, 2H), 6.87 (s, 1H), 4.35 (br s, 1H), 4.25 (br s, 1H), 3.90 (s, 3H), 3.89 (t, 1H), 3.73 (t, 1H), 2.40 (s, 3H), 2.31 (s, 3H), 2.19 and 2.16 (s, 3H).

### Example 107

### Ethyl 4-(2-(3-methoxy-4-(4-methyl-1H-imidazol-1-yl)phenylamino)-6-methyl-[1,2,4]triazolo[1,5-a]pyridin-8-yl)-5,6-dihydropyridine-1(2H)-carboxylate

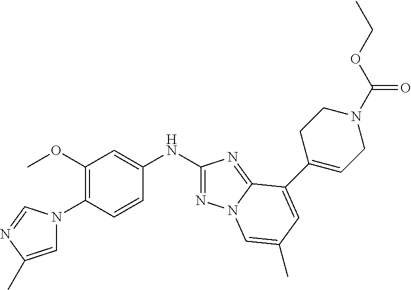

Prepared in analogy to example 99, starting from N-(3-methoxy-4-(4-methyl-1H-imidazol-1-yl)phenyl)-6-methyl-8-(1,2,3,6-tetrahydropyridin-4-yl)-[1,2,4]triazolo[1,5-a]pyridin-2-amine dihydrochloride and ethyl chloroformate. The title compound was obtained as a yellow foam (yield: 79%) after column chromatography on silica gel using a gradient from CH₂Cl₂ to CH₂Cl₂/MeOH 19:1 (v/v) as eluent.

MS ISP (m/e): 488.3 (100) [(M+H)⁺].

¹H NMR (CDCl₃, 300 MHz): δ (ppm)=8.16 (s, 1H), 7.66 (s, 1H), 7.62 (s, 1H), 7.25-7.16 (m, 3H), 7.00-6.96 (m, 2H), 6.87 (s, 1H), 4.23-4.18 (m, 4H), 3.90 (s, 3H), 3.75 (m, 2H), 2.69 (m, 2H), 2.39 (s, 3H), 2.30 (s, 3H), 1.30 (t, 3H).

### Example 108

### 2-(4-(2-(3-Methoxy-4-(4-methyl-1H-imidazol-1-yl)phenylamino)-6-methyl-[1,2,4]triazolo[1,5-a]pyridin-8-yl)-5,6-dihydropyridin-1(2H)-yl)acetonitrile

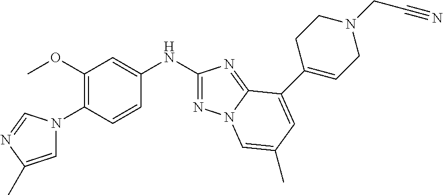

To suspension of N-(3-methoxy-4-(4-methyl-1H-imidazol-1-yl)phenyl)-6-methyl-8-(1,2,3,6-tetrahydropyridin-4-yl)-[1,2,4]triazolo[1,5-a]pyridin-2-amine (62.3 mg, 0.15 mmol) in acetonitrile (1.5 mL) was added 2-bromoacetonitrile (20.4 mg, 11.8 μA, 165 μmol) and potassium carbonate (41.5 mg, 300 μmol). The reaction was stirred at room temperature over night. Water was added and the reaction was extracted twice with ethyl acetate and twice with dichloromethane. The combined organic layers were washed with saturated aqueous sodium chloride solution, dried over sodium sulfate, filtered and the solvent was evaporated under reduced pressure. The precipitate was filtered off, washed with diethyl ether and dried under reduced pressure. The title compound was obtained as a yellow solid (61.3 mg, 90%) after stirring of the crude product with diethyl ether.

MS ISP (m/e): 455.4 (100) [(M+H)⁺].

¹H NMR (CDCl₃, 300 MHz): δ (ppm)=8.16 (s, 1H), 7.65 (m, 2H), 7.31 (br t 1H), 7.22 (s, 1H), 7.17 (d, 1H), 7.03 (br s, 1H), 7.01 (d, 1H), 6.88 (s, 1H), 3.90 (s, 3H), 3.70 (s, 2H), 3.47 (m, 2H), 2.92 (t, 2H), 2.79 (m, 2H), 2.39 (s, 3H), 2.32 (s, 3H).

### Example 109

### N-(3-Methoxy-4-(4-methyl-1H-imidazol-1-yl)phenyl)-7-phenyl-6,7-dihydro-5H-[1,2,4]triazolo[5,1-b][1,3]oxazin-2-amine

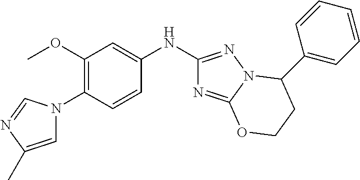

### (3-Bromo-3-phenylpropoxy)(tert-butyl)dimethylsilane

A suspension of tert-butyldimethyl(3-phenylpropoxy)silane (2.22 g, 8.86 mmol), N-bromosuccinimide (1.58 g, 8.86 mmol) and benzoyl peroxide (66.4 mg, 266 μmol) in carbon tetrachloride (17.8 mL) was heated to reflux for 3 hours. The reaction was filtered, the precipitate washed with carbon tetrachloride and the solvent was evaporated. Water was added and the reaction was extracted twice with diethyl ether. The combined organic layers were washed with saturated aqueous sodium chloride solution, dried over sodium sulfate, filtered and the solvent was evaporated under reduced pressure. The title compound was obtained as a light yellow oil (1.63 g, 55%) after column chromatography on silica gel using heptane/ethyl acetate 19:1 (v/v) as eluent.

¹H NMR (CDCl₃, 300 MHz): δ (ppm)=7.42-7.26 (m, 5H), 5.42 (dd, 1H), 3.76 (m, 1H), 3.68 (m, 1H), 2.48 (m, 1H), 2.28 (m, 1H), 0.90 (s, 9H), 0.06 (s, 3H), 0.03 (s, 3H).

### b) 5-Bromo-1-(3-(tert-butyldimethylsilyloxy)-1-phenylpropyl)-3-nitro-1H-1,2,4-triazole

A solution of (3-bromo-3-phenylpropoxy)(tert-butyl)dimethylsilane (934 mg, 2.84 mmol) in acetonitrile (27 mL) was stirred at room temperature under an athmosphere of nitrogen with sodium iodide (425 mg, 2.84 mmol) for 15 minutes. Potassium carbonate (560 mg, 4.05 mmol) was added and the reaction was heated 60° C. At this temperature 5-bromo-3-nitro-1H-1,2,4-triazole (532 mg, 2.7 mmol) dissolved in acetonitrile (5.3 mL) was added within 30 minutes. The reaction was stirred for 2 hours at 85° C. Water was added and the reaction was extracted twice with ethyl acetate. The combined organic layers were washed with saturated aqueous sodium chloride solution, dried over sodium sulfate, filtered and the solvent was evaporated under reduced pressure. The title compound was obtained as a colorless viscous oil (510 mg, 42%) after column chromatography on silica gel using a gradient from heptane to heptane/ethyl acetate 4:1 (v/v) as eluent.

¹H NMR (CDCl₃, 300 MHz): δ (ppm)=7.42-7.36 (m, 5H), 5.91 (dd, 1H), 3.58 (m, 1H), 3.48 (m, 1H), 2.72 (m, 1H), 2.39 (m, 1H), 0.91 (s, 9H), 0.00 (s, 6H).

### c) 2-Nitro-7-phenyl-6,7-dihydro-5H-[1,2,4]triazolo[5,1-b][1,3]oxazine

To a solution of 5-bromo-1-(3-(tert-butyldimethylsilyloxy)-1-phenylpropyl)-3-nitro-1H-1,2,4-triazole (510 mg, 1.16 mmol) in tetrahydrofurane (11.6 mL) was added under an athmosphere of nitrogen at room temperature 1M tetrabutyl ammonium fluoride solution in tetrahydrofurane (3.47 mL, 3.47 mmol). The yellow solution was stirred at room temperature over night. Water was added and the reaction was extracted twice with ethyl acetate. The combined organic layers were washed with saturated aqueous sodium hydrogen carbonate solution and with saturated aqueous sodium chloride solution, dried over sodium sulfate, filtered and the solvent was evaporated under reduced pressure. The title compound was obtained as a light yellow solid (174 mg, 61%) after column chromatography on silica gel using a gradient from heptane/ethyl acetate 4:1 to 1:1 (v/v) as eluent.

MS ISP (m/e): 247.2 (100) [(M+H)⁺], 264.1 (36).

¹H NMR (CDCl₃, 300 MHz): δ (ppm)=7.42-7.38 (m, 3H), 7.09 (d, 2H), 5.61 (t, 1H), 4.56 (m, 2H), 2.77 (m, 1H), 2.42 (m, 1H).

### d) 7-Phenyl-6,7-dihydro-5H-[1,2,4]triazolo[5,1-b][1,3]oxazin-2-ylamine

To a solution of 2-nitro-7-phenyl-6,7-dihydro-5H-[1,2,4]triazolo[5,1-b][1,3]oxazine (174 mg, 707 μmol) in ethly acetate (7 mL) was added Pd on carbon 10% (17.4 mg, 164 μmol). The reaction was hydrogenated at room temperature under an athmosphere of hydrogen over night. The catalyst was filtered off and washed with ethyl acetate. The title compound was obtained as a white solid (143.3 mg, 94%) after stirring with diethyl ether.

MS ISP (m/e): 217.3 (100) [(M+H)⁺].

¹H NMR (DMSO-D₆, 300 MHz): δ (ppm)=7.39-7.29 (m, 3H), 7.16 (d, 2H), 5.22 (t, 1H), 5.15 (br s, 2H), 4.35 (m, 1H), 4.21 (m, 1H), 2.50 (m, 1H), 2.15 (m, 1H).

### e) N-(3-Methoxy-4-(4-methyl-1H-imidazol-1-yl)phenyl)-7-phenyl-6,7-dihydro-5H-[1,2,4]triazolo[5,1-b][1,3]oxazin-2-amine

Prepared in analogy to example 8e, starting 7-phenyl-6,7-dihydro-5H-[1,2,4]triazolo[5,1-b][1,3]oxazin-2-ylamine and 1-(4-bromo-2-methoxy-phenyl)-4-methyl-1H-imidazole. The title compound was obtained as an off-white solid (yield: 58%) after column chromatography on silica gel using a gradient from CH₂Cl₂ to CH₂Cl₂/MeOH 19:1 (v/v) as eluent and precipitation from diethyl ether.

MS ISP (m/e): 403.4 (100) [(M+H)⁺].

¹H NMR (DMSO-D₆, 300 MHz): δ (ppm)=9.28 (s, 1H), 7.56 (s, 1H), 7.42-7.32 (m, 4H), 7.25 (d, 2H), 7.09 (d, 1H), 7.00 (d, 1H), 6.94 (s, 1H), 5.42 (t, 1H), 4.52 (m, 1H), 4.38 (m, 1H), 3.56 (s, 3H), 2.60 (m, 1H), 2.27 (m, 1H), 2.11 (s, 3H).

### Example 110

### 8-(2-chloro-4-fluorophenyl)-N-(3-fluoro-4-(4-methyl-1H-imidazol-1-yl)phenyl)-[1,2,4]triazolo[1,5-a]pyridin-2-amine

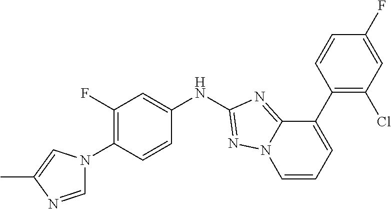

A suspension of 3-fluoro-4-(4-methyl-1H-imidazol-1-yl)aniline (96 mg, 0.5 mmol), 2-bromo-8-(2-chloro-4-fluorophenyl)-[1,2,4]triazolo[1,5-a]pyridine (180 mg, 0.6 0 mmol), 4,5-bis(diphenylphosphino)-9,9-dimethylxanthene (23 mg, 0.4 mmol), tris(dibenzylideneacetone)-dipalladium(o) chloroform adduct (21 mg, 0.2 mmol) and sodium phenoxide (87 mg, 0.75 mmol) in dry 1,4-dioxane (4 mL) was stirred under an argon atmosphere for 60 min at 130° C. (microwave heating). After cooling to ambient temperature it was concentrated and the residue purified by flash chromatography (SiO2,Heptane:EtOAc=1:1 to EtOAc: MeOH=9:1 affording the title product as a light yellow solid (60 mg, 27%)

MS ISP (m/e): 437.2 and 439.3 [M+H)⁺]

¹H NMR (CDCl₃, 300 MHz): δ (ppm)=8.52 (d, 1H), 7.77 (d, 1H), 7.65 (s, 1H), 7.56-7.479 (m, 2H), 7.33-7.24 (m, 4H), 7.16-7.10 (dt, 1H), 7.04 (t, 1H), 6.92 (s, 1H), 2.92 (s, 3H).

### Example 111

### 8-(2-chloro-4-fluorophenyl)-N-(3,5-difluoro-4-(4-methyl-1H-imidazol-1-yl)phenyl)-[1,2,4]triazolo[1,5-a]pyridin-2-amine

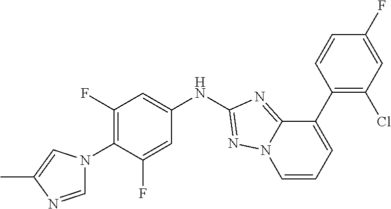

### a) 1-(2,6-difluoro-4-nitrophenyl)-4-methyl-1H-imidazole

A mixture of 1,2,3-trifluoro-5-nitrobenzene (15.0 g, 84.7 mmol), 4-methylimidazole (6.95 g, 84.7 mmol) and triethylamine (8.57 g, 11.8 mL, 84.7 mmol) in acetonitrile (85 mL) was stirred for 20 h at 70° C. Then it was diluted with ethyl acetate (200 mL) and washed with aqueous NaHCO₃ (saturated, 60 mL) and brine (60 ml). The aqueous layers were extracted with further ethyl acetate (100 mL). The organic layers were combined, dried over sodium sulfate, filtered off and evaporated. Recrystallization with a mixture of ethyl acetate (50 mL) and heptane (30 mL) afforded the title compound as light yellow crystals (8.76 g, 43%).

MS ISP (m/e): 240.2 [(M+H)⁺]

¹H NMR (CDCl₃, 300 MHz): δ (ppm)=8.04-8.01 (m, 2H), 7.74 (s, 1H), 6.98 (s, 1H), 2.32 (s, 3H).

### b) 3,5-difluoro-4-(4-methyl-1H-imidazol-1-yl)aniline

A solution of 1-(2,6-difluoro-4-nitrophenyl)-4-methyl-1H-imidazole (4.69 g, 19.6 mmol) in ethanol (160 ml) was treated with tin(II) chloride dihydrate (22.1 g, 98.1 mmol) and stirred for 2 h at reflux. Then it was cooled to ambient temperature and evaporated. The residue was treated with ice-water (120 ml) and set to pH=8 with aqueous Na₂CO₃ (saturated, 70 mL). The mixture was treated with ethyl acetate (200 mL) and then filtered through Dicalite®. The filtrate was separated. The aqueous layer was extracted with further ethyl acetate (200 mL). The organic layers were washed with brine (150 ml), combined, dried over sodium sulfate, filtered off and evaporated affording the title compound as light brown crystals (4.02 g, 98%)

MS ISP (m/e): 210.1 [(M+H)⁺]

¹H NMR (CDCl₃, 300 MHz): δ (ppm)=7.48 (s, 1H), 6.76 (s, 1H), 6.32-6.28 (m, 2H), 4.02 (s br, 2H), 2.29 (s, 3H).

### c) 8-(2-chloro-4-fluorophenyl)-N-(3,5-difluoro-4-(4-methyl-1H-imidazol-1-yl)phenyl)-[1,2,4]triazolo[1,5-a]pyridin-2-amine

Prepared in analogy to example 110 employing 3,5-difluoro-4-(4-methyl-1H-imidazol-1-yl)aniline instead of 3-fluoro-4-(4-methyl-1H-imidazol-1-yl)aniline the title compound was obtained as light yellow solid.

MS ISP (m/e): 455.2 and 457.2 [(M+H)⁺]

¹H NMR (CDCl₃, 300 MHz): δ (ppm)=8.98 (d, 1H), 7.80-7.63 (m, 4H), 7.51 (m, 2H), 7.39 (dt, 1H), 7.20 (m, 2H), 7.07 (s, 1H), 3.31 (s, 3H).

### Example 112

### 8-(2-chloro-4-fluorophenyl)-N-(3-fluoro-5-methoxy-4-(4-methyl-1H-imidazol-1-yl)phenyl)-[1,2,4]triazolo[1,5-a]pyridin-2-amine

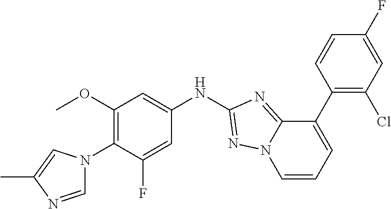

### a) 1-(2-fluoro-6-methoxy-4-nitrophenyl)-4-methyl-1H-imidazole

To a solution of 1-(2,6-difluoro-4-nitrophenyl)-4-methyl-1H-imidazole (4.00 g, 16.7 mmol) in dimethyl sulfoxide (20 mL) was added sodium methoxide solution (5.4 M, 3.1 mL, 16.7 mmol). The reaction mixture was stirred for 2 h at 80° C. Then ice-water (200 mL) was added and was extracted twice with ethyl acetate (100 mL). The organic layers were washed four times with water (50 mL) and once with brine (40 mL), combined, dried over sodium sulfate, filtered off and evaporated. Flash chromatography of the residue (SiO2, heptane: EtOAc=1:1 to 0:1) afforded the title compound as light yellow solid (3.22 g, 77%)

MS ISP (m/e): 252.3 [(M+H)⁺]

¹H NMR (CDCl₃, 300 MHz): δ (ppm)=7.77 (dd, 1H), 7.76 (dd, 1H), 7.64 (s, 1H), 6.87 (s, 1H), 3.99 (s, 3H), 2.32 (s, 3H).

### b) 3-fluoro-5-methoxy-4-(4-methyl-1H-imidazol-1-yl)aniline

Prepared in analogy to example 111 b) employing 1-(2-fluoro-6-methoxy-4-nitrophenyl)-4-methyl-1H-imidazole instead of 1-(2,6-difluoro-4-nitrophenyl)-4-methyl-1H-imidazole the title compound was obtained as yellow solid.

MS ISP (m/e): 222.2 [(M+H)⁺]

¹H NMR (CDCl₃, 300 MHz): δ (ppm)=7.42 (s, 1H), 6.69 (s, 1H), 6.12-6.07 (m, 2H), 3.93 (s br, 2H), 3.74 (s, 3H), 2.28 (s, 3H).

### c) 8-(2-chloro-4-fluorophenyl)-N-(3-fluoro-5-methoxy-4-(4-methyl-1H-imidazol-1-yl)phenyl)-[1,2,4]triazolo[1,5-a]pyridin-2-amine

Prepared in analogy to example 110 employing 3-fluoro-5-methoxy-4-(4-methyl-1H-imidazol-1-yl)aniline instead of 3-fluoro-4-(4-methyl-1H-imidazol-1-yl)aniline the title compound was obtained as light yellow solid.

MS ISP (m/e): 467.2 and 469.2 [(M+H)⁺]

¹H NMR (CDCl₃, 300 MHz): δ (ppm)=8.91 (d, 1H), 7.68-7.62 (m, 4H), 7.53 (s, 1H), 7.38-7.30 (m, 2H), 7.19 (m, 2H), 6.89 (s, 1H), 3.75 (s, 3H), 3.31 (s, 3H).

### Example 113

### 5-(8-(2-chloro-4-fluorophenyl)-[1,2,4]triazolo[1,5-a]pyridin-2-ylamino)-2-(4-methyl-1H-imidazol-1-yl)benzonitrile

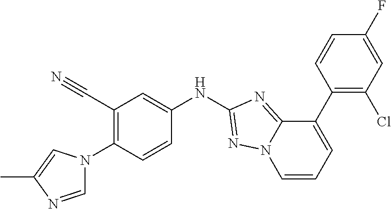

Prepared in analogy to example 110 employing 3-cyano-4-(4-methyl-1H-imidazol-1-yl)aniline instead of 3-fluoro-4-(4-methyl-1H-imidazol-1-yl)aniline the title compound was obtained as light yellow solid.

MS ISP (m/e): 444.2 and 446.1 [(M+H)⁺]

¹H NMR (CDCl₃, 300 MHz): δ (ppm)=8.97 (d, 1H), 8.22 (dd, 1H), 7.94-7.88 (m, 2H), 7.71-7.56 (m, 4H), 7.42-7.39 (m, 1H), 7.24-7.16 (m, 2H), 6.85 (s, 1H), 3.31 (s, 3H).

### Example 114

### 8-(2-chloro-4-fluorophenyl)-N-(3-methoxy-4-(6-methylpyrimidin-4-yl)phenyl)-[1,2,4]triazolo[1,5-a]pyridin-2-amine

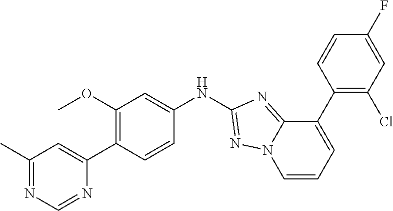

### a) 2-(2-methoxy-4-nitrophenyl)-4,4,5,5-tetramethyl-1,3,2-dioxaborolane

To a solution of 1-bromo-2-methoxy-4-nitrobenzene (5.8 g, 25 mmol) in 1,4-dioxane (125 mL) was added bis(pinacolato)diboron (9.52 g, 37.5 mmol), potassium acetate (7.36 g, 75.0 mmol) and bis(triphenylphosphine)palladium(II) dichloride (877 mg, 1.25 mmol). Then the reaction mixture was stirred for 3 h at reflux. Water (150 mL) was added and the mixture was extracted twice with ethyl acetate (200 mL). The organic layers were washed with brine (150 mL), combined, dried over sodium sulfate, filtered off and evaporated. Flash chromatography of the residue (SiO2, heptane: EtOAc=4:1 to 0:1) afforded the title compound as yellow solid (6.78 g, 97%).

MS ISP (m/e): 279.0 [(M)⁺]

¹H NMR (CDCl₃, 300 MHz): δ (ppm)=7.78 (s, 2H), 7.65 (s, 1H) 3.92 (s, 3H), 1.37 (s, 9H), 1.26 (s, 3H).

### b) 4-(2-methoxy-4-nitrophenyl)-6-methylpyrimidine

To a solution of 2-(2-methoxy-4-nitrophenyl)-4,4,5,5-tetramethyl-1,3,2-dioxaborolane (6.78 g, 24.3 mmol) and 4-chloro-6-methylpyrimidine (4.78 g, 36.4 mmol) in acetonitrile (272 mL) was added a solution of sodium carbonate (12.9 g, 121 mmol) in water (68 ml). This mixture was degassed and flushed with Ar and then tetrakis(triphenylphosphine)palladium(0) (1.4 g, 1.21 mmol) was added. It was stirred for 3 h at reflux and then poured onto water (300 mL). The mixture was extracted with ethyl acetate (350 mL) three times. The organic layers were washed with brine. (250 mL), combined, dried over sodium sulfate, filtered off and evaporated. Flash chromatography of the residue (SiO2, heptane: EtOAc=4:1 to 0:1) afforded the title compound yellow solid (5.92 g, 99%).

MS ISP (m/e): 246.3 [(M+H)⁺]

¹H NMR (CDCl₃, 300 MHz): δ (ppm)=9.20 (s, 1H), 8.12 (d, 1H), 7.96 (dd, 1H), 7.88 (d, 1H), 7.81 (s, 1H), 4.02 (s, 3H), 2.62 (s, 3H).

### c) 3-methoxy-4-(6-methylpyrimidin-4-yl)aniline

Prepared in analogy to example 111 b) employing 4-(2-methoxy-4-nitrophenyl)-6-methylpyrimidine instead of 1-(2,6-difluoro-4-nitrophenyl)-4-methyl-1H-imidazole the title compound was obtained as yellow solid.

MS ISP (m/e): 216.3 [(M+H)⁺]

¹H NMR (CDCl₃, 300 MHz): δ (ppm)=9.01 (dd, 1H), 7.91 (t, 1H), 7.79 (s, 1H), 6.69 (s, 1H), 6.60 (d, 1H), 3.89 (s, 3H), 2.53 (s, 3H).

### d) 8-(2-chloro-4-fluorophenyl)-N-(3-methoxy-4-(6-methylpyrimidin-4-yl)phenyl)-[1,2,4]triazolo[1,5-a]pyridin-2-amine

Prepared in analogy to example 110 employing 3-methoxy-4-(6-methylpyrimidin-4-yl)aniline instead of 3-fluoro-4-(4-methyl-1H-imidazol-1-yl)aniline the title compound was obtained as orange solid.

MS ISP (m/e): 461.2 and 463.2 [(M+H)⁺]

¹H NMR (CDCl₃, 300 MHz): δ (ppm)=8.97 (d, 1H), 8.99 (s, 1H), 8.89 (d, 1H), 8.01 (d, 1H), 7.89 (s, 1H), 7.73 (m, 1H), 7.70-7.61 (m, 2H), 7.39 (dt, 1H), 7.23 (dd, 1H), 7.15 (t, 1H), 3.88 (s, 3H), 2.47 (s, 3H).

### Example 115

### 8-(2-chloro-4-fluorophenyl)-N-(4-(2,6-dimethylpyrimidin-4-yl)phenyl)-[1,2,4]triazolo[1,5-a]pyridin-2-amine

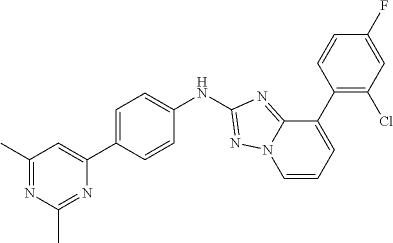

Prepared in analogy to example 110 employing 4-(2,6-dimethylpyrimidin-4-yl)aniline instead of 3-fluoro-4-(4-methyl-1H-imidazol-1-yl)aniline the title compound was obtained as a light brown solid.

MS ISP (m/e): 445.3 and 447.1 [(M+H)⁺]

¹H NMR (CDCl₃, 300 MHz): δ (ppm)=8.93 (d, 1H), 8.14 (d, 2H), 7.76 (d, 2H), 7.70-7.58 (m, 4H), 7.39 (dt, 1H), 7.16 (t, 1H), 2.59 (s, 3H9, 2.45 (s, 3H).

### Example 116

### 2-{8-(4-Chloro-phenyl)-2-[3-methoxy-4-(4-methyl-imidazol-1-yl)-phenylamino]-[1,2,4]triazolo[1,5-a]pyridin-5-yl}-propan-2-ol

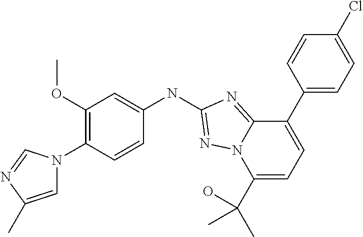

### a) N-(6-Methyl-pyridin-2-yl)-acetamide

To a solution of 6-methyl-pyridin-2-ylamine (50 g, 0.462 mol) in acetic anhydride (200 mL) was heated to 90° C. for 90 minutes. The reaction mixture was cooled to room temperature and evaporated. An aqueous saturated solution of NaHCO₃ was added to the residue until pH 8. The aqueous phase was extracted with ethyl acetate, the combined organic phases were dried over sodium sulfate, and the solvent was evaporated. The title compound was obtained as a white solid (68 g, 98%).

MS ESI (m/e): 151.2 [(M+H)⁺].

¹H NMR (DMSO, 400 MHz): δ (ppm)=10.38 (s, 1H), 7.86 (d, J=8.2 Hz, 2H), 7.62 (t, J=7.8 Hz, 1H), 6.92 (d, J=7.4 Hz, 1H), 2.38 (s, 3H), 2.06 (s, 3H).

### b) 6-Acetylamino-pyridine-2-carboxylic acid

A solution of N-(6-methyl-pyridin-2-yl)-acetamide (10 g, 0.067 mmol) in water (100 mL) was heated to 75° C. Potassium permanganate (37 g, 233 mmol) was added portion-wise at 75° C. After 4 hours at 75° C. for the reaction mixture was cooled to room temperature and the solid was filtered. The aqueous layer was evaporated to half of its original volume and acidified with HCl (12N) to pH 4-5. The precipitate was filtered and dried. The title compound was obtained as off white solid (4.5 g, 37%).

MS ESI (m/z): 181.2 [(M+H)⁺].

¹H NMR (DMSO, 400 MHz): δ (ppm)=13.0 (s, 1H), 10.78 (s, 1H), 8.26 (d, J=8.28 Hz, 1H), 7.92 (t, J=7.8 Hz, 1H), 7.72 (d, J=7.1 Hz, 1H), 2.10 (s, 3H).

### c) 6-Amino-pyridine-2-carboxylic acid methyl ester

A solution of 6-acetylamino-pyridine-2-carboxylic acid (16 g, 0.088 mol) in methanolic hydrochloride (4N, 50 mL) was heated to reflux for 18 hours. The reaction mixture was cooled to room temperature and evaporated. Water was added to the residue and alkalized with solid NaHCO₃. The aqueous phase was extracted with ethyl acetate, the combined organic phases were dried over sodium sulfate, and the solvent was evaporated. The title compound was obtained as a white solid (8 g, 59%).

MS ESI (m/z): 153.0 [(M+H)⁺].

¹H NMR (DMSO, 400 MHz): δ(ppm)=7.53 (t, J=7.52 Hz, 1H), 7.48 (d, J=7.28 Hz, 2H), 6.66 (d, J=8.04 Hz, 1H), 4.71 (s, 2H), 3.94 (s, 3H).

### d) 6-Amino-5-bromo-pyridine-2-carboxylic acid methyl ester

To a solution of 6-amino-pyridine-2-carboxylic acid methyl ester (10 g, 66.0 mmol) in chloroform (450 mL) was added bromine (3.4 mL, 66.0 mmol) in CHCl₃ (100 mL) at room temperature and stirred for 40 hours. The reaction mixture was diluted with CHCl₃ and washed with saturated sodium thiosulfate solution and water. The organic phase was dried over sodium sulfate, the solvent was evaporated and the residue purified by silica gel column chromatography using ethyl acetate/hexane as eluent. The title compound obtained as yellow solid (3.3 g, 22%).

MS ESI (m/e): 231.0 [(M+H)⁺].

¹H NMR (CDCl₃, 400 MHz): δ(ppm)=7.76 (d, J=7.88 Hz, 1H), 7.34 (d, J=7.92 Hz, 1H), 5.23 (s, 2H), 3.94 (s, 3H).

### d) 6-Amino-3-bromo-pyridine-2-carboxylic acid methyl ester

In step d) the isomeric 6-amino-3-bromo-pyridine-2-carboxylic acid methyl ester (3.0 g, 19%) was isolated as side product.

MS ESI (m/e): 231.2 [(M+H)⁺].

¹H NMR (CDCl₃, 400 MHz): δ(ppm)=7.60 (d, J=8.72 Hz, 1H), 6.47 (d, J=7.88 Hz, 1H), 4.71 (s, 2H), 3.94 (s, 3H).

### e) N-(3-bromo-6-ethoxycarbonyl-pyridin-2-yl)-N′-ethoxycarbonyl-thiourea

To a solution of 6-amino-5-bromo-pyridine-2-carboxylic acid methyl ester (3.3 g, 14.285 mmol) in dry 1,4-dioxane (20 mL) was added ethoxy carbonyl isothiocyanate (1.8 mL, 15.7 mmol) under an argon atmosphere and stirred at room temperature for 16 hours. The solvent was evaporated and the title compound was obtained as yellow solid (4.9 g, 95%).

MS ESI (m/e): 362.0 [(M+H)⁺].

¹H NMR (DMSO, 400 MHz): δ(ppm)=1.54 (s, 1H), 11.46 (s, 1H), 8.36 (d, J=8.16 Hz, 1H), 7.92 (d, J=8.16 Hz, 1H), 4.27-4.23 (m, 2H), 3.89 (s, 3H), 1.36-1.26 (m, 3H).

### f) 2-Amino-8-bromo-[1,2,4]triazolo[1,5-a]pyridine-5-carboxylic acid methyl ester

To a solution of N-(3-bromo-6-ethoxycarbonyl-pyridin-2-yl)-N′-ethoxycarbonyl-thiourea (2 g, 5.52 mmol) in dry methanol (10 mL) were added hydroxylamine hydrochloride (1.92 g, 27.62 mmol) and diisopropyl ethylamine (2.98 mL, 16.57 mmol) under an argon atmosphere and stirred at room temperature for 4 hours. The solid was filtered and methanol (40 mL) was added to residue. The reaction mixture was heated to reflux for 12 hours. The solvent was evaporated and the title compound was obtained as off white solid (800 mg, 53%).

MS ESI (m/e): 270.8 [(M+H)⁺].

¹H NMR (DMSO, 400 MHz): δ (ppm)=7.66 (d, J=8.04 Hz, 1H), 7.43 (d, J=8.12 Hz, 1H), 4.9 (s, 2H), 4.02 (s, 3H).

### g) 2-(2-Amino-8-bromo-[1,2,4]triazolo[1,5-a]pyridin-5-yl)-propan-2-ol

To a solution of 2-amino-8-bromo-[1,2,4]triazolo[1,5-a]pyridine-5-carboxylic acid methyl ester (900 mg, 3.32 mmol) in tetrahydrofuran was added methyl magnesium bromide (1.4 M solution in toluene/tetrahydrofuran; 75/25) (9.49 mL, 13.28 mmol) at −40° C. and stirred at −30° C. for 1 hour. The reaction mixture was warmed to room temperature and quenched with saturated aqueous NH₄Cl solution. The aqueous phase was extracted with ethyl acetate, the combined organic phases were dried over sodium sulfate, the solvent was evaporated and the residue was purified by silica gel column chromatography using ethyl acetate/hexane as eluent. The title compound was obtained as yellow solid (400 mg, 44%) which was contaminated with 1-(2-amino-8-bromo-[1,2,4]triazolo[1,5-a]pyridin-5-yl)-ethanone.

MS ESI (m/e): 273.2 [(M+H)⁺].

### h) 2-[2-Amino-8-(4-chloro-phenyl)-[1,2,4]triazolo[1,5-a]pyridin-5-yl]-propan-2-ol

To a solution of 2-(2-amino-8-bromo-[1,2,4]triazolo[1,5-a]pyridin-5-yl)-propan-2-ol (contaminated with ketone) (120 mg, 0.443 mmol) and 4-chlorophenyl boronic acid (155 mg, 0.9874 mmol) in dioxane (6 mL) was added aqueous solution of Na₂CO₃ (2M, 0.72 mL) and degassed with argon for 5 minute. To this was added PdCl₂ (dppf)₂.CH₂Cl₂ (30.34 mg, 0.04 mmol) and stirred at 90° C. for 90 minutes. The reaction mixture was cooled to room temperature and water (20 mL) was added. The aqueous phase was extracted with ethyl acetate, the combined organic phases were dried over sodium sulfate, the solvent was evaporated and the residue was purified by silica gel chromatography using ethyl acetate/hexane as eluent. The title compound was obtained as an off white solid (65 mg, 48%) which was contaminated with 1-[2-amino-8-(4-chloro-phenyl)-[1,2,4]triazolo[1,5-a]pyridin-5-yl]-ethanone.

MS ESI (m/e): 273.2 [(M+H)⁺].

### i) 2-[2-Bromo-8-(4-chloro-phenyl)-[1,2,4]triazolo[1,5-a]pyridin-5-yl]-propan-2-ol

To a solution of tert-butylnitrite (0.06 mL, 0.47 mmol) in dry acetonitrile (5 mL) was added Cu(II) Bromide (105 mg, 0.47 mmol) under an argon atmosphere and heated to 60° C. for 0.1 hour. 2-[2-Amino-8-(4-chloro-phenyl)-[1,2,4]triazolo[1,5-a]pyridin-5-yl]-propan-2-ol (mixture of alcohol and ketone) (90 mg, 0.32 mmol) in acetonitrile (5 mL) was added at 60° C. and stirred at 75° C. for 3 hour. The reaction mixture was cooled to room temperature and water (10 mL) was added. The aqueous phase was extracted with dichloromethane, the combined organic phases were dried over sodium sulfate, the solvent was evaporated and the residue was purified by silica gel chromatography using ethyl acetate/hexane as eluent. The title compound was obtained as off white solid (20 mg, 48%).

MS ESI (m/e): 368.0 [(M+H)⁺].

### k) 2-{8-(4-Chloro-phenyl)-2-[3-methoxy-4-(4-methyl-imidazol-1-yl)-phenylamino]-[1,2,4]triazolo[1,5-a]pyridin-5-yl}-propan-2-ol

A solution of 2-[2-bromo-8-(4-chloro-phenyl)-[1,2,4]triazolo[1,5-a]pyridin-5-yl]-propan-2-ol (35 mg, 0.096 mmol), 3-methoxy-4-(4-methyl-imidazol-1-yl)-phenylamine (16 mg, 0.08 mmol) and sodium phenoxide (14 mg, 0.12 mmol) in dry 1,4-dioxane (5 mL) in a sealed tube was purged with argon gas for 10 min. Pd₂(dba)₃.CHCl₃ (7 mg, 0.01 mmol) and xanthphos (2 mg) were added to the solution and continue the degassing another 5 min and heated to 160° C. for 15 hours.

The reaction mixture was cooled to room temperature and water (10 mL) was added. The aqueous phase was extracted with ethyl acetate, the combined organic phases were dried over sodium sulfate, the solvent was evaporated and the residue purified by silica gel chromatography using dichloromethane/methanol as eluent. The title compound was obtained as a light yellow solid (15 mg, 32%).

MS ESI (m/e): 489.0 [(M+H)⁺].

¹H NMR (DMSO, 400 MHz): δ (ppm)=10.08 (s, 1H), 8.19 (d, J=8.48 Hz, 1H), 7.98 (s, 1H), 7.95 (d, J=7.88 Hz, 1H), 7.66 (s, 1H), 7.59 (d, J=8.52 Hz, 2H), 7.33 (d, J=7.88 Hz, 1H), 7.25 (d, J=8.6 Hz, 1H) 7.09 (d, J=7.76 Hz, 1H), 7.04 (s, 1H), 5.87 (s, 1H), 3.87 (s, 3H), 2.15 (s, 3H), 1.83 (s, 6H).

### Example 117

### [8-(4-Chloro-phenyl)-6-cyclopropyl-[1,2,4]triazolo[1,5-a]pyridin-2-yl]-[3-methoxy-4-(4-methyl-imidazol-1-yl)-phenyl]-amine

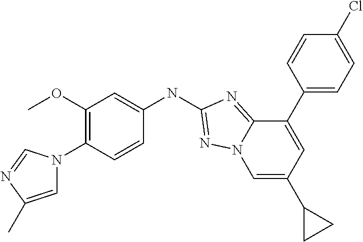

### a) 5-Cyclopropyl-pyridin-2-ylamine

To a solution of 5-bromo-pyridin-2-ylamine (2 g, 11.55 mmol) and cyclopropyl boronic acid (2.98 g, 34.68 mmol) in toluene (40 mL) and water (2 mL) was added K₃PO₄ (8.59 g, 40.46 mmol) under an argon atmosphere. A balloon containing argon was affixed, and the reaction flask was purged to ensure a argon atmosphere. To this were added Pd(OAc)₂, (259.52 mg, 1.16 mmol) and tricyclohexylphosphene (647.3 mg, 2.3 mmol) and stirred at 80° C. for 16 h. The reaction mixture was cooled to room temperature and water was added. The aqueous phase was extracted with ethyl acetate, the combined organic phases were dried over sodium sulfate, the solvent was evaporated and the residue purified by silica gel chromatography using ethyl acetate/hexane as eluent. The title compound was obtained as an off white solid (1.1 g, 71%).

¹H NMR (DMSO, 400 MHz): δ(ppm)=7.73 (s, 1H), 7.04-7.02 (dd, J=8.48 & 2.04 Hz, 1H), 6.34 (d, J=8.48 & 2.04 Hz, 1H), 5.60 (s, 2H), 1.78-1.66 (m, 1H), 0.822-0.77 (m, 2H), 0.52-0.313 (m, 2H)

### b) 3-Bromo-5-cyclopropyl-pyridin-2-ylamine

To a solution of 5-cyclopropyl-pyridin-2-ylamine (1.1 g, 8.19 mmol) in dry chloroform (100 mL) was added bromine (0.42 mL, 8.2 mmol) in chloroform (11 mL) at room temperature and stirred for 18 hour. An aqueous solution of sodium thiosulfate was added to the residue. The aqueous phase was extracted with dichloromethane, the combined organic phases were dried over sodium sulfate, the solvent was evaporated and the residue purified by silica gel chromatography using ethyl acetate/hexane as eluent. The title compound was obtained as light yellow oil (1.0 g, 57%).

MS ESI (m/z): 213.0 [(M+H)⁺].

¹H NMR (DMSO, 400 MHz): δ(ppm)=7.77 (d, J=1.44 Hz, 1H), 7.39 (d, J=1.44 Hz, 1H), 5.9 (s, 2H), 1.79-1.74 (m, 1H), 0.85-0.80 (m, 2H), 0.59-0.55 (m, 2H).

### c) N-(3-bromo-5-cyclopropyl-pyridin-2-yl)-N′-ethoxycarbonyl-thiourea

To a solution of 3-bromo-5-cyclopropyl-pyridin-2-ylamine (1.0 g, 4.69 mmol) in dry 1,4-dioxane (20 mL) was added ethoxy carbonyl isothiocyanate (0.55 mL, 5.16 mmol) under an argon atmosphere and stirred at room temperature for 6 hour. The solvent was evaporated and the title compound was obtained as light yellow oil (1.5 g, 98.2%).

MS ESI (m/z): 346.2 [(M+H)⁺].

¹H NMR (DMSO, 400 MHz): δ(ppm)=11.41 (s, 1H), 11.32 (s, 1H), 8.29 (s, 1H), 7.80 (s, 1H), 4.24-4.19 (q, J=7.08, 2H), 2.03-1.97 (m, 1H), 1.28-1.24 (t, J=7.12 Hz, 3H), 1.06-0.97 (m, 2H), 0.84-0.81 (m, 2H).

### d) 8-Bromo-6-cyclopropyl-[1,2,4]triazolo[1,5-a]pyridin-2-ylamine

To a solution of N-(3-bromo-5-cyclopropyl-pyridin-2-yl)-N′-ethoxycarbonyl-thiourea (1.5 g, 4.36 mmol) in dry methanol (20 mL) were added hydroxylamine hydrochloride (1.41 g, 21.8 mmol) and diisopropyl ethylamine (12.14 mL, 13.08 mmol) under an argon atmosphere and stirred at room temperature for 6 hour. Methanol was evaporated and the residue purified by silica gel chromatography using ethyl acetate/hexane as eluent. The title compound was obtained as off white solid (910 mg, 82.46%).

MS ESI (m/z): 252.6 [(M+H)⁺].

¹H NMR (DMSO, 400 MHz): δ (ppm)=8.41 (s, 1H), 7.48 (s, 1H), 6.12 (s, 2H), 1.99-1.90 (m, 1H), 0.93-0.84 (m, 2H), 0.80-0.75 (m, 2H).

### e) 8-(4-Chloro-phenyl)-6-cyclopropyl-[1,2,4]triazolo[1,5-a]pyridin-2-ylamine

To a solution of 8-bromo-6-cyclopropyl-[1,2,4]triazolo[1,5-a]pyridin-2-ylamine (300 mg, 1.29 mmol) and 4-chlorophenyl boronic acid (463 mg, 2.96 mmol) in dioxane (15 mL) was added an aqueous solution of Na₂CO₃ (2M, 2 ml) and degassed with argon for 5 minute. PdCl₂ (dppf)₂.CH₂Cl₂ (30.34 mg, 0.04 mmol) was added and stirred at 80° C. for 90 minute. The reaction mixture was cooled to room temperature and water (20 mL) was added. The aqueous phase was extracted with ethyl acetate, the combined organic phases were dried over sodium sulfate, the solvent was evaporated and the residue purified by silica gel chromatography using ethyl acetate/hexane as eluent. The title compound was obtained as an off white solid (252 mg, 75%).

MS ESI (m/z): 284.8 [(M+H)⁺].

¹H NMR (DMSO, 400 MHz): δ(ppm)=8.39 (s, 1H), 8.18 (d, 2H), 7.54 (d, 2H), 7.45 (s, 1H), 2.05-2.01 (m, 1H), 0.97-0.92 (m, 2H), 0.84-0.82 (m, 2H).

### f) 2-Bromo-8-(4-chloro-phenyl)-6-cyclopropyl-[1,2,4]triazolo[1,5-a]pyridine

To a solution of tert-butylnitrite (0.18 mL, 1.05 mmol) in dry acetonitrile (7 mL) was added copper(II) bromide (234 mg, 1.05 mmol) under an argon atmosphere and heated to 60° C. for 0.1 hour. 8-(4-Chloro-phenyl)-6-cyclopropyl-[1,2,4]triazolo[1,5-a]pyridin-2-ylamine (200 mg, 0.7 mmol) in acetonitrile (5 mL) was added at 60° C. The reaction mixture was stirred at 75° C. for 3 hour and then cooled to room temperature. Water (10 mL) was added. The aqueous phase was extracted with dichloromethane, the combined organic phases were dried over sodium sulfate, the solvent was evaporated and the residue purified by silica gel chromatography using ethyl acetate/hexane as eluent. The title compound was obtained as off white solid (150 mg, 61%).

MS ESI (m/z): 348.2 [(M+H)⁺].

¹H NMR (DMSO, 400 MHz): δ(ppm)=8.81 (s, 1H), 8.14 (d, J=8.52 Hz, 2H), 7.72 (s, 1H), 7.62 (d, J=8.4 Hz, 2H), 2.12-2.10 (m, 1H), 1.03-0.93 (m, 4H).

### g) [8-(4-Chloro-phenyl)-6-cyclopropyl-[1,2,4]triazolo[1,5-a]pyridin-2-yl]-[3-methoxy-4-(4-methyl-imidazol-1-yl)-phenyl]-amine

A solution of 2-bromo-8-(4-chloro-phenyl)-6-cyclopropyl-[1,2,4]triazolo[1,5-a]pyridine (110 mg, 0.32 mmol), 3-methoxy-4-(4-methyl-imidazol-1-yl)-phenylamine (50.6 mg, 0.26 mmol) and sodium phenoxide (48 mg, 0.4 mmol) in dry 1,4-dioxane (4 mL) in a sealed tube was purged with argon for 10 min. Pd₂(dba)₃.CHCl₃ (18.6 mg, 0.02 mmol) and xanthphos (4 mg) were added to the solution and stirred at 160° C. for 15 hours. The reaction mixture was cooled to room temperature and water (10 mL) was added. The aqueous phase was extracted with ethyl acetate, the combined organic phases were dried over sodium sulfate, the solvent was evaporated and the residue purified by silica gel chromatography using dichloromethane/methanol as eluent. The title compound was obtained as a light yellow solid (28 mg, 18%).

MS ESI (m/z): 470.8 [(M+H)⁺].

¹H NMR (DMSO, 400 MHz): δ(ppm)=9.92 (s, 1H), 8.65 (s, 1H), 8.24 (d, J=8.64 Hz, 2H), 7.78 (d, J=1.8 Hz, 1H), 7.61 (d, J=5.62 Hz, 2H), 7.57 (d, J=8.6 Hz, 2H), 7.25-7.21 (m, 2H), 7.01 (s, 1H), 3.82 (s, 3H), 2.13-2.06 (m, 4H), 0.99-0.96 (m, 2H), 0.91-0.85 (m, 2H).

### Example 118

### [6-Cyclopropyl-8-(4-fluoro-phenyl)-[1,2,4]triazolo[1,5-a]pyridin-2-yl]-[3-methoxy-4-(4-methyl-imidazol-1-yl)-phenyl]-amine

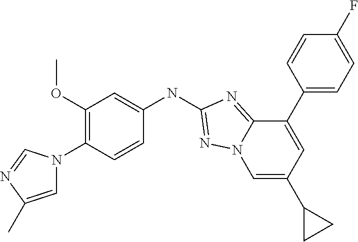

Prepared in analogy to example 117. The title compound was obtained as an off-white solid.

MS ESI (m/z): 455.2 [(M+H)⁺].

¹H NMR (DMSO, 400 MHz): δ(ppm)=9.93 (s, 1H), 8.64 (s, 1H), 8.24 (q, J=5.6 Hz, 2H), 7.8 (br s, 1H), 7.64 (br s, 1H), 7.50 (br s, 1H), 7.35 (t, J=8.84 Hz, 1H), 7.24 (br s, 2H), 7.02 (br s, 1H), 3.83 (s, 3H), 2.14 (s, 3H), 2.09-2.08 (m, 1H), 1.0-0.07 (m, 2H), 0.92-0.89 (m, 2H).

### Example 119

### 2-{8-(4-Fluoro-phenyl)-2-[3-methoxy-4-(4-methyl-imidazol-1-yl)-phenylamino]-[1,2,4]triazolo[1,5-a]pyridin-5-yl}-propan-2-ol

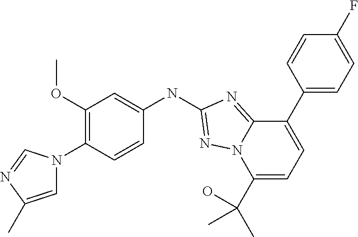

Prepared in analogy to example 116. The title compound was obtained as an off-white solid.

MS ESI (m/e): 473.3 [(M+H)⁺].

¹H NMR (DMSO, 400 MHz): δ (ppm)=10.05 (s, 1H), 8.2-8.16 (m, 2H), 7.99 (s, 1H), 7.89 (d, J=7.92 Hz, 1H), 7.64 (s, 1H), 7.38-7.30 (m, 3H), 7.23 (d, J=8.48 Hz, 1H), 7.07 (d, J=8.24 Hz, 1H), 7.02 (s, 1H), 5.84 (s, 1H), 3.86 (s, 3H), 2.14 (s, 3H), 1.82 (s, 6H).

### Example 120

### [6-Cyclopropyl-8-(2,3,4-trifluoro-phenyl)-[1,2,4]triazolo[1,5-a]pyridin-2-yl]-[3-methoxy-4-(4-methyl-imidazol-1-yl)-phenyl]-amine

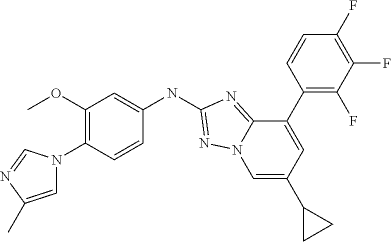

Prepared in analogy to example 117. The title compound was obtained as an off-white solid.

MS ESI (m/z): 491 [(M+H)⁺].

¹H NMR (DMSO, 400 MHz): δ (ppm)=9.92 (s, 1H), 8.73 (s, 1H), 7.73 (s, 1H), 7.71-7.6 (m, 1H), 7.62 (s, 1H), 7.52-7.48 (m, 2H), 3.78 (s, 3H), 2.32 (s, 3H), 2.12-2.06 (m, 1H), 1.01-0.97 (m, 2H), 0.86-0.84 (m, 2H).

### Example 121

### 2-{8-(2-Chloro-4-fluoro-phenyl)-2-[3-methoxy-4-(4-methyl-imidazol-1-yl)-phenylamino]-[1,2,4]triazolo[1,5-a]pyridin-5-yl}-propan-2-ol

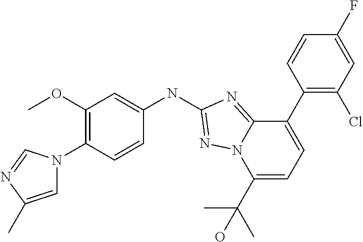

Prepared in analogy to example 116. The title compound was obtained as an off-white solid.

MS ESI (m/e): 507.0 [(M+H)⁺].

¹H NMR (DMSO, 400 MHz): δ (ppm)=10.06 (s, 1H), 7.99-7.88 (m, 2H), 7.68-7.60 (m, 3H), 7.40-7.30 (m, 2H), 7.25-7.20 (m, 1H), 7.01-6.96 (m, 2H), 5.86 (s, 1H) 3.82 (s, 3H), 2.13 (s, 3H), 1.81 (s, 1H).

### Example 122

### 2-[2-[3-Methoxy-4-(4-methyl-imidazol-1-yl)-phenylamino]-8-(2,3,4-trifluoro-phenyl)-[1,2,4]triazolo[1,5-a]pyridin-5-yl]-propan-2-ol

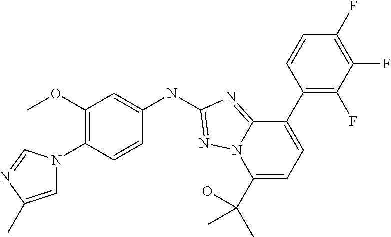

Prepared in analogy to example 116. The title compound was obtained as an off-white solid.

MS ESI (m/e): 509.2 [(M+H)⁺].

¹H NMR (DMSO, 400 MHz): δ (ppm)=10.06 (s, 1H), 7.94 (s, 1H), 7.77 (d, J=7.72 Hz, 1H), 7.7-7.67 (m, 1H), 7.69 (s, 1H), 7.52-7.45 (m, 1H), 7.33 (d, J=7.72 Hz, 1H), 7.22 (d, J=8.48 Hz, 1H), 7.02 (d, J=8.80 Hz, 2H), 5.87 (s, 1H), 3.83 (s, 3H), 2.13 (s, 3H), 1.83 (s, 6H).

### Example 123

### 2-{8-(3-Chloro-4-fluoro-phenyl)-2-[3-methoxy-4-(4-methyl-imidazol-1-yl)-phenylamino]-[1,2,4]triazolo[1,5-a]pyridin-5-yl}-propan-2-ol

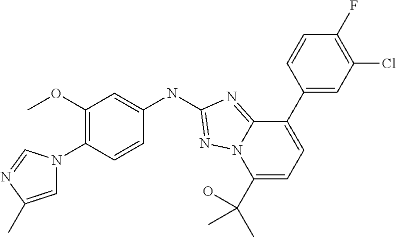

Prepared in analogy to example 116. The title compound was obtained as an off-white solid.

MS ESI (m/e): 507.2 [(M+H)⁺].

¹H NMR (DMSO, 400 MHz): δ (ppm)=10.07 (s, 1H), 8.42-8.40 (m, 1H), 8.17-8.11 (m, 1H), 7.98-7.95 (m, 2H), 7.64 (s, 1H), 7.57 (t, J=9.04 Hz, 1H), 7.31 (d, J=7.88 Hz, 1H), 7.24 (d, J=8.48 Hz, 1H), 7.09 (d, J=8.56 Hz, 1H), 5.86 (s, 1H), 3.86 (s, 3H), 2.14 (s, 3H), 1.82 (s, 6H).

### Example 124

### 5-(4-fluorophenyl)-N-(3-methoxy-4-(4-methyl-1H-imidazol-1-yl)phenyl)-7-methyl-5,6,7,8-tetrahydro-[1,2,4]triazolo[1,5-a]pyrazin-2-amine

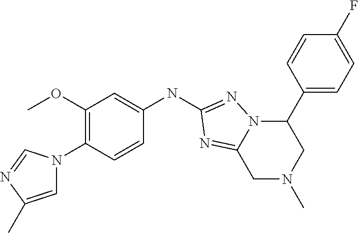

### a) tert-butyl 5-(4-fluorophenyl)-7-methyl-5,6,7,8-tetrahydro-[1,2,4]triazolo[1,5-a]pyrazin-2-ylcarbamate

To a solution of tert-butyl 5-(4-fluorophenyl)-5,6,7,8-tetrahydro-[1,2,4]triazolo[1,5-a]pyrazin-2-ylcarbamate (isolated as side product (9%) in example 57b, 100 mg, 0.30 mmol) in dichloroethane (2 mL) were added formaldehyde (30% in water, 23 μL, 0.30 mmol) and sodium triacetoxyborohydride (254 mg, 1.2 mmol) and the reaction mixture was stirred at rt. After 90 minutes further sodium triacetoxyborohydride (127 mg, 0.6 mmol) was added and stirred at rt for 90 minutes. Saturated aqueous sodium bicarbonate solution was added to the reaction mixture and the aqueous phase was extracted with ethyl acetate. The combined organic phases were dried over sodium sulfate, the solvent was evaporated and the residue purified by silica gel chromatography using CH₂Cl₂/MeOH (with 10% ammonia) as eluent. The title compound was obtained as a white solid (51 mg, 49%).

MS ISP (m/e): 348.3 (18) [(M+H)⁺], 292.1 (100) [(M−tBu)⁺].

¹H NMR (CDCl₃, 300 MHz): δ (ppm)=7.18-7.14 (m, 2H), 7.05-6.99 (m, 3H), 5.31-5.27 (m, 1H), 3.87-3.68 (m, 2H), 3.15-3.09 (m, 1H), 2.88-2.82 (m, 1H), 2.45 (s, 3H), 1.48 (s, 9H).

### b) 5-(4-fluorophenyl)-N-(3-methoxy-4-(4-methyl-1H-imidazol-1-yl)phenyl)-7-methyl-5,6,7,8-tetrahydro-[1,2,4]triazolo[1,5-a]pyrazin-2-amine

Prepared in analogy to example 57d-e) employing tert-butyl 5-(4-fluorophenyl)-7-methyl-5,6,7,8-tetrahydro-[1,2,4]triazolo[1,5-a]pyrazin-2-ylcarbamate. The title compound was obtained as a white solid.

MS ISP (m/e): 434.3 (100) [(M+H)⁺].

¹H NMR (CDCl₃, 300 MHz): δ (ppm)=7.56-7.55 (m, 1H), 7.34-7.33 (m, 1H), 7.26-7.22 (m, 2H), 7.08-7.02 (m, 3H), 6.81-6.75 (m, 2H), 6.67 (m, 1H), 5.31-5.27 (m, 1H), 3.78 (s, 2H), 3.64 (s, 3H), 3.23-3.17 (m, 1H), 2.90-2.83 (m, 1H), 2.51 (s, 3H), 2.28 (s, 3H).

### Example 125

### [8-(4-Chloro-phenyl)-[1,2,4]triazolo[1,5-a]pyridin-2-yl]-[3-methoxy-4-(4-methyl-imidazol-1-yl)-phenyl]-amine

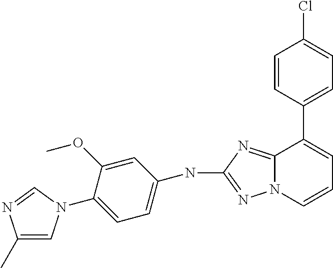

### a) N-(3-bromo-pyridin-2-yl)-N′-ethoxycarbonyl-thiourea

3-Bromopyridin-2-amine (30 g, 168 mmol) and ethoxycarbonyl isothiocyanate (24.8 g, 21.3 ml, 185 mmol) were dissolved in dioxane (300 ml) and stirred at rt. After 4 h further ethoxycarbonyl isothiocyanate (1 ml, 8.4 mmol) was added. After 1 hour the solvent was evaporated and residue dried in high vacuum for 12 h. The title compound was obtained as a light yellow solid (51.2 g, 100%) and was used crude for the next step.

MS ISP (m/e): 304.0/305.9 (100/73) [(M+H)⁺].

¹H NMR (CDCl₃, 300 MHz): δ (ppm)=8.41 (m, 1H) 7.99-7.96 (m, 1H), 7.11-7.07 (m, 1H), 4.32 (q, 2H), 1.36 (t, 3H).

### b) 8-bromo-[1,2,4]triazolo[1,5-a]pyridin-2-amine

Hydroxyl amine (58.5 g, 842 mmol) and N,N-diisopropylethylamine (65.3 g, 86.3 ml, 505 mmol) were dissolved in methanol (200 ml) and ethanol (200 ml). N-(3-bromo-pyridin-2-yl)-N′-ethoxycarbonyl-thiourea (51.2 g, 168 mmol) was added and the reaction mixture was stirred at rt for 1 hour and then at 60° C. for 3 hours.

The white precipitate was filtered off and triturated with water for 25 min, filtered and triturated two times with diethylether. The solid was dried by co-evaporation with toluene and dried in vacuum. The title compound was obtained as a white solid (27.9 g, 78%).

MS ISP (m/e): 213.0/215.1 (86/95) [(M+H)⁺].

¹H NMR (CDCl₃, 300 MHz): δ (ppm)=8.28 (dd, 1H) 7.62 (dd, 1H), 6.73 (t, 1H), 4.66 (bs, 2H).

### c) 8-(4-Chloro-phenyl)-[1,2,4]triazolo[1,5-a]pyridin-2-ylamine

A mixture of 8-bromo-[1,2,4]triazolo[1,5-a]pyridin-2-amine (500 mg, 2.35 mmol), 4-chlorophenyl boronic acid (757 mg, 4.69 mmol), dichloro[1,1′-bis(diphenylphosphino)-ferrocene]palladium(II) dichloromethane adduct (153 mg, 0.188 mmol) and an aqueous solution of Na₂CO₃ (2 N, 2.35 mL, 4.69 mmol) in dioxane (10 mL) was stirred at 110° C. for 2 hours. The reaction mixture was diluted with a 2N aqueous solution of sodium carbonate and extracted with diethyl ether, the combined organic phases were dried over sodium sulfate, the solvent was evaporated and the residue purified by silica gel chromatography using pentane/diethyl ether as eluent. The title compound was obtained as a white solid (572 mg, 99%).

MS ISP (m/e): 245.3/247.2 (100/38) [(M+H)⁺].

¹H NMR (CDCl₃, 300 MHz): δ (ppm)=8.30 (dd, 1H) 7.93-7.88 (m, 2H), 7.52-7.45 (m, 3H), 6.92 (t, 1H), 4.51 (bs, 2H).

### d) [8-(4-Chloro-phenyl)-[1,2,4]triazolo[1,5-a]pyridin-2-yl]-[3-methoxy-4-(4-methyl-imidazol-1-yl)-phenyl]-amine

Prepared in analogy to example 57e) employing 8-(4-chloro-phenyl)-[1,2,4]triazolo[1,5-a]pyridin-2-ylamine. The title compound was obtained as a white solid.

MS ISP (m/e): 431.3/433.2 (55/20) [(M+H)⁺].

¹H NMR (CDCl₃, 300 MHz): δ (ppm)=8.45 (dd, 1H), 7.99-7.96 (m, 2H), 7.64-7.59 (m, 3H), 7.51 (s, 1H), 7.46-7.42 (m, 2H), 7.16-7.13 (m, 1H), 7.04-6.97 (m, 2H), 6.87 (s, 1H), 3.88 (s, 3H), 2.31 (s, 3H).

### Example 126

### [8-(3,4-Difluoro-phenyl)-[1,2,4]triazolo[1,5-a]pyridin-2-yl]-[3-methoxy-4-(4-methyl-imidazol-1-yl)-phenyl]-amine

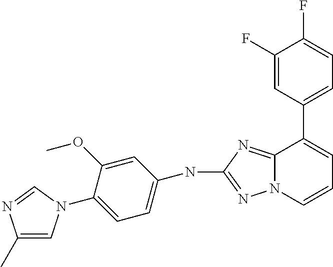

Prepared in analogy to example 125 starting from 3,4-difluorophenyl boronic acid. The title compound was obtained as light yellow solid.

MS ISP (m/e): 433.2 [(M+H)⁺].

¹H NMR (CDCl₃, 300 MHz): δ (ppm)=8.45 (dd, 1H), 8.09-8.03 (m, 1H), 7.77-7.71 (m, 2H), 7.64-7.60 (m, 2H), 7.34-7.28 (m, 1H), 7.21-7.18 (m, 1H), 7.05-6.98 (m, 3H), 6.87 (m, 1H), 3.92 (s, 3H), 2.31 (s, 3H).

### Example 127

### [8-(4-Chloro-phenyl)-[1,2,4]triazolo[1,5-a]pyridin-2-yl]-[3-methoxy-4-(2-methyl-pyridin-4-yl)-phenyl]-amine

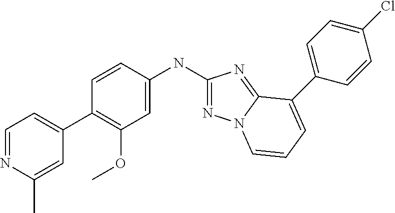

Prepared in analogy to example 53 employing 8-(4-chloro-phenyl)-[1,2,4]triazolo[1,5-a]pyridin-2-ylamine (see example 125c). The title compound was obtained as yellow solid.

MS ISP (m/e): 442.2/444.3 (100/36) [(M+H)⁺].

¹H NMR (CDCl₃, 300 MHz): δ (ppm)=8.50-8.44 (m, 2H), 8.01-7.98 (m, 2H), 7.63-7.59 (m, 2H), 7.50-7.47 (m, 2H), 7.34-7.29 (m, 3H), 7.09-6.99 (m, 3H), 3.92 (s, 3H), 2.60 (s, 3H).

### Example 128

### 8-(3,4-Difluorophenyl)-N-(3-methoxy-4-(2-methylpyridin-4-yl)phenyl)-[1,2,4]triazolo[1,5-a]pyridin-2-amine

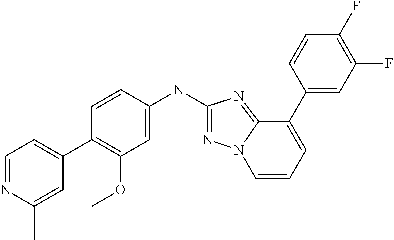

Prepared in analogy to example 53 employing 8-(3,4-difluoro-phenyl)-[1,2,4]triazolo[1,5-a]pyridin-2-ylamine (prepared in analogy to 125c). The title compound was obtained as white solid.

MS ISP (m/e): 444.3 (100) [(M+H)⁺].

¹H NMR (CDCl₃, 300 MHz): δ (ppm)=8.50-8.45 (m, 2H), 8.10-8.03 (m, 1H), 7.78-7.72 (m, 1H), 7.69-7.68 (m, 1H), 7.62-7.59 (m, 1H), 7.34-7.25 (m, 4H), 7.07-6.99 (m, 3H), 3.94 (s, 3H), 2.60 (s, 3H).

### Example 129

### 8-(4-fluorophenyl)-N-(3-methoxy-4-(4-methyl-1H-imidazol-1-yl)phenyl)-7-methyl-5,6,7,8-tetrahydro-[1,2,4]triazolo[1,5-a]pyrazin-2-amine

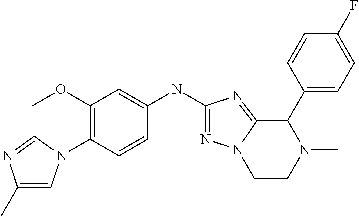

Prepared in analogy to example 124 starting with [8-(4-fluoro-phenyl)-5,6,7,8-tetrahydro-[1,2,4]triazolo[1,5-a]pyrazin-2-yl]-[di-(tert-butoxycarbonyl)]-amine (see example 59a) instead of tert-butyl 5-(4-fluorophenyl)-5,6,7,8-tetrahydro-[1,2,4]triazolo[1,5-a]pyrazin-2-ylcarbamate.

MS ISP (m/e): 434.4 (100) [(M+H)⁺].

¹H NMR (CDCl₃, 300 MHz): δ (ppm)=7.57 (m, 1H), 7.37-7.30 (m, 3H), 7.11-7.04 (m, 3H), 6.84-6.80 (m, 2H), 6.60 (m, 1H), 4.39-4.31 (m, 1H), 4.30 (s, 1H), 4.19-4.15 (m, 1H), 3.78 (s, 3H), 3.32-3.27 (m, 1H), 3.02-2.93 (m, 1H), 2.31 (s, 3H), 2.28 (s, 3H).

### Example 130

### 8-(3-chloro-4-fluorophenyl)-N-(3-methoxy-4-(4-methyl-1H-imidazol-1-yl)phenyl)-[1,2,4]triazolo[1,5-a]pyridin-2-amine

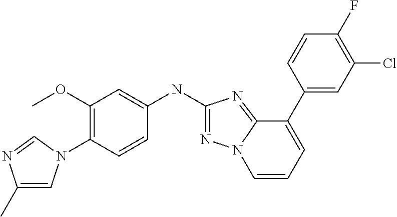

Prepared in analogy to example 125 starting from 3-chloro-4-fluorophenyl boronic acid.

The title compound was obtained as off-white solid.

MS ISP (m/e): 449.1/451.1 (100/33) [(M+H)⁺].

¹H NMR (CDCl₃, 300 MHz): δ (ppm)=8.46-8.44 (m, 1H), 8.19-8.16 (m, 1H), 7.93-7.87 (m, 1H), 7.66-7.58 (m, 3H), 7.30-7.27 (m, 1H), 7.20-7.17 (m, 1H), 7.11 (s, 1H), 7.04-7.00 (m, 2H), 6.88 (m, 1H), 3.90 (s, 3H), 2.31 (s, 3H).

### Example 131

### 8-(2-chloro-4-fluorophenyl)-N-(3-methoxy-4-(4-methyl-1H-imidazol-1-yl)phenyl)-[1,2,4]triazolo[1,5-a]pyridin-2-amine

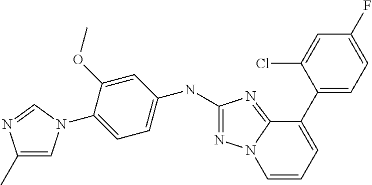

Prepared in analogy to example 125 starting from 2-chloro-4-fluorophenyl boronic acid. The title compound was obtained as white solid.

MS ISP (m/e): 449.2/451.2 (100/50) [(M+H)⁺].

¹H NMR (CDCl₃, 300 MHz): δ (ppm)=8.49 (dd, 1H), 7.61-7.48 (m, 4H), 7.32-7.28 (m, 1H), 7.18-6.95 (m, 5H), 6.86 (m, 1H), 3.86 (s, 3H), 2.30 (s, 3H).

### Example 132

### 8-(2,4-difluorophenyl)-N-(3-methoxy-4-(4-methyl-1H-imidazol-1-yl)phenyl)-[1,2,4]triazolo[1,5-a]pyridin-2-amine

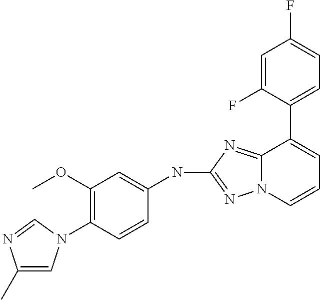

Prepared in analogy to example 125 starting from 2,4-difluorophenyl boronic acid. The title compound was obtained as white solid.

MS ISP (m/e): 433.2 (100) [(M+H)⁺].

¹H NMR (CDCl₃, 300 MHz): δ (ppm)=8.47 (dd, 1H), 7.92-7.84 (m, 1H), 7.63-7.58 (m, 3H), 7.19-7.16 (m, 1H), 7.05-6.95 (m, 5H), 6.87 (m, 1H), 3.88 (s, 3H), 2.30 (s, 3H).

### Example 133

### [8-(2-Chloro-4-fluoro-phenyl)-[1,2,4]triazolo[1,5-a]pyridin-2-yl]-[3-methoxy-4-(2-methyl-pyridin-4-yl)-phenyl]-amine

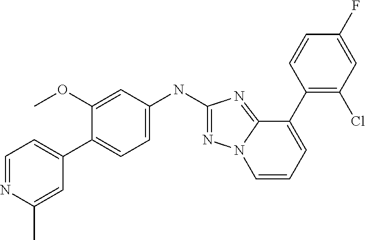

Prepared in analogy to example 53 employing 8-(2-chloro-4-fluoro-phenyl)-[1,2,4]triazolo[1,5-a]pyridin-2-ylamine (prepared in analogy to 125c). The title compound was obtained as white solid.

MS ISP (m/e): 460.3/462.2 (100/38) [(M+H)⁺].

¹H NMR (CDCl₃, 300 MHz): δ (ppm)=8.51-8.47 (m, 2H), 7.61-7.54 (m, 2H), 7.50-7.47 (m, 1H), 7.33-7.28 (m, 4H), 7.16-7.09 (m, 1H), 7.05-6.98 (m, 3H), 3.88 (s, 3H), 2.59 (s, 3H).

### Example 134

### 1-(5-(4-fluorophenyl)-2-(3-methoxy-4-(4-methyl-1H-imidazol-1-yl)phenylamino)-5,6-dihydro-[1,2,4]triazolo[1,5-a]pyrazin-7(8H)-yl)-2-methylpropan-1-one

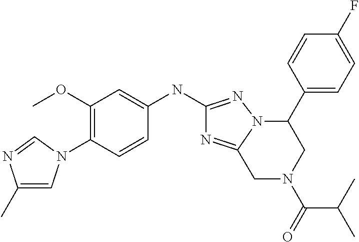

Prepared in analogy to example 59b-c) employing 5-(4-fluorophenyl)-N-(3-methoxy-4-(4-methyl-1H-imidazol-1-yl)phenyl)-7-(2,2,2-trifluoroethyl)-5,6,7,8-tetrahydro-[1,2,4]triazolo[1,5-a]pyrazin-2-amine (see example 57a-b). The title compound was obtained as white solid.

MS ISP (m/e): 490.3 (100) [(M+H)⁺].

¹H NMR (CDCl₃, 300 MHz): δ (ppm)=mixture of rotamers: 7.56 (m, 1H), 7.28 (m, 1H), 7.16-6.80 (m, 8H), 5.41-3.94 (m, 5H), 3.67 (s, 3H), 2.82 & 2.12 (m, 1H), 2.27 (s, 3H), 1.10-1.08 & 0.77 (m, 6H).

### Example 135

### 8-(3,4-difluorophenyl)-N-(3-methoxy-4-(4-methyl-1H-imidazol-1-yl)phenyl)-5-(trifluoromethyl)-[1,2,4]triazolo[1,5-a]pyridin-2-amine

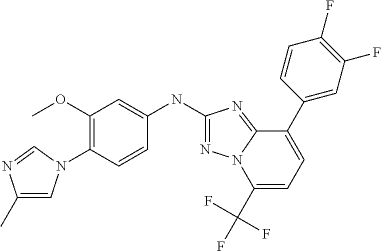

### a) 3-Bromo-6-(trifluoromethyl)pyridin-2-amine

A solution of 6-(trifluoromethyl)pyridin-2-amine (200 mg, 1.23 mmol) in dichloromethane (2.47 ml) was cooled to 0° C. and bromine (197 mg, 63.4 μl, 1.23 mmol) was slowly added within 30 min. After 25 h at 0° C. the reaction mixture was extracted with saturated Na₂S₂O₃ solution, water and brine, dried over Na₂SO₄ and concentrated in vacuo. The crude material was purified by flash chromatography over silica gel using CH₂Cl₂/MeOH (with 10% ammonia) as eluent. The title compound was obtained as a white solid (711 mg, 24%).

¹H NMR (CDCl₃, 300 MHz): δ (ppm)=7.80-7.77 (m, 1H), 6.91-6.89 (m, 1H).

### b) 8-(3,4-difluorophenyl)-N-(3-methoxy-4-(4-methyl-1H-imidazol-1-yl)phenyl)-5-(trifluoromethyl)-[1,2,4]triazolo[1,5-a]pyridin-2-amine

Prepared in analogy to example 125a-d) starting with 3-bromo-6-(trifluoromethyl)pyridin-2-amine in step 125a) and employing 3,4-difluorophenylboronic acid in step 125c).

MS ISP (m/e): 501.1 (100) [(M+H)⁺].

¹H NMR (CDCl₃, 300 MHz): δ (ppm)=8.10-8.03 (m, 2H), 7.80-7.75 (m, 1H), 7.69-7.61 (m, 2H), 7.44-7.41 (m, 1H), 7.38-7.29 (m, 1H), 7.20-7.15 (m, 2H), 6.89 (m, 1H), 6.81-6.77 (m, 1H), 3.94 (s, 3H), 2.31 (s, 3H).

### Example 136

### N-(3-methoxy-4-(4-methyl-1H-imidazol-1-yl)phenyl)-8-phenoxy-[1,2,4]triazolo[1,5-a]pyridin-2-amine

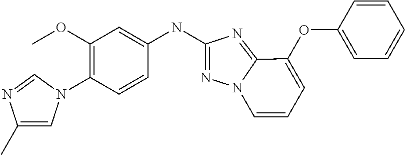

### a) 8-phenoxy-[1,2,4]triazolo[1,5-a]pyridin-2-amine

A suspension of 8-bromo-[1,2,4]triazolo[1,5-a]pyridin-2-amine (500 mg, 2.35 mmol), phenol (442 mg, 4.69 mmol), copper (I) iodide (44.7 mg, 235 μmol), picolinic acid (57.8 mg, 469 μmol) and potassium phosphate tribasic (1.49 g, 7.04 mmol) in DMSO (10 ml) was heated to 120° C. for 12 h. Further phenol (442 mg, 4.69 mmol), copper (I) iodide (44.7 mg, 235 μmol), picolinic acid (57.8 mg, 469 μmol) and potassium phosphate tribasic (1.49 g, 7.04 mmol) were added and heated to 120° C. for 18 h. The reaction mixture was cooled to rt and water was added. The aqueous phase was extracted three times with ethyl acetate. The combined organic phases were dried over sodium sulfate, the solvent was evaporated and the residue purified by preparative HPLC (Gemini 5μ, 30×100 mm) using MeOH/H₂O (with 0.1% NEt₃) as eluent. The title compound was obtained as a off-white solid (200 mg; 38%).

MS ISP (m/e): 227.2 (100) [(M+H)⁺].

¹H NMR (CDCl₃, 300 MHz): δ (ppm)=8.10-8.07 (m, 1H), 7.42-7.37 (m, 2H), 7.22-7.11 (m, 3H), 6.78-6.67 (m, 2H), 4.53 (bs, 2H).

### b) N-(3-methoxy-4-(4-methyl-1H-imidazol-1-yl)phenyl)-8-phenoxy-[1,2,4]triazolo[1,5-a]pyridin-2-amine

Prepared in analogy to example 57e) employing 8-phenoxy-[1,2,4]triazolo[1,5-a]pyridin-2-amine. The title compound was obtained as a white solid.

MS ISP (m/e): 413.4 (100) [(M+H)⁺].

¹H NMR (CDCl₃, 300 MHz): δ (ppm)=8.25-8.22 (m, 1H), 7.62-7.61 (m, 1H), 7.51-7.50 (m, 1H), 7.44-7.38 (m, 2H), 7.24-7.13 (m, 4H), 7.08-7.04 (m, 2H), 6.90-6.77 (m, 3H), 3.85 (s, 3H), 2.30 (s, 3H).

### Example 137

### 8-(3-chlorophenoxy)-N-(3-methoxy-4-(4-methyl-1H-imidazol-1-yl)phenyl)-[1,2,4]triazolo[1,5-a]pyridin-2-amine

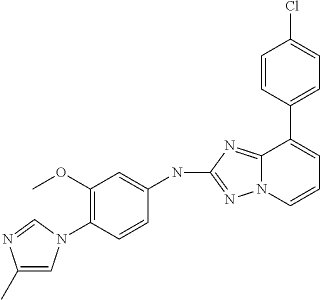

Prepared in analogy to example 136 employing 3-chlorophenol. The title compound was obtained as an off-white solid.

MS ISP (m/e): 447.2/449.2 (100/36) [(M+H)⁺].

¹H NMR (CDCl₃, 300 MHz): δ (ppm)=8.31-8.29 (m, 1H), 7.62-7.61 (m, 1H), 7.50 (m, 1H), 7.34-7.29 (m, 1H), 7.19-7.16 (m, 2H), 7.11-7.10 (m, 1H), 7.06-7.00 (m, 4H), 6.87-6.85 (m 2H), 3.84 (s. 3H), 2.30 (s, 3H).

### Example 138

### 8-(4-chlorophenoxy)-N-(3-methoxy-4-(4-methyl-1H-imidazol-1-yl)phenyl)-[1,2,4]triazolo[1,5-a]pyridin-2-amine

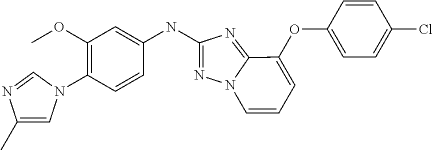

Prepared in analogy to example 136 employing 4-chlorophenol. The title compound was obtained as an off-white solid.

MS ISP (m/e): 447.2/449.2 (100/32) [(M+H)⁺].

¹H NMR (CDCl₃, 300 MHz): δ (ppm)=8.28-8.26 (m, 1H), 7.62 (m, 1H), 7.49-7.48 (m, 1H), 7.37-7.34 (m, 2H), 7.19-7.17 (m, 1H), 7.08-7.02 (m, 4H), 6.95-6.92 (m 1H), 6.86-6.80 (m, 2H), 3.85 (s. 3H), 2.30 (s, 3H).

### Example 139

### 8-(4-fluoropiperidin-1-yl)-N-(3-methoxy-4-(4-methyl-1H-imidazol-1-yl)phenyl)-[1,2,4]triazolo[1,5-a]pyridin-2-amine

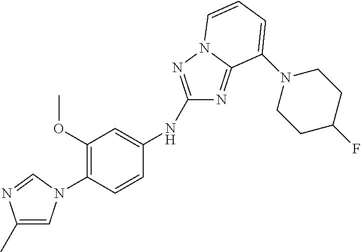

### a) 2-nitropyridin-3-yl trifluoromethanesulfonate

To an ice-cold solution of 2-nitropyridin-3-ol (10.0 g, 71 mmol) and triethylamine (14.9 ml, 107 mmol) in methylene chloride (150 ml) was added, dropwise, triflic anhydride (14.5 ml, 86 mmol) and the mixture was stirred for 2 h. Water was added and the mixture extracted with methylene chloride. The organic phase was dried with sodium sulfate and the solvent was evaporated in vacuo. The residue was purified by column chromatography on silica gel using n-heptane/ethyl acetate (v/v 2:8 to 3:7) as eluent. The title compound was obtained as a light brown liquid (18.4 g, 95%).

MS ISP (m/e): 273.1 [(M+H)⁺].

¹H NMR (CDCl₃, 400 MHz): δ (ppm)=8.65 (dd, 1H), 8.00 (dd, 1H), 7.80 (dd, 1H).

### b) 3-(4-fluoropiperidin-1-yl)-2-nitropyridine

To a solution of 4-fluoropiperidine hydrochloride (1.54 g, 11 mmol) and triethylamine (4.5 ml, 33 mmol) in dimethylacetamide (30 ml) was added 2-nitropyridin-3-yl trifluoro-methanesulfonate (3.00 g, 11 mmol) and the mixture heated to 110° C. for 1 h. Water was then added and the mixture extracted with ethyl acetate. The organic phase was washed with brine and dried with sodium sulfate. The solvent was evaporated in vacuo and the product used without further purification. The title compound was obtained as a yellow oil (2.22 g, 89%).

MS ISP (m/e): 226.0 [(M+H)⁺].

¹H NMR (CDCl₃, 400 MHz): δ (ppm)=8.10 (dd, 1H), 7.55 (dd, 1H), 7.47 (dd, 1H), 4.95-4.75 (m, 1H), 3.25-3.17 (m, 2H), 3.07-3.00 (m, 2H), 2.10-1.95 (m, 4H).

### c) 3-(4-fluoropiperidin-1-yl)pyridin-2-amine

To a solution of 3-(4-fluoropiperidin-1-yl)-2-nitropyridine (2.0 g, 8.9 mmol) and in methanol (25 ml) was added a generous spoon of rainey-nickel and the mixture stirred under an atmosphere of hydrogen for 5 h. The reaction was then filtered over Hyflo and the solvent was evaporated in vacuo to afford the product used without need for further purification. The title compound was obtained as a dark brown solid (1.7 g, 100%).

MS ISP (m/e): 196.2 [(M+H)⁺].

### d) N-(3-(4-Fluoropiperidin-1-yl)-pyridin-2-yl)-N′-ethoxycarbonyl-thiourea

Prepared in analogy to example 1b, starting from 3-(4-fluoropiperidin-1-yl)pyridin-2-amine. The residue was purified by column chromatography on silica gel using n-heptane/ethyl acetate (v/v 1:1 to 3:7) as eluent to afford the title compound as a yellow solid (yield: 73%).

MS ISP (m/e): 327.1 [(M+H)⁺].

¹H NMR (DMSO-D₆, 400 MHz): δ (ppm)=12.0 (brs, 1H), 11.3 (bs, 1H), 8.13 (dd, 1H), 7.60 (dd, 1H), 7.34 (dd, 1H), 4.95-4.75 (m, 1H), 4.22 (q, 2H), 3.01 (t, 2H), 2.87-2.80 (m, 2H), 2.07-1.81 (m, 4H), 1.26 (t, 3H).

### e) 5-(4-Fluoro-phenyl)-[1,2,4]triazolo[1,5-a]pyridin-2-ylamine

Prepared in analogy to example 1c, starting from N-(3-(4-Fluoropiperidin-1-yl)-pyridin-2-yl)-N′-ethoxycarbonyl-thiourea affording the title compound without need for purification as a light yellow solid (yield: 100%).

MS ISP (m/e): 236.2 [(M+H)⁺].

¹H NMR (CDCl₃, 400 MHz): δ (ppm)=7.95 (dd, 1H), 6.74-6.75 (m, 2H), 4.97-4.79 (m, 1H), 4.40 (brs, 2H), 3.54-3.43 (m, 4H), 2.87-2.80 (m, 2H), 2.21-2.02 (m, 4H).

### f) 8-(4-fluoropiperidin-1-yl)-N-(3-methoxy-4-(4-methyl-1H-imidazol-1-yl)phenyl)-[1,2,4]triazolo[1,5-a]pyridin-2-amine

Prepared in analogy to example 57e, starting from 5-(4-fluoro-phenyl)-[1,2,4]triazolo[1,5-a]pyridin-2-ylamine. The residue was purified by column chromatography on silica gel using methanol/ethyl acetate (v/v 2:98 to 5:95) as eluent to afford the title compound as a colourless solid (yield: 33%).

MS ISP (m/e): 422.2 [(M+H)⁺].

¹H NMR (CDCl₃, 400 MHz): δ (ppm)=8.08 (dd, 1H), 7.64 (dd, 1H), 7.17 (d, 1H), 7.05 (brs, 1H), 7.01 (dd, 1H), 6.87 (brs, 1H), 6.81 (apt, 1H), 6.75 (d, 1H), 4.99-4.81 (m, 1H), 3.90 (s, 3H), 3.56 (apt, 4H), 2.30 (s, 3H), 3.01 (t, 2H), 2.20-2.06 (m, 4H).

### Example 140

### N-(3-methoxy-4-(4-methyl-1H-imidazol-1-yl)phenyl)-8-(4-(trifluoromethyl)piperidin-1-yl)-[1,2,4]triazolo[1,5-a]pyridin-2-amine

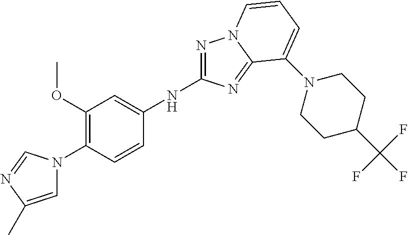

Prepared in analogy to example 139, starting from added 2-nitropyridin-3-yl trifluoro-methanesulfonate (example 139b) and 4-trifluoromethylpiperidine hydrochloride. The residue was purified by preparative HPLC to afford the title compound as a light yellow gum (yield: 55%).

MS ISP (m/e): 472.6 [(M+H)⁺].

¹H NMR (CDCl₃, 400 MHz): δ (ppm)=8.10 (dd, 1H), 7.81 (s, 1H), 7.67 (s, 1H), 7.61 (d, 1H), 7.19 (dd, 1H), 7.09 (dd, 1H), 6.87 (brs, 1H), 6.54 (t, 1H), 6.77 (d, 1H), 4.19 (brd, 2H), 3.90 (s, 3H), 2.76 (td, 2H), 2.32 (s, 3H), 2.03 (brd, 2H), 1.92 (dd, 2H).

### Example 141

### 8-(4,4-difluoropiperidin-1-yl)-N-(3-methoxy-4-(4-methyl-1H-imidazol-1-yl)phenyl)-[1,2,4]triazolo[1,5-a]pyridin-2-amine

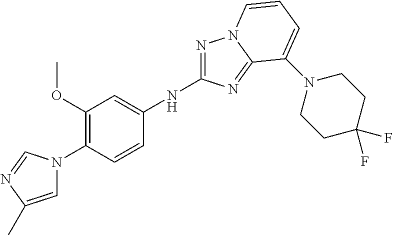

Prepared in analogy to example 139, starting from added 2-nitropyridin-3-yl trifluoro-methanesulfonate (example 139b) and 4,3-difluorolpiperidine hydrochloride. The residue was purified by preparative HPLC to afford the title compound as a colourless gum (yield: 15%).

MS ISP (m/e): 440.2 [(M+H)⁺].

¹H NMR (CDCl₃, 400 MHz): δ (ppm)=8.12 (dd, 1H), 7.67 (s, 1H), 7.58 (dd, 1H), 7.18 (d, 1H), 7.08 (dd, 1H), 6.87 (d, 1H), 6.87 (br, 1H), 6.82 (t, 1H), 6.77 (d, 1H), 3.89 (s, 3H), 3.60 (t, 4H), 2.31 (s, 3H), 2.28-2.17 (m, 4H).

### Example 142

### N-(3-methoxy-4-(4-methyl-1H-imidazol-1-yl)phenyl)-8-phenyl-6,8-dihydro-5H-[1,2,4]triazolo[5,1-c][1,4]oxazin-2-amine

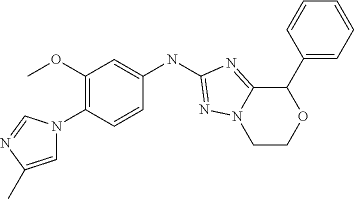

### a) (2-Oxo-ethoxy)-phenyl-acetic acid methyl ester

Allyloxy-phenyl-acetic acid methyl ester (described in EJOC 2000, 3145-3163; 3 g, 14.5 mmol) was dissolved in 300 mL DCM and cooled to −75° C. O₃ was blubbled through the solution for 6 h until the solution turned blue. Argon was blubbled through the solution for 1 hour, then dimethyl sulfide (9.04 g, 10.8 ml, 145 mmol) was added to the reaction mixture and kept at rt for 12 h. The reaction mixture was evaporated and the residue purified by flash-chromatography over 50 g SiO₂-flash pack using gradient 10-100% EtOAc in heptane over 60 min to give the title compound as light yellow oil (2.72 g, 90%).

¹H NMR (CDCl₃, 300 MHz): δ (ppm)=9.75 (s, 1H), 7.47-7.36 (m, 5H), 5.03 (s, 1H), 4.13 (s, 2H), 3.74 (s, 3H).

### b) tert-butyl 2-(2-(2-methoxy-2-oxo-1-phenylethoxy)ethylidene)hydrazinecarboxylate

(2-Oxo-ethoxy)-phenyl-acetic acid methyl ester (2.7 g, 13.0 mmol) and tert-butyl carbazate (1.75 g, 13.0 mmol) were dissolved in toluene (290 ml) and heated to 65° C. over night. The reaction mixture was concentrated in vacuo and the residue was purified by flash chromatography (silica gel, 100 g, 0% to 100% EtOAc in heptane over 60 min) to give the title compound as yellow viscous oil (2.81 g, 67%).

MS ISP (m/e): 323.3 (42) [(M+H)⁺], 267.1 (100)) [(M−tBu)⁺].

¹H NMR (CDCl₃, 300 MHz): δ (ppm)=7.85 (bs, 1H), 7.44-7.34 (m, 5H), 4.94 (s, 1H), 4.23-4.21 (m, 2H), 3.71 (s, 3H), 1.50 (s, 9H).

### c) tert-Butyl 2-(2-(2-methoxy-2-oxo-1-phenylethoxy)ethyl)hydrazinecarboxylate

tert-Butyl 2-(2-(2-methoxy-2-oxo-1-phenylethoxy)ethylidene)hydrazinecarboxylate (2.81 g, 8.72 mmol) in MeOH (105 ml) was hydrogenated at 3.5 bar and 30° C. for 48 hours in a Parr bottle in the presence of nickel (1.4 g, 157 μl, 11.1 mmol). The reaction mixture was filtered and washed with MeOH. The solvent was evaporated and the residue was purified by flash-chromatography (70 g, EtOAc/Heptane) to give the title compound as colorless oil (600 mg, 21%).

¹H NMR (CDCl₃, 300 MHz): δ (ppm)=7.47-7.33 (m, 5H), 6.30 (bs, 1H), 4.93 (s, 1H), 4.20 (bs, 1H), 3.72 (s, 3H), 3.70-3.57 (m, 2H), 3.12-3.07 (m, 2H), 1.46 (s, 9H).

### d) 4-Amino-2-phenylmorpholin-3-one

tert-Butyl 2-(2-(2-methoxy-2-oxo-1-phenylethoxy)ethyl)hydrazinecarboxylate (390 mg, 1.2 mmol) in water (84.7 ml) was heated to 95° C. for 12 h. The reaction mixture was extracted with DCM, the organic layers were combined, dried over Na₂SO₄ and the solvent was evaporated to give the title compound as light yellow oil (183 mg, 79%).

MS ISP (m/e): 193.2 (100) [(M+H)⁺].

¹H NMR (CDCl₃, 300 MHz): δ (ppm)=7.45-7.33 (m, 5H), 5.24 (s, 1H), 4.54 (bs, 2H), 4.12-4.05 (m, 1H), 3.99-3.91 (m, 1H), 3.83-3.75 (m, 1H), 3.65-3.58 (m, 1H).

### e) 8-Phenyl-6,8-dihydro-5H-[1,2,4]triazolo[5,1-c][1,4]oxazin-2-amine

4-amino-2-phenylmorpholin-3-one (175 mg, 910 μmol) and cyanamide (230 mg, 179 μl, 5.46 mmol) were dissolved in ethanol (4 ml). p-Toluenesulfonic acid monohydrate (260 mg, 209 μl, 1.37 mmol) was added and the mixture was heated under reflux at 80° C. for 24 hours. After cooling the rt, triethylamine (461 mg, 634 μl, 4.55 mmol) was added and the mixture was heated under reflux at 80° C. for 3 days.

The reaction mixture was extracted with saturated sodium bicarbonate solution and EtOAc. The combined organic layers were washed with brine, dried over Na₂SO₄ and the solvent was evaporated. The residue was purified by chromatography over 10 g NH₂-flash pack using gradient 0-15% MeOH/NH₃(9:1) in DCM to give the title compound as off-white solid (41 mg, 21%).

MS ISP (m/e): 217.3 (100) [(M+H)⁺].

¹H NMR (CDCl₃, 300 MHz): δ (ppm)=7.40-7.37 (m, 5H), 5.75 (s, 1H), 4.31-4.16 (m, 2H), 4.13-4.06 (m, 4H).

### f) N-(3-methoxy-4-(4-methyl-1H-imidazol-1-yl)phenyl)-8-phenyl-6,8-dihydro-5H-[1,2,4]triazolo[5,1-c][1,4]oxazin-2-amine

Prepared in analogy to example 1e). The title compound was obtained as an off-white solid.

MS ISP (m/e): 403.4 (100) [(M+H)⁺].

¹H NMR (CDCl₃, 300 MHz): δ (ppm)=7.59 (m, 1H), 7.45-7.38 (m, 5H), 7.35-7.34 (m, 1H), 7.14-7.11 (m, 1H), 6.92-6.88 (m, 1H), 6.84 (m, 1H), 6.63 (s, 1H), 5.83 (s, 1H), 4.37-4.11 (m, 4H), 3.82 (s, 3H), 2.29 (s, 3H).

### Example 143

### 4-(3,4-Difluorophenyl)-N-(3-methoxy-4-(4-methyl-1H-imidazol-1-yl)phenyl)-4,5,6,7-tetrahydro-[1,2,4]triazolo[1,5-a]pyrimidin-2-amine

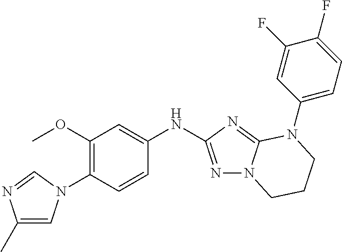

### a) (Z)-phenyl N′-cyano-N-(3,4-difluorophenyl)carbamimidate

To a solution of 3,4-difluoroaniline (646 mg, 5 mmol) in isopropanol (10 mL) was added diphenyl cyanocarbonimidate (1.19 g, 5.00 mmol) and the suspension was stirred at room temperature over night. The precipitate was filtered off, washed with isopropanol and dried under reduced pressure to yield the title compound as a white solid (1.18 g, 86%).

MS ISP (m/e): 274.1 (100) [(M+H)⁺].

¹H NMR (DMSO-D₆, 300 MHz): 6(ppm)=10.92 (s, 1H), 7.65 (m, 1H), 7.43 (m, 3H), 7.29 (m, 4H).

### b) (Z)-Phenyl N′-cyano-N-(3,4-difluorophenyl)-N-(3-(tetrahydro-2H-pyran-2-yloxy)propyl)carbamimidate

To a solution of (Z)-phenyl N′-cyano-N-(3,4-difluorophenyl)carbamimidate (286 mg, 1.05 mmol) and 2-(3-bromopropoxy)tetrahydro-2H-pyran (369 mg, 277 μL, 1.57 mmol) in DMF (10.5 mL) was added at room temperature under an athmosphere of nitrogen potassium carbonate (289 mg, 2.09 mmol). The suspension was heated to 85° C. over night. Additional 2-(3-bromopropoxy)tetrahydro-2H-pyran (140 μL, 0.8 mmol) and potassium carbonate (145 mg, 1.05 mmol) was added and the reaction was heated for 5 hours to 85° C. Water was added and the reaction was extracted twice with diethyl ether. The combined organic layers were washed with water and with saturated aqueous sodium chloride solution, dried over sodium sulfate, filtered and the solvent was evaporated under reduced pressure. The title compound was obtained as a light yellow viscous oil (202 mg, 46%) after column chromatography on silica gel using a gradient of heptane/ethyl acetate 4:1 to 1:1 (v/v) as eluent.

MS ISP (m/e): 332.1 (100) [(M−THP+H)⁺], 416.3 (5) [(M+H)⁺].

¹H NMR (DMSO-D₆, 300 MHz): δ (ppm)=7.38 (t, 2H), 7.26-7.16 (m, 3H), 7.05 (m, 3H), 4.52 (t, 1H), 3.97 (t, 2H), 3.85 (m, 2H), 3.48 (m, 2H), 2.00 (pent, 2H), 1.79 (m, 1H), 1.68 (m, 1H), 1.55 (m, 4H)).

### c) N3-(3,4-Difluorophenyl)-N3-(3-(tetrahydro-2H-pyran-2-yloxy)propyl)-4H-1,2,4-triazole-3,5-diamine

To a solution of (Z)-phenyl N′-cyano-N-(3,4-difluorophenyl)-N-(3-(tetrahydro-2H-pyran-2-yloxy)propyl)carbamimidate (73 mg, 176 μmol) in methanol (0.5 mL) was added hydrazine hydrate 25% in water (35.2 mg, 34.8 μl, 176 μmol). The reaction was stirred at room temperature over night. The solvent was evaporated under reduced pressure and the residue purified by column chromatography on silica gel using methylene chloride/methanol 19:1 (v/v) as eluent. The title compound was obtained as a light yellow viscous oil (46 mg, 74%).

MS ISP (m/e): 354.2 (25) [(M+H)⁺], 270.3 (100) [(M−THP+H)⁺].

¹H NMR (DMSO-D₆, 300 MHz): δ (ppm)=7.56 (m, 1H), 7.26 (q, 1H), 7.16 (m, 1H), 5.96 (br s, 2H), 4.49 (t, 1H), 3.87 (m, 2H), 3.37 (m, 2H), 1.84 (m, 2H), 1.74 (m, 1H), 1.62 (m, 2H), 1.45 (m, 4H).

### d) 3-((5-Amino-4H-1,2,4-triazol-3-yl)(3,4-difluorophenyl)amino)propan-1-ol

To a solution of N3-(3,4-difluorophenyl)-N3-(3-(tetrahydro-2H-pyran-2-yloxy)propyl)-4H-1,2,4-triazole-3,5-diamine (43 mg, 122 μmol) in methanol (1 mL) was added 2N aqueous hydrogen chloride solution. The solution was stirred at room temperature over night. The solvent was evaporated under reduced pressure and the residue was taken up in saturated aqueous sodium hydrogen carbonate solution. It was extracted twice with ethyl acetate. The combined organic layers were washed with saturated aqueous sodium hydrogen carbonate solution and with saturated aqueous sodium chloride solution, dried over sodium sulfate, filtered and the solvent was evaporated under reduced pressure to yield the title compound as a as a white solid (34 mg, quant) without further purification.

MS ISP (m/e): 270.3 (100) [(M+H)⁺].

¹H NMR (DMSO-D₆, 300 MHz): δ (ppm)=7.56 (m, 1H), 7.26 (q, 1H), 7.15 (m, 1H), 6.00 (br s, 2H), 4.67 (t, 1H), 3.87 (t, 1H), 3.42 (q, 2H), 1.71 (t, 2H).

### e) 4-(3,4-Difluoro-phenyl)-4,5,6,7-tetrahydro-[1,2,4]triazolo[1,5-a]pyrimidin-2-ylamine

To a solution of 3-((5-amino-4H-1,2,4-triazol-3-yl)(3,4-difluorophenyl)amino)propan-1-ol (31 mg, 115 μmol) in tetrahydrofurane (1.15 mL) was added at 0° C. under an athmosphere of nitrogen triphenylphosphine (45.3 mg, 173 μmol). The reaction was stirred for 15 minutes and then DEAD ((31.0 mg, 28.2 μl, 173 μmol) was added. The reaction was stirred for 30 minutes at 0° C. and then at room temperature over night. The same procedure was repeated with additional triphenylphosphine (45.3 mg, 173 μmol) and DEAD (31.0 mg, 28.2 μA, 173 μmol). Water was added and the reaction was extracted twice with ethyl acetate. The combined organic layers were washed with saturated aqueous sodium hydrogene carbonate solution and with saturated aqueous sodium chloride solution, dried over sodium sulfate, filtered and the solvent was evaporated under reduced pressure. The title compound was obtained as a colorless solid (14 mg, 48%) after column chromatography on silica gel using a gradient from methylene chloride to methylene chloride/methanol 19:1 (v/v) as eluent.

MS ISP (m/e): 252.3 (100) [(M+H)⁺].

¹H NMR (CDCl₃, 300 MHz): δ (ppm)=7.82 (m, 1H), 7.41-7.33 (m, 2H), 4.00 (t, 2H), 3.93 (b s, 2H), 3.72 (t, 2H), 2.30 (pent, 2H).

### f) 4-(3,4-Difluorophenyl)-N-(3-methoxy-4-(4-methyl-1H-imidazol-1-yl)phenyl)-4,5,6,7-tetrahydro-[1,2,4]triazolo[1,5-a]pyrimidin-2-amine

Prepared in analogy to example 8e, starting 4-(3,4-difluoro-phenyl)-4,5,6,7-tetrahydro-[1,2,4]triazolo[1,5-a]pyrimidin-2-ylamine and 1-(4-bromo-2-methoxy-phenyl)-4-methyl-1H-imidazole. The title compound was obtained as a light brown solid (yield: 48%) after column chromatography on silica gel using a gradient from CH₂Cl₂ to CH₂Cl₂/MeOH 19:1 (v/v) as eluent and precipitation from diethyl ether.

MS ISP (m/e): 438.3 (100) [(M+H)⁺].

¹H NMR (CDCl₃, 300 MHz): δ (ppm)=7.59 (s, 1H), 7.48 (m, 1H), 7.39 (s, 1H), 7.17-7.10 (m, 3H), 6.86 (d, 1H), 6.84 (s, 1H), 6.54 (s, 1H), 4.13 (t, 2H), 3.83 (s, 3H), 3.79 (t, 2H), 2.37 (pent, 2H), 2.29 (s, 3H).

### Example 144

### 2-{8-(4-Chloro-phenyl)-2-[3-methoxy-4-(4-methyl-imidazol-1-yl)-phenylamino]-6-methyl-[1,2,4]triazolo[1,5-a]pyridin-5-yl}-propan-2-ol

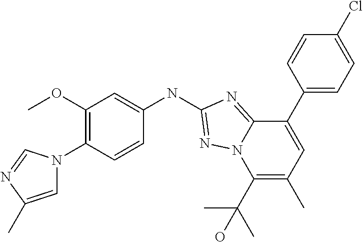

### a) 6-Amino-3-methyl-pyridine-2-carboxylic acid methyl ester

To a solution of 6-amino-3-bromo-pyridine-2-carboxylic acid methyl ester (3 g, 12.99 mmol) and trimethyl boroxine (1.8 mL, 2.99 mmol) in 1,4 dioxane (30 mL) was added K₂CO₃ (3.5 g, 25.97 mmol) under an argon atmosphere. To this was added PdCl₂ (dppf)₂.CH₂Cl₂ (530 mg, 0.65 mmol) and stirred at 115° C. for 4 h. The reaction mixture was cooled to room temperature and water was added to residue. The aqueous phase was extracted with ethyl acetate, the combined organic phases were dried over sodium sulfate, the solvent was evaporated and the residue purified by silica gel chromatography using ethyl acetate/hexane as eluent. The title compound was obtained as an off white solid (1.9 g, 88%).

MS ESI (m/e): 166.8 [(M+H)⁺].

¹H NMR (DMSO, 400 MHz): δ(ppm)=7.31 (d, J=8.4 Hz, 1H), 6.53 (d, J=8.36 Hz, 1H), 5.99 (s, 2H), 3.77 (s, 3H), 2.21 (s, 3H).

### b) 6-Amino-5-bromo-3-methyl-pyridine-2-carboxylic acid methyl ester

To a solution of 6-amino-3-methyl-pyridine-2-carboxylic acid methyl ester (1.9 g, 11.43 mmol) in dry chloroform (80 mL) was added bromine (0.9 mL, 17.45 mmol) in chloroform (10 mL) at room temperature and stirred for 14 hours. The reaction mixture was quenched with water. The aqueous phase was extracted with dichloromethane, the combined organic phases were dried over sodium sulfate, the solvent was evaporated and the residue purified by silica gel chromatography using ethyl acetate/hexane as eluent. The title compound was obtained as yellow solid (2.1 g, 75%).

MS ESI (m/e): 244.8 [(M+H)⁺].

¹H NMR (DMSO, 400 MHz): δ(ppm)=7.77 (s, 1H), 6.31 (s, 2H), 3.79 (s, 3H), 2.42 (s, 3H).

### c) N-(3-Bromo-6-ethoxycarbonyl-5-methyl-pyridin-2-yl)-N′-ethoxycarbonyl-thiourea

To a solution of 6-amino-5-bromo-3-methyl-pyridine-2-carboxylic acid methyl ester (2.1 g, 8.57 mmol) in dry 1,4-dioxane (40 mL) was added ethoxy carbonyl isothiocyanate (1.104 mL, 9.43 mmol) under an argon atmosphere and stirred at room temperature for 6 hours. The solvent was evaporated and the title compound was obtained as off white solid (2.9 g, 90%).

MS ESI (m/e): 151.2 [(M+H)⁺].

¹H NMR (DMSO, 400 MHz): δ(ppm)=11.50 (s, 1H), 11.36 (s, 1H), 8.25 (s, 1H), 4.22 (q, J=6.96 Hz, 2H), 3.85 (s, 3H), 1.27 (t, J=7.16 Hz, 3H).

### d) 2-Amino-8-bromo-6-methyl-[1,2,4]triazolo[1,5-a]pyridine-5-carboxylic acid methyl ester

To a solution of N-(3-bromo-6-ethoxycarbonyl-5-methyl-pyridin-2-yl)-N′-ethoxycarbonyl-thiourea (2 g, 5.32 mmol) in dry methanol (20 mL) were added hydroxylamine hydrochloride (1.8 g, 26.6 mmol) and diisopropyl ethylamine (2.79 mL, 15.96 mmol) under an argon atmosphere and stirred at room temperature for 4 hours. The solvent was evaporated and methanol (40 mL) was added to residue. The reaction mixture was heated to reflux for 12 hours. The solvent was evaporated and the residue purified by silica gel chromatography using ethyl acetate/hexane as eluent. The title compound was obtained as light yellow solid (700 mg, 46%).

MS ESI (m/e): 285.0 [(M+H)⁺].

¹H NMR (DMSO, 400 MHz): δ(ppm)=7.79 (s, 1H), 6.38 (s, 2H), 3.96 (s, 3H), 2.26 (s, 2H).

### e) 2-(2-Amino-8-bromo-6-methyl-[1,2,4]triazolo[1,5-a]pyridin-5-yl)-propan-2-ol

To a solution of 2-amino-8-bromo-6-methyl-[1,2,4]triazolo[1,5-a]pyridine-5-carboxylic acid methyl ester (1.2 g, 3.6 mmol) in tetrahydrofuran (20 mL) was added methyl magnesium bromide (1M solution in toluene/tetrahydrofuran; 75/25) (14.41 mL, 14.41 mmol) at −40° C. and stirred at −30° C. for 3.5 hour. The reaction mixture was warmed to room temperature and quenched with saturated aqueous NH₄Cl solution. The aqueous phase extracted with ethyl acetate, the combined organic phases were dried over sodium sulfate, the solvent was evaporated and the residue purified by silica gel column chromatography using ethyl acetate/hexane as eluent. The title compound was obtained as off white solid (500 mg, 49%) which was contaminated with keto-product.

MS ESI (m/e): 287.0 [(M+H)⁺].

### f) 2-[2-Amino-8-(4-chloro-phenyl)-6-methyl-[1,2,4]triazolo[1,5-a]pyridin-5-yl]-propan-2-ol

To a solution of 2-(2-amino-8-bromo-6-methyl-[1,2,4]triazolo[1,5-a]pyridin-5-yl)-propan-2-ol (contaminated with ketone) (200 mg, 0.72 mmol) and 4-chlorophenyl boronic acid (433 mg, 2.77 mmol) in dioxane (10 mL) was added aqueous solution of Na₂CO₃ (2M, 2 mL) and degassed with argon for 5 minute. To this was added PdCl₂ (dppf)₂.CH₂Cl₂ (42 mg, 0.05 mmol) and stirred at 80° C. for 90 minute. The reaction mixture was cooled to room temperature and water was added. The aqueous phase was extracted with ethyl acetate, the combined organic phases were dried over sodium sulfate, the solvent was evaporated and the residue purified by silica gel chromatography using ethyl acetate/hexane as eluent. The title compound was obtained as an off white solid (150 mg) as mixture of alcohol and ketone.

MS ESI (m/e): 316.8 [(M+H)⁺].

### g) 2-[2-Bromo-8-(4-chloro-phenyl)-6-methyl-[1,2,4]triazolo[1,5-a]pyridin-5-yl]-propan-2-ol

A solution of tert-butylnitrile (0.09 mL, 0.75 mmol) and copper (II) bromide (167.5 mg, 0.75 mmol) in acetonitrile (5 mL) was heated to 60° C. and 2-[2-amino-8-(4-chloro-phenyl)-6-methyl-[1,2,4]triazolo[1,5-a]pyridin-5-yl]-propan-2-ol (mixture of alcohol and keto) (150 mg, 0.5 mmol) in acetonitrile (10 mL) was added portion wise and stirred at 60° C. for 3 hours. The reaction mixture was cooled to room temperature and water was added. The aqueous phase was extracted with ethyl acetate, the combined organic phases were dried over sodium sulfate, and the solvent was evaporated. The title compound was obtained as a off white solid (93 mg, 49%).

MS ESI (m/e): 382.0 [(M+H)⁺].

¹H NMR (DMSO, 400 MHz): δ (ppm)=8.10 (d, J=8.48 Hz, 2H), 7.87 (s, 1H), 7.60 (d, J=8.44 Hz, 2H), 5.66 (s, 2H), 2.75 (s, 3H), 1.81 (s, 6H).

### h) 2-{8-(4-Chloro-phenyl)-2-[3-methoxy-4-(4-methyl-imidazol-1-yl)-phenylamino]-6-methyl-[1,2,4]triazolo[1,5-a]pyridin-5-yl}-propan-2-ol

A solution of 2-[2-bromo-8-(4-chloro-phenyl)-6-methyl-[1,2,4]triazolo[1,5-a]pyridin-5-yl]-propan-2-ol (100 mg, 0.27 mmol), 3-methoxy-4-(4-methyl-imidazol-1-yl)-phenyl amine (44 mg, 0.22 mmol) and sodium phenoxide (38 mg, 0.33 mmol) in dry 1,4-dioxane (8 mL) in a sealed tube was purged with argon gas for 10 minute. Pd₂(dba)₃.CHCl₃ (9 mg, 0.01 mmol) and xanthphos (10 mg, 0.02 mmol) were added and heated to 160° C. for 15 hours. The reaction mixture was cooled to room temperature and water was added. The aqueous phase extracted with ethyl acetate, the combined organic phases were dried over sodium sulfate, the solvent was evaporated and the residue purified by silica gel column chromatography using ethyl acetate/hexane as eluent. The title compound was obtained as white solid (15 mg, 11%).

MS ESI (m/e): 503.4 [(M+H)⁺].

### Example 145

### 2-{8-(3,4-Difluoro-phenyl)-2-[3-methoxy-4-(4-methyl-imidazol-1-yl)-phenylamino]-[1,2,4]triazolo[1,5-a]pyridin-5-yl}-propan-2-ol

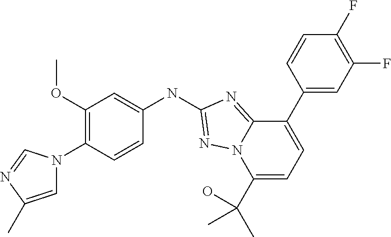

Prepared in analogy to example 116. The title compound was obtained as an off-white solid.

MS ESI (m/e): 491.2 [(M+H)⁺].

¹H NMR (DMSO, 400 MHz): δ (ppm)=10.09 (s, 1H), 8.32-8.30 (m, 1H), 7.99 (m, 2H), 7.64-7.58 (m, 3H), 7.32 (d, J=7.96 Hz, 1H), 7.24 (d, J=8.56 Hz, 1H), 7.08-7.06 (m, 1H), 7.03 (s, 1H), 5.87 (s, 1H), 3.86 (s, 3H), 2.14 (s, 3H), 1.82 (s, 6H).

### Example 146

### [6-Cyclopropyl-8-(3,4-difluoro-phenyl)-[1,2,4]triazolo[1,5-a]pyridin-2-yl]-[3-methoxy-4-(4-methyl-imidazol-1-yl)-phenyl]-amine

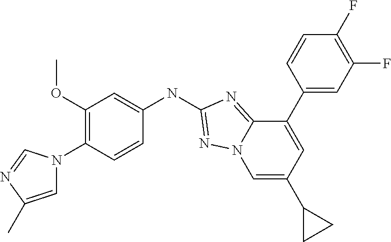

Prepared in analogy to example 117. The title compound was obtained as an off-white solid.

MS ESI (m/z): 473.2 [(M+H)⁺].

¹H NMR (DMSO, 400 MHz): δ(ppm)=9.98 (s, 1H), 8.41-835 (m, 1H), 8.11-8.09 (m, 1H), 7.08 (s, 1H), 7.74-7.55 (m, 3H), 7.23 (s, 2H), 7.01 (s, 1H), 3.83 (s, 3H) 2.32 (s, 3H), 2.13-2.07 (m, 1H), 1.01-0.95 (m, 2H), 0.92-0.90 (m, 2H).

### Example 147

### [8-(3-Chloro-4-fluoro-phenyl)-6-cyclopropyl-[1,2,4]triazolo[1,5-a]pyridin-2-yl]-[3-methoxy-4-(4-methyl-imidazol-1-yl)-phenyl]-amine

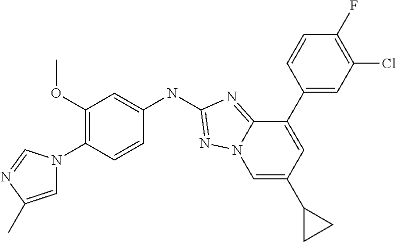

Prepared in analogy to example 117. The title compound was obtained as an off-white solid.

MS ESI (m/z): 489.0 [(M+H)⁺].

¹H NMR (DMSO, 400 MHz): d (ppm)=9.93 (s, 1H), 8.67 (s, 1H), 8.47-8.45 (m, 1H), 8.24-8.22 (m, 1H), 7.75 (br d, J=1.8 Hz, 1H), 7.64 (dd, J=7.72 & 1.12 Hz, 2H), 7.56 (t, J=8.92 Hz, 1H), 7.26-7.22 (m, 2H), 7.01 (s, 1H), 3.82 (s, 3H), 2.13 (s, 3H), 2.12-2.08 (m, 1H), 0.99-0.96 (m, 2H), 0.92-0.90 (m, 2H).

### Example 148

### [8-(2-Chloro-4-fluoro-phenyl)-6-cyclopropyl-[1,2,4]triazolo[1,5-a]pyridin-2-yl]-[3-methoxy-4-(4-methyl-imidazol-1-yl)-phenyl]-amine

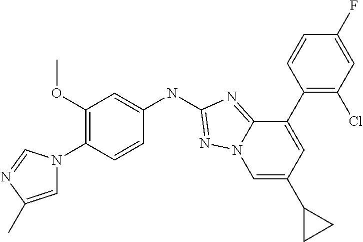

Prepared in analogy to example 117. The title compound was obtained as an off-white solid.

MS ESI (m/z): 489.1 [(M+H)⁺].

¹H NMR (DMSO, 400 MHz): δ(ppm)=9.89 (s, 1H), 7.68-7.64 (m, 2H), 7.62-7.6 (m, 2H), 7.38-7.24 (m, 2H), 7.22-718 (m, 2H), 6.99 (s, 1H), 3.82 (s, 2H), 2.13 (s, 3H), 2.09-2.05 (m, 1H), 0.99-.0.96 (m, 2H), 0.85 (m, 2H).

### Example 149

### [8-(3,6-Dihydro-2H-pyran-4-yl)-[1,2,4]triazolo[1,5-a]pyridin-2-yl]-[3-methoxy-4-(4-methyl-imidazol-1-yl)-phenyl]-amine

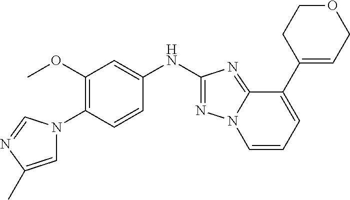

### a) N-(3-(4-Hydroxytetrahydro-2H-pyran-4-yl)pyridin-2-yl)pivalamide

To a solution of N-(pyridin-2-yl)pivalamide (184 mg, 1 mmol) in tetrahydrofurane (10 mL) was added at −78° C. under an athmosphere of nitrogen 1.6M butyl lithium in hexane (1.31 mL, 2.1 mmol). The reaction is slightly exothermic and a yellow color appears. The reaction is warmed to 0° C. within 15 minutes and stirred at 0° C. for 2 hours. A white suspension is formed. The reaction is cooled to −78° C. and dihydro-2H-pyran-4(3H)-one (123 mg, 113 μl, 1.2 mmol) was added in tetrahydrofurane (665 μL). The reaction was warmed to room temperature over night to yield an orange suspension. Saturated aqueous ammonium chloride solution was added and the reaction was extracted twice with ethyl acetate. The combined organic layers were washed with saturated aqueous sodium chloride solution, dried over sodium sulfate, filtered and the solvent was evaporated under reduced pressure. The title compound was obtained as a light yellow powder (145 mg, 52%) after precipitation with diethyl ether.

MS ISP (m/e): 279.3 (92) [(M+H)⁺], 261.2 (100), 301.3 (34).

¹H NMR (DMSO-D₆, 300 MHz): δ (ppm)=10.34 (br s, 1H), 8.26 (d, 1H), 7.74 (d, 1H), 7.13 (dd, 1H), 6.18 (s, 1H), 3.87-3.69 (m, 4H), 2.00 (m, 2H), 1.86 (m, 2H), 1.21 (s, 9H).

### b) 3-(3,6-Dihydro-2H-pyran-4-yl)pyridin-2-amine

To a solution of N-(3-(4-hydroxytetrahydro-2H-pyran-4-yl)pyridin-2-yl)pivalamide (141 mg, 507 μmol) in ethanol (7.6 mL) was added 2N aqueous sodium hydroxide solution. The reaction was heated to 100° C. over night. The solvent was evaporated under reduced pressure and the residue was taken up in water. It was extracted twice with ethyl acetate. The combined organic layers were washed with saturated aqueous sodium chloride solution, dried over sodium sulfate, filtered and the solvent was evaporated under reduced pressure to yield the title compound as a as a white solid (89 mg, quant) without further purification.

MS ISP (m/e): 177.2 (100) [(M+H)⁺].

¹H NMR (DMSO-D₆, 300 MHz): δ (ppm)=7.84 (d, 1H), 7.21 (d, 1H), 6.53 (dd, 1H), 5.82 (s, 1H), 5.61 (br s, 2H), 4.17 (m, 2H), 3.82 (t, 2H), 2.27 (m, 2H).

### c) 8-(3,6-Dihydro-2H-pyran-4-yl)-[1,2,4]triazolo[1,5-a]pyridin-2-amine

Prepared in analogy to example 1b-c), starting from 3-(3,6-dihydro-2H-pyran-4-yl)pyridin-2-amine. The crude product was purified by column chromatography on silica gel gradient from CH₂Cl₂ to CH₂Cl₂/MeOH 19:1 (v/v) as eluent. The title compound was obtained as a white solid (yield: 39% over 2 steps).

MS ISP (m/e): 217.3 (100) [(M+H)⁺].

¹H NMR (CDCl₃, 300 MHz): δ (ppm)=8.20 (d, 1H), 7.35 (s, 1H), 7.29 (d, 1H), 6.83 (t, 1H), 4.45 (m, 4H), 3.99 (t, 2H), 2.62 (m, 2H).

### d) [8-(3,6-Dihydro-2H-pyran-4-yl)-[1,2,4]triazolo[1,5-a]pyridin-2-yl]-[3-methoxy-4-(4-methyl-imidazol-1-yl)-phenyl]-amine

Prepared in analogy to example 8e, starting 8-(3,6-dihydro-2H-pyran-4-yl)-[1,2,4]triazolo[1,5-a]pyridin-2-amine and 1-(4-bromo-2-methoxy-phenyl)-4-methyl-1H-imidazole. The title compound was obtained as a light brown solid (yield: 54%) after column chromatography on silica gel using a gradient from CH₂Cl₂ to CH₂Cl₂/MeOH 19:1 (v/v) as eluent and precipitation from diethyl ether.

MS ISP (m/e): 403.4 (100) [(M+H)⁺].

¹H NMR (DMSO-D₆, 300 MHz): δ (ppm)=9.93 (s, 1H), 8.70 (d, 1H), 7.81 (s, 1H), 7.67 (s, 1H), 7.55 (s, 1H), 7.53 (d, 1H), 7.26 (d, 2H), 7.07 (t, 1H), 7.04 (s, 1H), 4.35 (m, 2H), 3.89 (t, 2H), 3.83 (s, 3H), 2.62 (m, 2H), 2.15 (s, 3H).

### Example 150

### [3-Methoxy-4-(4-methyl-imidazol-1-yl)-phenyl]-(6-phenyl-[1,2,4]triazolo[1,5-a]pyridin-2-yl)-amine

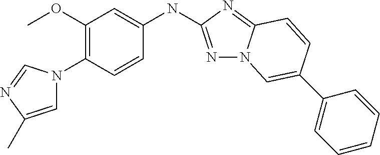

Prepared in analogy to example 125 employing 5-bromopyridin-2-amine instead of 3-bromopyridin-2-amine in step a) and phenyl boronic acid instead of 4-chlorophenyl boronic acid in step c). The title compound was obtained as white solid.

MS ISP (m/e): 397.3 [(M+H)⁺].

¹H NMR (CDCl₃, 300 MHz): δ (ppm)=8.66-8.65 (m 1H), 7.76-7.73 (m, 1H), 7.63-7.40 (m, 8H), 7.22-7.20 (m, 2H), 7.15-7.11 (m, 1H), 6.88 (m, 1H), 3.91 (s, 3H), 2.31 (s, 3H).

The following examples are not encompassed by the present scope of the invention: 1, 2, 51, 52, 55 and 56.

1. A compound of formula I 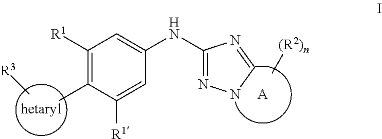
wherein
R¹ and R^{1′} are each independently hydrogen, halogen, lower alkoxy or cyano;
R² is lower alkyl, halogen, lower alkoxy, lower alkyl substituted by halogen, lower alkoxy substituted by halogen, lower alkyl substituted by OR, ═O, —C(O)O-lower alkyl, —C(O)NH-lower alkyl, cyano, CH₂—O-lower alkyl, cycloalkyl, NRR′ or is —O—(CH₂)ₒ-phenyl optionally substituted by halogen,
or is —(CH₂)ₒ-phenyl optionally substituted by one, two or three substituents, selected from halogen, —(CH₂)ₒ-cyano, lower alkyl, lower alkyl substituted by halogen, lower alkyl substituted by hydroxy, C(O)H, —CH₂—NH₂—, —CH₂—NH—C(O)O-lower alkyl, —CH₂—NH—C(O)-lower alkyl, —CH₂—NH-lower alkyl, —CH₂—NH—S(O)₂-lower alkyl, lower alkoxy and lower alkoxy substituted by halogen,
or is —(CH₂)ₒ-cycloalkyl,
or is —(CH₂)ₒ-heterocycloalkyl which is optionally substituted by halogen, CF₃, lower alkyl, —CH₂CN, —C(O)-lower alkyl, —C(O)O-lower alkyl or S(O)₂-lower alkyl,
or is heteroaryl selected from the group consisting of furanyl, pyrazinyl, pyridinyl, benzooxazolyl and benzoimidazolyl, each of which is optionally substituted by lower alkyl,
or is 4-methyl-3,4-dihydro-2H-benzo[1,4]oxazine;
R and R′ are each independently hydrogen or lower alkyl, and
o is 0 or 1;
R³ occurs once or twice and is lower alkyl;
A is 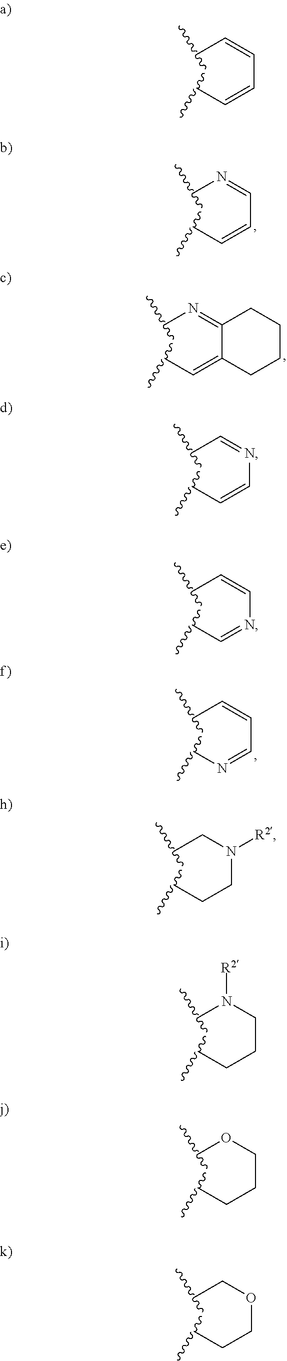
and
R^{2′} is hydrogen, lower alkyl, lower alkyl substituted by halogen, C(O)-lower alkyl, S(O)₂-lower alkyl or phenyl optionally substituted by halogen;
Hetaryl is a 5 or 6 membered N, S or O-containing heteroaryl group;
N is 0, 1, 2 or 3; when n is 2 or 3, each R² is the same or different;
or a pharmaceutically active acid addition salt thereof.

2. The compound of claim 1, wherein hetaryl is imidazolyl, pyrimidinyl or pyridinyl.

3. The compound of claim 1, wherein A is the ring a).

4. The compound of claim 3, wherein the compound is selected from the group consisting of
5-(8-methoxy-5-phenyl-[1,2,4]triazolo[1,5-a]pyridin-2-ylamino)-2-(4-methyl-imidazol-1-yl)-benzonitrile;
[3-methoxy-4-(4-methyl-imidazol-1-yl)-phenyl]-(5-phenyl-[1,2,4]triazolo[1,5-a]pyridin-2-yl)-amine;
[5-(4-fluoro-phenyl)-[1,2,4]triazolo[1,5-a]pyridin-2-yl]-[3-methoxy-4-(4-methyl-imidazol-1-yl)-phenyl]-amine;
[8-(4-fluoro-phenyl)-[1,2,4]triazolo[1,5-a]pyridin-2-yl]-[3-methoxy-4-(4-methyl-imidazol-1-yl)-phenyl]-amine;
[3-methoxy-4-(4-methyl-imidazol-1-yl)-phenyl]-(5-pyrrolidin-1-yl-[1,2,4]triazolo[1,5-a]pyridin-2-yl)-amine;
[3-methoxy-4-(4-methyl-imidazol-1-yl)-phenyl]-(5-propyl-[1,2,4]triazolo[1,5-a]pyridin-2-yl)-amine;
[3-methoxy-4-(4-methyl-imidazol-1-yl)-phenyl]-(5-trifluoromethyl-[1,2,4]triazolo[1,5-a]pyridin-2-yl)-amine;
[5-(4-fluoro-phenyl)-8-methoxy-[1,2,4]triazolo[1,5-a]pyridin-2-yl]-[3-methoxy-4-(4-methyl-imidazol-1-yl)-phenyl]-amine;
[3-methoxy-4-(4-methyl-imidazol-1-yl)-phenyl]-(5-morpholin-4-yl-[1,2,4]triazolo[1,5-a]pyridin-2-yl)-amine; and
[8-(4-fluoro-phenyl)-6-methyl-[1,2,4]triazolo[1,5-a]pyridin-2-yl]-[3-methoxy-4-(4-methyl-imidazol-1-yl)-phenyl]-amine.

5. The compound of claim 3, wherein the compound is selected from the group consisting of
(5,6-dimethyl-[1,2,4]triazolo[1,5-a]pyridin-2-yl)-[3-methoxy-4-(4-methyl-imidazol-1-yl)-phenyl]-amine;
8-(4-fluorophenyl)-N-(3-methoxy-4-(2-methylpyridin-4-yl)phenyl)-[1,2,4]triazolo[1,5-a]pyridin-2-amine;
5-(4-fluorophenyl)-N-(3-methoxy-4-(2-methylpyridin-4-yl)phenyl)-[1,2,4]triazolo[1,5-a]pyridin-2-amine;
[8-(3-chloro-4-fluoro-phenyl)-6-methyl-[1,2,4]triazolo[1,5-a]pyridin-2-yl]-[3-methoxy-4-(4-methyl-imidazol-1-yl)-phenyl]-amine;
[8-(3,4-difluoro-phenyl)-6-methyl-[1,2,4]triazolo[1,5-a]pyridin-2-yl]-[3-methoxy-4-(4-methyl-imidazol-1-yl)-phenyl]-amine;
[8-(4-fluoro-2-methoxy-phenyl)-6-methyl-[1,2,4]triazolo[1,5-a]pyridin-2-yl]-[3-methoxy-4-(4-methyl-imidazol-1-yl)-phenyl]-amine;
[8-(2-chloro-4-fluoro-phenyl)-6-methyl-[1,2,4]triazolo[1,5-a]pyridin-2-yl]-[3-methoxy-4-(4-methyl-imidazol-1-yl)-phenyl]-amine;
[8-(4-fluoro-2-methyl-phenyl)-6-methyl-[1,2,4]triazolo[1,5-a]pyridin-2-yl]-[3-methoxy-4-(4-methyl-imidazol-1-yl)-phenyl]-amine;
8-(2,4-difluorophenyl)-N-(3-methoxy-4-(4-methyl-1H-imidazol-1-yl)phenyl)-6-methyl-[1,2,4]triazolo[1,5-a]pyridin-2-amine; and
8-(4-fluoro-3-(trifluoromethyl)phenyl)-N-(3-methoxy-4-(4-methyl-1H-imidazol-1-yl)phenyl)-6-methyl-[1,2,4]triazolo[1,5-a]pyridin-2-amine.

6. The compound of claim 3, wherein the compound is selected from the group consisting of
8-(4-fluoro-3-methylphenyl)-N-(3-methoxy-4-(4-methyl-1H-imidazol-1-yl)phenyl)-6-methyl-[1,2,4]triazolo[1,5-a]pyridin-2-amine;
2-fluoro-5-(2-(3-methoxy-4-(4-methyl-1H-imidazol-1-yl)phenylamino)-6-methyl-[1,2,4]triazolo[1,5-a]pyridin-8-yl)benzonitrile;
8-(3,4-difluorophenyl)-N-(3-methoxy-4-(4-methyl-1H-imidazol-1-yl)phenyl)-6-(trifluoromethyl)-[1,2,4]triazolo[1,5-a]pyridin-2-amine;
6-chloro-8-(3,4-difluorophenyl)-N-(3-methoxy-4-(4-methyl-1H-imidazol-1-yl)phenyl)-[1,2,4]triazolo[1,5-a]pyridin-2-amine;
N-(3-methoxy-4-(4-methyl-1H-imidazol-1-yl)phenyl)-6-methyl-8-(4-morpholinophenyl)-[1,2,4]triazolo[1,5-a]pyridin-2-amine;
2-(4-(2-(3-methoxy-4-(4-methyl-1H-imidazol-1-yl)phenylamino)-6-methyl-[1,2,4]triazolo[1,5-a]pyridin-8-yl)phenyl)acetonitrile;
8-(2,4-dimethoxyphenyl)-N-(3-methoxy-4-(4-methyl-1H-imidazol-1-yl)phenyl)-6-methyl-[1,2,4]triazolo[1,5-a]pyridin-2-amine;
8-(3,4-difluorophenyl)-6-fluoro-N-(3-methoxy-4-(4-methyl-1H-imidazol-1-yl)phenyl)-[1,2,4]triazolo[1,5-a]pyridin-2-amine;
8-(2-chloro-4-fluorophenyl)-6-fluoro-N-(3-methoxy-4-(4-methyl-1H-imidazol-1-yl)phenyl)-[1,2,4]triazolo[1,5-a]pyridin-2-amine; and
N-(3-methoxy-4-(4-methyl-1H-imidazol-1-yl)phenyl)-6-methyl-8-(2-methylbenzo[d]oxazol-6-yl)-[1,2,4]triazolo[1,5-a]pyridin-2-amine.

7. The compound of claim 3, wherein the compound is selected from the group consisting of
N-(3-methoxy-4-(4-methyl-1H-imidazol-1-yl)phenyl)-6-methyl-8-(1-methyl-1H-benzo[d]imidazol-6-yl)-[1,2,4]triazolo[1,5-a]pyridin-2-amine;
N-(3-methoxy-4-(4-methyl-1H-imidazol-1-yl)phenyl)-6-methyl-8-(4-methyl-3,4-dihydro-2H-benzo[b][1,4]oxazin-7-yl)-[1,2,4]triazolo[1,5-a]pyridin-2-amine;
2-(2-fluoro-4-(2-(3-methoxy-4-(4-methyl-1H-imidazol-1-yl)phenylamino)-6-methyl-[1,2,4]triazolo[1,5-a]pyridin-8-yl)phenyl)propan-2-ol;
5-chloro-2-(2-(3-methoxy-4-(4-methyl-1H-imidazol-1-yl)phenylamino)-6-methyl-[1,2,4]triazolo[1,5-a]pyridin-8-yl)benzaldehyde;
(5-chloro-2-(2-(3-methoxy-4-(4-methyl-1H-imidazol-1-yl)phenylamino)-6-methyl-[1,2,4]triazolo[1,5-a]pyridin-8-yl)phenyl)methanol;
tert-butyl 3-(2-(3-methoxy-4-(4-methyl-1H-imidazol-1-yl)phenylamino)-6-methyl-[1,2,4]triazolo[1,5-a]pyridin-8-yl)benzylcarbamate;
tert-butyl 4-(2-(3-methoxy-4-(4-methyl-1H-imidazol-1-yl)phenylamino)-6-methyl-[1,2,4]triazolo[1,5-a]pyridin-8-yl)-5,6-dihydropyridine-1(2H)-carboxylate;
8-(3-(aminomethyl)phenyl)-N-(3-methoxy-4-(4-methyl-1H-imidazol-1-yl)phenyl)-6-methyl-[1,2,4]triazolo[1,5-a]pyridin-2-amine dihydrochloride;
N-(3-(2-(3-methoxy-4-(4-methyl-1H-imidazol-1-yl)phenylamino)-6-methyl-[1,2,4]triazolo[1,5-a]pyridin-8-yl)benzyl)methanesulfonamide; and
N-(3-(2-(3-methoxy-4-(4-methyl-1H-imidazol-1-yl)phenylamino)-6-methyl-[1,2,4]triazolo[1,5-a]pyridin-8-yl)benzyl)acetamide.

8. The compound of claim 3, wherein the compound is selected from the group consisting of
N-(3-methoxy-4-(4-methyl-1H-imidazol-1-yl)phenyl)-6-methyl-8-(1-(methylsulfonyl)piperidin-4-yl)-[1,2,4]triazolo[1,5-a]pyridin-2-amine;
N-(3-methoxy-4-(4-methyl-1H-imidazol-1-yl)phenyl)-8-morpholino-[1,2,4]triazolo[1,5-a]pyridin-2-amine;
ethyl 4-(2-(3-methoxy-4-(4-methyl-1H-imidazol-1-yl)phenylamino)-6-methyl-[1,2,4]triazolo[1,5-a]pyridin-8-yl)-5,6-dihydropyridine-1(2H)-carboxylate;
2-(4-(2-(3-methoxy-4-(4-methyl-1H-imidazol-1-yl)phenylamino)-6-methyl-[1,2,4]triazolo[1,5-a]pyridin-8-yl)-5,6-dihydropyridin-1(2H)-yl)acetonitrile;
8-(2-chloro-4-fluorophenyl)-N-(3-fluoro-4-(4-methyl-1H-imidazol-1-yl)phenyl)-[1,2,4]triazolo[1,5-a]pyridin-2-amine;
8-(2-chloro-4-fluorophenyl)-N-(3,5-difluoro-4-(4-methyl-1H-imidazol-1-yl)phenyl)-[1,2,4]triazolo[1,5-a]pyridin-2-amine;
8-(2-chloro-4-fluorophenyl)-N-(3-fluoro-5-methoxy-4-(4-methyl-1H-imidazol-1-yl)phenyl)-[1,2,4]triazolo[1,5-a]pyridin-2-amine;
5-(8-(2-chloro-4-fluorophenyl)-[1,2,4]triazolo[1,5-a]pyridin-2-ylamino)-2-(4-methyl-1H-imidazol-1-yl)benzonitrile;
8-(2-chloro-4-fluorophenyl)-N-(3-methoxy-4-(6-methylpyrimidin-4-yl)phenyl)-[1,2,4]triazolo[1,5-a]pyridin-2-amine; and
8-(2-chloro-4-fluorophenyl)-N-(4-(2,6-dimethylpyrimidin-4-yl)phenyl)-[1,2,4]triazolo[1,5-a]pyridin-2-amine.

9. The compound of claim 3, wherein the compound is selected from the group consisting of
2-{8-(4-chloro-phenyl)-2-[3-methoxy-4-(4-methyl-imidazol-1-yl)-phenylamino]-[1,2,4]triazolo[1,5-a]pyridin-5-yl}-propan-2-ol;
[8-(4-chloro-phenyl)-6-cyclopropyl-[1,2,4]triazolo[1,5-a]pyridin-2-yl]-[3-methoxy-4-(4-methyl-imidazol-1-yl)-phenyl]-amine;
[6-cyclopropyl-8-(4-fluoro-phenyl)-[1,2,4]triazolo[1,5-a]pyridin-2-yl]-[3-methoxy-4-(4-methyl-imidazol-1-yl)-phenyl]-amine;
2-{8-(4-fluoro-phenyl)-2-[3-methoxy-4-(4-methyl-imidazol-1-yl)-phenylamino]-[1,2,4]triazolo[1,5-a]pyridin-5-yl}-propan-2-ol;
[6-cyclopropyl-8-(2,3,4-trifluoro-phenyl)-[1,2,4]triazolo[1,5-a]pyridin-2-yl]-[3-methoxy-4-(4-methyl-imidazol-1-yl)-phenyl]-amine;
2-{8-(2-chloro-4-fluoro-phenyl)-2-[3-methoxy-4-(4-methyl-imidazol-1-yl)-phenylamino]-[1,2,4]triazolo[1,5-a]pyridin-5-yl}-propan-2-ol;
2-[2-[3-methoxy-4-(4-methyl-imidazol-1-yl)-phenylamino]-8-(2,3,4-trifluoro-phenyl)-[1,2,4]triazolo[1,5-a]pyridin-5-yl]-propan-2-ol;
2-{8-(3-chloro-4-fluoro-phenyl)-2-[3-methoxy-4-(4-methyl-imidazol-1-yl)-phenylamino]-[1,2,4]triazolo[1,5-a]pyridin-5-yl}-propan-2-ol;
[8-(4-chloro-phenyl)-[1,2,4]triazolo[1,5-a]pyridin-2-yl]-[3-methoxy-4-(4-methyl-imidazol-1-yl)-phenyl]-amine; and
[8-(3,4-difluoro-phenyl)-[1,2,4]triazolo[1,5-a]pyridin-2-yl]-[3-methoxy-4-(4-methyl-imidazol-1-yl)-phenyl]-amine.

10. The compound of claim 3, wherein the compound is selected from the group consisting of
[8-(4-chloro-phenyl)-[1,2,4]triazolo[1,5-a]pyridin-2-yl]-[3-methoxy-4-(2-methyl-pyridin-4-yl)-phenyl]-amine;
8-(3,4-difluorophenyl)-N-(3-methoxy-4-(2-methylpyridin-4-yl)phenyl)-[1,2,4]triazolo[1,5-a]pyridin-2-amine;
8-(3-chloro-4-fluorophenyl)-N-(3-methoxy-4-(4-methyl-1H-imidazol-1-yl)phenyl)-[1,2,4]triazolo[1,5-a]pyridin-2-amine;
8-(2-chloro-4-fluorophenyl)-N-(3-methoxy-4-(4-methyl-1H-imidazol-1-yl)phenyl)-[1,2,4]triazolo[1,5-a]pyridin-2-amine;
8-(2,4-difluorophenyl)-N-(3-methoxy-4-(4-methyl-1H-imidazol-1-yl)phenyl)-[1,2,4]triazolo[1,5-a]pyridin-2-amine;
[8-(2-chloro-4-fluoro-phenyl)-[1,2,4]triazolo[1,5-a]pyridin-2-yl]-[3-methoxy-4-(2-methyl-pyridin-4-yl)-phenyl]-amine;
8-(3,4-difluorophenyl)-N-(3-methoxy-4-(4-methyl-1H-imidazol-1-yl)phenyl)-5-(trifluoromethyl)-[1,2,4]triazolo[1,5-a]pyridin-2-amine;
N-(3-methoxy-4-(4-methyl-1H-imidazol-1-yl)phenyl)-8-phenoxy-[1,2,4]triazolo[1,5-a]pyridin-2-amine;
8-(3-chlorophenoxy)-N-(3-methoxy-4-(4-methyl-1H-imidazol-1-yl)phenyl)-[1,2,4]triazolo[1,5-a]pyridin-2-amine; and
8-(4-chlorophenoxy)-N-(3-methoxy-4-(4-methyl-1H-imidazol-1-yl)phenyl)-[1,2,4]triazolo[1,5-a]pyridin-2-amine.

11. The compound of claim 3, wherein the compound is selected from the group consisting of
8-(4-fluoropiperidin-1-yl)-N-(3-methoxy-4-(4-methyl-1H-imidazol-1-yl)phenyl)-[1,2,4]triazolo[1,5-a]pyridin-2-amine;
N-(3-methoxy-4-(4-methyl-1H-imidazol-1-yl)phenyl)-8-(4-(trifluoromethyl)piperidin-1-yl)-[1,2,4]triazolo[1,5-a]pyridin-2-amine;
8-(4,4-difluoropiperidin-1-yl)-N-(3-methoxy-4-(4-methyl-1H-imidazol-1-yl)phenyl)-[1,2,4]triazolo[1,5-a]pyridin-2-amine;
2-{8-(4-chloro-phenyl)-2-[3-methoxy-4-(4-methyl-imidazol-1-yl)-phenylamino]-6-methyl-[1,2,4]triazolo[1,5-a]pyridin-5-yl}-propan-2-ol;
[8-(3,6-dihydro-2H-pyran-4-yl)-[1,2,4]triazolo[1,5-a]pyridin-2-yl]-[3-methoxy-4-(4-methyl-imidazol-1-yl)-phenyl]-amine;
2-{8-(3,4-difluoro-phenyl)-2-[3-methoxy-4-(4-methyl-imidazol-1-yl)-phenylamino]-[1,2,4]triazolo[1,5-a]pyridin-5-yl}-propan-2-ol;
[6-cyclopropyl-8-(3,4-difluoro-phenyl)-[1,2,4]triazolo[1,5-a]pyridin-2-yl]-[3-methoxy-4-(4-methyl-imidazol-1-yl)-phenyl]-amine;
[8-(3-chloro-4-fluoro-phenyl)-6-cyclopropyl-[1,2,4]triazolo[1,5-a]pyridin-2-yl]-[3-methoxy-4-(4-methyl-imidazol-1-yl)-phenyl]-amine; and
[8-(2-chloro-4-fluoro-phenyl)-6-cyclopropyl-[1,2,4]triazolo[1,5-a]pyridin-2-yl]-[3-methoxy-4-(4-methyl-imidazol-1-yl)-phenyl]-amine.

12. The compound of claim 1, wherein A is the ring b).

13. The compound of claim 12, wherein the compound is selected from the group consisting of
[3-methoxy-4-(4-methyl-imidazol-1-yl)-phenyl]-(5-methyl-7-phenyl-[1,2,4]triazolo[1,5-a]pyrimidin-2-yl)-amine;
[7-(4-chloro-phenyl)-5-trifluoromethyl-[1,2,4]triazolo[1,5-a]pyrimidin-2-yl]-[3-methoxy-4-(4-methyl-imidazol-1-yl)-phenyl]-amine;
[3-methoxy-4-(4-methyl-imidazol-1-yl)-phenyl]-[7-(4-trifluoromethoxy-phenyl)-[1,2,4]triazolo[1,5-a]pyrimidin-2-yl]-amine;
(5,7-bis-trifluoromethyl-[1,2,4]triazolo[1,5-a]pyrimidin-2-yl)-[3-methoxy-4-(4-methyl-imidazol-1-yl)-phenyl]-amine;
2-[3-methoxy-4-(4-methyl-imidazol-1-yl)-phenylamino]-7-phenyl-[1,2,4]triazolo[1,5-a]pyrimidine-6-carbonitrile;
7-(4-chloro-phenyl)-2-[3-methoxy-4-(4-methyl-imidazol-1-yl)-phenylamino]-[1,2,4]triazolo[1,5-a]pyrimidine-6-carbonitrile;
2-[3-methoxy-4-(4-methyl-imidazol-1-yl)-phenylamino]-7-m-tolyl-[1,2,4]triazolo[1,5-a]pyrimidine-6-carbonitrile;
2-(3-methoxy-4-(4-methyl-1H-imidazol-1-yl)phenylamino)-7-o-tolyl-[1,2,4]triazolo[1,5-a]pyrimidine-6-carbonitrile;
7-(2-fluorophenyl)-2-(3-methoxy-4-(4-methyl-1H-imidazol-1-yl)phenylamino)-[1,2,4]triazolo[1,5-a]pyrimidine-6-carbonitrile; and
[7-(4-fluoro-phenyl)-[1,2,4]triazolo[1,5-a]pyrimidin-2-yl]-[3-methoxy-4-(4-methyl-imidazol-1-yl)-phenyl]-amine; 7-(3-chloro-4-fluorophenyl)-N-(3-methoxy-4-(4-methyl-1H-imidazol-1-yl)phenyl)-[1,2,4]triazolo[1,5-a]pyrimidin-2-amine hydrochloride;
7-(3-chloro-4-fluorophenyl)-N-(3-methoxy-4-(4-methyl-1H-imidazol-1-yl)phenyl)-5-methyl-[1,2,4]triazolo[1,5-a]pyrimidin-2-amine; and
ethyl 7-(3-chloro-4-fluorophenyl)-2-(3-methoxy-4-(4-methyl-1H-imidazol-1-yl)phenylamino)-[1,2,4]triazolo[1,5-a]pyrimidine-5-carboxylate.

14. The compound of claim 1, wherein A is the ring c).

15. The compound of claim 14, wherein the compound is
[3-methoxy-4-(4-methyl-imidazol-1-yl)-phenyl]-(9-phenyl-5,6,7,8-tetrahydro-[1,2,4]triazolo[5,1-b]quinazolin-2-yl)-amine.

16. The compound of claim 1, wherein A is the ring d).

17. The compound of claim 16, wherein the compound is selected from the group consisting of
[5-(4-fluoro-phenyl)-[1,2,4]triazolo[1,5-a]pyrazin-2-yl]-[3-methoxy-4-(4-methyl-imidazol-1-yl)-phenyl]-amine and
[3-methoxy-4-(4-methyl-imidazol-1-yl)-phenyl]-(5-phenyl-[1,2,4]triazolo[1,5-a]pyrazin-2-yl)-amine.

18. The compound of claim 1, wherein A is the ring e).

19. The compound of claim 11, wherein the compound is
[3-methoxy-4-(4-methyl-imidazol-1-yl)-phenyl]-[8-methyl-5-(2,2,2-trifluoro-ethoxy)-[1,2,4]triazolo[1,5-c]pyrimidin-2-yl]-amine.

20. The compound of claim 1, wherein A is the ring f).

21. The compound of claim 13, wherein the compound is
7-(3-chloro-4-fluorophenyl)-N-(3-methoxy-4-(4-methyl-1H-imidazol-1-yl)phenyl)-5-(trifluoromethyl)-[1,2,4]triazolo[1,5-a]pyrimidin-2-amine acetate.

22. The compound of claim 1, wherein A is the ring h).

23. The compound of claim 15, wherein the compound is selected from the group consisting of
5-(4-fluorophenyl)-N-(3-methoxy-4-(4-methyl-1H-imidazol-1-yl)phenyl)-7-(methylsulfonyl)-5,6,7,8-tetrahydro-[1,2,4]triazolo[1,5-a]pyrazin-2-amine;
5-(4-fluorophenyl)-N-(3-methoxy-4-(4-methyl-1H-imidazol-1-yl)phenyl)-7-methyl-5,6,7,8-tetrahydro-[1,2,4]triazolo[1,5-a]pyrazin-2-amine;
8-(4-fluorophenyl)-N-(3-methoxy-4-(4-methyl-1H-imidazol-1-yl)phenyl)-7-methyl-5,6,7,8-tetrahydro-[1,2,4]triazolo[1,5-a]pyrazin-2-amine; and
1-(5-(4-fluorophenyl)-2-(3-methoxy-4-(4-methyl-1H-imidazol-1-yl)phenylamino)-5,6-dihydro-[1,2,4]triazolo[1,5-a]pyrazin-7(8H)-yl)-2-methylpropan-1-one.

24. The compound of claim 1, wherein A is the ring i).

25. The compound of claim 17, wherein the compound is selected from the group consisting of
7-(4-chlorophenyl)-2-(3-methoxy-4-(4-methyl-1H-imidazol-1-yl)phenylamino)-4-propyl-6,7-dihydro-[1,2,4]triazolo[1,5-a]pyrimidin-5(4H)-one and
4-(3,4-difluorophenyl)-N-(3-methoxy-4-(4-methyl-1H-imidazol-1-yl)phenyl)-4,5,6,7-tetrahydro-[1,2,4]triazolo[1,5-a]pyrimidin-2-amine.

26. The compound of claim 1, wherein A is the ring j).

27. The compound of claim 19, wherein the compound is
N-(3-methoxy-4-(4-methyl-1H-imidazol-1-yl)phenyl)-7-phenyl-6,7-dihydro-5H-[1,2,4]triazolo[5,1-b][1,3]oxazin-2-amine.

28. The compound of claim 1, wherein A is the ring k).

29. The compound of claim 21, wherein the compound is
N-(3-methoxy-4-(4-methyl-1H-imidazol-1-yl)phenyl)-8-phenyl-6,8-dihydro-5H-[1,2,4]triazolo[5,1-c][1,4]oxazin-2-amine.

30. A pharmaceutical composition comprising a therapeutically effective amount of a compound of formula 1 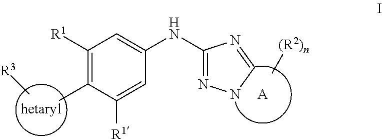
wherein
R¹ and R^{1′} are each independently hydrogen, halogen, lower alkoxy or cyano;
R² is lower alkyl, halogen, lower alkoxy, lower alkyl substituted by halogen, lower alkoxy substituted by halogen, lower alkyl substituted by OR, ═O, —C(O)O-lower alkyl, —C(O)NH-lower alkyl, cyano, CH₂—O-lower alkyl, cycloalkyl, NRR′ or is —O—(CH₂)ₒ-phenyl optionally substituted by halogen,
or is —(CH₂)ₒ-phenyl optionally substituted by one, two or three substituents, selected from halogen, —(CH₂)ₒ-cyano, lower alkyl, lower alkyl substituted by halogen, lower alkyl substituted by hydroxy, C(O)H, —CH₂—NH₂—, —CH₂—NH—C(O)O-lower alkyl, —CH₂—NH—C(O)-lower alkyl, —CH₂—NH-lower alkyl, —CH₂—NH—S(O)₂-lower alkyl, lower alkoxy and lower alkoxy substituted by halogen,
or is —(CH₂)ₒ-cycloalkyl,
or is —(CH₂)ₒ-heterocycloalkyl which is optionally substituted by halogen, CF₃, lower alkyl, —CH₂CN, —C(O)-lower alkyl, —C(O)O-lower alkyl or S(O)₂-lower alkyl,
or is heteroaryl selected from the group consisting of furanyl, pyrazinyl, pyridinyl, benzooxazolyl and benzoimidazolyl, each of which is optionally substituted by lower alkyl, or is 4-methyl-3,4-dihydro-2H-benzo[1,4]oxazine;
R and R′ are each independently hydrogen or lower alkyl, and
o is 0 or 1;
R³ occurs once or twice and is lower alkyl;
A is 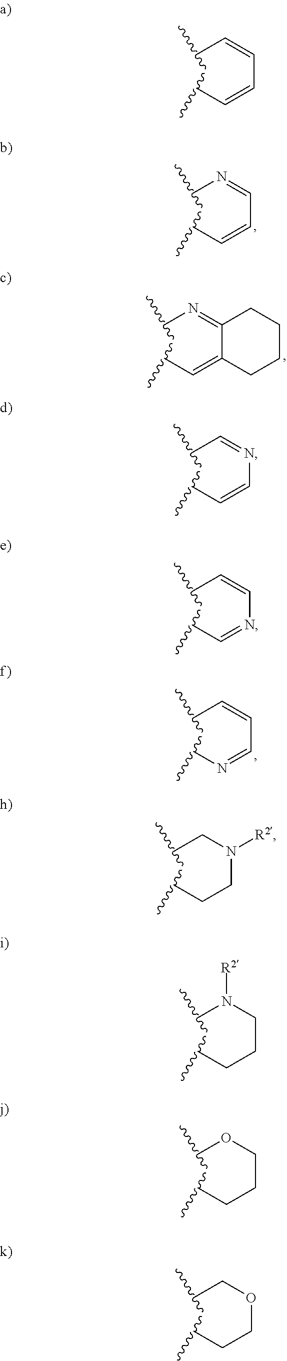
and
R^{2′} is hydrogen, lower alkyl, lower alkyl substituted by halogen, C(O)-lower alkyl, S(O)₂-lower alkyl or phenyl optionally substituted by halogen;
Hetaryl is a 5 or 6 membered N, S or O-containing heteroaryl group;
N is 0, 1, 2 or 3; when n is 2 or 3, each R² is the same or different;
or a pharmaceutically active acid addition salt thereof
and a pharmaceutically acceptable carrier.
